SEQUENCE LISTING (346 sequences)

SEQ ID NO: 1 - protein, 164 aa, synthetic construct
  source 1..164: note = Human IL31 with signal sequence
MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 2 - protein, 141 aa, synthetic construct [sequence omitted]

SEQ ID NO: 3 - protein, 118 aa, synthetic construct
  source 1..118: note = 1C10 VHH1 single binder
EVQLVESGGGLVQAGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTQVTVSS

SEQ ID NO: 4 - protein, 118 aa, synthetic construct
  source 1..118: note = Humanized 1C10 VHH1 single binder
EVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSS

SEQ ID NO: 5 - protein, 5 aa, synthetic construct
  source 1..5: note = 1C10 VHH1 CDR1
SYTMG

SEQ ID NO: 6 - protein, 17 aa, synthetic construct
  source 1..17: note = 1C10 VHH1 CDR2
GISSSGYVMYNSESMKG

SEQ ID NO: 7 - protein, 9 aa, synthetic construct
  source 1..9: note = 1C10 VHH1 CDR3
GTIGRPYDY

SEQ ID NO: 8 - protein, 119 aa, synthetic construct
  source 1..119: note = 1C10 VHH2 single binder
VKLEESGGGLVQPGGSLILSCAASGDISSIVAMGWYRQAPGKQRELVAAITSGGRTHYRDSVKGRFTISGNNDNSALYLHMNSLKPEDTAVYYCAADRGWTSVGEYDYWGKGTLVTVSS

SEQ ID NO: 9 - protein, 119 aa, synthetic construct
  source 1..119: note = Humanized 1C10 VHH2 single binder
VQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 10 - protein, 5 aa, synthetic construct
  source 1..5: note = 1C10 VHH2 CDR1
IVAMG

SEQ ID NO: 11 - protein, 16 aa, synthetic construct
  source 1..16: note = 1C10 VHH2 CDR2
AITSGGRTHYRDSVKG

SEQ ID NO: 12 - protein, 12 aa, synthetic construct
  source 1..12: note = 1C10 VHH2 CDR3
DRGWTSVGEYDY

SEQ ID NO: 13 - protein, 275 aa, synthetic construct
  source 1..275: note = 1C10 co-binder
EVQLVESGGGLVQAGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTQVTVSSGGGSGGGGSGGGGSGGGGSGGGGSGGGGSGGGGSGMTGVKLEESGGGLVQPGGSLILSCAASGDISSIVAMGWYRQAPGKQRELVAAITSGGRTHYRDSVKGRFTISGNNDNSALYLHMNSLKPEDTAVYYCAADRGWTSVGEYDYWGKGTLVTVSS

SEQ ID NO: 14 - protein, 275 aa, synthetic construct
  source 1..275: note = Humanized 1C10 co binder
EVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 15 - protein, 507 aa, synthetic construct
  source 1..507: note = 1C10 co-binder + huIgG1Fc_YTE_C S
EVQLVESGGGLVQAGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTQVTVSSGGGSGGGGSGGGGSGGGGSGGGGSGGGGSGGGGSGMTGVKLEESGGGLVQPGGSLILSCAASGDISSIVAMGWYRQAPGKQRELVAAITSGGRTHYRDSVKGRFTISGNNDNSALYLHMNSLKPEDTAVYYCAADRGWTSVGEYDYWGKGTLVTVSSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 507 aa, synthetic construct
  source 1..507: note = Humanized 1C10 co binder + huIgG1Fc_YTE_C S
EVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 118 aa, synthetic construct
  source 1..118: note = 1D1 VHH1 single binder
EVQLVESGGGLVQAGGSLRLSCAASGGTFSSYAMAWFRQAPGKEREYVAGISSSGYTLYKSDSMKGRFTISRENAKNMVYLQMNSLKPEDTAVYSCAAGIFGRPYEYWGQGTLVTVSS

SEQ ID NO: 18 - protein, 118 aa, synthetic construct
  source 1..118: note = Humanized 1D1 VHH1 single binder
EVQLVESGGGLVQPGGSLRLSCAASGGTFSSYAMAWFRQAPGKEREYVAGISSSGYTLYKSDSMKGRFTISRDNAKNTLYLQMNSLRAEDTAVYYCAAGIFGRPYEYWGQGTLVTVSS

SEQ ID NO: 19 - protein, 5 aa, synthetic construct
  source 1..5: note = 1D1 VHH1 CDR1
SYAMA

SEQ ID NO: 20 - protein, 17 aa, synthetic construct
  source 1..17: note = 1D1 VHH1 CDR2
GISSSGYTLYKSDSMKG

SEQ ID NO: 21 - protein, 9 aa, synthetic construct
  source 1..9: note = 1D1 VHH1 CDR3
GIFGRPYEY

SEQ ID NO: 22 - protein, 119 aa, synthetic construct
  source 1..119: note = 1D1 VHH2 single binder
VKLEESGGGLVQAGGSLILSCAASGDIPSIVAMGWYRQAPGKQRELVAAITSGGRTHYRDSVKGRFTISGNNDNSALYLHMNSLKPEDTAVYYCAADMGLTAVGEYDYWGQGTQVTVSS

SEQ ID NO: 23 - protein, 119 aa, synthetic construct
  source 1..119: note = Humanized 1D1 VHH2 single binder
VQLVESGGGLVQPGGSLRLSCAASGDIPSIVAMGWYRQAPGKQRELVSAITSGGRTHYRDSVKGRFTISRDNSKNTLYLQMNSLRAEDTAVYYCAADMGLTAVGEYDYWGQGTQVTVSS

SEQ ID NO: 24 - protein, 5 aa, synthetic construct
  source 1..5: note = 1D1 VHH2 CDR1
IVAMG

SEQ ID NO: 25 - protein, 16 aa, synthetic construct
  source 1..16: note = 1D1 VHH2 CDR2
AITSGGRTHYRDSVKG

SEQ ID NO: 26 - protein, 12 aa, synthetic construct
  source 1..12: note = 1D1 VHH2 CDR3
DMGLTAVGEYDY

SEQ ID NO: 27 - protein, 265 aa, synthetic construct
  source 1..265: note = 1D1 co-binder
EVQLVESGGGLVQAGGSLRLSCAASGGTFSSYAMAWFRQAPGKEREYVAGISSSGYTLYKSDSMKGRFTISRENAKNMVYLQMNSLKPEDTAVYSCAAGIFGRPYEYWGQGTLVTVSSGGGSGGGGSGGGGSGGGGSGGGGSPSMGVKLEESGGGLVQAGGSLILSCAASGDIPSIVAMGWYRQAPGKQRELVAAITSGGRTHYRDSVKGRFTISGNNDNSALYLHMNSLKPEDTAVYYCAADMGLTAVGEYDYWGQGTQVTVSS

SEQ ID NO: 28 - protein, 265 aa, synthetic construct
  source 1..265: note = Humanized 1D1 co binder
EVQLVESGGGLVQPGGSLRLSCAASGGTFSSYAMAWFRQAPGKEREYVAGISSSGYTLYKSDSMKGRFTISRDNAKNTLYLQMNSLRAEDTAVYYCAAGIFGRPYEYWGQGTLVTVSSGGGSGGGGSGGGGSGGGGSGGGGSPSMGVQLVESGGGLVQPGGSLRLSCAASGDIPSIVAMGWYRQAPGKQRELVSAITSGGRTHYRDSVKGRFTISRDNSKNTLYLQMNSLRAEDTAVYYCAADMGLTAVGEYDYWGQGTQVTVSS

SEQ ID NO: 29 - protein, 497 aa, synthetic construct
  source 1..497: note = 1D1 co-binder + huIgG1Fc_YTE_C S
EVQLVESGGGLVQAGGSLRLSCAASGGTFSSYAMAWFRQAPGKEREYVAGISSSGYTLYKSDSMKGRFTISRENAKNMVYLQMNSLKPEDTAVYSCAAGIFGRPYEYWGQGTLVTVSSGGGSGGGGSGGGGSGGGGSGGGGSPSMGVKLEESGGGLVQAGGSLILSCAASGDIPSIVAMGWYRQAPGKQRELVAAITSGGRTHYRDSVKGRFTISGNNDNSALYLHMNSLKPEDTAVYYCAADMGLTAVGEYDYWGQGTQVTVSSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 497 aa, synthetic construct
  source 1..497: note = Humanized 1D1 co binder + huIgG1Fc_YTE_CS
EVQLVESGGGLVQPGGSLRLSCAASGGTFSSYAMAWFRQAPGKEREYVAGISSSGYTLYKSDSMKGRFTISRDNAKNTLYLQMNSLRAEDTAVYYCAAGIFGRPYEYWGQGTLVTVSSGGGSGGGGSGGGGSGGGGSGGGGSPSMGVQLVESGGGLVQPGGSLRLSCAASGDIPSIVAMGWYRQAPGKQRELVSAITSGGRTHYRDSVKGRFTISRDNSKNTLYLQMNSLRAEDTAVYYCAADMGLTAVGEYDYWGQGTQVTVSSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 118 aa, synthetic construct
  source 1..118: note = 1H5 VHH1 single binder
EVQLVESGGGSVQAGGSLRLSCAASGGTFSSYAMAWFRQAPGKEREYVAGISSSGYTLYKSDSMKGRFTISRENAKNMVYLQMNSLKPEDTAVYSCAAGIFGRPYEYWGQGTQVTVSS

SEQ ID NO: 32 - protein, 118 aa, synthetic construct
  source 1..118: note = Humanized 1H5 VHH1
EVQLVESGGGLVQPGGSLRLSCAASGGTFSSYAMAWFRQAPGKEREYVAGISSSGYTLYKSDSMKGRFTISRDNAKNTLYLQMNSLRAEDTAVYYCAAGIFGRPYEYWGQGTQVTVSS

SEQ ID NO: 33 - protein, 5 aa, synthetic construct
  source 1..5: note = 1H5 VHH1 CDR1
SYAMA

SEQ ID NO: 34 - protein, 17 aa, synthetic construct
  source 1..17: note = 1H5 VHH1 CDR2
GISSSGYTLYKSDSMKG

SEQ ID NO: 35 - protein, 9 aa, synthetic construct
  source 1..9: note = 1H5 VHH1 CDR3
GIFGRPYEY

SEQ ID NO: 36 - protein, 119 aa, synthetic construct
  source 1..119: note = 1H5 VHH2 single binder
VQLVESGGGLVQAGGSLRLSCAASGSISSIHAMGWYRQAPGKQRELIAAITSGGRRHYRDSVKGRFTISGDNAKTNLYLQMNSLKPEDTAVYYCAADTGRVAVGEYDYWGQGTLVTVSS

SEQ ID NO: 37 - protein, 119 aa, synthetic construct
  source 1..119: note = Humanized 1H5 VHH2
VQLVESGGGLVQPGGSLRLSCAASGSISSIHAMGWYRQAPGKQRELIAAITSGGRRHYRDSVKGRFTISRDNSKNTLYLQMNSLRAEDTAVYYCAADTGRVAVGEYDYWGQGTLVTVSS

SEQ ID NO: 38 - protein, 5 aa, synthetic construct
  source 1..5: note = 1H5 VHH2 CDR1
IHAMG

SEQ ID NO: 39 - protein, 16 aa, synthetic construct
  source 1..16: note = 1H5 VHH2 CDR2
AITSGGRRHYRDSVKG

SEQ ID NO: 40 - protein, 12 aa, synthetic construct
  source 1..12: note = 1H5 VHH2 CDR3
DTGRVAVGEYDY

SEQ ID NO: 41 - protein, 256 aa, synthetic construct
  source 1..256: note = 1H5 co-binder
EVQLVESGGGSVQAGGSLRLSCAASGGTFSSYAMAWFRQAPGKEREYVAGISSSGYTLYKSDSMKGRFTISRENAKNMVYLQMNSLKPEDTAVYSCAAGIFGRPYEYWGQGTQVTVSSGGGGSGGGGSGGGGSRSVGVQLVESGGGLVQAGGSLRLSCAASGSISSIHAMGWYRQAPGKQRELIAAITSGGRRHYRDSVKGRFTISGDNAKTNLYLQMNSLKPEDTAVYYCAADTGRVAVGEYDYWGQGTLVTVSS

SEQ ID NO: 42 - protein, 256 aa, synthetic construct
  source 1..256: note = Humanized 1H5 co-binder
EVQLVESGGGLVQPGGSLRLSCAASGGTFSSYAMAWFRQAPGKEREYVAGISSSGYTLYKSDSMKGRFTISRDNAKNTLYLQMNSLRAEDTAVYYCAAGIFGRPYEYWGQGTQVTVSSGGGGSGGGGSGGGGSRSVGVQLVESGGGLVQPGGSLRLSCAASGSISSIHAMGWYRQAPGKQRELIAAITSGGRRHYRDSVKGRFTISRDNSKNTLYLQMNSLRAEDTAVYYCAADTGRVAVGEYDYWGQGTLVTVSS

SEQ ID NO: 43 - protein, 488 aa, synthetic construct
  source 1..488: note = 1H5 co-binder + huIgG1Fc_YTE_CS
EVQLVESGGGSVQAGGSLRLSCAASGGTFSSYAMAWFRQAPGKEREYVAGISSSGYTLYKSDSMKGRFTISRENAKNMVYLQMNSLKPEDTAVYSCAAGIFGRPYEYWGQGTQVTVSSGGGGSGGGGSGGGGSRSVGVQLVESGGGLVQAGGSLRLSCAASGSISSIHAMGWYRQAPGKQRELIAAITSGGRRHYRDSVKGRFTISGDNAKTNLYLQMNSLKPEDTAVYYCAADTGRVAVGEYDYWGQGTLVTVSSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 488 aa, synthetic construct
  source 1..488: note = Humanized 1H5 co binder + huIgG1Fc_YTE_CS
EVQLVESGGGLVQPGGSLRLSCAASGGTFSSYAMAWFRQAPGKEREYVAGISSSGYTLYKSDSMKGRFTISRDNAKNTLYLQMNSLRAEDTAVYYCAAGIFGRPYEYWGQGTQVTVSSGGGGSGGGGSGGGGSRSVGVQLVESGGGLVQPGGSLRLSCAASGSISSIHAMGWYRQAPGKQRELIAAITSGGRRHYRDSVKGRFTISRDNSKNTLYLQMNSLRAEDTAVYYCAADTGRVAVGEYDYWGQGTLVTVSSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 118 aa, synthetic construct; identical to SEQ ID NO: 3
  source 1..118: note = 21H7 VHH1 single binder

SEQ ID NO: 46 - protein, 118 aa, synthetic construct; identical to SEQ ID NO: 4
  source 1..118: note = Humanized 21H7 VHH1

SEQ ID NO: 47 - protein, 5 aa, synthetic construct
  source 1..5: note = 21H7 VHH1 CDR1
SYTMG

SEQ ID NO: 48 - protein, 17 aa, synthetic construct
  source 1..17: note = 21H7 VHH1 CDR2
GISSSGYVMYNSESMKG

SEQ ID NO: 49 - protein, 9 aa, synthetic construct
  source 1..9: note = 21H7 VHH1 CDR3
GTIGRPYDY

SEQ ID NO: 50 - protein, 120 aa, synthetic construct
  source 1..120: note = 21H7 VHH2 single binder
LVESGGGSVQAGDSLRLSCAASGRTFSTYIIGWFRQAPGKEREFVASISWGGRGTYYADSVKDRFTISRDNPKNMVYLQMNSLKPEDTAVYYCAAGDLPGGRRALDYDYWGQGTLVTVSS

SEQ ID NO: 51 - protein, 120 aa, synthetic construct
  source 1..120: note = Humanized 21H7 VHH2
LVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 52 - protein, 5 aa, synthetic construct
  source 1..5: note = 21H7 VHH2 CDR1
TYIIG

SEQ ID NO: 53 - protein, 17 aa, synthetic construct
  source 1..17: note = 21H7 VHH2 CDR2
SISWGGRGTYYADSVKD

SEQ ID NO: 54 - protein, 14 aa, synthetic construct
  source 1..14: note = 21H7 VHH2 CDR3
GDLPGGRRALDYDY

SEQ ID NO: 55 - protein, 276 aa, synthetic construct
  source 1..276: note = 21H7 co-binder
EVQLVESGGGLVQAGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTQVTVSSGGGSGGGGSGGGGSGGGGSGGGGSGGGGSGGGGSEGVGLVESGGGSVQAGDSLRLSCAASGRTFSTYIIGWFRQAPGKEREFVASISWGGRGTYYADSVKDRFTISRDNPKNMVYLQMNSLKPEDTAVYYCAAGDLPGGRRALDYDYWGQGTLVTVSS

SEQ ID NO: 56 - protein, 276 aa, synthetic construct
  source 1..276: note = Humanized 21H7 co binder
EVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 57 - protein, 508 aa, synthetic construct
  source 1..508: note = 21H7 co binder + huIgG1Fc_YTE_C S
EVQLVESGGGLVQAGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTQVTVSSGGGSGGGGSGGGGSGGGGSGGGGSGGGGSGGGGSEGVGLVESGGGSVQAGDSLRLSCAASGRTFSTYIIGWFRQAPGKEREFVASISWGGRGTYYADSVKDRFTISRDNPKNMVYLQMNSLKPEDTAVYYCAAGDLPGGRRALDYDYWGQGTLVTVSSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 508 aa, synthetic construct
  source 1..508: note = Humanized 21H7 co binder + huIgG1Fc_YTE_C S
EVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - skipped

SEQ ID NO: 60 - protein, 17 aa, synthetic construct
  source 1..17: note = Exemplary VHH1 CDR2 consensus sequence (Kabat numbering)
  VARIANT 8: note = X is T or V
  VARIANT 9: note = X is L or M
  VARIANT 11: note = X is K or N
  VARIANT 13: note = X is D or E
GISSSGYXXYXSXSMKG

SEQ ID NO: 61 - protein, 9 aa, synthetic construct
  source 1..9: note = Exemplary VHH1 CDR3 consensus sequence (Kabat numbering)
  VARIANT 2: note = X is I or T
  VARIANT 3: note = X is F or I
  VARIANT 8: note = X is E or D
GXXGRPYXY

SEQ ID NO: 62 - protein, 232 aa, synthetic construct
  source 1..232: note = huIgG1Fc_YTE_C S
EPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 17 aa, synthetic construct
  source 1..17: note = Exemplary VHH CDR2 consensus sequence (Kabat numbering)
  VARIANT 8: note = X is T or V
  VARIANT 9: note = X is L or M
  VARIANT 11: note = X is K or N
  VARIANT 12: note = X is S or A
  VARIANT 13: note = X is D or E
GISSSGYXXYXXXSMKG

SEQ ID NO: 64 - protein, 9 aa, synthetic construct
  source 1..9: note = Exemplary VHH CDR3 consensus sequence (Kabat numbering)
  VARIANT 2: note = X is I, T, or V
  VARIANT 3: note = X is F or I
  VARIANT 8: note = X is E or D
  VARIANT 9: note = X is Y or F
GXXGRPYXX

SEQ ID NO: 65 - protein, 118 aa, synthetic construct
  source 1..118: note = 20C7 VHH1 single binder
EVQLVESGGGSVQAGDSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTLVTVSS

SEQ ID NO: 66 - protein, 118 aa, synthetic construct
  source 1..118: note = 1H4 VHH2 single binder
EVQLVESGGDLVQAGGSLRLSCAASGGTFSSYAMAWFRQAPGKEREYVAGISSSGYTLYKADSMKGRFTISRENAKNMVYLQMNSLKPEDTAVYSCAAGVFGRPYEYWGRGTQVTVSS

SEQ ID NO: 67 - protein, 118 aa, synthetic construct
  source 1..118: note = 1C2 VHH2 single binder
EVQLVESGGGSVQAGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTQVTVSS

SEQ ID NO: 68 - protein, 118 aa, synthetic construct
  source 1..118: note = 1E3 VHH1 single binder
EVQLVESGGGSVQAGGSLRLSCAASGGTFSSYAMAWFRQAPGKEREYVAGISSSGYTLYKSDSMKGRFTISRENAKNMVYLQMNSLKPEDTAVYSCAAGIFGRPYEYWGQGTLVTVSS

SEQ ID NO: 69 - protein, 118 aa, synthetic construct
  source 1..118: note = 1D12 VHH1 single binder
EVQLVESGGGSVQPGGSLRLSCAASGGTFSSYTMGWFRQAPGKEREYVGGISSSGYVMYNSDSMKGRFTISRENAKNMVYLQMNSLKPEDTAVYYCAAGTIGRPYDFWGQGTLVTVSS

SEQ ID NO: 70 - protein, 118 aa, synthetic construct
  source 1..118: note = 2H2 VHH1 single binder
EVQLVESGGGSVQPGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTLVTVSS

SEQ ID NO: 71 - protein, 118 aa, synthetic construct; identical to SEQ ID NO: 67
  source 1..118: note = 1H2 VHH1 single binder

SEQ ID NO: 72 - protein, 118 aa, synthetic construct
  source 1..118: note = 1C7 VHH1 single binder
EVQLVESGGGSVQPGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTQVTVSS

SEQ ID NO: 73 - protein, 118 aa, synthetic construct; identical to SEQ ID NO: 69
  source 1..118: note = 1B10 VHH1 single binder

SEQ ID NO: 74 - protein, 118 aa, synthetic construct; identical to SEQ ID NO: 31
  source 1..118: note = 1G6 VHH1 single binder

SEQ ID NO: 75 - protein, 118 aa, synthetic construct; identical to SEQ ID NO: 67
  source 1..118: note = 1E2 VHH1 single binder

SEQ ID NO: 76 - protein, 118 aa, synthetic construct
  source 1..118: note = 1B12 VHH2 single binder
QVKLEESGGGSVQAGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTLVTVSS

SEQ ID NO: 77 - protein, 118 aa, synthetic construct; identical to SEQ ID NO: 31
  source 1..118: note = 1C3 VHH1 single binder

SEQ ID NO: 78 - protein, 8 aa, synthetic construct
  source 1..8: note = 1C10 VHH1 CDR1 (IMGT numbering)
GGTFSSYT

SEQ ID NO: 79 - protein, 8 aa, synthetic construct
  source 1..8: note = 1C10 VHH1 CDR2 (IMGT numbering)
ISSSGYVM

SEQ ID NO: 80 - protein, 11 aa, synthetic construct
  source 1..11: note = 1C10 VHH1 CDR3 (IMGT numbering)
AAGTIGRPYDY

SEQ ID NO: 81 - protein, 7 aa, synthetic construct
  source 1..7: note = 1C10 VHH1 CDR1 (Chothia numbering)
GGTFSSY

SEQ ID NO: 82 - protein, 6 aa, synthetic construct
  source 1..6: note = 1C10 VHH1 CDR2 (Chothia numbering)
SSSGYV

SEQ ID NO: 83 - protein, 9 aa, synthetic construct
  source 1..9: note = 1C10 VHH1 CDR3 (Chothia numbering)
GTIGRPYDY

SEQ ID NO: 84 - protein, 8 aa, synthetic construct
  source 1..8: note = 1C10 VHH2 CDR1 (IMGT numbering)
GDISSIVA

SEQ ID NO: 85 - protein, 7 aa, synthetic construct
  source 1..7: note = 1C10 VHH2 CDR2 (IMGT numbering)
ITSGGRT

SEQ ID NO: 86 - protein, 14 aa, synthetic construct
  source 1..14: note = 1C10 VHH2 CDR3 (IMGT numbering)
AADRGWTSVGEYDY

SEQ ID NO: 87 - protein, 7 aa, synthetic construct
  source 1..7: note = 1C10 VHH2 CDR1 (Chothia numbering)
GDISSIV

SEQ ID NO: 88 - protein, 5 aa, synthetic construct
  source 1..5: note = 1C10 VHH2 CDR2 (Chothia numbering)
TSGGR

SEQ ID NO: 89 - protein, 12 aa, synthetic construct
  source 1..12: note = 1C10 VHH2 CDR3 (Chothia numbering)
DRGWTSVGEYDY

SEQ ID NO: 90 - protein, 232 aa, synthetic construct
  source 1..232: note = huIgG1Fc_YTE_LALA_C S
EPKSSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 507 aa, synthetic construct
  source 1..507: note = 1C10 co-binder + huIgG1Fc_ YTE_LALA_C S
EVQLVESGGGLVQAGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTQVTVSSGGGSGGGGSGGGGSGGGGSGGGGSGGGGSGGGGSGMTGVKLEESGGGLVQPGGSLILSCAASGDISSIVAMGWYRQAPGKQRELVAAITSGGRTHYRDSVKGRFTISGNNDNSALYLHMNSLKPEDTAVYYCAADRGWTSVGEYDYWGKGTLVTVSSEPKSSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 507 aa, synthetic construct
  source 1..507: note = Humanized 1C10 co binder + huIgG1Fc_ YTE_LALA_C S
EVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSSEPKSSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 118 aa, synthetic construct
  source 1..118: note = Humanized 1C10 VHH1 with N-terminal consensus residue
  VARIANT 1: note = X is E or Q
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSS

SEQ ID NO: 94 - protein, 118 aa, synthetic construct
  VARIANT 1: note = X is E or Q
  VARIANT 33: note = X is A or T
  VARIANT 35: note = X is A or G
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGTIGRPYDYWGQGTQVTVSS

SEQ ID NO: 95 - protein, 118 aa, synthetic construct
  source 1..118: note = Humanized 1C10 VHH1 with N-terminal consensus residue, and consensus CDR2A
  VARIANT 1: note = X is E or Q
  VARIANT 57: note = X is T or V
  VARIANT 58: note = X is L or M
  VARIANT 60: note = X is K or N
  VARIANT 62: note = X is D or E
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGTIGRPYDYWGQGTQVTVSS

SEQ ID NO: 96 - protein, 118 aa, synthetic construct
  source 1..118: note = Humanized 1C10 VHH1 with N-terminal consensus residue, and consensus CDR3A
  VARIANT 1: note = X is E or Q
  VARIANT 100: note = X is I or T
  VARIANT 101: note = X is F or I
  VARIANT 106: note = X is E or D
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXYWGQGTQVTVSS

SEQ ID NO: 97 - protein, 118 aa, synthetic construct
  source 1..118: note = Humanized 1C10 VHH1 with N-terminal consensus residue, and consensus CDRs1 and 2A
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGTIGRPYDYWGQGTQVTVSS

SEQ ID NO: 98 - protein, 118 aa, synthetic construct
  source 1..118: note = Humanized 1C10 VHH1 with N-terminal consensus residue, and consensus CDRs1 and 3A
  VARIANT 1: note = X is E or Q
  VARIANT 33: note = X is A or T
  VARIANT 35: note = X is A or G
  VARIANT 100: note = X is I or T
  VARIANT 101: note = X is F or I
  VARIANT 106: note = X is E or D
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXYWGQGTQVTVSS

SEQ ID NO: 99 - protein, 118 aa, synthetic construct
  source 1..118: note = Humanized 1C10 VHH1 with N-terminal consensus residue, and consensus CDRs2A and 3A
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGXXGRPYXYWGQGTQVTVSS

SEQ ID NO: 100 - protein, 118 aa, synthetic construct
  source 1..118: note = Humanized 1C10 VHH1 with N-terminal consensus residue, and consensus CDRs1, 2A and 3A
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGXXGRPYXYWGQGTQVTVSS

SEQ ID NO: 101 - protein, 120 aa, synthetic construct
  source 1..120: note = Humanized 1C10 VHH2 with one amino acid consensus addition at N-terminus
  VARIANT 1: note = X is E or Q
XVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 102 - protein, 118 aa, synthetic construct
  source 1..118: note = Humanized 1C10 VHH2 with one amino acid deletion at N-terminus
QLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 103 - protein, 117 aa, synthetic construct
  source 1..117: note = Humanized 1C10 VHH2 with two amino acid deletion at N-terminus
LVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 104 - protein, 38 aa, synthetic construct
  source 1..38: note = Humanized 1C10 linker connecting VHH1 and VHH2
GGGSGGGGSGGGGSGGGGSGGGGSGGGGSGGGGSGMTG

SEQ ID NO: 105 - protein, 276 aa, synthetic construct
  source 1..276: note = Humanized 1C10 co-binder with VHH2 having one amino acid consensus addition at N-terminus
  VARIANT 157: note = X is E or Q
EVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 106 - protein, 274 aa, synthetic construct
  source 1..274: note = Humanized 1C10 co-binder with VHH2 having one amino acid deletion at N-terminus
EVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 107 - protein, 273 aa, synthetic construct
  source 1..273: note = Humanized 1C10 co-binder with VHH2 having two amino acid deletion at N-terminus
EVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 108 - protein, 275 aa, synthetic construct
  source 1..275: note = Humanized 1C10 co-binder with VHH1 N-terminal consensus residue
  VARIANT 1: note = X is E or Q
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 109 - protein, 276 aa, synthetic construct
  source 1..276: note = Humanized 1C10 co-binder with VHH1 N-terminal consensus residue, and VHH2 with one amino acid consensus addition at N-terminus
  VARIANT 1: note = X is E or Q
  VARIANT 157: note = X is E or Q
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 110 - protein, 274 aa, synthetic construct
  source 1..274: note = Humanized 1C10 co-binder with VHH1 N-terminal consensus residue, and VHH2 with one amino acid deletion at N-terminus
  VARIANT 1: note = X is E or Q
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 111 - protein, 273 aa, synthetic construct
  source 1..273: note = Humanized 1C10 co-binder with VHH1 N-terminal consensus residue, and VHH2 with two amino acid deletion at N-terminus
  VARIANT 1: note = X is E or Q
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 112 - protein, 275 aa, synthetic construct
  source 1..275: note = Humanized 1C10 co-binder with VHH1 N-terminal consensus residue and consensus CDR1
  VARIANT 1: note = X is E or Q
  VARIANT 33: note = X is A or T
  VARIANT 35: note = X is A or G
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 113 - protein, 276 aa, synthetic construct
  source 1..276: note = Humanized 1C10 co-binder with VHH1 N-terminal consensus residue and consensus CDR1, and VHH2 with one amino acid consensus addition at N-terminus
  VARIANT 1: note = X is E or Q
  VARIANT 33: note = X is A or G
  VARIANT 35: note = X is E or Q
  VARIANT 157: note = X is E or Q
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 114 - protein, 274 aa, synthetic construct
  source 1..274: note = Humanized 1C10 co-binder with VHH1 N-terminal consensus residue and consensus CDR1, and VHH2 with one amino acid deletion at N-terminus
  VARIANT 1: note = X is E or Q
  VARIANT 33: note = X is A or T
  VARIANT 35: note = X is A or G
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 115 - protein, 273 aa, synthetic construct
  source 1..273: note = Humanized 1C10 co-binder with VHH1 N-terminal consensus residue and consensus CDR1, and VHH2 with two amino acid deletion at N-terminus
  VARIANT 1: note = X is E or Q
  VARIANT 33: note = X is A or T
  VARIANT 35: note = X is A or G
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 116 - protein, 275 aa, synthetic construct
  source 1..275: note = Humanized 1C10 co-binder with VHH1 N-terminal consensus residue and consensus CDR2A
  VARIANT 1: note = X is E or Q
  VARIANT 57: note = X is T or V
  VARIANT 58: note = X is L or M
  VARIANT 60: note = X is L or M
  VARIANT 62: note = X is D or E
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 117 - protein, 276 aa, synthetic construct
  source 1..276: note = Humanized 1C10 co-binder with VHH1 N-terminal consensus residue and consensus CDR2A, and VHH2 with one amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 157: note = X is E or Q
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 118 - protein, 274 aa, synthetic construct
  source 1..274: note = Humanized 1C10 co-binder with VHH1 N-terminal consensus residue and consensus CDR2A, and VHH2 with one amino acid deletion at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 119 - protein, 273 aa, synthetic construct
  source 1..273: note = Humanized 1C10 co-binder with VHH1 N-terminal consensus residue and consensus CDR2A, and VHH2 with two amino acid deletion at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 120 - protein, 275 aa, synthetic construct
  source 1..275: note = Humanized 1C10 co-binder with VHH1 N-terminal consensus residue and consensus CDR3A
  VARIANT 1: note = X is E or Q
  VARIANT 100: note = X is I or T
  VARIANT 101: note = X is F or I
  VARIANT 106: note = X is E or D
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 121 - protein, 276 aa, synthetic construct
  source 1..276: note = Humanized 1C10 co-binder with VHH1 N-terminal consensus residue and consensus CDR3A, and VHH2 with one amino acid consensus addition at N-terminus
  VARIANT 1: note = X is E or Q
  VARIANT 100: note = X is I or T
  VARIANT 101: note = X is F or I
  VARIANT 106: note = X is E or D
  VARIANT 157: note = X is E or Q
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 122 - protein, 274 aa, synthetic construct
  source 1..274: note = Humanized 1C10 co-binder with VHH1 N-terminal consensus residue and consensus CDR3A, and VHH2 with one amino acid deletion at N-terminus
  VARIANT 1: note = X is E or Q
  VARIANT 100: note = X is I or T
  VARIANT 101: note = X is F or I
  VARIANT 106: note = X is E or D
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 123 - protein, 273 aa, synthetic construct
  source 1..273: note = Humanized 1C10 co-binder with VHH1 N-terminal consensus residue and consensus CDR3A, and VHH2 with two amino acid deletion at N-terminus
  VARIANT 1: note = X is E or Q
  VARIANT 100: note = X is I or T
  VARIANT 101: note = X is F or I
  VARIANT 106: note = X is E or D
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 124 - protein, 275 aa, synthetic construct
  source 1..275: note = Humanized 1C10 co-binder with VHH1 N-terminal consensus residue and consensus CDRs1 and 2A
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 125 - protein, 276 aa, synthetic construct
  source 1..276: note = Humanized 1C10 co-binder with VHH1 N-terminal consensus residue and consensus CDRs1 and 2A, and VHH2 with one amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 157: note = X is E or Q
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 126 - protein, 274 aa, synthetic construct
  source 1..274: note = Humanized 1C10 co-binder with VHH1 N-terminal consensus residue and consensus CDRs1 and 2A, and VHH2 with one amino acid deletion at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 127 - protein, 273 aa, synthetic construct
  source 1..273: note = Humanized 1C10 co-binder with VHH1 N-terminal consensus residue and consensus CDRs1 and 2A, and VHH2 with two amino acid deletion at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 128 - protein, 275 aa, synthetic construct
  source 1..275: note = Humanized 1C10 co-binder with VHH1 N-terminal consensus residue and consensus CDRs1 and 3A
  VARIANT 1: note = X is E or Q
  VARIANT 33: note = X is A or T
  VARIANT 35: note = X is A or G
  VARIANT 100: note = X is I or T
  VARIANT 101: note = X is F or I
  VARIANT 106: note = X is E or D
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 129 - protein, 276 aa, synthetic construct
  source 1..276: note = Humanized 1C10 co-binder with VHH1 N-terminal consensus residue and consensus CDRs1 and 3A, and VHH2 with one amino acid consensus addition at N-terminus
  VARIANT 1: note = X is E or Q
  VARIANT 33: note = X is A or T
  VARIANT 35: note = X is A or G
  VARIANT 100: note = X is I or T
  VARIANT 101: note = X is F or I
  VARIANT 106: note = X is E or D
  VARIANT 157: note = X is E or Q
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 130 - protein, 274 aa, synthetic construct
  source 1..274: note = Humanized 1C10 co-binder with VHH1 N-terminal consensus residue and consensus CDRs1 and 3A, and VHH2 with one amino acid deletion at N-terminus
  VARIANT 1: note = X is E or Q
  VARIANT 33: note = X is A or T
  VARIANT 35: note = X is A or G
  VARIANT 100: note = X is I or T
  VARIANT 101: note = X is F or I
  VARIANT 106: note = X is E or D
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 131 - protein, 273 aa, synthetic construct
  source 1..273: note = Humanized 1C10 co-binder with VHH1 N-terminal consensus residue and consensus CDRs1 and 3A, and VHH2 with two amino acid deletion at N-terminus
  VARIANT 1: note = X is E or Q
  VARIANT 33: note = X is A or T
  VARIANT 35: note = X is A or G
  VARIANT 100: note = X is I or T
  VARIANT 101: note = X is F or I
  VARIANT 106: note = X is E or D
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 132 - protein, 275 aa, synthetic construct
  source 1..275: note = Humanized 1C10 co-binder with VHH1 N-terminal consensus residue and consensus CDRs2A and 3A
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 133 - protein, 276 aa, synthetic construct
  source 1..276: note = Humanized 1C10 co-binder with VHH1 N-terminal consensus residue and consensus CDRs2A and 3A, and VHH2 with one amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
  VARIANT 157: note = X is E or Q
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 134 - protein, 274 aa, synthetic construct
  source 1..274: note = Humanized 1C10 co-binder with VHH1 N-terminal consensus residue and consensus CDRs2A and 3A, and VHH2 with one amino acid deletion at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 135 - protein, 273 aa, synthetic construct
  source 1..273: note = Humanized 1C10 co-binder with VHH1 N-terminal consensus residue and consensus CDRs2A and 3A, and VHH2 with two amino acid deletion at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 136 - protein, 275 aa, synthetic construct
  source 1..275: note = Humanized 1C10 co-binder with VHH1 N-terminal consensus residue and consensus CDRs1, 2A and 3A
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 137 - protein, 276 aa, synthetic construct
  source 1..276: note = Humanized 1C10 co-binder with VHH1 N-terminal consensus residue and consensus CDRs1, 2A and 3A, and VHH2 with one amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
  VARIANT 157: note = X is E or Q
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 138 - protein, 274 aa, synthetic construct
  source 1..274: note = Humanized 1C10 co-binder with VHH1 N-terminal consensus residue and consensus CDRs1, 2A and 3A, and VHH2 with one amino acid deletion at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 139 - protein, 273 aa, synthetic construct
  source 1..273: note = Humanized 1C10 co-binder with VHH1 N-terminal consensus residue and consensus CDRs1, 2A and 3A, and VHH2 with two amino acid deletion at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 140 - protein, 118 aa, synthetic construct
  source 1..118: note = Humanized 1C10 VHH1 with N-terminal consensus residue, and consensus CDR2B
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGTIGRPYDYWGQGTQVTVSS

SEQ ID NO: 141 - protein, 118 aa, synthetic construct
  source 1..118: note = Humanized 1C10 VHH1 with N-terminal consensus residue, and consensus CDR3B
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXXWGQGTQVTVSS

SEQ ID NO: 142 - protein, 118 aa, synthetic construct
  source 1..118: note = Humanized 1C10 VHH1 with N-terminal consensus residue, and consensus CDRs1 and 2B
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGTIGRPYDYWGQGTQVTVSS

SEQ ID NO: 143 - protein, 118 aa, synthetic construct
  source 1..118: note = Humanized 1C10 VHH1 with N-terminal consensus residue, and consensus CDRs1 and 3B
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 107: note = X is Y or F
  VARIANT 106: note = X is E or D
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXXWGQGTQVTVSS

SEQ ID NO: 144 - protein, 118 aa, synthetic construct
  source 1..118: note = Humanized 1C10 VHH1 with N-terminal consensus residue, and consensus CDRs2B and 3B
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGXXGRPYXXWGQGTQVTVSS

SEQ ID NO: 145 - protein, 118 aa, synthetic construct
  source 1..118: note = Humanized 1C10 VHH1 with N-terminal consensus residue, and consensus CDRs1, 2B and 3B
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGXXGRPYXXWGQGTQVTVSS

SEQ ID NO: 146 - protein, 120 aa, synthetic construct
  source 1..120: note = Humanized 1C10 VHH2 with N-terminal consensus residues at position 1 and 3
  VARIANT 1: note = X is E or Q
  VARIANT 3: note = X is Q or K
XVXLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 147 - protein, 275 aa, synthetic construct
  source 1..275: note = Humanized 1C10 co-binder with VHH1 N-terminal consensus residue, and consensus CDR2B
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 148 - protein, 276 aa, synthetic construct
  source 1..276: note = Humanized 1C10 co-binder with VHH1 N-terminal consensus residue and consensus CDR2B, and VHH2 with one amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 157: note = X is E or Q
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 149 - protein, 274 aa, synthetic construct
  source 1..274: note = Humanized 1C10 co-binder with VHH1 N-terminal consensus residue and consensus CDR2B, and VHH2 with one amino acid deletion at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 150 - protein, 273 aa, synthetic construct
  source 1..273: note = Humanized 1C10 co-binder with VHH1 N-terminal consensus residue and consensus CDR2B, and VHH2 with two amino acid deletion at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 151 - protein, 276 aa, synthetic construct
  source 1..276: note = Humanized 1C10 co-binder with VHH1 N-terminal consensus residue and consensus CDR2B, and VHH2 with N-terminal consensus residues at position 1 and 3
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 157: note = X is E or Q
  VARIANT 159: note = X is Q or K
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGXVXLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 152 - protein, 275 aa, synthetic construct
  source 1..275: note = Humanized 1C10 co-binder with VHH1 N-terminal consensus residue, and consensus CDR3B
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 153 - protein, 276 aa, synthetic construct
  source 1..276: note = Humanized 1C10 co-binder with VHH1 N-terminal consensus residue and consensus CDR3B, and VHH2 with one amino acid consensus addition at N-terminus
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 154 - protein, 274 aa, synthetic construct
  source 1..274: note = Humanized 1C10 co-binder with VHH1 N-terminal consensus residue and consensus CDR3B, and VHH2 with one amino acid deletion at N-terminus
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 155 - protein, 273 aa, synthetic construct
  source 1..273: note = Humanized 1C10 co-binder with VHH1 N-terminal consensus residue and consensus CDR3B, and VHH2 with two amino acid deletion at N-terminus
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 156 - protein, 276 aa, synthetic construct
  source 1..276: note = Humanized 1C10 co-binder with VHH1 N-terminal consensus residue and consensus CDR3B, and VHH2 with N-terminal consensus residues at position 1 and 3
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
  VARIANT 159: note = X is Q or K
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGXVXLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 157 - protein, 275 aa, synthetic construct
  source 1..275: note = Humanized 1C10 co-binder with VHH1 N-terminal consensus residue, and consensus CDRs1 and 2B
  VARIANT 1: note = X is E or Q
  VARIANT 33: note = X is A or T
  VARIANT 35: note = X is A or G
  VARIANT 58: note = X is T or V
  VARIANT 58: note = X is L or M
  VARIANT 60: note = X is K or N
  VARIANT 61: note = X is S or A
  VARIANT 62: note = X is D or E
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 158 - protein, 276 aa, synthetic construct
  source 1..276: note = Humanized 1C10 co-binder with VHH1 N-terminal consensus residue and consensus CDRs1 and 2B, and VHH2 with one amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 157: note = X is E or Q
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 159 - protein, 274 aa, synthetic construct
  source 1..274: note = Humanized 1C10 co-binder with VHH1 N-terminal consensus residue and consensus CDRs1 and 2B, and VHH2 with one amino acid deletion at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 160 - protein, 273 aa, synthetic construct
  source 1..273: note = Humanized 1C10 co-binder with VHH1 N-terminal consensus residue and consensus CDRs1 and 2B, and VHH2 with two amino acid deletion at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 161 - protein, 276 aa, synthetic construct
  source 1..276: note = Humanized 1C10 co-binder with VHH1 N-terminal consensus residue and consensus CDRs1 and 2B, and VHH2 with N-terminal consensus residues at position 1 and 3
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 157: note = X is E or Q
  VARIANT 159: note = X is Q or K
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSGMTGXVXLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 162 - protein, 275 aa, synthetic construct
  source 1..275: note = Humanized 1C10 co-binder with VHH1 N-terminal consensus residue, and consensus CDRs1 and 3B
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 163 - protein, 276 aa, synthetic construct
  source 1..276: note = Humanized 1C10 co-binder with VHH1 N-terminal consensus residue, and consensus CDRs1 and 3B, and VHH2 with one amino acid consensus addition at N-terminus
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 164 - protein, 274 aa, synthetic construct
  source 1..274: note = Humanized 1C10 co-binder with VHH1 N-terminal consensus residue, and consensus CDRs1 and 3B, and VHH2 with one amino acid deletion at N-terminus
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 165 - protein, 273 aa, synthetic construct
  source 1..273: note = Humanized 1C10 co-binder with VHH1 N-terminal consensus residue, and consensus CDRs1 and 3B, and VHH2 with two amino acid deletion at N-terminus
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 166 - protein, 276 aa, synthetic construct
  source 1..276: note = Humanized 1C10 co-binder with VHH1 N-terminal consensus residue, and consensus CDRs1 and 3B, and VHH2 with N-terminal consensus residues at position 1 and 3
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
  VARIANT 159: note = X is Q or K
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGXVXLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 167 - protein, 275 aa, synthetic construct
  source 1..275: note = Humanized 1C10 co-binder with VHH1 N-terminal consensus residue, and consensus CDRs2B and 3B
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 62: note = X is D or E
  VARIANT 101: note = X is F or I
  VARIANT 106: note = X is E or D
  VARIANT 107: note = X is Y or F
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 168 - protein, 276 aa, synthetic construct
  source 1..276: note = Humanized 1C10 co-binder with VHH1 N-terminal consensus residue, and consensus CDRs2B and 3B, and VHH2 with one amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 169 - protein, 274 aa, synthetic construct
  source 1..274: note = Humanized 1C10 co-binder with VHH1 N-terminal consensus residue, and consensus CDRs2B and 3B, and VHH2 with one amino acid deletion at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 170 - protein, 273 aa, synthetic construct
  source 1..273: note = Humanized 1C10 co-binder with VHH1 N-terminal consensus residue, and consensus CDRs2B and 3B, and VHH2 with two amino acid deletion at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 171 - protein, 276 aa, synthetic construct
  source 1..276: note = Humanized 1C10 co-binder with VHH1 N-terminal consensus residue, and consensus CDRs2B and 3B, and VHH2 with N-terminal consensus residues at position 1 and 3
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
  VARIANT 159: note = X is Q or K
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGXVXLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 172 - protein, 275 aa, synthetic construct
  source 1..275: note = Humanized 1C10 co-binder with VHH1 N-terminal consensus residue, and consensus CDRs1, 2B and 3B
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 173 - protein, 276 aa, synthetic construct
  source 1..276: note = Humanized 1C10 co-binder with VHH1 N-terminal consensus residue, and consensus CDRs1, 2B and 3B, and VHH2 with one amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGXVQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 174 - protein, 274 aa, synthetic construct
  source 1..274: note = Humanized 1C10 co-binder with VHH1 N-terminal consensus residue, and consensus CDRs1, 2B and 3B, and VHH2 with one amino acid deletion at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGQLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 175 - protein, 273 aa, synthetic construct
  source 1..273: note = Humanized 1C10 co-binder with VHH1 N-terminal consensus residue, and consensus CDRs1, 2B and 3B, and VHH2 with two amino acid deletion at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 176 - protein, 276 aa, synthetic construct
  source 1..276: note = Humanized 1C10 co-binder with VHH1 N-terminal consensus residue, and consensus CDRs1, 2B and 3B, and VHH2 with N-terminal consensus residues at position 1 and 3
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
  VARIANT 159: note = X is Q or K
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSGMTGXVXLVESGGGLVQPGGSLRLSCAASGDISSIVAMGWYRQAPGKQRELVSAITSGGRTHYRDSVKGRFTISRDNAKNTLYLQMNSLRAEDTAVYYCAADRGWTSVGEYDYWGQGTQVTVSS

SEQ ID NO: 177 - protein, 118 aa, synthetic construct; identical to SEQ ID NO: 93
  source 1..118: note = Humanized 21H7 VHH1 with N-terminal consensus residue
  VARIANT 1: note = X is E or Q

SEQ ID NO: 178 - protein, 118 aa, synthetic construct; identical to SEQ ID NO: 94
  source 1..118: note = Humanized 21H7 VHH1 with N-terminal consensus residue, and consensus CDR1
  VARIANT 1: note = X is E or Q
  VARIANT 33: note = X is A or T
  VARIANT 35: note = X is A or G

SEQ ID NO: 179 - protein, 118 aa, synthetic construct; identical to SEQ ID NO: 95
  source 1..118: note = Humanized 21H7 VHH1 with N-terminal consensus residue, and consensus CDR2A
  VARIANT 1: note = X is E or Q
  VARIANT 57: note = X is T or V
  VARIANT 58: note = X is L or M
  VARIANT 60: note = X is K or N
  VARIANT 62: note = X is D or E

SEQ ID NO: 180 - protein, 118 aa, synthetic construct; identical to SEQ ID NO: 96
  source 1..118: note = Humanized 21H7 VHH1 with N-terminal consensus residue, and consensus CDR3A
  VARIANT 1: note = X is E or Q
  VARIANT 100: note = X is I or T
  VARIANT 101: note = X is F or I
  VARIANT 106: note = X is E or D

SEQ ID NO: 181 - protein, 118 aa, synthetic construct; identical to SEQ ID NO: 97
  source 1..118: note = Humanized 21H7 VHH1 with N-terminal consensus residue, and consensus CDRs1 and 2A
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E

SEQ ID NO: 182 - protein, 118 aa, synthetic construct; identical to SEQ ID NO: 98
  source 1..118: note = Humanized 21H7 VHH1 with N-terminal consensus residue, and consensus CDRs1 and 3A
  VARIANT 1: note = X is E or Q
  VARIANT 33: note = X is A or T
  VARIANT 35: note = X is A or G
  VARIANT 100: note = X is I or T
  VARIANT 101: note = X is F or I
  VARIANT 106: note = X is E or D

SEQ ID NO: 183 - protein, 118 aa, synthetic construct; identical to SEQ ID NO: 99
  source 1..118: note = Humanized 21H7 VHH1 with N-terminal consensus residue, and consensus CDRs2A and 3A
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D

SEQ ID NO: 184 - protein, 118 aa, synthetic construct; identical to SEQ ID NO: 100
  source 1..118: note = Humanized 21H7 VHH1 with N-terminal consensus residue, and consensus CDRs1, 2A and 3A
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D

SEQ ID NO: 185 - protein, 121 aa, synthetic construct
  source 1..121: note = Humanized 21H7 VHH2 with one amino acid consensus addition at N-terminus
  VARIANT 1: note = X is Q or K
XLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 186 - protein, 122 aa, synthetic construct
  source 1..122: note = Humanized 21H7 VHH2 with two amino acid consensus addition at N-terminus
  VARIANT 1: note = X is V
  VARIANT 2: note = X is Q or K
XXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 187 - protein, 123 aa, synthetic construct
  source 1..123: note = Humanized 21H7 VHH2 with three amino acid consensus addition at N-terminus
  VARIANT 1: note = X is E or Q
  VARIANT 2: note = X is V
  VARIANT 3: note = X is Q or K
XXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 188 - protein, 38 aa, synthetic construct
  source 1..38: note = Humanized 21H7 linker connecting VHH1 and VHH2
GGGSGGGGSGGGGSGGGGSGGGGSGGGGSGGGGSEGVG

SEQ ID NO: 189 - protein, 276 aa, synthetic construct
  source 1..276: note = Humanized 21H7 co-binder with VHH1 N-terminal consensus residue
  VARIANT 1: note = X is E or Q
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 190 - protein, 277 aa, synthetic construct
  source 1..277: note = Humanized 21H7 co-binder with VHH1 N-terminal consensus residue, and VHH2 with one amino acid consensus addition at N-terminus
  VARIANT 1: note = X is E or Q
  VARIANT 157: note = X is Q or K
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 191 - protein, 278 aa, synthetic construct
  source 1..278: note = Humanized 21H7 co-binder with VHH1 N-terminal consensus residue, and VHH2 with two amino acid consensus addition at N-terminus
  VARIANT 1: note = X is E or Q
  VARIANT 157: note = X is V
  VARIANT 158: note = X is Q or K
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 192 - protein, 279 aa, synthetic construct
  source 1..279: note = Humanized 21H7 co-binder with VHH1 N-terminal consensus residue, and VHH2 with three amino acid consensus addition at N-terminus
  VARIANT 1: note = X is E or Q
  VARIANT 157: note = X is E or Q
  VARIANT 159: note = X is Q or K
  VARIANT 158: note = X is V
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 193 - protein, 276 aa, synthetic construct
  source 1..276: note = Humanized 21H7 co-binder with VHH1 N-terminal consensus residue and consensus CDR1
  VARIANT 1: note = X is E or Q
  VARIANT 33: note = X is A or T
  VARIANT 35: note = X is A or G
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 194 - protein, 277 aa, synthetic construct
  source 1..277: note = Humanized 21H7 co-binder with VHH1 N-terminal consensus residue and consensus CDR1, and VHH2 with one amino acid consensus addition at N-terminus
  VARIANT 1: note = X is E or Q
  VARIANT 33: note = X is A or T
  VARIANT 35: note = X is A or G
  VARIANT 157: note = X is Q or K
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 195 - protein, 278 aa, synthetic construct
  source 1..278: note = Humanized 21H7 co-binder with VHH1 N-terminal consensus residue and consensus CDR1, and VHH2 with two amino acid consensus addition at N-terminus
  VARIANT 1: note = X is E or Q
  VARIANT 33: note = X is A or T
  VARIANT 35: note = X is A or G
  VARIANT 157: note = X is V
  VARIANT 158: note = X is Q or K
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 196 - protein, 279 aa, synthetic construct
  source 1..279: note = Humanized 21H7 co-binder with VHH1 N-terminal consensus residue and consensus CDR1, and VHH2 with three amino acid consensus addition at N-terminus
  VARIANT 1: note = X is E or Q
  VARIANT 33: note = X is A or T
  VARIANT 35: note = X is A or G
  VARIANT 157: note = X is E or Q
  VARIANT 158: note = X is V
  VARIANT 159: note = X is Q or K
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 197 - protein, 276 aa, synthetic construct
  source 1..276: note = Humanized 21H7 co-binder with VHH1 N-terminal consensus residue and consensus CDR2A
  VARIANT 1: note = X is E or Q
  VARIANT 57: note = X is T or V
  VARIANT 58: note = X is L or M
  VARIANT 60: note = X is K or N
  VARIANT 62: note = X is D or E
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 198 - protein, 277 aa, synthetic construct
  source 1..277: note = Humanized 21H7 co-binder with VHH1 N-terminal consensus residue and consensus CDR2A, and VHH2 with one amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 157: note = X is Q or K
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 199 - protein, 278 aa, synthetic construct
  source 1..278: note = Humanized 21H7 co-binder with VHH1 N-terminal consensus residue and consensus CDR2A, and VHH2 with two amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 158: note = X is Q or K
  VARIANT 157: note = X is V
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 200 - protein, 279 aa, synthetic construct
  source 1..279: note = Humanized 21H7 co-binder with VHH1 N-terminal consensus residue and consensus CDR2A, and VHH2 with three amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 157: note = X is E or Q
  VARIANT 158: note = X is V
  VARIANT 159: note = X is Q or K
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 201 - protein, 276 aa, synthetic construct
  source 1..276: note = Humanized 21H7 co binder with VHH1 N-terminal consensus residue and consensus CDR3A
  VARIANT 1: note = X is E or Q
  VARIANT 100: note = X is I or T
  VARIANT 101: note = X is F or I
  VARIANT 106: note = X is E or D
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 202 - protein, 277 aa, synthetic construct
  source 1..277: note = Humanized 21H7 co-binder with VHH1 N-terminal consensus residue and consensus CDR3A, and VHH2 with one amino acid consensus addition at N-terminus
  VARIANT 1: note = X is E or Q
  VARIANT 100: note = X is I or T
  VARIANT 101: note = X is F or I
  VARIANT 106: note = X is E or D
  VARIANT 157: note = X is Q or K
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 203 - protein, 278 aa, synthetic construct
  source 1..278: note = Humanized 21H7 co-binder with VHH1 N-terminal consensus residue and consensus CDR3A, and VHH2 with two amino acid consensus addition at N-terminus
  VARIANT 1: note = X is E or Q
  VARIANT 100: note = X is I or T
  VARIANT 101: note = X is F or I
  VARIANT 106: note = X is E or D
  VARIANT 157: note = X is V
  VARIANT 158: note = X is Q or K
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 204 - protein, 279 aa, synthetic construct
  source 1..279: note = Humanized 21H7 co-binder with VHH1 N-terminal consensus residue and consensus CDR3A, and VHH2 with three amino acid consensus addition at N-terminus
  VARIANT 1: note = X is E or Q
  VARIANT 100: note = X is I or T
  VARIANT 101: note = X is F or I
  VARIANT 106: note = X is E or D
  VARIANT 157: note = X is E or Q
  VARIANT 158: note = X is V
  VARIANT 159: note = X is Q or K
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 205 - protein, 276 aa, synthetic construct
  source 1..276: note = Humanized 21H7 co-binder with VHH1 N-terminal consensus residue and consensus CDRs1 and 2A
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 206 - protein, 277 aa, synthetic construct
  source 1..277: note = Humanized 21H7 co-binder with VHH1 N-terminal consensus residue and consensus CDRs1 and 2A, and VHH2 with one amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = 5 is K or N
  VARIANT 62: note = X is D or E
  VARIANT 157: note = X is Q or K
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 207 - protein, 278 aa, synthetic construct
  source 1..278: note = Humanized 21H7 co-binder with VHH1 N-terminal consensus residue and consensus CDRs1 and 2A, and VHH2 with two amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 158: note = X is Q or K
  VARIANT 157: note = X is V
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 208 - protein, 279 aa, synthetic construct
  source 1..279: note = Humanized 21H7 co-binder with VHH1 N-terminal consensus residue and consensus CDRs1 and 2A, and VHH2 with three amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 157: note = X is E or Q
  VARIANT 158: note = X is V
  VARIANT 159: note = X is Q or K
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 209 - protein, 276 aa, synthetic construct
  source 1..276: note = Humanized 21H7 co-binder with VHH1 N-terminal consensus residue and consensus CDRs1 and 3A
  VARIANT 1: note = X is E or Q
  VARIANT 33: note = X is A or T
  VARIANT 35: note = X is A or G
  VARIANT 100: note = X is I or T
  VARIANT 101: note = X is F or I
  VARIANT 106: note = X is E or D
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 210 - protein, 277 aa, synthetic construct
  source 1..277: note = Humanized 21H7 co-binder with VHH1 N-terminal consensus residue and consensus CDRs1 and 3A, and VHH2 with one amino acid consensus addition at N-terminus
  VARIANT 1: note = X is E or Q
  VARIANT 33: note = X is A or T
  VARIANT 35: note = X is A or G
  VARIANT 100: note = X is I or T
  VARIANT 101: note = X is F or I
  VARIANT 106: note = X is E or D
  VARIANT 157: note = X is Q or K
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 211 - protein, 278 aa, synthetic construct
  source 1..278: note = Humanized 21H7 co-binder with VHH1 N-terminal consensus residue and consensus CDRs1 and 3A, and VHH2 with two amino acid consensus addition at N-terminus
  VARIANT 1: note = X is E or Q
  VARIANT 33: note = X is A or T
  VARIANT 35: note = X is A or G
  VARIANT 100: note = X is I or T
  VARIANT 101: note = X is F or I
  VARIANT 106: note = X is E or D
  VARIANT 157: note = X is V
  VARIANT 158: note = X is Q or K
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 212 - protein, 279 aa, synthetic construct
  source 1..279: note = Humanized 21H7 co-binder with VHH1 N-terminal consensus residue and consensus CDRs1 and 3A, and VHH2 with three amino acid consensus addition at N-terminus
  VARIANT 1: note = X is E or Q
  VARIANT 33: note = X is A or T
  VARIANT 35: note = X is A or G
  VARIANT 100: note = X is I or T
  VARIANT 101: note = X is F or I
  VARIANT 106: note = X is E or D
  VARIANT 157: note = X is E or Q
  VARIANT 158: note = X is V
  VARIANT 159: note = X is Q or K
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 213 - protein, 276 aa, synthetic construct
  source 1..276: note = Humanized 21H7 co-binder with VHH1 N-terminal consensus residue and consensus CDRs2A and 3A
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 214 - protein, 277 aa, synthetic construct
  source 1..277: note = Humanized 21H7 co-binder with VHH1 N-terminal consensus residue and consensus CDRs2A and 3A, and VHH2 with one amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
  VARIANT 157: note = X is Q or K
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 215 - protein, 278 aa, synthetic construct
  source 1..278: note = Humanized 21H7 co-binder with VHH1 N-terminal consensus residue and consensus CDRs2A and 3A, and VHH2 with two amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
  VARIANT 157: note = X is V
  VARIANT 158: note = X is Q or K
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 216 - protein, 279 aa, synthetic construct
  source 1..279: note = Humanized 21H7 co-binder with VHH1 N-terminal consensus residue and consensus CDRs2A and 3A, and VHH2 with three amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
  VARIANT 157: note = X is E or Q
  VARIANT 158: note = X is V
  VARIANT 159: note = X is Q or K
XVQLVESGGGLVQPGGSLRLSCAASGGTFSSYTMGWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 217 - protein, 276 aa, synthetic construct
  source 1..276: note = Humanized 21H7 co-binder with VHH1 N-terminal consensus residue and consensus CDRs1, 2A and 3A
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 218 - protein, 277 aa, synthetic construct
  source 1..277: note = Humanized 21H7 co-binder with VHH1 N-terminal consensus residue and consensus CDRs1, 2A and 3A, and VHH2 with one amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
  VARIANT 157: note = X is Q or K
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 219 - protein, 278 aa, synthetic construct
  source 1..278: note = Humanized 21H7 co-binder with VHH1 N-terminal consensus residue and consensus CDRs1, 2A and 3A, and VHH2 with two amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = 9 is E or D
  VARIANT 157: note = X is V
  VARIANT 158: note = X is Q or K
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 220 - protein, 279 aa, synthetic construct
  source 1..279: note = Humanized 21H7 co-binder with VHH1 N-terminal consensus residue and consensus CDRs1, 2A and 3A, and VHH2 with three amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 62: note = X is D or E
  VARIANT 100: note = X is I or T
  VARIANT 101: note = X is F or I
  VARIANT 106: note = X is E or D
  VARIANT 157: note = X is E or Q
  VARIANT 158: note = X is V
  VARIANT 159: note = X is Q or K
XVQLVESGGGLVQPGGSLRLSCAASGGTFSSYXMXWFRQAPGKEREYVGGISSSGYXXYXSXSMKGRFTISRDNAKNTLYLQMNSLRAEDTAVYYCAAGXXGRPYXYWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 221 - protein, 118 aa, synthetic construct; identical to SEQ ID NO: 140
  source 1..118: note = Humanized 21H7 VHH1 with N-terminal consensus residue, and consensus CDR2B
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E

SEQ ID NO: 222 - protein, 118 aa, synthetic construct; identical to SEQ ID NO: 141
  source 1..118: note = Humanized 21H7 VHH1 with N-terminal consensus residue, and consensus CDR3B
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F

SEQ ID NO: 223 - protein, 118 aa, synthetic construct; identical to SEQ ID NO: 142
  source 1..118: note = Humanized 21H7 VHH1 with N-terminal consensus residue, and consensus CDRs1 and 2B
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E

SEQ ID NO: 224 - protein, 118 aa, synthetic construct; identical to SEQ ID NO: 143
  source 1..118: note = Humanized 21H7 VHH1 with N-terminal consensus residue, and consensus CDRs1 and 3B
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 106: note = X is E or D
  VARIANT 107: note = X is Y or F

SEQ ID NO: 225 - protein, 118 aa, synthetic construct; identical to SEQ ID NO: 144
  source 1..118: note = Humanized 21H7 VHH1 with N-terminal consensus residue, and consensus CDRs2B and 3B
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F

SEQ ID NO: 226 - protein, 118 aa, synthetic construct; identical to SEQ ID NO: 145
  source 1..118: note = Humanized 21H7 VHH1 with N-terminal consensus residue, and consensus CDRs1, 2B and 3B
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F

SEQ ID NO: 227 - protein, 276 aa, synthetic construct
  source 1..276: note = Humanized 21H7 co-binder with VHH1 N-terminal consensus residue, and consensus CDR2B
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 228 - protein, 277 aa, synthetic construct
  source 1..277: note = Humanized 21H7 co-binder with VHH1 N-terminal consensus residue, and consensus CDR2B, and VHH2 with one amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 157: note = X is Q or K
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 229 - protein, 278 aa, synthetic construct
  source 1..278: note = Humanized 21H7 co-binder with VHH1 N-terminal consensus residue, and consensus CDR2B, and VHH2 with two amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 158: note = X is Q or K
  VARIANT 157: note = X is V
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 230 - protein, 279 aa, synthetic construct
  source 1..279: note = Humanized 21H7 co-binder with VHH1 N-terminal consensus residue, and consensus CDR2B, and VHH2 with three amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 157: note = X is E or Q
  VARIANT 158: note = X is V
  VARIANT 159: note = X is Q or K
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 231 - protein, 276 aa, synthetic construct
  source 1..276: note = Humanized 21H7 co-binder with VHH1 N-terminal consensus residue, and consensus CDR3B
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 232 - protein, 277 aa, synthetic construct
  source 1..277: note = Humanized 21H7 co-binder with VHH1 N-terminal consensus residue, and consensus CDR3B, and VHH2 with one amino acid consensus addition at N-terminus
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F
  VARIANT 157: note = X is Q or K
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 233 - protein, 278 aa, synthetic construct
  source 1..278: note = Humanized 21H7 co-binder with VHH1 N-terminal consensus residue, and consensus CDR3B, and VHH2 with two amino acid consensus addition at N-terminus
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F
  VARIANT 157: note = X is V
  VARIANT 158: note = X is Q or K
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 234 - protein, 279 aa, synthetic construct
  source 1..279: note = Humanized 21H7 co-binder with VHH1 N-terminal consensus residue, and consensus CDR3B, and VHH2 with three amino acid consensus addition at N-terminus
  VARIANT 1: note = X is E or Q
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
  VARIANT 158: note = X is V
  VARIANT 159: note = X is Q or K
XVQLVESGGGLVQPGGSLRLSCAASGGTFSSYTMGWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 235 - protein, 276 aa, synthetic construct
  source 1..276: note = Humanized 21H7 co-binder with VHH1 N-terminal consensus residue, and consensus CDRs1 and 2B
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 236 - protein, 277 aa, synthetic construct
  source 1..277: note = Humanized 21H7 co-binder with VHH1 N-terminal consensus residue, and consensus CDRs1 and 2B, and VHH2 with one amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 157: note = X is Q or K
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 237 - protein, 278 aa, synthetic construct
  source 1..278: note = Humanized 21H7 co-binder with VHH1 N-terminal consensus residue, and consensus CDRs1 and 2B, and VHH2 with two amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 157: note = X is V
  VARIANT 158: note = X is Q or K
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 238 - protein, 279 aa, synthetic construct
  source 1..279: note = Humanized 21H7 co-binder with VHH1 N-terminal consensus residue, and consensus CDRs1 and 2B, and VHH2 with three amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 157: note = X is E or Q
  VARIANT 158: note = X is V
  VARIANT 159: note = X is Q or K
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGTIGRPYDYWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 239 - protein, 276 aa, synthetic construct
  source 1..276: note = Humanized 21H7 co-binder with VHH1 N-terminal consensus residue, and consensus CDRs1 and 3B
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 240 - protein, 277 aa, synthetic construct
  source 1..277: note = Humanized 21H7 co-binder with VHH1 N-terminal consensus residue, and consensus CDRs1 and 3B, and VHH2 with one amino acid consensus addition at N-terminus
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 106: note = X is E or D
  VARIANT 107: note = X is Y or F
  VARIANT 157: note = X is Q or K
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 241 - protein, 278 aa, synthetic construct
  source 1..278: note = Humanized 21H7 co-binder with VHH1 N-terminal consensus residue, and consensus CDRs1 and 3B, and VHH2 with two amino acid consensus addition at N-terminus
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 106: note = X is E or D
  VARIANT 107: note = X is Y or F
  VARIANT 157: note = X is V
  VARIANT 158: note = X is Q or K
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 242 - protein, 279 aa, synthetic construct
  source 1..279: note = Humanized 21H7 co-binder with VHH1 N-terminal consensus residue, and consensus CDRs1 and 3B, and VHH2 with three amino acid consensus addition at N-terminus
  VARIANT 1: note = X is E or Q
  VARIANT 33: note = X is A or T
  VARIANT 35: note = X is A or G
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
  VARIANT 158: note = X is V
  VARIANT 159: note = X is Q or K
XVQLVESGGGLVQPGGSLRLSCAASGGTFSSYXMXWFRQAPGKEREYVGGISSSGYVMYNSESMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 243 - protein, 276 aa, synthetic construct
  source 1..276: note = Humanized 21H7 co-binder with VHH1 N-terminal consensus residue, and consensus CDRs2B and 3B
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 244 - protein, 277 aa, synthetic construct
  source 1..277: note = Humanized 21H7 co-binder with VHH1 N-terminal consensus residue, and consensus CDRs2B and 3B, and VHH2 with one amino acid consensus addition at N-terminus
  VARIANT 1: note = X is E
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
  VARIANT 157: note = X is Q or K
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 245 - protein, 278 aa, synthetic construct
  source 1..278: note = Humanized 21H7 co-binder with VHH1 N-terminal consensus residue, and consensus CDRs2B and 3B, and VHH2 with two amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
  VARIANT 157: note = X is V
  VARIANT 158: note = X is Q or K
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 246 - protein, 279 aa, synthetic construct
  source 1..279: note = Humanized 21H7 co-binder with VHH1 N-terminal consensus residue, and consensus CDRs2B and 3B, and VHH2 with three amino acid consensus addition at N-terminus
  VARIANT 1: note = X is E or Q
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
  VARIANT 158: note = X is V
  VARIANT 159: note = X is Q or K
XVQLVESGGGLVQPGGSLRLSCAASGGTFSSYTMGWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 247 - protein, 276 aa, synthetic construct
  source 1..276: note = Humanized 21H7 co-binder with VHH1 N-terminal consensus residue, and consensus CDRs1, 2B and 3B
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 248 - protein, 277 aa, synthetic construct
  source 1..277: note = Humanized 21H7 co-binder with VHH1 N-terminal consensus residue, and consensus CDRs1, 2B and 3B, VHH2 with one amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  NON_STD 60: note = X is K or N
  NON_STD 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
  VARIANT 157: note = X is Q or K
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 249 - protein, 278 aa, synthetic construct
  source 1..278: note = Humanized 21H7 co-binder with VHH1 N-terminal consensus residue, and consensus CDRs1, 2B and 3B, and VHH2 with two amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
  VARIANT 157: note = X is V
  VARIANT 158: note = X is Q or K
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 250 - protein, 279 aa, synthetic construct
  source 1..279: note = Humanized 21H7 co-binder with VHH1 N-terminal consensus residue, and consensus CDRs1, 2B and 3B, and VHH2 with three amino acid consensus addition at N-terminus
  VARIANT 1: note = X is E or Q
  VARIANT 33: note = X is A or T
  VARIANT 35: note = X is A or G
  VARIANT 57: note = X is T or V
  VARIANT 58: note = X is L or M
  VARIANT 60: note = X is K or N
  VARIANT 61: note = X is S or A
  VARIANT 62: note = X is D or E
  VARIANT 100: note = X is I, T, or V
  VARIANT 101: note = X is F or I
  VARIANT 106: note = X is E or D
  VARIANT 107: note = X is Y or F
  VARIANT 157: note = X is E or Q
  VARIANT 158: note = X is V
  VARIANT 159: note = X is Q or K
XVQLVESGGGLVQPGGSLRLSCAASGGTFSSYXMXWFRQAPGKEREYVGGISSSGYXXYXXXSMKGRFTISRDNAKNTLYLQMNSLRAEDTAVYYCAAGXXGRPYXXWGQGTQVTVSSGGGSGGGGSGGGGSGGGGSGGGGSGGGGSGGGGSEGVGXXXLVESGGGLVQPGGSLRLSCAASGRTFSTYIIGWFRQAPGKEREFVASISWGGRGTYYADSVKDRFTISRDNPKNMVYLQMNSLRAEDTAVYYCAAGDLPGGRRALDYDYWGQGTLVTVSS

SEQ ID NO: 251 - protein, 5 aa, synthetic construct
  source 1..5: note = Exemplary VHH2 consensus CDR1 (Kabat numbering)
  VARIANT 2: note = X is V, H, or Q
IXAMG

SEQ ID NO: 252 - protein, 16 aa, synthetic construct
  source 1..16: note = Exemplary VHH2 consensus CDR2 (Kabat numbering)
  VARIANT 8: note = X is T or R
AITSGGRXHYRDSVKG

SEQ ID NO: 253 - protein, 12 aa, synthetic construct
  source 1..12: note = Exemplary VHH2 consensus CDR3 (Kabat numbering)
  VARIANT 2: note = X is R, M, or T
  VARIANT 4: note = X is W, L, or R
  VARIANT 5: note = X is T or V
  VARIANT 6: note = X is S or A
DXGXXXVGEYDY

SEQ ID NO: 254 - protein, 119 aa, synthetic construct
  source 1..119: note = 20C7 VHH2 single binder
VQLVESGGGLVQAGGSLRLSCAASGSISSIYAMGWYRQAPGKQRELVAAITYGGRSHYRDSVKGRFTISGNSDNSALYLHMNSLKPEDTAVYYCAADRGLTAVGEYDYWGQGTQVTVSS

SEQ ID NO: 255 - protein, 114 aa, synthetic construct
  source 1..114: note = 1H4 VHH1 single binder
EVQLVESGGGSVQPGGSLRLSCAASGSIFILNVMGWYRQAPGNQRELVALMSSGGSTKYADSVKGRFTISRDNVRDTVYLQMNSLKPEDTAVYYCTLRTENDYWGQGTQVTVSS

SEQ ID NO: 256 - protein, 123 aa, synthetic construct
  source 1..123: note = 1C2 VHH1 single binder
QVQLVESGGGLVQAGGSLRLSCAASGRTFSSYAMGWFRQGPGKEREFVAAISWSGGGTYYADSVKGRFTISRDNAKNTVYLQMNSLKPEDTAVYYCAAGDLPWSRSDLEYDYWGQGTLVTVSS

SEQ ID NO: 257 - protein, 121 aa, synthetic construct
  source 1..121: note = 1E3 VHH2 single binder
LEESGGGSVQAGGSLRLSCAASGRTFSTYAMGWLRQAPGKEREFVAGISWGGGRTDYADSVKDRFTISRDNPKNTVYLQMNSLKPEDTAVYYCAAKDLPSGWGRTSEYDYWGQGTQVTVSS

SEQ ID NO: 258 - protein, 114 aa, synthetic construct
  source 1..114: note = 1D12 VHH2 single binder
EVQLVESGGGSVQPGGSLRLSCAGSGSIFPLNVMGWYRQAPGKEREFIALITSGGSTKHADSVKGRFTISRDNFKNTVYLQMNSLKPEDTAVYYCYLDQSRQYWGQGTLVTVSS

SEQ ID NO: 259 - protein, 111 aa, synthetic construct
  source 1..111: note = 2H2 VHH2 single binder
LVESGGGSVQPGGSLRLSCAASGSIFVLNSMGWYREAPGKERELVAVSTSGGSTGYADSVKGRFTISRENFKNTVYLQMNSLKPEDTAVYYCYLETDRRFWGQGTLVTVSS

SEQ ID NO: 260 - protein, 119 aa, synthetic construct
  source 1..119: note = 1H2 VHH2 single binder
LVESGGGSVQAGGSLRLSCAASGRTFSSYAMGWFRLAPGKEREFVAAISWGGRGTYYADSVKGRFTISRDNAKNTVYLQMNSLKPEDTAVYYCAAGDLPVVAGTEYEYWGQGTQVTVSS

SEQ ID NO: 261 - protein, 122 aa, synthetic construct
  source 1..122: note = 1C7 VHH2 single binder
VQLVESGGGLVQPGGSLRLSCAASGFTFSTYAMGWFRQAPGKEREFVAAISWGGRGTYYADFAKGRFTISRDNAKNTVYLQMNSLKPEDTAAYYCGAGDLPGATVQEEYNYWGQGTQVTVSS

SEQ ID NO: 262 - protein, 114 aa, synthetic construct; identical to SEQ ID NO: 258
  source 1..114: note = 1B10 VHH2 single binder

SEQ ID NO: 263 - protein, 121 aa, synthetic construct
  source 1..121: note = 1G6 VHH2 single binder
QLVESGGCSVQPGESLELSCAASGRIFSLNVMGWYRQVPGKQRELVARISSGGSTSYADSVKGRFTISRGNARDNALNTVFLQMNSVKPEDTAVYYCKLEDFTGGYAMDYWGKGTLVTVSS

SEQ ID NO: 264 - protein, 122 aa, synthetic construct
  source 1..122: note = 1E2 VHH2 single binder
EVQLVESVGGSVQAGGSLRLSCAASGRTFSTYAMGWFRQAPGKEREFVASISWGGRATYYADSVKGRFTISRDNSKNTAYLQMNSLKPEDTAVYYCAAADLPTRVSTAYTFWGQGTQVTVSS

SEQ ID NO: 265 - protein, 114 aa, synthetic construct
  source 1..114: note = 1B12 VHH1 single binder
EVQLVESGGGSVQAGGSLRLSCAASGSIFILNVMGWYRQAPGNQRELVALMSSGGSTKYADSVKGRFTISRDNVRNTVYLQMNSLKPEDTAVYYCTLRTENDYWGQGTQVTVSS

SEQ ID NO: 266 - protein, 118 aa, synthetic construct
  source 1..118: note = 1C3 VHH2 single binder
QLVESGGGSVQAGGSLILSCAASGDIPSIQAMGWYRQAPGKQRELVAAITSGGRTHYRDSVKGRFTISGSNDKSALYLQMNSLKPEDTAVYYCAADRGLTAVGEYDYWGQGTQVTVSS

SEQ ID NO: 267 - protein, 118 aa, synthetic construct; identical to SEQ ID NO: 67
  source 1..118: note = 21A4 VHH1 single binder

SEQ ID NO: 268 - protein, 5 aa, synthetic construct
  source 1..5: note = 21A4 VHH1 CDR1 (Kabat numbering)
SYTMG

SEQ ID NO: 269 - protein, 17 aa, synthetic construct
  source 1..17: note = 21A4 VHH1 CDR2 (Kabat numbering)
GISSSGYVMYNSESMKG

SEQ ID NO: 270 - protein, 9 aa, synthetic construct
  source 1..9: note = 21A4 VHH1 CDR3 (Kabat numbering)
GTIGRPYDY

SEQ ID NO: 271 - protein, 115 aa, synthetic construct
  source 1..115: note = 21A4 VHH2 single binder
VQLVESGGGLVQPGGSLRLSCAASGSIFRLNVMGWYRQAPGEQRELVAAISTGGSTKYADSVKGRFTISRDNVKNTVYLEMNSLKPEDAAVYFCNVRLVGAYDYWGQGTLVTVSS

SEQ ID NO: 272 - protein, 5 aa, synthetic construct
  source 1..5: note = 21A4 VHH2 CDR1 (Kabat numbering)
LNVMG

SEQ ID NO: 273 - protein, 16 aa, synthetic construct
  source 1..16: note = 21A4 VHH2 CDR2 (Kabat numbering)
AISTGGSTKYADSVKG

SEQ ID NO: 274 - protein, 8 aa, synthetic construct
  source 1..8: note = 21A4 VHH2 CDR3 (Kabat numbering)
RLVGAYDY

SEQ ID NO: 275 - protein, 118 aa, synthetic construct; identical to SEQ ID NO: 3
  source 1..118: note = 21C11 VHH1 single binder

SEQ ID NO: 276 - protein, 5 aa, synthetic construct
  source 1..5: note = 21C11 VHH1 CDR1 (Kabat numbering)
SYTMG

SEQ ID NO: 277 - protein, 17 aa, synthetic construct
  source 1..17: note = 21C11 VHH1 CDR2 (Kabat numbering)
GISSSGYVMYNSESMKG

SEQ ID NO: 278 - protein, 9 aa, synthetic construct
  source 1..9: note = 21C11 VHH1 CDR3 (Kabat numbering)
GTIGRPYDY

SEQ ID NO: 279 - protein, 121 aa, synthetic construct
  source 1..121: note = 21C11 VHH2 single binder
VQLVESGGDLVQAGGSLRLSCAASGRTFSTYIIGWFRQAPGKEREFVTAISWGGRGTYYADSVKGRFTISRDNAKNTVYLQMNSLRPEDTAVYFCAAGDLMVSTRSQYDYWGQGTLVTVSS

SEQ ID NO: 280 - protein, 5 aa, synthetic construct
  source 1..5: note = 21C11 VHH2 CDR1 (Kabat numbering)
TYIIG

SEQ ID NO: 281 - protein, 17 aa, synthetic construct
  source 1..17: note = 21C11 VHH2 CDR2 (Kabat numbering)
AISWGGRGTYYADSVKG

SEQ ID NO: 282 - protein, 13 aa, synthetic construct
  source 1..13: note = 21C11 VHH2 CDR3 (Kabat numbering)
GDLMVSTRSQYDY

SEQ ID NO: 283 - protein, 118 aa, synthetic construct
  source 1..118: note = 21C8 VHH1 single binder
EVQLVESGGGSVQAGGSLRLSCAASGGTFSSYTMGWFRQAPGKEREYVGGISSSGYVMYNSESMKGRFTISRENAKNMVYLQMNSLKPEDTAVYYCAAGTIGRPYDYWGQGTLVTVSS

SEQ ID NO: 284 - protein, 5 aa, synthetic construct
  source 1..5: note = 21C8 VHH1 CDR1 (Kabat numbering)
SYTMG

SEQ ID NO: 285 - protein, 17 aa, synthetic construct
  source 1..17: note = 21C8 VHH1 CDR2 (Kabat numbering)
GISSSGYVMYNSESMKG

SEQ ID NO: 286 - protein, 9 aa, synthetic construct
  source 1..9: note = 21C8 VHH1 CDR3 (Kabat numbering)
GTIGRPYDY

SEQ ID NO: 287 - protein, 121 aa, synthetic construct
  source 1..121: note = 21C8 VHH2 single binder
QLVESGGDLVQPGGSLRLSCAASGRTFSTYAMGWFRQAPGKEREFVAAISWGGRGTYYADSVRGRFTISRDNPKNTVFLQMNSLKPDDTAVYYCAAGDLPGGRRALDYDYWGQGTQVTVSS

SEQ ID NO: 288 - protein, 5 aa, synthetic construct
  source 1..5: note = 21C8 VHH2 CDR1 (Kabat numbering)
TYAMG

SEQ ID NO: 289 - protein, 17 aa, synthetic construct
  source 1..17: note = 21C8 VHH2 CDR2 (Kabat numbering)
AISWGGRGTYYADSVRG

SEQ ID NO: 290 - protein, 14 aa, synthetic construct
  source 1..14: note = 21C8 VHH2 CDR3 (Kabat numbering)
GDLPGGRRALDYDY

SEQ ID NO: 291 - protein, 120 aa, synthetic construct
  source 1..120: note = 21B11 VHH1 single binder
EVQLVESGGGLVQPGGSLSLSCTASGSITSIVAMGWYRQTPGKQRELVAAITSGGRTHYRDSVKGRFTISGNNDNSALYLHMNSLKPEDTAVYYCAADRGWTSVGEYDYWGQGTLVTVSS

SEQ ID NO: 292 - protein, 5 aa, synthetic construct
  source 1..5: note = 21B11 VHH1 CDR1 (Kabat numbering)
IVAMG

SEQ ID NO: 293 - protein, 16 aa, synthetic construct
  source 1..16: note = 21B11 VHH1 CDR2 (Kabat numbering)
AITSGGRTHYRDSVKG

SEQ ID NO: 294 - protein, 12 aa, synthetic construct
  source 1..12: note = 21B11 VHH1 CDR3 (Kabat numbering)
DRGWTSVGEYDY

SEQ ID NO: 295 - protein, 123 aa, synthetic construct
  source 1..123: note = 21B11 VHH2 single binder
QLVESGGGSVQAGASLRLSCVSSGPTYGFYVTAWFRQAPGKEREFVAAVRGIASRVNYADSVKGRFTISRDNAANTIFLQMNSLKPEDTAVYYCALRRQYSTNYDSSTAYDVWGQGTLVTVSS

SEQ ID NO: 296 - protein, 5 aa, synthetic construct
  source 1..5: note = 21B11 VHH2 CDR1 (Kabat numbering)
FYVTA

SEQ ID NO: 297 - protein, 17 aa, synthetic construct
  source 1..17: note = 21B11 VHH2 CDR2 (Kabat numbering)
AVRGIASRVNYADSVKG

SEQ ID NO: 298 - protein, 16 aa, synthetic construct
  source 1..16: note = 21B11 VHH2 CDR3 (Kabat numbering)
RRQYSTNYDSSTAYDV

SEQ ID NO: 299 - protein, 125 aa, synthetic construct
  source 1..125: note = 21G5 VHH1 single binder
EVQLVESGGGSVQAGASLRLSCVSSGPTYGFYVTAWFRQAPGKEREFVAAVRGIASRVNYADSVKGRFTISRDNAANTIFLQMNSLKPEDTAVYYCALRRQYSTNYDSSTAYDVWGQGTLVTVSS

SEQ ID NO: 300 - protein, 5 aa, synthetic construct
  source 1..5: note = 21G5 VHH1 CDR1 (Kabat numbering)
FYVTA

SEQ ID NO: 301 - protein, 17 aa, synthetic construct
  source 1..17: note = 21G5 VHH1 CDR2 (Kabat numbering)
AVRGIASRVNYADSVKG

SEQ ID NO: 302 - protein, 16 aa, synthetic construct
  source 1..16: note = 21G5 VHH1 CDR3 (Kabat numbering)
RRQYSTNYDSSTAYDV

SEQ ID NO: 303 - protein, 117 aa, synthetic construct
  source 1..117: note = 21G5 VHH2 single binder
LEESGGGLVQPGGSLILSCAASGDIPSIVAMGWYRQAPGKQRELVAAITSGGRTHYRDSVKGRFTISGNNDNSALYLHMNSLKPEDTAVYYCAADRGLTAVGEYDYWGQGTQVTVSS

SEQ ID NO: 304 - protein, 5 aa, synthetic construct
  source 1..5: note = 21G5 VHH2 CDR1 (Kabat numbering)
IVAMG

SEQ ID NO: 305 - protein, 16 aa, synthetic construct
  source 1..16: note = 21G5 VHH2 CDR2 (Kabat numbering)
AITSGGRTHYRDSVKG

SEQ ID NO: 306 - protein, 12 aa, synthetic construct
  source 1..12: note = 21G5 VHH2 CDR3 (Kabat numbering)
DRGLTAVGEYDY

SEQ ID NO: 307 - protein, 115 aa, synthetic construct
  source 1..115: note = 21G7 VHH1 single binder
EVQLVESGGGLVQPGGSLRLSCAASGSGFGIGRMGWYRQAPGKQRELVAIISSLTGTTYADSVKGRFTGSRDSAKNMVYLRMDSLKPEDTAVYYCYANRFTVDYWGQGTLVTVSS

SEQ ID NO: 308 - protein, 5 aa, synthetic construct
  source 1..5: note = 21G7 VHH1 CDR1 (Kabat numbering)
IGRMG

SEQ ID NO: 309 - protein, 16 aa, synthetic construct
  source 1..16: note = 21G7 VHH1 CDR2 (Kabat numbering)
IISSLTGTTYADSVKG

SEQ ID NO: 310 - protein, 7 aa, synthetic construct
  source 1..7: note = 21G7 VHH1 CDR3 (Kabat numbering)
NRFTVDY

SEQ ID NO: 311 - protein, 120 aa, synthetic construct
  source 1..120: note = 21G7 VHH2 single binder
EVQLVESGGGLVQAGGSLRLSCAASGDIPSIVAMGWYRQAPGKQRELVAAITSGGRTHYRDSVKGRFTISGNNDNSALYLHMNSLKPEDTAVYYCAADRGLTAVGEYDYWGQGTLVTVSS

SEQ ID NO: 312 - protein, 5 aa, synthetic construct
  source 1..5: note = 21G7 VHH2 CDR1 (Kabat numbering)
IVAMG

SEQ ID NO: 313 - protein, 16 aa, synthetic construct
  source 1..16: note = 21G7 VHH2 CDR2 (Kabat numbering)
AITSGGRTHYRDSVKG

SEQ ID NO: 314 - protein, 12 aa, synthetic construct
  source 1..12: note = 21G7 VHH2 CDR3 (Kabat numbering)
DRGLTAVGEYDY

SEQ ID NO: 315 - protein, 125 aa, synthetic construct
  source 1..125: note = 21H2 VHH1 single binder
EVQLVESGGGLVQAGGSLRLSCAASGRTSSVFSTAWFRRAPGKEREFVGNIRGIADRTDYADSVKGRFTISRDNAKNTVYLQMNTLKPEDTAVYYCAAKRSYTRDYVTDYNYDYWGQGTLVTVSS

SEQ ID NO: 316 - protein, 5 aa, synthetic construct
  source 1..5: note = 21H2 VHH1 CDR1 (Kabat numbering)
VFSTA

SEQ ID NO: 317 - protein, 17 aa, synthetic construct
  source 1..17: note = 21H2 VHH1 CDR2 (Kabat numbering)
NIRGIADRTDYADSVKG

SEQ ID NO: 318 - protein, 16 aa, synthetic construct
  source 1..16: note = 21H2 VHH1 CDR3 (Kabat numbering)
KRSYTRDYVTDYNYDY

SEQ ID NO: 319 - protein, 117 aa, synthetic construct
  source 1..117: note = 21H2 VHH2 single binder
LVESGGGLVQAGGSLILSCAASGDISSIVAMGWYRQAPGKQRELVAAITSGGRTHYRDSVKGRFTISGNNDNSALYLHMNSLKPEDTAVYYCAADRGWTSVGEYDYWGQGTLVTVSS

SEQ ID NO: 320 - protein, 5 aa, synthetic construct
  source 1..5: note = 21H2 VHH2 CDR1 (Kabat numbering)
IVAMG

SEQ ID NO: 321 - protein, 16 aa, synthetic construct
  source 1..16: note = 21H2 VHH2 CDR2 (Kabat numbering)
AITSGGRTHYRDSVKG

SEQ ID NO: 322 - protein, 12 aa, synthetic construct
  source 1..12: note = 21H2 VHH2 CDR3 (Kabat numbering)
DRGWTSVGEYDY

SEQ ID NO: 323 - protein, 115 aa, synthetic construct
  source 1..115: note = 19B5 VHH1 single binder
EVQLVESGGGLVQPGGSLRLSCAASGSGFGIGRMGWYRQAPGKQRELVAIISSLSGTTYTDSVKGRFTISRDIAKNMVYLRMDSLKPEDTAVYYCYANRFTVDYWGQGTQVTVSS

SEQ ID NO: 324 - protein, 5 aa, synthetic construct
  source 1..5: note = 19B5 VHH1 CDR1 (Kabat numbering)
IGRMG

SEQ ID NO: 325 - protein, 16 aa, synthetic construct
  source 1..16: note = 19B5 VHH1 CDR2 (Kabat numbering)
IISSLSGTTYTDSVKG

SEQ ID NO: 326 - protein, 7 aa, synthetic construct
  source 1..7: note = 19B5 VHH1 CDR3 (Kabat numbering)
NRFTVDY

SEQ ID NO: 327 - protein, 119 aa, synthetic construct
  source 1..119: note = 19B5 VHH2 single binder
VQLVESVGGLVQAGGSLRLSCAASGDIPSIQAMGWYRQAPGKQRELVAAITSGGRTHYRDSVKGRFTISGSNDKSALYLQMNSLKPEDTAVYYCAADRGLTAVGEYDYWGQGTLVTVSS

SEQ ID NO: 328 - protein, 5 aa, synthetic construct
  source 1..5: note = 19B5 VHH2 CDR1 (Kabat numbering)
IQAMG

SEQ ID NO: 329 - protein, 16 aa, synthetic construct
  source 1..16: note = 19B5 VHH2 CDR2 (Kabat numbering)
AITSGGRTHYRDSVKG

SEQ ID NO: 330 - protein, 12 aa, synthetic construct
  source 1..12: note = 19B5 VHH2 CDR3 (Kabat numbering)
DRGLTAVGEYDY

SEQ ID NO: 331 - protein, 115 aa, synthetic construct
  source 1..115: note = 22D1 VHH1 single binder
EVQLVESGGGSVQPGGSLRLSCAASGSGFGVGRMGWYRQAPGNKRELVAIVSSLTGTTYADSAKGRFTISRDSAKNMVYLRMDSLKPEDTAVYYCYANRFTVDYWGQGTLVTVSS

SEQ ID NO: 332 - protein, 5 aa, synthetic construct
  source 1..5: note = 22D1 VHH1 CDR1 (Kabat numbering)
VGRMG

SEQ ID NO: 333 - protein, 16 aa, synthetic construct
  source 1..16: note = 22D1 VHH1 CDR2 (Kabat numbering)
IVSSLTGTTYADSAKG

SEQ ID NO: 334 - protein, 7 aa, synthetic construct
  source 1..7: note = 22D1 VHH1 CDR3 (Kabat numbering)
NRFTVDY

SEQ ID NO: 335 - protein, 120 aa, synthetic construct
  source 1..120: note = 22D1 VHH2 single binder
EVQLVESGGGLVQPGGSLTLSCAASGDILSIVAMGWYRQAPGKQRELVAAITSGGRTHYRDSVKGRFTISGNNDNSALYLHMNSLKPEDTAVYYCAADRGLTAVGEYDYWGQGTQVTVSS

SEQ ID NO: 336 - protein, 5 aa, synthetic construct
  source 1..5: note = 22D1 VHH2 CDR1 (Kabat numbering)
IVAMG

SEQ ID NO: 337 - protein, 16 aa, synthetic construct
  source 1..16: note = 22D1 VHH2 CDR2 (Kabat numbering)
AITSGGRTHYRDSVKG

SEQ ID NO: 338 - protein, 12 aa, synthetic construct
  source 1..12: note = 22D1 VHH2 CDR3 (Kabat numbering)
DRGLTAVGEYDY

SEQ ID NO: 339 - protein, 125 aa, synthetic construct; identical to SEQ ID NO: 299
  source 1..125: note = 22D8 VHH1 single binder

SEQ ID NO: 340 - protein, 5 aa, synthetic construct
  source 1..5: note = 22D8 VHH1 CDR1 (Kabat numbering)
FYVTA

SEQ ID NO: 341 - protein, 17 aa, synthetic construct
  source 1..17: note = 22D8 VHH1 CDR2 (Kabat numbering)
AVRGIASRVNYADSVKG

SEQ ID NO: 342 - protein, 16 aa, synthetic construct
  source 1..16: note = 22D8 VHH1 CDR3 (Kabat numbering)
RRQYSTNYDSSTAYDV

SEQ ID NO: 343 - protein, 117 aa, synthetic construct
  source 1..117: note = 22D8 VHH2 single binder
LVESGGGLVQAGGSLILSCAASGDIPSIVAMGWYRQAPGKQRELVAAITSGGRTHYRDSVKGRFTISGNNDNSALYLHMNSLKPEDTAVYYCAADRGLTAVGEYDYWGQGTLVTVSS

SEQ ID NO: 344 - protein, 5 aa, synthetic construct
  source 1..5: note = 22D8 VHH2 CDR1 (Kabat numbering)
IVAMG

SEQ ID NO: 345 - protein, 16 aa, synthetic construct
  source 1..16: note = 22D8 VHH2 CDR2 (Kabat numbering)
AITSGGRTHYRDSVKG

SEQ ID NO: 346 - protein, 12 aa, synthetic construct
  source 1..12: note = 22D8 VHH2 CDR3 (Kabat numbering)
DRGLTAVGEYDY

(19) United States
(12) Patent Application Publication
(10) Pub. No.: US 2026/0250374 A1
(43) Pub. Date: Aug. 27, 2026

(54) IL31-BINDING POLYPEPTIDES AND USES THEREOF

(71) Applicant: Attovia Therapeutics, Inc., San Carlos, CA (US)

(72) Inventors: Jin Wook CHOI, Pleasanton, CA (US); Lam NGUYEN, Union City, CA (US); Shyr Jiann LI, Millbrae, CA (US); Jing ZHANG, San Jose, CA (US); David BELLOVIN, San Jose, CA (US); Ole Petter VEIBY, Redwood City, CA (US); Hangjun ZHAN, Foster City, CA (US)

(73) Assignee: Attovia Therapeutics, Inc., San Carlos, CA (US)

(21) Appl. No.: 19/400,178

(22) Filed: Nov. 25, 2025

Related U.S. Application Data
(63) Continuation of application No. 19/187,342, filed on Apr. 23, 2025, now Pat. No. 12,509,510.
(60) Provisional application No. 63/637,775, filed on Apr. 23, 2024.
(60) Provisional application No. 63/690,297, filed on Sep. 3, 2024.
(60) Provisional application No. 63/770,198, filed on Mar. 11, 2025.

(51) Int. Cl. C07K 16/24; A61K 39/00

(52) U.S. Cl. CPC C07K 16/244; A61K 2039/505; C07K 2317/24; C07K 2317/52; C07K 2317/569; C07K 2317/92; C07K 2317/94

(57) ABSTRACT

Provided herein are polypeptides that bind IL31 and methods of using IL31-binding polypeptides to modulate the biological activity of IL31. Also provided herein are nucleic acids, host cells, and methods of preparing polypeptides that bind IL31. Uses and methods provided herein include, but are not limited to, methods of treating an IL31-associated condition, such as a pruritic condition, atopic dermatitis, prurigo nodularis, and chronic spontaneous urticaria. Also provided herein are methods of detecting using the polypeptides that bind IL31.

### CROSS-REFERENCE TO RELATED APPLICATIONS

This application is a continuation of U.S. application Ser. No. 19/187,342, filed Apr. 23, 2025, which claims the benefit of U.S. provisional patent application No. 63/637,775, filed Apr. 23, 2024, U.S. provisional patent application No. 63/690,297, filed Sep. 3, 2024, and U.S. provisional patent application No. 63/770,198, filed Mar. 11, 2025, each of which is incorporated herein by reference in its entirety.

### REFERENCE TO SEQUENCE LISTING SUBMITTED ELECTRONICALLY

The content of the electronically submitted sequence listing 1 (Name: 5614_0020004_SequenceListing_ST26.xml; Size: 843,079 bytes; and Date of Creation: Apr. 23, 2025), filed with the application, is incorporated herein by reference in its entirety.

### FIELD

The present disclosure relates to IL31-binding polypeptides, and methods of using IL31-binding polypeptides to modulate the biological activity of IL31. Such methods include, but are not limited to, methods of treating pruritic conditions, atopic dermatitis, prurigo nodularis, and chronic spontaneous urticaria.

### BACKGROUND

Interleukin 31 (IL31) is a cytokine mostly produced by Th2 cells and has been implicated in the pathophysiology of several skin disorders, such as pruritic conditions, allergic diseases, and other inflammatory conditions. IL31 functions by binding IL31 receptor A (IL31RA)/oncostatin M receptor beta (OSMRB) and activation of downstream JAK/STAT and PI3K/AKT pathways, which mediates inflammatory responses, initiating immunoregulatory circuits, stimulating itch, and neuronal outgrowth. Many of the clinical problems associated with dermatitis and other skin disorders are thought to be associated with IL31 activity.

Chronic inflammatory skin diseases such as atopic dermatitis (AD) and prurigo nodularis (PN) lead to intensely pruritic skin lesions resulting in severe scratching. Topical steroids and calcineurin inhibitors are not sufficient to manage symptoms in AD or PN patients. Nemolizumab, an antibody that targets IL31RA, has shown promising anti-pruritic efficacy, validating the IL31-IL31RA pathway in providing symptom relief in AD and PN patients. Overall, however, there remains a strong need for improved therapies for treating IL31-associated conditions, including therapies that have the potential to induce fast and prolonged control of symptoms in patients with chronic inflammatory skin disease.

### SUMMARY

Provided herein are high affinity and potent biparatopic polypeptides that bind to the IL31 ligand, which has the potential to induce fast and prolonged control of pruritus in patients with chronic inflammatory skin disease.

The polypeptides disclosed herein provide for multiple advantages and improvements over standard-of-care treatments and/or treatments still in development. Such advantages and improvements include, but are not limited to, greater efficacy against lesions and itches, greater safety (e.g., less conjunctivitis), rapid itch relief (days to weeks), extended half-life, avoidance of target-mediated drug disposition (TMDD), and more convenient and/or less frequent dosing, which leads to reduced injection burden. Such advantages and improvements allow the polypeptides disclosed herein to be used as treatment options for nonresponding and/or relapsed patients who received standard-of-care treatments and/or treatments still in development.

In some aspects, polypeptides that bind IL31 comprising one or more IL31 binder are provided herein.

In some aspects, provided herein is a polypeptide that binds IL31 comprising two or more IL31 binders, wherein the first IL31 binder comprises a VHH1 which comprises a CDR1 comprising the amino acid sequence of SEQ ID NO: 59; a CDR2 comprising the amino acid sequence of SEQ ID NO: 63; and a CDR3 comprising the amino acid sequence of SEQ ID NO: 64 and the second IL31 binder comprises a VHH2 that binds to IL31.

In some aspects, provided herein is a polypeptide that binds IL31 comprising two or more IL31 binders, wherein the first IL31 binder comprises a VHH1 which comprises a CDR1 comprising the amino acid sequence of SEQ ID NO: 59; a CDR2 comprising the amino acid sequence of SEQ ID NO: 60 or SEQ ID NO: 63; and a CDR3 comprising the amino acid sequence of SEQ ID NO: 61 or SEQ ID NO: 64 and the second IL31 binder comprising a VHH2 which binds to IL31. In some aspects, the VHH1 comprises a CDR1 comprising the amino acid sequence of SEQ ID NO: 59; a CDR2 comprising the amino acid sequence of SEQ ID NO: 63; and a CDR3 comprising the amino acid sequence of SEQ ID NO: 61. In some aspects, the VHH1 comprises a CDR1 comprising the amino acid sequence of SEQ ID NO: 59; a CDR2 comprising the amino acid sequence of SEQ ID NO: 60; and a CDR3 comprising the amino acid sequence of SEQ ID NO: 61. In some aspects, the VHH1 comprises a CDR1 comprising the amino acid sequence of SEQ ID NO: 59; a CDR2 comprising the amino acid sequence of SEQ ID NO: 60; and a CDR3 comprising the amino acid sequence of SEQ ID NO: 64.

In some aspects, the polypeptide that binds IL31 comprises a first IL31 binder and a second IL31 binder.

In some aspects, the first IL31 binder comprises a VHH1 comprising a CDR1 comprising the amino acid sequence of SEQ ID NO: 5, SEQ ID NO: 19, SEQ ID NO: 33, SEQ ID NO: 47, SEQ ID NO: 78, SEQ ID NO: 81, or SEQ ID NO: 59; a CDR2 comprising the amino acid sequence of SEQ ID NO: 6, SEQ ID NO: 20, SEQ ID NO: 34, SEQ ID NO: 48, SEQ ID NO: 79, SEQ ID NO: 82, SEQ ID NO: 60, or SEQ ID NO: 63; and a CDR3 comprising the amino acid sequence of SEQ ID NO: 7, SEQ ID NO: 21, SEQ ID NO: 35, SEQ ID NO: 49, SEQ ID NO: 80, SEQ ID NO: 83, SEQ ID NO: 61, or SEQ ID NO: 64.

In some aspects, the first IL31 binder comprises a VHH1 comprising a CDR1 comprising the amino acid sequence of SEQ ID NO: 268, SEQ ID NO: 276, SEQ ID NO: 284, SEQ ID NO: 292, SEQ ID NO: 300, SEQ ID NO: 308, SEQ ID NO: 316, SEQ ID NO: 324, SEQ ID NO: 332, and SEQ ID NO: 340; a CDR2 comprising the amino acid sequence of SEQ ID NO: 269, SEQ ID NO: 277, SEQ ID NO: 285, SEQ ID NO: 293, SEQ ID NO: 301, SEQ ID NO: 309, SEQ ID NO: 317, SEQ ID NO: 325, SEQ ID NO: 333, and SEQ ID NO: 341; and a CDR3 comprising the amino acid sequence of SEQ ID NO: 270; SEQ ID NO: 278, SEQ ID NO: 286, SEQ ID NO: 294, SEQ ID NO: 302, SEQ ID NO: 310, SEQ ID NO: 318, SEQ ID NO: 326, SEQ ID NO: 334, and SEQ ID NO: 342.

In some aspects, the second IL31 binder comprises a VHH2 comprising a CDR1 comprising the amino acid sequence of SEQ ID NO: 10, SEQ ID NO: 24, SEQ ID NO: 38, SEQ ID NO: 52, SEQ ID NO: 84, SEQ ID NO: 87, or SEQ ID NO: 251; a CDR2 comprising the amino acid sequence of SEQ ID NO: 11, SEQ ID NO: 25, SEQ ID NO: 39, SEQ ID NO: 53, SEQ ID NO: 85, SEQ ID NO: 88, or SEQ ID NO: 252; and a CDR3 comprising the amino acid sequence of SEQ ID NO: 12, SEQ ID NO: 26, SEQ ID NO: 40, SEQ ID NO: 54, SEQ ID NO: 86, SEQ ID NO: 89, or SEQ ID NO: 253.

In some aspects, the second IL31 binder comprises a VHH2 comprising a CDR1 comprising the amino acid sequence of SEQ ID NO: 272, SEQ ID NO: 280, SEQ ID NO: 288, SEQ ID NO: 296, SEQ ID NO: 304, SEQ ID NO: 312, SEQ ID NO: 320, SEQ ID NO: 328, SEQ ID NO: 336, SEQ ID NO: 344; a CDR2 comprising the amino acid sequence of SEQ ID NO: 273, SEQ ID NO: 281, SEQ ID NO: 289, SEQ ID NO: 297, SEQ ID NO: 305, SEQ ID NO: 313, SEQ ID NO: 321, SEQ ID NO: 329, SEQ ID NO: 337, and SEQ ID NO: 345; and a CDR3 comprising the amino acid sequence of SEQ ID NO: 274, SEQ ID NO: 282, SEQ ID NO: 290, SEQ ID NO: 298, SEQ ID NO: 306, SEQ ID NO: 314, SEQ ID NO: 322, SEQ ID NO: 330, SEQ ID NO: 338, and SEQ ID NO: 346.

In some aspects, a) the first IL31 binder comprises a VHH1 comprising a CDR1 comprising the amino acid sequence of SEQ ID NO: 5, SEQ ID NO: 19, SEQ ID NO: 33, SEQ ID NO: 47, SEQ ID NO: 78, SEQ ID NO: 81, or SEQ ID NO: 59; a CDR2 comprising the amino acid sequence of SEQ ID NO: 6, SEQ ID NO: 20, SEQ ID NO: 34, SEQ ID NO: 48, SEQ ID NO: 79, SEQ ID NO: 82, SEQ ID NO: 60, or SEQ ID NO: 63; and a CDR3 comprising the amino acid sequence of SEQ ID NO: 7, SEQ ID NO: 21, SEQ ID NO: 35, SEQ ID NO: 49, SEQ ID NO: 80, SEQ ID NO: 83, SEQ ID NO: 61, or SEQ ID NO: 64; and b) the second IL31 binder comprises a VHH2 comprising a CDR1 comprising the amino acid sequence of SEQ ID NO: 10, SEQ ID NO: 24, SEQ ID NO: 38, SEQ ID NO: 52, SEQ ID NO: 84, SEQ ID NO: 87, SEQ ID NO: 251; a CDR2 comprising the amino acid sequence of SEQ ID NO: 11, SEQ ID NO: 25, SEQ ID NO: 39, SEQ ID NO: 53, SEQ ID NO: 85, SEQ ID NO: 88, or SEQ ID NO: 252; and a CDR3 comprising the amino acid sequence of SEQ ID NO: 12, SEQ ID NO: 26, SEQ ID NO: 40, SEQ ID NO: 54, SEQ ID NO: 86, SEQ ID NO: 89, or SEQ ID NO: 253.

In some aspects, a) the first IL31 binder comprises a VHH1 comprising a CDR1 comprising the amino acid sequence of SEQ ID NO: 268, SEQ ID NO: 276, SEQ ID NO: 284, SEQ ID NO: 292, SEQ ID NO:300, SEQ ID NO:308, SEQ ID NO:316, SEQ ID NO:324, SEQ ID NO: 332, and SEQ ID NO:340; a CDR2 comprising the amino acid sequence of SEQ ID NO: 269, SEQ ID NO: 277, SEQ ID NO: 285, SEQ ID NO: 293, SEQ ID NO:301, SEQ ID NO:309, SEQ ID NO: 317, SEQ ID NO:325, SEQ ID NO:333, and SEQ ID NO:341; and a CDR3 comprising the amino acid sequence of SEQ ID NO: 270; SEQ ID NO: 278, SEQ ID NO: 286, SEQ ID NO: 294, SEQ ID NO:302, SEQ ID NO:310, SEQ ID NO:318, SEQ ID NO:326, SEQ ID NO:334, and SEQ ID NO: 342; and b) the second IL31 binder comprises a VHH2 comprising a CDR1 comprising the amino acid sequence of SEQ ID NO: 272, SEQ ID NO: 280, SEQ ID NO: 288, SEQ ID NO: 296, SEQ ID NO:304, SEQ ID NO:312, SEQ ID NO:320, SEQ ID NO:328, SEQ ID NO:336, SEQ ID NO: 344; a CDR2 comprising the amino acid sequence of SEQ ID NO: 273, SEQ ID NO: 281, SEQ ID NO: 289, SEQ ID NO: 297, SEQ ID NO:305, SEQ ID NO:313, SEQ ID NO:321, SEQ ID NO: 329, SEQ ID NO:337, and SEQ ID NO:345; and a CDR3 comprising the amino acid sequence of SEQ ID NO: 274, SEQ ID NO: 282, SEQ ID NO: 290, SEQ ID NO: 298, SEQ ID NO:306, SEQ ID NO: 314, SEQ ID NO:322, SEQ ID NO:330, SEQ ID NO:338, and SEQ ID NO:346.

In some aspects, the VHH1 comprises: a) a CDR1 comprising the amino acid sequence of SEQ ID NO: 5, a CDR2 comprising the amino acid sequence of SEQ ID NO: 6, and a CDR3 comprising the amino acid sequence of SEQ ID NO: 7; b) a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21; c) a CDR1 comprising the amino acid sequence of SEQ ID NO: 33, a CDR2 comprising the amino acid sequence of SEQ ID NO: 34, and a CDR3 comprising the amino acid sequence of SEQ ID NO: 35; d) a CDR1 comprising the amino acid sequence of SEQ ID NO: 47, a CDR2 comprising the amino acid sequence of SEQ ID NO: 48, and a CDR3 comprising the amino acid sequence of SEQ ID NO: 49; e) a CDR1 comprising the amino acid sequence of SEQ ID NO: 78, a CDR2 comprising the amino acid sequence of SEQ ID NO: 79, and a CDR3 comprising the amino acid sequence of SEQ ID NO: 80; or f) a CDR1 comprising the amino acid sequence of SEQ ID NO: 81, a CDR2 comprising the amino acid sequence of SEQ ID NO: 82, and a CDR3 comprising the amino acid sequence of SEQ ID NO: 83.

In some aspects, provided herein is a polypeptide that binds IL31 comprising two or more IL31 binders, wherein the first IL31 binder comprises a VHH1 binding to IL31, and the second IL31 binder comprises a VHH2 which comprises:
a. a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12;
b. a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26;
c. a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40;
d. a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54;
e. a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86; or
f. a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89.

In some aspects, provided herein is a polypeptide that binds IL31 comprising two or more IL31 binders, wherein the first IL31 binder comprises a VHH1 binding to IL31, and the second IL31 binder comprises a VHH2 which comprises a CDR1 comprising the amino acid sequence of SEQ ID NO: 251, a CDR2 comprising the amino acid sequence of SEQ ID NO: 252, and a CDR3 comprising the amino acid sequence of SEQ ID NO: 253.

In some aspects, the VHH2 comprises: a) a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12; b) a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26; c) a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40; d) a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54; e) a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86; or f) a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89. In some aspects, the VHH2 comprises a CDR1 comprising the amino acid sequence of SEQ ID NO: 251, a CDR2 comprising the amino acid sequence of SEQ ID NO: 252, and a CDR3 comprising the amino acid sequence of SEQ ID NO: 253.

In some aspects, the VHH1 comprises a CDR1 comprising the amino acid sequence of SEQ ID NO: 5, a CDR2 comprising the amino acid sequence of SEQ ID NO: 6, and a CDR3 comprising the amino acid sequence of SEQ ID NO: 7; and the VHH2 comprises a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12.

In some aspects, the VHH1 comprises a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21; and the VHH2 comprises a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26.

In some aspects, the VHH1 comprises a CDR1 comprising the amino acid sequence of SEQ ID NO: 33, a CDR2 comprising the amino acid sequence of SEQ ID NO: 34, and a CDR3 comprising the amino acid sequence of SEQ ID NO: 35; and the VHH2 comprises a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40.

In some aspects, the VHH1 comprises a CDR1 comprising the amino acid sequence of SEQ ID NO: 47, a CDR2 comprising the amino acid sequence of SEQ ID NO: 48, and a CDR3 comprising the amino acid sequence of SEQ ID NO: 49; and the VHH2 comprises a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54.

In some aspects, the VHH1 comprises a CDR1 comprising the amino acid sequence of SEQ ID NO: 78, a CDR2 comprising the amino acid sequence of SEQ ID NO: 79, and a CDR3 comprising the amino acid sequence of SEQ ID NO: 80; and the VHH2 comprises a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86.

In some aspects, the VHH1 comprises a CDR1 comprising the amino acid sequence of SEQ ID NO: 81, a CDR2 comprising the amino acid sequence of SEQ ID NO: 82, and a CDR3 comprising the amino acid sequence of SEQ ID NO: 83; and the VHH2 comprises a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89.

In some aspects, the VHH1 and/or the VHH2 are humanized.

In some aspects, the VHH1 comprises an amino acid sequence at least 85%, at least 90%, at least 95%, at least 96%, at least 97%, at least 98%, or at least 99% identical to the amino acid sequence of SEQ ID NO: 3, SEQ ID NO: 17, SEQ ID NO: 31, or SEQ ID NO: 45. In some aspects, the VHH1 comprises the amino acid sequence of SEQ ID NO: 3, SEQ ID NO: 17, SEQ ID NO: 31, or SEQ ID NO: 45.

In some aspects, the VHH1 comprises an amino acid sequence at least 85%, at least 90%, at least 95%, at least 96%, at least 97%, at least 98%, or at least 99% identical to the amino acid sequence of SEQ ID NO: 4, SEQ ID NO: 18, SEQ ID NO: 32, or SEQ ID NO: 46. In some aspects, the VHH1 comprises the amino acid sequence of SEQ ID NO: 4, SEQ ID NO: 18, SEQ ID NO: 32, or SEQ ID NO: 46.

In some aspects, the VHH2 comprises an amino acid sequence at least 85%, at least 90%, at least 95%, at least 96%, at least 97%, at least 98%, or at least 99% identical to the amino acid sequence of SEQ ID NO: 8, SEQ ID NO: 22, SEQ ID NO: 36, or SEQ ID NO: 50. In some aspects, the VHH2 comprises the amino acid sequence of SEQ ID NO: 8, SEQ ID NO: 22, SEQ ID NO: 36, or SEQ ID NO: 50.

In some aspects, the VHH2 comprises an amino acid sequence at least 85%, at least 90%, at least 95%, at least 96%, at least 97%, at least 98%, or at least 99% identical to the amino acid sequence of SEQ ID NO: 9, SEQ ID NO: 23, SEQ ID NO: 37, or SEQ ID NO: 51. In some aspects, the VHH2 comprises the amino acid sequence of SEQ ID NO: 9, SEQ ID NO: 23, SEQ ID NO: 37, or SEQ ID NO: 51.

In some aspects, the VHH1 comprises the amino acid sequence of SEQ ID NO: 4 and the VHH2 comprises the amino acid sequence of SEQ ID NO: 9. In some aspects, the VHH1 comprises the amino acid sequence of SEQ ID NO: 18 and the VHH2 comprises the amino acid sequence of SEQ ID NO: 23. In some aspects, the VHH1 comprises the amino acid sequence of SEQ ID NO: 32 and the VHH2 comprises the amino acid sequence of SEQ ID NO: 37. In some aspects, the VHH1 comprises the amino acid sequence of SEQ ID NO: 46 and the VHH2 comprises the amino acid sequence of SEQ ID NO: 51.

In some aspects, the VHH1 comprises: an amino acid sequence at least 85%, at least 90%, at least 95%, at least 96%, at least 97%, at least 98%, or at least 99% identical to the amino acid sequence of SEQ ID NO:3, SEQ ID NO:4, SEQ ID NO: 17, SEQ ID NO: 18, SEQ ID NO:31, SEQ ID NO: 32, SEQ ID NO:45, SEQ ID NO:46, SEQ ID NO: 65, SEQ ID NO: 68, SEQ ID NO:69, SEQ ID NO: 70, SEQ ID NO:71, SEQ ID NO: 72, SEQ ID NO: 73, SEQ ID NO:74, SEQ ID NO:75, SEQ ID NO: 77, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 140, SEQ ID NO: 141, SEQ ID NO: 142, SEQ ID NO: 143, SEQ ID NO: 144, SEQ ID NO: 145, SEQ ID NO: 177, SEQ ID NO: 178, SEQ ID NO: 179, SEQ ID NO: 180, SEQ ID NO:181, SEQ ID NO:182, SEQ ID NO:183, SEQ ID NO:184, SEQ ID NO: 221, SEQ ID NO: 222, SEQ ID NO: 223, SEQ ID NO: 224, SEQ ID NO: 225, SEQ ID NO: 226, SEQ ID NO: 255, SEQ ID NO: 256, SEQ ID NO: 265, SEQ ID NO: 267, SEQ ID NO: 275, SEQ ID NO: 283, SEQ ID NO: 291, SEQ ID NO: 299, SEQ ID NO: 307, SEQ ID NO:315, SEQ ID NO:323, SEQ ID NO:331, or SEQ ID NO:339.

In some aspects, the VHH1 comprises the amino acid sequence of SEQ ID NO:3, SEQ ID NO: 4, SEQ ID NO: 17, SEQ ID NO: 18, SEQ ID NO:31, SEQ ID NO:32, SEQ ID NO:45, SEQ ID NO: 46, SEQ ID NO: 65, SEQ ID NO: 68, SEQ ID NO: 69, SEQ ID NO: 70, SEQ ID NO:71, SEQ ID NO: 72, SEQ ID NO:73, SEQ ID NO:74, SEQ ID NO:75, SEQ ID NO:77, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 140, SEQ ID NO: 141, SEQ ID NO: 142, SEQ ID NO: 143, SEQ ID NO: 144, SEQ ID NO: 145, SEQ ID NO: 177, SEQ ID NO: 178, SEQ ID NO: 179, SEQ ID NO: 180, SEQ ID NO:181, SEQ ID NO: 182, SEQ ID NO: 183, SEQ ID NO: 184, SEQ ID NO: 221, SEQ ID NO: 222, SEQ ID NO: 223, SEQ ID NO: 224, SEQ ID NO: 225, SEQ ID NO: 226, SEQ ID NO: 255, SEQ ID NO: 256, SEQ ID NO: 265, SEQ ID NO: 267, SEQ ID NO: 275, SEQ ID NO: 283, SEQ ID NO: 291, SEQ ID NO: 299, SEQ ID NO:307, SEQ ID NO:315, SEQ ID NO:323, SEQ ID NO:331, or SEQ ID NO:339. In some aspects, the VHH2 comprises the amino acid sequence of SEQ ID NO: 8, SEQ ID NO:9, SEQ ID NO: 22, SEQ ID NO: 23, SEQ ID NO:36, SEQ ID NO: 37, SEQ ID NO:50, SEQ ID NO:51, SEQ ID NO:66, SEQ ID NO:67, SEQ ID NO:76, SEQ ID NO: 101, SEQ ID NO: 102, SEQ ID NO: 103, SEQ ID NO: 146, SEQ ID NO: 185, SEQ ID NO: 186, SEQ ID NO:187, SEQ ID NO: 254, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 266, SEQ ID NO: 271, SEQ ID NO: 279, SEQ ID NO: 287, SEQ ID NO: 295, SEQ ID NO: 303, SEQ ID NO:311, SEQ ID NO:319, SEQ ID NO:327, SEQ ID NO:335, or SEQ ID NO: 343.

In some aspects, the VHH1 comprises: an amino acid sequence at least 85%, at least 90%, at least 95%, at least 96%, at least 97%, at least 98%, or at least 99% identical to the amino acid sequence of SEQ ID NO:3, SEQ ID NO:4, SEQ ID NO: 17, SEQ ID NO: 18, SEQ ID NO:31, SEQ ID NO: 32, SEQ ID NO:45, SEQ ID NO:46, SEQ ID NO: 65, SEQ ID NO: 68, SEQ ID NO:69, SEQ ID NO: 70, SEQ ID NO:71, SEQ ID NO:72, SEQ ID NO:73, SEQ ID NO:74, SEQ ID NO:75, SEQ ID NO: 77, SEQ ID NO:93, SEQ ID NO:94, SEQ ID NO:95, SEQ ID NO:96, SEQ ID NO:97, SEQ ID NO: 98, SEQ ID NO:99, SEQ ID NO: 100, SEQ ID NO: 140, SEQ ID NO: 141, SEQ ID NO: 142, SEQ ID NO: 143, SEQ ID NO: 144, SEQ ID NO: 145, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 221, SEQ ID NO: 222, SEQ ID NO: 223, SEQ ID NO: 224, SEQ ID NO: 225, SEQ ID NO: 226, SEQ ID NO: 255, SEQ ID NO: 256, SEQ ID NO: 265, SEQ ID NO: 267, SEQ ID NO: 275, SEQ ID NO: 283, SEQ ID NO: 291, SEQ ID NO: 299, SEQ ID NO:307, SEQ ID NO: 315, SEQ ID NO:323, SEQ ID NO:331, or SEQ ID NO:339. In some aspects, the VHH2 comprises: an amino acid sequence at least 85%, at least 90%, at least 95%, at least 96%, at least 97%, at least 98%, or at least 99% identical to the amino acid sequence of SEQ ID NO:8, SEQ ID NO: 9, SEQ ID NO: 22, SEQ ID NO: 23, SEQ ID NO:36, SEQ ID NO:37, SEQ ID NO:50, SEQ ID NO: 51, SEQ ID NO:66, SEQ ID NO:67, SEQ ID NO:76, SEQ ID NO: 101, SEQ ID NO: 102, SEQ ID NO:103, SEQ ID NO:146, SEQ ID NO: 185, SEQ ID NO: 186, SEQ ID NO: 187, SEQ ID NO: 254, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 266, SEQ ID NO: 271, SEQ ID NO: 279, SEQ ID NO: 287, SEQ ID NO: 295, SEQ ID NO:303, SEQ ID NO:311, SEQ ID NO:319, SEQ ID NO:327, SEQ ID NO:335, or SEQ ID NO:343.

In some aspects, the VHH1 comprises the amino acid sequence of SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 221, SEQ ID NO: 222, SEQ ID NO: 223, SEQ ID NO: 224, SEQ ID NO: 225, SEQ ID NO: 226, SEQ ID NO: 255, SEQ ID NO: 256, SEQ ID NO: 265, SEQ ID NO: 267, SEQ ID NO: 275, SEQ ID NO: 283, SEQ ID NO: 291, SEQ ID NO: 299, SEQ ID NO: 307, SEQ ID NO:315, SEQ ID NO:323, SEQ ID NO:331, or SEQ ID NO:339. In some aspects, the VHH2 comprises the amino acid sequence of SEQ ID NO:8, SEQ ID NO:9, SEQ ID NO: 22, SEQ ID NO: 23, SEQ ID NO:36, SEQ ID NO:37, SEQ ID NO:50, SEQ ID NO:51, SEQ ID NO: 66, SEQ ID NO:67, SEQ ID NO: 76, SEQ ID NO: 101, SEQ ID NO: 102, SEQ ID NO: 103, SEQ ID NO:146, SEQ ID NO: 185, SEQ ID NO: 186, SEQ ID NO: 187, SEQ ID NO: 254, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 266, SEQ ID NO: 271, SEQ ID NO: 279, SEQ ID NO: 287, SEQ ID NO: 295, SEQ ID NO:303, SEQ ID NO:311, SEQ ID NO:319, SEQ ID NO:327, SEQ ID NO:335, or SEQ ID NO:343.

In some aspects, the VHH1 comprises: an amino acid sequence at least 85%, at least 90%, at least 95%, at least 96%, at least 97%, at least 98%, or at least 99% identical to the amino acid sequence of SEQ ID NO:3, SEQ ID NO:4, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 140, SEQ ID NO: 141, SEQ ID NO: 142, SEQ ID NO: 143, SEQ ID NO: 144, or SEQ ID NO: 145. In some aspects, the VHH2 comprises an amino acid sequence at least 85%, at least 90%, at least 95%, at least 96%, at least 97%, at least 98%, or at least 99% identical to the amino acid sequence of SEQ ID NO:8, SEQ ID NO:9, SEQ ID NO: 101, SEQ ID NO: 102, SEQ ID NO: 103, or SEQ ID NO: 146.

In some aspects, provided herein is a polypeptide that binds IL31 comprising two or more IL31 binders, wherein the first IL31 binder comprises a VHH1 which comprises the amino acid sequence of SEQ ID NO:3, SEQ ID NO:4, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 140, SEQ ID NO: 141, SEQ ID NO: 142, SEQ ID NO: 143, SEQ ID NO: 144, SEQ ID NO: 145, and the second IL31 binder comprises a VHH2 that binds to IL31.

In some aspects, provided herein is a polypeptide that binds IL31 comprising two or more IL31 binders, wherein the first IL31 binder comprises a VHH1 that binds to IL31 and the second IL31 binder comprises a VHH2 comprising the amino acid sequence of SEQ ID NO: 8, SEQ ID NO: 9, SEQ ID NO: 101, SEQ ID NO: 102, SEQ ID NO: 103, or SEQ ID NO: 146.

In some aspects, provided herein is a polypeptide that binds IL31 comprising two or more IL31 binders, wherein the first IL31 binder comprises a VHH1 comprising the amino acid sequence of SEQ ID NO:3, SEQ ID NO:4, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 140, SEQ ID NO: 141, SEQ ID NO: 142, SEQ ID NO: 143, SEQ ID NO: 144, or SEQ ID NO: 145 and the second IL31 binder comprises a VHH2 comprising the amino acid sequence of SEQ ID NO:8, SEQ ID NO:9, SEQ ID NO: 101, SEQ ID NO: 102, SEQ ID NO: 103, or SEQ ID NO: 146.

In some aspects, the polypeptide has the structure: [VHH1]-linker-[VHH2].

In some aspects, the linker is from about 5 to about 50 amino acids in length. In some aspects, the linker is from about 8 to about 40 amino acids in length. In some aspects, the linker is from about 12 to about 37 amino acids in length. In some aspects, the linker is a flexible linker. In some aspects, the linker is a rigid linker.

In some aspects, the linker comprises an amino acid sequence having at least 85%, at least 90%, at least 95%, at least 96%, at least 97%, at least 98%, at least 99%, or about 100% sequence identity to SEQ ID NO: 104 or SEQ ID NO: 188.

In some aspects, the polypeptide comprises an amino acid sequence at least 85%, at least 90%, at least 95%, at least 96%, at least 97%, at least 98%, or at least 99% identical to the amino acid sequence of SEQ ID NO: 13, SEQ ID NO: 27, SEQ ID NO: 41, SEQ ID NO: 55, SEQ ID NO: 14, SEQ ID NO: 28, SEQ ID NO: 42, or SEQ ID NO: 56. In some aspects, the polypeptide comprises the amino acid sequence of SEQ ID NO: 13, SEQ ID NO: 27, SEQ ID NO: 41, SEQ ID NO: 55, SEQ ID NO: 14, SEQ ID NO: 28, SEQ ID NO: 42, or SEQ ID NO: 56. In some aspects, the polypeptide comprises the amino acid sequence of SEQ ID NO: 14, SEQ ID NO: 28, SEQ ID NO: 42, or SEQ ID NO: 56. In some aspects, the polypeptide comprises the amino acid sequence of SEQ ID NO: 14. In some aspects, the polypeptide comprises the amino acid sequence of SEQ ID NO: 28. In some aspects, the polypeptide comprises the amino acid sequence of SEQ ID NO: 42. In some aspects, the polypeptide comprises the amino acid sequence of SEQ ID NO: 56.

In some aspects, the polypeptide comprises an amino acid sequence having at least 85%, at least 90%, at least 95%, at least 96%, at least 97%, at least 98%, at least 99%, or about 100% sequence identity to the amino acid sequence of SEQ ID NO:13, SEQ ID NO:14, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, or SEQ ID NO: 176.

In some aspects, the polypeptide comprises the amino acid sequence of SEQ ID NO:13, SEQ ID NO:14, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, or SEQ ID NO: 176.

In some aspects, the polypeptide comprises an amino acid sequence having at least 85%, at least 90%, at least 95%, at least 96%, at least 97%, at least 98%, at least 99%, or about 100% sequence identity to the amino acid sequence of SEQ ID NO:55, SEQ ID NO:56, SEQ ID NO: 189, SEQ ID NO: 190, SEQ ID NO: 191, SEQ ID NO: 192, SEQ ID NO: 193, SEQ ID NO:194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO: 211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249 or SEQ ID NO: 250.

In some aspects, the polypeptide comprises the amino acid sequence of SEQ ID NO:55, SEQ ID NO:56, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO:200, SEQ ID NO:201, SEQ ID NO:202, SEQ ID NO:203, SEQ ID NO: 204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO: 215, SEQ ID NO:216, SEQ ID NO:217, SEQ ID NO:218, SEQ ID NO:219, SEQ ID NO:220, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ IDNO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249 or SEQ ID NO: 250.

In some aspects, the polypeptide further comprises an Fc region. In some aspects, the polypeptide has the structure: [VHH1]-linker-[VHH2]-Fc region.

In some aspects, the polypeptide of the present disclosure binds to an antigen comprising at least two binding moieties that comprise VHH1, VHH2, and a linker, wherein the polypeptide has the structure: [VHH1]-linker-[VHH2], wherein the linker comprises an amino acid sequence comprising (X)n-G, and the VHH2 comprises at the N-terminus Xa, Xb, and Xc, wherein X is an amino acid, G is glycine, n is any integer between 0 and 100, Xa is E (Glutamic acid), Q (Glutamine), or missing, Xb is V (Valine) or missing, and Xc is Q (Glutamine), K (Lysine), or missing. In some aspects, the VHH1 comprises any VHH1 disclosed herein. In some aspects, the VHH2 comprises any VHH2 disclosed herein.

In some aspects, the polypeptide further comprises an Fc region. In some aspects, the X in the linker comprises (G2S)n, (G3S)n, (G4S)n, (G5S)n, or any combination thereof, and n is an integer between 1 and 10. In some aspects, the X in the linker comprises (G4S)n, and n is 4, 5, or 6. In some aspects, the linker comprises the amino acid sequence of SEQ ID NO: 104 or SEQ ID NO: 188.

In some aspects, the polypeptide comprises an IgG1, IgG2, IgG3, or IgG4 Fc region. In some aspects, the polypeptide comprises a human Fc region. In some aspects, the Fc region comprises one or more substitutions, deletions, insertions, or any combination thereof. In some aspects, the one or more substitutions comprise C220S (hinge region), M252Y (CH2 region), S254T (CH2 region), T256E (CH2 region), L234A (CH2 region), L235A (CH2 region), M428L (CH3 region), N434S (CH3 region), or any combination thereof, according to the EU numbering system. In some aspects, the C terminus of the Fc region is Lysine. In some aspects, the C terminus of the Fc region is not Lysine. In some aspects, the C terminal Lysine of the Fc region has been removed, e.g., enzymatically removed during upstream production process.

In some aspects, the polypeptide comprises an Fc region with a modified effector function. In some aspects, the polypeptide comprises an effector null Fc region. In some aspects, the polypeptide comprises an Fc region comprising a full or partial hinge, no CH1 region, a full or partial CH2 region, and a full or partial CH3 region. In some aspects, the polypeptide comprises an Fc region comprising a full hinge region, no CH1 region, full CH2 region and full CH3 region. In some aspects, the full or partial hinge region comprises a cysteine (C) to serine(S) mutation at position 220 according to EU numbering (C220S). In some aspects, the Fc region comprises the amino acid sequence of SEQ ID NO: 62 or the amino acid residues 1-231 of SEQ ID NO: 62. In some aspects, the Fc region comprises the amino acid sequence of SEQ ID NO: 90 or the amino acid residues 1-231 of SEQ ID NO: 90.

In some aspects, the polypeptide disclosed herein is monospecific. In some aspects, the polypeptide disclosed herein is bispecific. In some aspects, the polypeptide disclosed herein is trispecific. In some aspects, the polypeptide disclosed herein is tetraspecific.

In some aspects, the polypeptide further comprises a binder that binds to a protein, but does not bind to IL31 (“third binder). In some aspects, the polypeptide further comprises a binder that binds to the same protein as the third binder or a different protein from the third binder or IL31 (“fourth binder”). In some aspects, the third binder comprises a VHH (VHH3). In some aspects, the fourth binder comprises a VHH (VHH4). In some aspects, the VHH3 and the VHH4 are different. In some aspects, the VHH3 and the VHH4 are the same. In some aspects, the VHH3 and VHH4 single binders bind to the same target antigen biparatopically or recognize two different epitopes on the same target antigen. In some aspects, the biparatopic VHH3 and VHH4 single binders bind the same target antigen at nonoverlapping epitopes, which allows both VHH3 and VHH4 single binders to bind the same target antigen simultaneously.

In some aspects, the polypeptide comprises an amino acid sequence at least 85%, at least 90%, at least 95%, at least 96%, at least 97%, at least 98%, or at least 99% identical to the amino acid sequence of SEQ ID NO: 15 or the amino acid residues 1-506 of SEQ ID NO: 15, SEQ ID NO: 29 or the amino acid residues 1-496 of SEQ ID NO: 29, SEQ ID NO: 43 or the amino acid residues 1-487 of SEQ ID NO: 43, SEQ ID NO: 57 or the amino acid residues 1-507 of SEQ ID NO: 57, SEQ ID NO: 16 or the amino acid residues 1-506 of SEQ ID NO: 16, SEQ ID NO: 30 or the amino acid residues 1-496 of SEQ ID NO: 30, SEQ ID NO: 44 or the amino acid residues 1-487 of SEQ ID NO: 44, SEQ ID NO: 58 or the amino acid residues 1-507 of SEQ ID NO: 58, SEQ ID NO: 91 or the amino acid residues 1-506 of SEQ ID NO: 91, or SEQ ID NO: 92 or the amino acid residues 1-506 of SEQ ID NO: 92. In some aspects, the polypeptide comprises the amino acid sequence of SEQ ID NO: 15 or the amino acid residues 1-506 of SEQ ID NO: 15, SEQ ID NO: 29 or the amino acid residues 1-496 of SEQ ID NO: 29, SEQ ID NO: 43 or the amino acid residues 1-487 of SEQ ID NO: 43, SEQ ID NO: 57 or the amino acid residues 1-507 of SEQ ID NO: 57, SEQ ID NO: 91 or the amino acid residues 1-506 of SEQ ID NO: 91, SEQ ID NO: 16 or the amino acid residues 1-506 of SEQ ID NO: 16, SEQ ID NO: 30 or the amino acid residues 1-496 of SEQ ID NO: 30, SEQ ID NO: 44 or the amino acid residues 1-487 of SEQ ID NO: 44, SEQ ID NO: 58 or the amino acid residues 1-507 of SEQ ID NO: 58, or SEQ ID NO: 92 or the amino acid residues 1-506 of SEQ ID NO: 92. In some aspects, the polypeptide comprises the amino acid sequence of SEQ ID NO: 16 or the amino acid residues 1-506 of SEQ ID NO: 16, SEQ ID NO: 30 or the amino acid residues 1-496 of SEQ ID NO: 30, SEQ ID NO: 44 or the amino acid residues 1-487 of SEQ ID NO: 44, SEQ ID NO: 58 or the amino acid residues 1-507 of SEQ ID NO: 58, or SEQ ID NO: 92 or the amino acid residues 1-506 of SEQ ID NO: 92. In some aspects, the polypeptide comprises the amino acid sequence of SEQ ID NO: 16 or the amino acid residues 1-506 of SEQ ID NO: 16. In some aspects, the polypeptide comprises the amino acid sequence of SEQ ID NO: 30 or the amino acid residues 1-496 of SEQ ID NO: 30. In some aspects, the polypeptide comprises the amino acid sequence of SEQ ID NO: 44 or the amino acid residues 1-487 of SEQ ID NO: 44. In some aspects, the polypeptide comprises the amino acid sequence of SEQ ID NO: 58 or the amino acid residues 1-507 of SEQ ID NO: 58. In some aspects, the polypeptide comprises the amino acid sequence of SEQ ID NO: 92 or the amino acid residues 1-506 of SEQ ID NO: 92.

In some aspects, the polypeptide binds to human IL31. In some aspects, the human IL31 comprises the amino acid sequence of SEQ ID NO: 1 or SEQ ID NO: 2.

In some aspects, the polypeptide binds to human IL31 at a kD of less than or equal to 1×10⁻⁹ M, less than or equal to 5×10⁻¹⁰ M, less than or equal to 1×10⁻¹⁰ M, less than or equal to 5×10⁻¹¹ M, less than or equal to 1×10⁻¹¹ M, less than or equal to 5×10⁻¹² M, or less than or equal to 1×10⁻¹² M, as measured by surface plasmon resonance. In some aspects, the polypeptide binds to human IL31 with a k_{off} rate of less than or equal to 5×10⁻⁴ M per second, less than or equal to 1×10⁻⁴ M per second, less than or equal to 5×10⁻⁵ M per second, less than or equal to 1×10⁻⁵ M per second, less than or equal to 5×10⁻⁶ M per second, or less than or equal to 1×10⁻⁶ M per second, as measured by surface plasmon resonance.

In some aspects, polypeptides comprising a Fc region are provided herein. In some aspects, the Fc region comprises an amino acid sequence at least 85%, at least 90%, at least 95%, at least 96%, at least 97%, at least 98%, or at least 99% identical to the amino acid sequence of SEQ ID NO: 62 or the amino acid residues 1-231 of SEQ ID NO: 62. In some aspects, the Fc region comprises an amino acid sequence at least 85%, at least 90%, at least 95%, at least 96%, at least 97%, at least 98%, or at least 99% identical to the amino acid sequence of SEQ ID NO: 62 or the amino acid residues 1-231 of SEQ ID NO: 62, and wherein position 5 is serine, position 37 is tyrosine, position 39 is threonine, and position 41 is glutamate according to SEQ ID NO: 62 or the amino acid residues 1-231 of SEQ ID NO: 62. In some aspects, the polypeptide and/or the Fc region comprises the amino acid sequence of SEQ ID NO: 62 or the amino acid residues 1-231 of SEQ ID NO: 62.

In some aspects, polypeptides comprising a Fc region are provided herein. In some aspects, the Fc region comprises an amino acid sequence at least 85%, at least 90%, at least 95%, at least 96%, at least 97%, at least 98%, or at least 99% identical to the amino acid sequence of SEQ ID NO: 90 or the amino acid residues 1-231 of SEQ ID NO: 90. In some aspects, the Fc region comprises an amino acid sequence at least 85%, at least 90%, at least 95%, at least 96%, at least 97%, at least 98%, or at least 99% identical to the amino acid sequence of SEQ ID NO: 90 or the amino acid residues 1-231 of SEQ ID NO: 90, and wherein position 5 is serine, position 37 is tyrosine, position 39 is threonine, and position 41 is glutamate according to SEQ ID NO: 90 or the amino acid residues 1-231 of SEQ ID NO: 90. In some aspects, the polypeptide and/or the Fc region comprises the amino acid sequence of SEQ ID NO: 90 or the amino acid residues 1-231 of SEQ ID NO: 90.

Also provided herein are multimeric polypeptides, each comprising two or more of any one of the polypeptides provided herein.

Also provided herein are pharmaceutical compositions comprising any one or more of the polypeptides or any one or more of the multimeric polypeptides described herein and a pharmaceutically acceptable carrier.

Also provided herein are isolated nucleic acids encoding any one or more of the polypeptides or any one or more of the multimeric polypeptides described herein. Further provided herein are vectors comprising any one or more of the nucleic acids described herein. Further provided herein are in vitro or ex vivo cells comprising any one or more of the nucleic acids described herein. Further provided herein are in vitro or ex vivo cells comprising any one or more of the vectors described herein.

Also provided herein are methods of producing any one or more of the polypeptides or any one or more of the multimeric polypeptides described herein. In some aspects, the method comprises culturing an in vitro or ex vivo cell comprising any one or more of the nucleic acids described herein under conditions suitable for expression of the polypeptide(s). In some aspects, the method comprises culturing an in vitro or ex vivo cell comprising any one or more of the vectors described herein under conditions suitable for expression of the polypeptide(s) or multimeric polypeptide(s). In some aspects, the method further comprises isolating the polypeptide(s) or multimeric polypeptide(s).

Also provided herein are methods for treating a subject having an IL31-associated condition comprising administering to the subject a therapeutically effective amount of any one or more the polypeptides or any one or more of the multimeric polypeptides described herein or a pharmaceutical composition thereof.

In some aspects, the subject is a human subject.

In some aspects, the IL31-associated condition is a pruritic condition. In some aspects, the IL31-associated condition is atopic dermatitis, prurigo nodularis, or chronic spontaneous urticaria.

In some aspects, the polypeptide, the multimeric polypeptide, or the pharmaceutical composition is administered parenterally. In some aspects, the polypeptide, the multimeric polypeptide, or the pharmaceutical composition is administered by an intravenous route, a subcutaneous route, an intramuscular route, or an intraperitoneal route. In some aspects, the polypeptide, the multimeric polypeptide, or the pharmaceutical composition is administered by intravenous route. In some aspects, the polypeptide, the multimeric polypeptide, or the pharmaceutical composition is administered by subcutaneous route.

In some aspects, the method comprises administering one or more therapeutic agent in combination with the polypeptide, the multimeric polypeptide, or the pharmaceutical composition. In some aspects, the one or more therapeutic agent is an antibody, a small molecule inhibitor, and/or a systemic immunosuppressant agent. In some aspects, the one or more therapeutic agent is a topical corticosteroid, a calcineurin inhibitor, an anti-IL4RA antibody, an anti-IL13RA antibody, an anti-OSMR antibody, an anti-Ox40 antibody, an anti-Ox40L antibody, an anti-TSLP antibody an anti-IL 17 antibody, an anti-TNFα antibody, an anti-CD20 antibody, an anti-CD19 antibody, an anti-CD25 antibody, an anti-IL4 antibody, an anti-IL13 antibody, an anti-IL23 antibody, anti-IL23p19 antibody, an anti-IL31RA antibody, an anti-IgE antibody, an anti-CD11a antibody, anti-IL6R antibody, anti-α4-Intergrin antibody, an anti-IL12 antibody, an anti-IL1β antibody, an anti-BlyS antibody, an anti-CKIT antibody, an anti-MRGPRX2 antibody, an anti-Fc epsilon receptor (FcaRI) antibody, an anti-SIGLEC-6 antibody, and/or an anti-SIGLEC-8 antibody. In some aspects, the one or more therapeutic agent is a small molecule inhibitor of IL4RA, IL13RA, IL13, IL31RA, OSMR, Ox40, Ox40L, TSLP, IL17, TNFα, CD20, CD19, CD25, IL4, IL23, IgE, CD11Δ, IL6R, a4-Intergrin, IL12, IL1β, BlyS, CKIT, MRGPRX2, Fc epsilon receptor (FcERI), and/or SIGLEC-6. In some aspects, the one or more therapeutic agent is a STAT inhibitor, a JAK inhibitor, a PI3K inhibitor, an AKT inhibitor, a MAPK inhibitor, a MRGPRX2 inhibitor, a topical corticosteroid, a calcineurin inhibitor, and/or a fusion protein comprising a portion of CTLA-4 and an Fc region of an immunoglobulin, optionally wherein the fusion protein is abatacept or belatacept.

Also provided herein are methods of detecting human IL31 in a biological sample comprising contacting the biological sample with any one or more of the polypeptides or multimeric polypeptides described herein under conditions permissive for binding of the polypeptide or multimeric polypeptide to a human IL31 and detecting whether a complex is formed between the polypeptide or multimeric polypeptide and the human IL31 in the biological sample. In some aspects, wherein the biological sample is a serum sample.

### BRIEF DESCRIPTION OF THE DRAWINGS

FIG. 1 shows flow cytometry plots for Campaign 1 anti-IL31 single binder selection. Round 1 (R1) MACS flow plot images were taken from flow cytometry analysis of R1 MACS eluates for 0 nM IL31 and 100 nM IL31 stained samples. Round 2 (R2) FACS and round 3 (R3), FACS images represent flow plots for sorting human IL31 specific single binder displaying yeast cells at 100 nM and 30 nM human IL31 concentrations, respectively.

FIG. 2 shows flow cytometry plots for Campaign 1 anti-IL31 co-binder selection. Round 1 (R1) MACS flow plot images were taken from flow cytometry analysis of R1 MACS eluates for 0 nM IL31 and 10 nM IL31 stained samples. Round 2 (R2) FACS, round 3 (R3) FACS, round 4 (R4) FACS images represent flow plots for sorting human IL31 specific co-binder displaying yeast cells at 10 nM, 3 nM, and 1 nM human IL31 concentrations, respectively. In R4 FACS plot, AI gate represents populations with higher affinity to human IL31 than AJ gate.

FIG. 3 shows flow cytometry plots for Campaign 2 anti-IL31 single binder selection. Round 1 (R1) MACS flow plot images were taken from flow cytometry analysis of R1 MACS eluates for 0 nM IL31 and 30 nM IL31 stained samples. Round 2 (R2) FACS and round 3 (R3) FACS images represent flow plots for sorting human anti-IL31 VHH displaying yeast cells at 30 nM and 10 nM human IL31 concentrations, respectively. A negative selection was performed in round 4 (R4) FACS to avoid potential non-specific binding. Round 5 (R5) FACS was performed for positive selection of anti-IL31 VHH displaying cells with 10 nM IL31 concentration.

FIG. 4 shows flow cytometry plots for Campaign 2 anti-IL31 co-binder selection. Round 1 (R1) MACS flow plot images were taken from flow cytometry analysis of R1 MACS eluates for 0 nM IL31 and 0.3 nM IL31 stained samples. Round 2 (R2) FACS was performed for sorting human anti-IL31 single-binder displaying yeast cells at 3 nM human IL31 concentrations. A negative selection was performed in round 3 (R3) FACS to avoid potential non-specific binding. Round 4 (R4) and round 5 (R5) FACS were performed for positive selection of anti-IL31 VHH displaying cells with 1 nM and 0.1 nM human IL31 concentrations, respectively.

FIG. 5 is a sequence alignment of VHH1 or VHH2 regions of certain co-binder clone candidates.

FIGS. 6A-6B are plots showing anti-pruritic activity in mice. Female transgenic hIL31/hIL31RA/hOSMR mice were administered 21H7, 1C10, or 1D1 at increasing dose ranging from 0.3 to 3 mg/kg levels then treated intravenously with hIL31 after 24 hours. FIG. 6A is a plot showing pruritic activity as percent scratching counts relative to pre-study treatment with IL31.

FIG. 6B is a plot showing plasma concentrations of co-binders as assessed on the day of hIL31 administration.

FIGS. 7A-7B are plots showing anti-pruritic activity in non-human primates. Healthy male cynomolgus monkeys were administered co-binder 1C10 at either 1 or 5 mg/kg. Animals were then treated intravenously with cynomolgus IL31 after 24 hours. FIG. 7A is a plot showing pruritic activity as percent duration of scratching relative to pre-study treatment with IL31. FIG. 7B is a plot showing the relative duration of pruritic activity to the calculated serum drug concentration.

DESCRIPTION OF THE SEQUENCES

Table 1 provides a listing of certain sequences referenced herein.

TABLE 1
Description of Certain Sequences
Description of Certain Sequences

| SEQ | | |
|---|---|---|
| ID NO | Sequence | Description |
| 1 | MASHSGPSTSVLFLFCCLGGWLASHTLPVRLLRPSDDVQKIVEEL | Human IL31 with |
| | QSLSKMLLKDVEEEKGVLVSQNYTLPCLSPDAQPPNNIHSPAIRA | signal sequence |
| | YLKTIRQLDNKSVIDEIIEHLDKLIFQDAPETNISVPTDTHECKR | |
| | FILTISQQFSECMDLALKSLTSGAQQATT | |
| 2 | SHTLPVRLLRPSDDVQKIVEELQSLSKMLLKDVEEEKGVLVSQNY | Human IL31 |
| | TLPCLSPDAQPPNNIHSPAIRAYLKTIRQLDNKSVIDEIIEHLDK | without signal |
| | LIFQDAPETNISVPTDTHECKRFILTISQQFSECMDLALKSLTSG | sequence |
| | AQQATT | |
| 3 | EVQLVESGGGLVQAGGSLRLSCAASGGTFSSYTMGWFRQAPGKER | 1C10 VHH1 single |
| | EYVGGISSSGYVMYNSESMKGRFTISRENAKNMVYLQMNSLKPED | binder |
| | TAVYYCAAGTIGRPYDYWGQGTQVTVSS | |
| 4 | EVQLVESGGGLVQPGGSLRLSCAASGGTFSSYTMGWFRQAP | Humanized 1C10 |
| | GKEREYVGGISSSGYVMYNSESMKGRFTISRDNAKNTLYLQ | VHH1 single |
| | MNSLRAEDTAVYYCAAGTIGRPYDYWGQGTQVTVSS | binder |
| 5 | SYTMG | 1C10 VHH1 |
| | | CDR1 |
| | | (Kabat numbering) |
| 6 | GISSSGYVMYNSESMKG | 1C10 VHH1 |
| | | CDR2 |
| | | (Kabat numbering) |
| 7 | GTIGRPYDY | 1C10 VHH1 |
| | | CDR3 |
| | | (Kabat numbering) |
| 8 | VKLEESGGGLVQPGGSLILSCAASGDISSIVAMGWYRQAPGKQRE | 1C10 VHH2 single |
| | LVAAITSGGRTHYRDSVKGRFTISGNNDNSALYLHMNSLKPEDTA | binder |
| | VYYCAADRGWTSVGEYDYWGKGTLVTVSS | |
| 9 | VQLVESGGGLVQPGGSLRLSCAASGDISSIVAMGWYRQAPG | Humanized 1C10 |
| | KQRELVSAITSGGRTHYRDSVKGRFTISRDNAKNTLYLQMN | VHH2 single |
| | SLRAEDTAVYYCAADRGWTSVGEYDYWGQGTQVTVSS | binder |
| 10 | IVAMG | 1C10 VHH2 |
| | | CDR1 |
| | | (Kabat numbering) |
| 11 | AITSGGRTHYRDSVKG | 1C10 VHH2 |
| | | CDR2 |
| | | (Kabat numbering) |
| 12 | DRGWTSVGEYDY | 1C10 VHH2 |
| | | CDR3 |
| | | (Kabat numbering) |
| 13 | EVQLVESGGGLVQAGGSLRLSCAASGGTFSSYTMGWFRQAPGKER | 1C10 co-binder |
| | EYVGGISSSGYVMYNSESMKGRFTISRENAKNMVYLQMNSLKPED | |
| | TAVYYCAAGTIGRPYDYWGQGTQVTVSSGGGSGGGGSGGGGSGGG | |
| | GSGGGGSGGGGSGGGGSGMTGVKLEESGGGLVQPGGSLILSCAAS | |
| | GDISSIVAMGWYRQAPGKQRELVAAITSGGRTHYRDSVKGRFTIS | |
| | GNNDNSALYLHMNSLKPEDTAVYYCAADRGWTSVGEYDYWGKGTL | |
| | VTVSS | |
| 14 | EVQLVESGGGLVQPGGSLRLSCAASGGTFSSYTMGWFRQAPGKER | Humanized 1C10 |
| | EYVGGISSSGYVMYNSESMKGRFTISRDNAKNTLYLQMNSLRAED | co-binder |
| | TAVYYCAAGTIGRPYDYWGQGTQVTVSSGGGSGGGGSGGGGSGGG | |
| | GSGGGGSGGGGSGGGGSGMTGVQLVESGGGLVQPGGSLRLSCAAS | |
| | GDISSIVAMGWYRQAPGKQRELVSAITSGGRTHYRDSVKGRFTIS | |
| | RDNAKNTLYLQMNSLRAEDTAVYYCAADRGWTSVGEYDYWGQGTQ | |
| | VTVSS | |
| 15 | EVQLVESGGGLVQAGGSLRLSCAASGGTFSSYTMGWFRQAPGKER | 1C10 co-binder + |
| | EYVGGISSSGYVMYNSESMKGRFTISRENAKNMVYLQMNSLKPED | huIgG1Fc_YTE_C→S |
| | TAVYYCAAGTIGRPYDYWGQGTQVTVSSGGGSGGGGSGGGGSGGG | |
| | GSGGGGSGGGGSGGGGSGMTGVKLEESGGGLVQPGGSLILSCAAS | |
| | GDISSIVAMGWYRQAPGKQRELVAAITSGGRTHYRDSVKGRFTIS | |
| | GNNDNSALYLHMNSLKPEDTAVYYCAADRGWTSVGEYDYWGKGTL | |
| | VTVSSEPKSSDKTHTCPPCPAPELLGGPSVFLFPPKPKDTLYITR | |
| | EPEVTCVVVDVSHEDPEVKFNWYVDGVEVHNAKTKPREEQYNSTY | |
| | RVVSVLTVLHQDWLNGKEYKCKVSNKALPAPIEKTISKAKGQPRE | |
| | PQVYTLPPSRDELTKNQVSLTCLVKGFYPSDIAVEWESNGQPENN | |
| | YKTTPPVLDSDGSFFLYSKLTVDKSRWQQGNVFSCSVMHEALHNH | |
| | YTQKSLSLSPGK | |
| 16 | EVQLVESGGGLVQPGGSLRLSCAASGGTFSSYTMGWFRQAPGKER | Humanized 1C10 |
| | EYVGGISSSGYVMYNSESMKGRFTISRDNAKNTLYLQMNSLRAED | co-binder + |
| | TAVYYCAAGTIGRPYDYWGQGTQVTVSSGGGSGGGGSGGGGSGGG | huIgG1Fc_YTE_C→S |
| | GSGGGGSGGGGSGGGGSGMTGVQLVESGGGLVQPGGSLRLSCAAS | |
| | GDISSIVAMGWYRQAPGKQRELVSAITSGGRTHYRDSVKGRFTIS | |
| | RDNAKNTLYLQMNSLRAEDTAVYYCAADRGWTSVGEYDYWGQGTQ | |
| | VTVSSEPKSSDKTHTCPPCPAPELLGGPSVFLFPPKPKDTLYITR | |
| | EPEVTCVVVDVSHEDPEVKFNWYVDGVEVHNAKTKPREEQYNSTY | |
| | RVVSVLTVLHQDWLNGKEYKCKVSNKALPAPIEKTISKAKGQPRE | |
| | PQVYTLPPSRDELTKNQVSLTCLVKGFYPSDIAVEWESNGQPENN | |
| | YKTTPPVLDSDGSFFLYSKLTVDKSRWQQGNVFSCSVMHEALHNH | |
| | YTQKSLSLSPGK | |
| 17 | EVQLVESGGGLVQAGGSLRLSCAASGGTFSSYAMAWFRQAPGKER | 1D1 VHH1 single |
| | EYVAGISSSGYTLYKSDSMKGRFTISRENAKNMVYLQMNSLKPED | binder |
| | TAVYSCAAGIFGRPYEYWGQGTLVTVSS | |
| 18 | EVQLVESGGGLVQPGGSLRLSCAASGGTFSSYAMAWFRQAPGKER | Humanized 1D1 |
| | EYVAGISSSGYTLYKSDSMKGRFTISRDNAKNTLYLQMNSLRAED | VHH1 single |
| | TAVYYCAAGIFGRPYEYWGQGTLVTVSS | binder |
| 19 | SYAMA | 1D1 VHH1 CDR1 |
| 20 | GISSSGYTLYKSDSMKG | 1D1 VHH1 CDR2 |
| 21 | GIFGRPYEY | 1D1 VHH1 CDR3 |
| 22 | VKLEESGGGLVQAGGSLILSCAASGDIPSIVAMGWYRQAPGKQRE | 1D1 VHH2 single |
| | LVAAITSGGRTHYRDSVKGRFTISGNNDNSALYLHMNSLKPEDTA | binder |
| | VYYCAADMGLTAVGEYDYWGQGTQVTVSS | |
| 23 | VQLVESGGGLVQPGGSLRLSCAASGDIPSIVAMGWYRQAPG | Humanized 1D1 |
| | KQRELVSAITSGGRTHYRDSVKGRFTISRDNSKNTLYLQMN | VHH2 single |
| | SLRAEDTAVYYCAADMGLTAVGEYDYWGQGTQVTVSS | binder |
| 24 | IVAMG | 1D1 VHH2 CDR1 |
| 25 | AITSGGRTHYRDSVKG | 1D1 VHH2 CDR2 |
| 26 | DMGLTAVGEYDY | 1D1 VHH2 CDR3 |
| 27 | EVQLVESGGGLVQAGGSLRLSCAASGGTFSSYAMAWFRQAPGKER | 1D1 co-binder |
| | EYVAGISSSGYTLYKSDSMKGRFTISRENAKNMVYLQMNSLKPED | |
| | TAVYSCAAGIFGRPYEYWGQGTLVTVSSGGGSGGGGSGGGGSGGG | |
| | GSGGGGSPSMGVKLEESGGGLVQAGGSLILSCAASGDIPSIVAMG | |
| | WYRQAPGKQRELVAAITSGGRTHYRDSVKGRFTISGNNDNSALYL | |
| | HMNSLKPEDTAVYYCAADMGLTAVGEYDYWGQGTQVTVSS | |
| 28 | EVQLVESGGGLVQPGGSLRLSCAASGGTFSSYAMAWFRQAPGKER | Humanized 1D1 |
| | EYVAGISSSGYTLYKSDSMKGRFTISRDNAKNTLYLQMNSLRAED | co-binder |
| | TAVYYCAAGIFGRPYEYWGQGTLVTVSSGGGSGGGGSGGGGSGGG | |
| | GSGGGGSPSMGVQLVESGGGLVQPGGSLRLSCAASGDIPSIVAMG | |
| | WYRQAPGKQRELVSAITSGGRTHYRDSVKGRFTISRDNSKNTLYL | |
| | QMNSLRAEDTAVYYCAADMGLTAVGEYDYWGQGTQVTVSS | |
| 29 | EVQLVESGGGLVQAGGSLRLSCAASGGTFSSYAMAWFRQAPGKER | 1D1 co-binder + |
| | EYVAGISSSGYTLYKSDSMKGRFTISRENAKNMVYLQMNSLKPED | huIgG1Fc_YTE_C→S |
| | TAVYSCAAGIFGRPYEYWGQGTLVTVSSGGGSGGGGSGGGGSGGG | |
| | GSGGGGSPSMGVKLEESGGGLVQAGGSLILSCAASGDIPSIVAMG | |
| | WYRQAPGKQRELVAAITSGGRTHYRDSVKGRFTISGNNDNSALYL | |
| | HMNSLKPEDTAVYYCAADMGLTAVGEYDYWGQGTQVTVSSEPKSS | |
| | DKTHTCPPCPAPELLGGPSVFLFPPKPKDTLYITREPEVTCVVVD | |
| | VSHEDPEVKFNWYVDGVEVHNAKTKPREEQYNSTYRVVSVLTVLH | |
| | QDWLNGKEYKCKVSNKALPAPIEKTISKAKGQPREPQVYTLPPSR | |
| | DELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GK | |
| 30 | EVQLVESGGGLVQPGGSLRLSCAASGGTFSSYAMAWFRQAPGKER | Humanized 1D1 |
| | EYVAGISSSGYTLYKSDSMKGRFTISRDNAKNTLYLQMNSLRAED | co-binder + |
| | TAVYYCAAGIFGRPYEYWGQGTLVTVSSGGGSGGGGSGGGGSGGG | huIgG1Fc_YTE_C→S |
| | GSGGGGSPSMGVQLVESGGGLVQPGGSLRLSCAASGDIPSIVAMG | |
| | WYRQAPGKQRELVSAITSGGRTHYRDSVKGRFTISRDNSKNTLYL | |
| | QMNSLRAEDTAVYYCAADMGLTAVGEYDYWGQGTQVTVSSEPKSS | |
| | DKTHTCPPCPAPELLGGPSVFLFPPKPKDTLYITREPEVTCVVVD | |
| | VSHEDPEVKFNWYVDGVEVHNAKTKPREEQYNSTYRVVSVLTVLH | |
| | QDWLNGKEYKCKVSNKALPAPIEKTISKAKGQPREPQVYTLPPSR | |
| | DELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GK | |
| 31 | EVQLVESGGGSVQAGGSLRLSCAASGGTFSSYAMAWFRQAPGKER | 1H5 VHH1 single |
| | EYVAGISSSGYTLYKSDSMKGRFTISRENAKNMVYLQMNSLKPED | binder |
| | TAVYSCAAGIFGRPYEYWGQGTQVTVSS | |
| 32 | EVQLVESGGGLVQPGGSLRLSCAASGGTFSSYAMAWFRQAPGKER | Humanized 1H5 |
| | EYVAGISSSGYTLYKSDSMKGRFTISRDNAKNTLYLQMNSLRAED | VHH1 |
| | TAVYYCAAGIFGRPYEYWGQGTQVTVSS | |
| 33 | SYAMA | 1H5 VHH1 CDR1 |
| 34 | GISSSGYTLYKSDSMKG | 1H5 VHH1 CDR2 |
| 35 | GIFGRPYEY | 1H5 VHH1 CDR3 |
| 36 | VQLVESGGGLVQAGGSLRLSCAASGSISSIHAMGWYRQAPGKQRE | 1H5 VHH2 single |
| | LIAAITSGGRRHYRDSVKGRFTISGDNAKTNLYLQMNSLKPEDTA | binder |
| | VYYCAADTGRVAVGEYDYWGQGTLVTVSS | |
| 37 | VQLVESGGGLVQPGGSLRLSCAASGSISSIHAMGWYRQAPGKQRE | Humanized 1H5 |
| | LIAAITSGGRRHYRDSVKGRFTISRDNSKNTLYLQMNSLRAEDTA | VHH2 |
| | VYYCAADTGRVAVGEYDYWGQGTLVTVSS | |
| 38 | IHAMG | 1H5 VHH2 CDR1 |
| 39 | AITSGGRRHYRDSVKG | 1H5 VHH2 CDR2 |
| 40 | DTGRVAVGEYDY | 1H5 VHH2 CDR3 |
| 41 | EVQLVESGGGSVQAGGSLRLSCAASGGTFSSYAMAWFRQAPGKER | 1H5 co-binder |
| | EYVAGISSSGYTLYKSDSMKGRFTISRENAKNMVYLQMNSLKPED | |
| | TAVYSCAAGIFGRPYEYWGQGTQVTVSSGGGGSGGGGSGGGGSRS | |
| | VGVQLVESGGGLVQAGGSLRLSCAASGSISSIHAMGWYRQAPGKQ | |
| | RELIAAITSGGRRHYRDSVKGRFTISGDNAKTNLYLQMNSLKPED | |
| | TAVYYCAADTGRVAVGEYDYWGQGTLVTVSS | |
| 42 | EVQLVESGGGLVQPGGSLRLSCAASGGTFSSYAMAWFRQAPGKER | Humanized 1H5 |
| | EYVAGISSSGYTLYKSDSMKGRFTISRDNAKNTLYLQMNSLRAED | co-binder |
| | TAVYYCAAGIFGRPYEYWGQGTQVTVSSGGGGSGGGGSGGGGSRS | |
| | VGVQLVESGGGLVQPGGSLRLSCAASGSISSIHAMGWYRQAPGKQ | |
| | RELIAAITSGGRRHYRDSVKGRFTISRDNSKNTLYLQMNSLRAED | |
| | TAVYYCAADTGRVAVGEYDYWGQGTLVTVSS | |
| 43 | EVQLVESGGGSVQAGGSLRLSCAASGGTFSSYAMAWFRQAPGKER | 1H5 co-binder + |
| | EYVAGISSSGYTLYKSDSMKGRFTISRENAKNMVYLQMNSLKPED | huIgG1Fc_YTE_C→S |
| | TAVYSCAAGIFGRPYEYWGQGTQVTVSSGGGGSGGGGSGGGGSRS | |
| | VGVQLVESGGGLVQAGGSLRLSCAASGSISSIHAMGWYRQAPGKQ | |
| | RELIAAITSGGRRHYRDSVKGRFTISGDNAKTNLYLQMNSLKPED | |
| | TAVYYCAADTGRVAVGEYDYWGQGTLVTVSSEPKSSDKTHTCPPC | |
| | PAPELLGGPSVFLFPPKPKDTLYITREPEVTCVVVDVSHEDPEVK | |
| | FNWYVDGVEVHNAKTKPREEQYNSTYRVVSVLTVLHQDWLNGKEY | |
| | KCKVSNKALPAPIEKTISKAKGQPREPQVYTLPPSRDELTKNQVS | |
| | LTCLVKGFYPSDIAVEWESNGQPENNYKTTPPVLDSDGSFFLYSK | |
| | LTVDKSRWQQGNVFSCSVMHEALHNHYTQKSLSLSPGK | |
| 44 | EVQLVESGGGLVQPGGSLRLSCAASGGTFSSYAMAWFRQAPGKER | Humanized 1H5 |
| | EYVAGISSSGYTLYKSDSMKGRFTISRDNAKNTLYLQMNSLRAED | co-binder + |
| | TAVYYCAAGIFGRPYEYWGQGTQVTVSSGGGGSGGGGSGGGGSRS | huIgG1Fc_YTE_C→S |
| | VGVQLVESGGGLVQPGGSLRLSCAASGSISSIHAMGWYRQAPGKQ | |
| | RELIAAITSGGRRHYRDSVKGRFTISRDNSKNTLYLQMNSLRAED | |
| | TAVYYCAADTGRVAVGEYDYWGQGTLVTVSSEPKSSDKTHTCPPC | |
| | PAPELLGGPSVFLFPPKPKDTLYITREPEVTCVVVDVSHEDPEVK | |
| | FNWYVDGVEVHNAKTKPREEQYNSTYRVVSVLTVLHQDWLNGKEY | |
| | KCKVSNKALPAPIEKTISKAKGQPREPQVYTLPPSRDELTKNQVS | |
| | LTCLVKGFYPSDIAVEWESNGQPENNYKTTPPVLDSDGSFFLYSK | |
| | LTVDKSRWQQGNVFSCSVMHEALHNHYTQKSLSLSPGK | |
| 45 | EVQLVESGGGLVQAGGSLRLSCAASGGTFSSYTMGWFRQAPGKER | 21H7 VHH1 single |
| | EYVGGISSSGYVMYNSESMKGRFTISRENAKNMVYLQMNSLKPED | binder |
| | TAVYYCAAGTIGRPYDYWGQGTQVTVSS | |
| 46 | EVQLVESGGGLVQPGGSLRLSCAASGGTFSSYTMGWFRQAP | Humanized 21H7 |
| | GKEREYVGGISSSGYVMYNSESMKGRFTISRDNAKNTLYLQ | VHH1 |
| | MNSLRAEDTAVYYCAAGTIGRPYDYWGQGTQVTVSS | |
| 47 | SYTMG | 21H7 VHH1 |
| | | CDR1 |
| 48 | GISSSGYVMYNSESMKG | 21H7 VHH1 |
| | | CDR2 |
| 49 | GTIGRPYDY | 21H7 VHH1 |
| | | CDR3 |
| 50 | LVESGGGSVQAGDSLRLSCAASGRTFSTYIIGWFRQAPGKEREFV | 21H7 VHH2 single |
| | ASISWGGRGTYYADSVKDRFTISRDNP KNMVYLQMNSLKPEDTAV | binder |
| | YYCAAGDLPGGRRALDYDYWGQGTLVTVSS | |
| 51 | LVESGGGLVQPGGSLRLSCAASGRTFSTYIIGWFRQAPGKE | Humanized 21H7 |
| | REFVASISWGGRGTYYADSVKDRFTISRDNPKNMVYLQMNS | VHH2 |
| | LRAEDTAVYYCAAGDLPGGRRALDYDYWGQGTLVTVSS | |
| 52 | TYIIG | 21H7 VHH2 |
| | | CDR1 |
| 53 | SISWGGRGTYYADSVKD | 21H7 VHH2 |
| | | CDR2 |
| 54 | GDLPGGRRALDYDY | 21H7 VHH2 |
| | | CDR3 |
| 55 | EVQLVESGGGLVQAGGSLRLSCAASGGTFSSYTMGWFRQAPGKER | 21H7 co-binder |
| | EYVGGISSSGYVMYNSESMKGRFTISRENAKNMVYLQMNSLKPED | |
| | TAVYYCAAGTIGRPYDYWGQGTQVTVSSGGGSGGGGSGGGGSGGG | |
| | GSGGGGSGGGGSGGGGSEGVGLVESGGGSVQAGDSLRLSCAASGR | |
| | TFSTYIIGWFRQAPGKEREFVASISWGGRGTYYADSVKDRFTISR | |
| | DNPKNMVYLQMNSLKPEDTAVYYCAAGDLPGGRRALDYDYWGQGT | |
| | LVTVSS | |
| 56 | EVQLVESGGGLVQPGGSLRLSCAASGGTFSSYTMGWFRQAPGKER | Humanized 21H7 |
| | EYVGGISSSGYVMYNSESMKGRFTISRDNAKNTLYLQMNSLRAED | co-binder |
| | TAVYYCAAGTIGRPYDYWGQGTQVTVSSGGGSGGGGSGGGGSGGG | |
| | GSGGGGSGGGGSGGGGSEGVGLVESGGGLVQPGGSLRLSCAASGR | |
| | TFSTYIIGWFRQAPGKEREFVASISWGGRGTYYADSVKDRFTISR | |
| | DNPKNMVYLQMNSLRAEDTAVYYCAAGDLPGGRRALDYDYWGQGT | |
| | LVTVSS | |
| 57 | EVQLVESGGGLVQAGGSLRLSCAASGGTFSSYTMGWFRQAPGKER | 21H7 co-binder + |
| | EYVGGISSSGYVMYNSESMKGRFTISRENAKNMVYLQMNSLKPED | huIgG1Fc_YTE_C→S |
| | TAVYYCAAGTIGRPYDYWGQGTQVTVSSGGGSGGGGSGGGGSGGG | |
| | GSGGGGSGGGGSGGGGSEGVGLVESGGGSVQAGDSLRLSCAASGR | |
| | TFSTYIIGWFRQAPGKEREFVASISWGGRGTYYADSVKDRFTISR | |
| | DNPKNMVYLQMNSLKPEDTAVYYCAAGDLPGGRRALDYDYWGQGT | |
| | LVTVSSEPKSSDKTHTCPPCPAPELLGGPSVFLFPPKPKDTLYIT | |
| | REPEVTCVVVDVSHEDPEVKFNWYVDGVEVHNAKTKPREEQYNST | |
| | YRVVSVLTVLHQDWLNGKEYKCKVSNKALPAPIEKTISKAKGQPR | |
| | EPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHN | |
| | HYTQKSLSLSPGK | |
| 58 | EVQLVESGGGLVQPGGSLRLSCAASGGTFSSYTMGWFRQAPGKER | Humanized 21H7 |
| | EYVGGISSSGYVMYNSESMKGRFTISRDNAKNTLYLQMNSLRAED | co-binder + |
| | TAVYYCAAGTIGRPYDYWGQGTQVTVSSGGGSGGGGSGGGGSGGG | huIgG1Fc_YTE_C→S |
| | GSGGGGSGGGGSGGGGSEGVGLVESGGGLVQPGGSLRLSCAASGR | |
| | TFSTYIIGWFRQAPGKEREFVASISWGGRGTYYADSVKDRFTISR | |
| | DNPKNMVYLQMNSLRAEDTAVYYCAAGDLPGGRRALDYDYWGQGT | |
| | LVTVSSEPKSSDKTHTCPPCPAPELLGGPSVFLFPPKPKDTLYIT | |
| | REPEVTCVVVDVSHEDPEVKFNWYVDGVEVHNAKTKPREEQYNST | |
| | YRVVSVLTVLHQDWLNGKEYKCKVSNKALPAPIEKTISKAKGQPR | |
| | EPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHN | |
| | HYTQKSLSLSPGK | |
| 59 | SYX₁MX₂ | Exemplary VHH1 |
| | Wherein X₁ is A or T, and X₂ is A or G. | CDR1 consensus |
| | | sequence (Kabat |
| | | numbering) |
| 60 | GISSSGYX₃X₄YX₅SX₆SMKG | Exemplary VHH1 |
| | wherein X₃ is T or V, X₄ is L or M, X₅ is K or | CDR2 consensus |
| | N, and X₆ is D or E. | sequence (Kabat |
| | | numbering) |
| 61 | GX₇X₈GRPYX₉Y | Exemplary VHH1 |
| | wherein X₇ is I or T, X₈ is F or I, and X₉ is | CDR3 consensus |
| | E or D. | sequence (Kabat |
| | | numbering) |
| 62 | EPKSSDKTHTCPPCPAPELLGGPSVFLFPPKPKDTLYITREPEVT | huIgG1Fc_YTE_C→S |
| | CVVVDVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSRDELTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPGK | |
| 63 | GISSSGYX₁₀X₁₁YX₁₂X₁₃X₁₄SMKG | Exemplary VHH |
| | wherein X₁₀ is T or V, X₁₁ is L or M, X₁₂ | CDR2 consensus |
| | is K or N, X₁₃ is S or A, and X₁₄ is D or | sequence (Kabat |
| | E. | numbering) |
| 64 | GX₁₅X₁₆GRPYX₁₇X₁₈ | Exemplary VHH |
| | wherein X₁₅ is I, T, or V, X₁₆ is F or I, and | CDR3 consensus |
| | X₁₇ is E or D, and X₁₈ is Y or F. | sequence (Kabat |
| | | numbering) |
| 65 | EVQLVESGGGSVQAGDSLRLSCAASGGTFSSYTMGWFRQAPGKER | 20C7 VHH1 single |
| | EYVGGISSSGYVMYNSESMKGRFTISRENAKNMVYLQMNSLKPED | binder |
| | TAVYYCAAGTIGRPYDYWGQGTLVTVSS | |
| 66 | EVQLVESGGDLVQAGGSLRLSCAASGGTFSSYAMAWFRQAPGKER | 1H4 VHH2 single |
| | EYVAGISSSGYTLYKADSMKGRFTISRENAKNMVYLQMNSLKPED | binder |
| | TAVYSCAAGVFGRPYEYWGRGTQVTVSS | |
| 67 | EVQLVESGGGSVQAGGSLRLSCAASGGTFSSYTMGWFRQAPGKER | 1C2 VHH2 single |
| | EYVGGISSSGYVMYNSESMKGRFTISRENAKNMVYLQMNSLKPED | binder |
| | TAVYYCAAGTIGRPYDYWGQGTQVTVSS | |
| 68 | EVQLVESGGGSVQAGGSLRLSCAASGGTFSSYAMAWFRQAPGKER | 1E3 VHH1 single |
| | EYVAGISSSGYTLYKSDSMKGRFTISRENAKNMVYLQMNSLKPED | binder |
| | TAVYSCAAGIFGRPYEYWGQGTLVTVSS | |
| 69 | EVQLVESGGGSVQPGGSLRLSCAASGGTFSSYTMGWFRQAPGKER | 1D12 VHH1 single |
| | EYVGGISSSGYVMYNSDSMKGRFTISRENAKNMVYLQMNSLKPED | binder |
| | TAVYYCAAGTIGRPYDFWGQGTLVTVSS | |
| 70 | EVQLVESGGGSVQPGGSLRLSCAASGGTFSSYTMGWFRQAPGKER | 2H2 VHH1 single |
| | EYVGGISSSGYVMYNSESMKGRFTISRENAKNMVYLQMNSLKPED | binder |
| | TAVYYCAAGTIGRPYDYWGQGTLVTVSS | |
| 71 | EVQLVESGGGSVQAGGSLRLSCAASGGTFSSYTMGWFRQAPGKER | 1H2 VHH1 single |
| | EYVGGISSSGYVMYNSESMKGRFTISRENAKNMVYLQMNSLKPED | binder |
| | TAVYYCAAGTIGRPYDYWGQGTQVTVSS | |
| 72 | EVQLVESGGGSVQPGGSLRLSCAASGGTFSSYTMGWFRQAPGKER | 1C7 VHH1 single |
| | EYVGGISSSGYVMYNSESMKGRFTISRENAKNMVYLQMNSLKPED | binder |
| | TAVYYCAAGTIGRPYDYWGQGTQVTVSS | |
| 73 | EVQLVESGGGSVQPGGSLRLSCAASGGTFSSYTMGWFRQAPGKER | 1B10 VHH1 single |
| | EYVGGISSSGYVMYNSDSMKGRFTISRENAKNMVYLQMNSLKPED | binder |
| | TAVYYCAAGTIGRPYDFWGQGTLVTVSS | |
| 74 | EVQLVESGGGSVQAGGSLRLSCAASGGTFSSYAMAWFRQAPGKER | 1G6 VHH1 single |
| | EYVAGISSSGYTLYKSDSMKGRFTISRENAKNMVYLQMNSLKPED | binder |
| | TAVYSCAAGIFGRPYEYWGQGTQVTVSS | |
| 75 | EVQLVESGGGSVQAGGSLRLSCAASGGTFSSYTMGWFRQAPGKER | 1E2 VHH1 single |
| | EYVGGISSSGYVMYNSESMKGRFTISRENAKNMVYLQMNSLKPED | binder |
| | TAVYYCAAGTIGRPYDYWGQGTQVTVSS | |
| 76 | QVKLEESGGGSVQAGGSLRLSCAASGGTFSSYTMGWFRQAPGKER | 1B12 VHH2 single |
| | EYVGGISSSGYVMYNSESMKGRFTISRENAKNMVYLQMNSLKPED | binder |
| | TAVYYCAAGTIGRPYDYWGQGTLVTVSS | |
| 77 | EVQLVESGGGSVQAGGSLRLSCAASGGTFSSYAMAWFRQAPGKER | 1C3 VHH1 single |
| | EYVAGISSSGYTLYKSDSMKGRFTISRENAKNMVYLQMNSLKPED | binder |
| | TAVYSCAAGIFGRPYEYWGQGTQVTVSS | |
| 78 | GGTFSSYT | 1C10 VHH1 |
| | | CDR1 |
| | | (IMGT numbering) |
| 79 | ISSSGYVM | 1C10 VHH1 |
| | | CDR2 |
| | | (IMGT numbering) |
| 80 | AAGTIGRPYDY | 1C10 VHH1 |
| | | CDR3 |
| | | (IMGT numbering) |
| 81 | GGTFSSY | 1C10 VHH1 |
| | | CDR1 |
| | | (Chothia |
| | | numbering) |
| 82 | SSSGYV | 1C10 VHH1 |
| | | CDR2 |
| | | (Chothia |
| | | numbering) |
| 83 | GTIGRPYDY | 1C10 VHH1 |
| | | CDR3 |
| | | (Chothia |
| | | numbering) |
| 84 | GDISSIVA | 1C10 VHH2 |
| | | CDR1 |
| | | (IMGT numbering) |
| 85 | ITSGGRT | 1C10 VHH2 |
| | | CDR2 |
| | | (IMGT numbering) |
| 86 | AADRGWTSVGEYDY | 1C10 VHH2 |
| | | CDR3 |
| | | (IMGT numbering) |
| 87 | GDISSIV | 1C10 VHH2 |
| | | CDR1 |
| | | (Chothia |
| | | numbering) |
| 88 | TSGGR | 1C10 VHH2 |
| | | CDR2 |
| | | (Chothia |
| | | numbering) |
| 89 | DRGWTSVGEYDY | 1C10 VHH2 |
| | | CDR3 |
| | | (Chothia |
| | | numbering) |
| 90 | EPKSSDKTHT CPPCPAPEAA GGPSVFLFPP | huIgG1Fc_YTE_L |
| | KPKDTLYITR EPEVTCVVVD VSHEDPEVKF | ALA_C→S |
| | NWYVDGVEVH NAKTKPREEQ YNSTYRVVSV | |
| | LTVLHQDWLN GKEYKCKVSN KALPAPIEKT | |
| | ISKAKGQPRE PQVYTLPPSR DELTKNQVSL | |
| | TCLVKGFYPS DIAVEWESNG QPENNYKTTP | |
| | PVLDSDGSFF LYSKLTVDKS RWQQGNVFSC | |
| | SVMHEALHNH YTQKSLSLSP GK | |
| 91 | EVQLVESGGGLVQAGGSLRLSCAASGGTFSSYTMGWFRQAPGKER | 1C10 co-binder + |
| | EYVGGISSSGYVMYNSESMKGRFTISRENAKNMVYLQMNSLKPED | huIgG1Fc_ |
| | TAVYYCAAGTIGRPYDYWGQGTQVTVSSGGGSGGGGSGGGGSGGG | YTE_LALA_C→S |
| | GSGGGGSGGGGSGGGGSGMTGVKLEESGGGLVQPGGSLILSCAAS | |
| | GDISSIVAMGWYRQAPGKQRELVAAITSGGRTHYRDSVKGRFTIS | |
| | GNNDNSALYLHMNSLKPEDTAVYYCAADRGWTSVGEYDYWGKGTL | |
| | VTVSSEPKSSDKTHTCPPCPAPEAAGGPSVFLFPPKPKDTLYITR | |
| | EPEVTCVVVDVSHEDPEVKFNWYVDGVEVHNAKTKPREEQYNSTY | |
| | RVVSVLTVLHQDWLNGKEYKCKVSNKALPAPIEKTISKAKGQPRE | |
| | PQVYTLPPSRDELTKNQVSLTCLVKGFYPSDIAVEWESNGQPENN | |
| | YKTTPPVLDSDGSFFLYSKLTVDKSRWQQGNVFSCSVMHEALHNH | |
| | YTQKSLSLSPGK | |
| 92 | EVQLVESGGGLVQPGGSLRLSCAASGGTFSSYTMGWFRQAPGKER | Humanized 1C10 |
| | EYVGGISSSGYVMYNSESMKGRFTISRDNAKNTLYLQMNSLRAED | co-binder + |
| | TAVYYCAAGTIGRPYDYWGQGTQVTVSSGGGSGGGGSGGGGSGGG | huIgG1Fc_ |
| | GSGGGGSGGGGSGGGGSGMTGVQLVESGGGLVQPGGSLRLSCAAS | YTE_LALA_C→S |
| | GDISSIVAMGWYRQAPGKQRELVSAITSGGRTHYRDSVKGRFTIS | |
| | RDNAKNTLYLQMNSLRAEDTAVYYCAADRGWTSVGEYDYWGQGTQ | |
| | VTVSSEPKSSDKTHTCPPCPAPEAAGGPSVFLFPPKPKDTLYITR | |
| | EPEVTCVVVDVSHEDPEVKFNWYVDGVEVHNAKTKPREEQYNSTY | |
| | RVVSVLTVLHQDWLNGKEYKCKVSNKALPAPIEKTISKAKGQPRE | |
| | PQVYTLPPSRDELTKNQVSLTCLVKGFYPSDIAVEWESNGQPENN | |
| | YKTTPPVLDSDGSFFLYSKLTVDKSRWQQGNVFSCSVMHEALHNH | |
| | YTQKSLSLSPGK | |
| 93 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | VHH1 with N- |
| | DTAVYYCAAGTIGRPYDYWGQGTQVTVSS wherein X₁₉ is E | terminal consensus |
| | or Q | residue |
| 94 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | VHH1 with N- |
| | AEDTAVYYCAAGTIGRPYDYWGQGTQVTVSS wherein X₁ is | terminal consensus |
| | A or T, X₂ is A or G, and X₁₉ is E or Q | residue, and |
| | | consensus CDR1 |
| 95 | X₁₉VQLVESGGGLVQPGGSLRLSCAASGGTFSSYTMGWFRQAPGKE | Humanized 1C10 |
| | REYVGGISSSGYX₃X₄YX₅SX₆SMKG | VHH1 with N- |
| | RFTISRDNAKNTLYLQMNSLRAEDTAVYYCAAGTIGRPYDYWGQG | terminal consensus |
| | TQVTVSS wherein X₃ is T or V, X₄ is L or M, X₅ | residue, and |
| | is K or N, X₆ is D or E, and X₁₉ is E or Q | consensus CDR2A |
| 96 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | VHH1 with N- |
| | DTAVYYCAAGX₇X₈GRPYX₉YWGQGTQVTVSS wherein X₇ is | terminal consensus |
| | I or T, X₈ is F or I, X₉ is E or D, and X₁₉ is | residue, and |
| | E or Q | consensus CDR3A |
| 97 | X₁₉VQLVESGGGLVQPGGSLRLSCAASGGTFSSYX₁MX₂WFRQAPG | Humanized 1C10 |
| | KEREYVGGISSSGYX₃X₄YX₅X₆SMKGRFTISRDNAKNTLYLQMN | VHH1 with N- |
| | SLRAEDTAVYYCAAGTIGRPYDYWGQGTQVTVSS wherein X₁ | terminal consensus |
| | is A or T, X₂ is A or G, X₃ is T or V, X₄ is L | residue, and |
| | or M, X₅ is K or N, X₆ is D or E, and X₁₉ is E | consensus CDRs1 |
| | or Q | and 2A |
| 98 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | VHH1 with N- |
| | AEDTAVYYCAAGX₇X₈GRPYX₉YWGQGTQVTVSS wherein X₁ | terminal consensus |
| | is A or T, X₂ is A or G, X₇ is I or T, X₈ is F | residue, and |
| | or I, X₉ is E or D, and X₁₉ is E or Q | consensus CDRs1 |
| | | and 3A |
| 99 | X₁₉VQLVESGGGLVQPGGSLRLSCAASGGTFSSYTMGWFRQAPGKE | Humanized 1C10 |
| | REYVGGISSSGYX₃X₄YX₅SX₆SMKGRFTISRDNAKNTLYLQMNSL | VHH1 with N- |
| | RAEDTAVYYCAAGX₇X₈GRPYX₉YWGQGTQVTVSS wherein X₃ | terminal consensus |
| | is T or V, X₄ is L or M, X₅ is K or N, X₆ is D | residue, and |
| | or E, X₇ is I or T, X₈ is F or I, Xe is E or | consensus |
| | D, and X₁₉ is E or Q | CDRs2A and 3A |
| 100 | X₁₉VQLVESGGGLVQPGGSLRLSCAASGGTFSSYX₁MX₂WFRQAPG | Humanized 1C10 |
| | KEREYVGGISSSGYX₃X₄YX₅X₆SMKGRFTISRDNAKNTLYLQMN | VHH1 with N- |
| | SLRAEDTAVYYCAAGX₇X₈GRPYX₉YWGQGTQVTVSS wherein | terminal consensus |
| | X₁ is A or T, X₂ is A or G, X₃ is T or V, X₄ | residue, and |
| | is L or M, X₅ is K or N, X₆ is D or E, X₇ is I | consensus CDRs1, |
| | or T, X₈ is F or I, X₉ is E or D, and X₁₉ is E | 2A and 3A |
| | or Q | |
| 101 | X₂₀VQLVESGGGLVQPGGSLRLSCAASGDISSIVAMGWYRQAPGKQ | Humanized 1C10 |
| | RELVSAITSGGRTHYRDSVKGRFTISRDNAKNTLYLQMNSLRAED | VHH2 with one |
| | TAVYYCAADRGWTSVGEYDYWGQGTQVTVSS wherein X₂₀ is | amino acid |
| | E or Q | consensus addition |
| | | at N-terminus |
| 102 | QLVESGGGLVQPGGSLRLSCAASGDISSIVAMGWYRQAPGKQREL | Humanized 1C10 |
| | VSAITSGGRTHYRDSVKGRFTISRDNAKNTLYLQMNSLRAEDTAV | VHH2 with one |
| | YYCAADRGWTSVGEYDYWGQGTQVTVSS | amino acid |
| | | deletion at N- |
| | | terminus |
| 103 | LVESGGGLVQPGGSLRLSCAASGDISSIVAMGWYRQAPGKQRELV | Humanized 1C10 |
| | SAITSGGRTHYRDSVKGRFTISRDNAKNTLYLQMNSLRAEDTAVY | VHH2 with two |
| | YCAADRGWTSVGEYDYWGQGTQVTVSS | amino acid |
| | | deletion at N- |
| | | terminus |
| 104 | GGGSGGGGSGGGGSGGGGSGGGGSGGGGSGGGGSGMTG | Humanized 1C10 |
| | | linker connecting |
| | | VHH1 and VHH2 |
| 105 | EVQLVESGGGLVQPGGSLRLSCAASGGTFSSYTMGWFRQAPGKER | Humanized 1C10 |
| | EYVGGISSSGYVMYNSESMKGRFTISRDNAKNTLYLQMNSLRAED | co-binder with |
| | TAVYYCAAGTIGRPYDYWGQGTQVTVSSGGGSGGGGSGGGGSGGG | VHH2 having one |
| | GSGGGGSGGGGSGGGGSGMTGX₂₀VQLVESGGGLVQPGGSLRLSCA | amino acid |
| | ASGDISSIVAMGWYRQAPGKQRELVSAITSGGRTHYRDSVKGRFT | consensus addition |
| | ISRDNAKNTLYLQMNSLRAEDTAVYYCAADRGWTSVGEYDYWGQG | at N-terminus |
| | TQVTVSS wherein X₂₀ is E or Q | |
| 106 | EVQLVESGGGLVQPGGSLRLSCAASGGTFSSYTMGWFRQAPGKER | Humanized 1C10 |
| | EYVGGISSSGYVMYNSESMKGRFTISRDNAKNTLYLQMNSLRAED | co-binder with |
| | TAVYYCAAGTIGRPYDYWGQGTQVTVSSGGGSGGGGSGGGGSGGG | VHH2 having one |
| | GSGGGGSGGGGSGGGGSGMTGQLVESGGGLVQPGGSLRLSCAASG | amino acid |
| | DISSIVAMGWYRQAPGKQRELVSAITSGGRTHYRDSVKGRFTISR | deletion at N- |
| | DNAKNTLYLQMNSLRAEDTAVYYCAADRGWTSVGEYDYWGQGTQV | terminus |
| | TVSS | |
| 107 | EVQLVESGGGLVQPGGSLRLSCAASGGTFSSYTMGWFRQAPGKER | Humanized 1C10 |
| | EYVGGISSSGYVMYNSESMKGRFTISRDNAKNTLYLQMNSLRAED | co-binder with |
| | TAVYYCAAGTIGRPYDYWGQGTQVTVSSGGGSGGGGSGGGGSGGG | VHH2 having two |
| | GSGGGGSGGGGSGGGGSGMTGLVESGGGLVQPGGSLRLSCAASGD | amino acid |
| | ISSIVAMGWYRQAPGKQRELVSAITSGGRTHYRDSVKGRFTISRD | deletion at N- |
| | NAKNTLYLQMNSLRAEDTAVYYCAADRGWTSVGEYDYWGQGTQVT | terminus |
| | VSS | |
| 108 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGTIGRPYDYWGQGTQVTVSSGGGSGGGGSGGGGSGG | VHH1 N-terminal |
| | GGSGGGGSGGGGSGGGGSGMTGVQLVESGGGLVQPGGSLRLSCAA | consensus residue |
| | SGDISSIVAMGWYRQAPGKQRELVSAITSGGRTHYRDSVKGRFTI | |
| | SRDNAKNTLYLQMNSLRAEDTAVYYCAADRGWTSVGEYDYWGQGT | |
| | QVTVSS wherein X₁₉ is E or Q | |
| 109 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGTIGRPYDYWGQGTQVTVSSGGGSGGGGSGGGGSGG | VHH1 N-terminal |
| | GGSGGGGSGGGGSGGGGSGMTGX₂₀VQLVESGGGLVQPGGSLRLSC | consensus residue, |
| | AASGDISSIVAMGWYRQAPGKQRELVSAITSGGRTHYRDSVKGRF | and VHH2 with |
| | TISRDNAKNTLYLQMNSLRAEDTAVYYCAADRGWTSVGEYDYWGQ | one amino acid |
| | GTQVTVSS wherein X₁₉ is E or Q, and X₂₀ is E | consensus addition |
| | or Q | at N-terminus |
| 110 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGTIGRPYDYWGQGTQVTVSSGGGSGGGGSGGGGSGG | VHH1 N-terminal |
| | GGSGGGGSGGGGSGGGGSGMTGQLVESGGGLVQPGGSLRLSCAAS | consensus residue, |
| | GDISSIVAMGWYRQAPGKQRELVSAITSGGRTHYRDSVKGRFTIS | and VHH2 with |
| | RDNAKNTLYLQMNSLRAEDTAVYYCAADRGWTSVGEYDYWGQGTQ | one amino acid |
| | VTVSS wherein X₁₉ is E or Q | deletion at N- |
| | | terminus |
| 111 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGTIGRPYDYWGQGTQVTVSSGGGSGGGGSGGGGSGG | VHH1 N-terminal |
| | GGSGGGGSGGGGSGGGGSGMTGLVESGGGLVQPGGSLRLSCAASG | consensus residue, |
| | DISSIVAMGWYRQAPGKQRELVSAITSGGRTHYRDSVKGRFTISR | and VHH2 with |
| | DNAKNTLYLQMNSLRAEDTAVYYCAADRGWTSVGEYDYWGQGTQV | two amino acid |
| | TVSS wherein X₁₉ is E or Q | deletion at N- |
| | | terminus |
| 112 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGTIGRPYDYWGQGTQVTVSSGGGSGGGGSGGGGS | VHH1 N-terminal |
| | GGGGSGGGGSGGGGSGGGGSGMTGVQLVESGGGLVQPGGSLRLSC | consensus residue |
| | AASGDISSIVAMGWYRQAPGKQRELVSAITSGGRTHYRDSVKGRF | and consensus |
| | TISRDNAKNTLYLQMNSLRAEDTAVYYCAADRGWTSVGEYDYWGQ | CDR1 |
| | GTQVTVSS wherein X₁ is A or T, X₂ is A or G, | |
| | and X₁₉ is E or Q | |
| 113 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGTIGRPYDYWGQGTQVTVSSGGGSGGGGSGGGGS | VHH1 N-terminal |
| | GGGGSGGGGSGGGGSGGGGSGMTGX₂₀VQLVESGGGLVQPGGSLRL | consensus residue |
| | SCAASGDISSIVAMGWYRQAPGKQRELVSAITSGGRTHYRDSVKG | and consensus |
| | RFTISRDNAKNTLYLQMNSLRAEDTAVYYCAADRGWTSVGEYDYW | CDR1, and VHH2 |
| | GQGTQVTVSS wherein X₁ is A or T, X₂ is A or G, | with one amino |
| | X₁₉ is E or Q, and X₂₀ is E or Q | acid consensus |
| | | addition at N- |
| | | terminus |
| 114 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGTIGRPYDYWGQGTQVTVSSGGGSGGGGSGGGGS | VHH1 N-terminal |
| | GGGGSGGGGSGGGGSGGGGSGMTGQLVESGGGLVQPGGSLRLSCA | consensus residue |
| | ASGDISSIVAMGWYRQAPGKQRELVSAITSGGRTHYRDSVKGRFT | and consensus |
| | ISRDNAKNTLYLQMNSLRAEDTAVYYCAADRGWTSVGEYDYWGQG | CDR1, and VHH2 |
| | TQVTVSS wherein X₁ is A or T, X₂ is A or G, | with one amino |
| | and X₁₉ is E or Q | acid deletion at N- |
| | | terminus |
| 115 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGTIGRPYDYWGQGTQVTVSSGGGSGGGGSGGGGS | VHH1 N-terminal |
| | GGGGSGGGGSGGGGSGGGGSGMTGLVESGGGLVQPGGSLRLSCAA | consensus residue |
| | SGDISSIVAMGWYRQAPGKQRELVSAITSGGRTHYRDSVKGRFTI | and consensus |
| | SRDNAKNTLYLQMNSLRAEDTAVYYCAADRGWTSVGEYDYWGQGT | CDR1, and VHH2 |
| | QVTVSS wherein X₁ is A or T, X₂ is A or G, and | with two amino |
| | X₁₉ is E or Q | acid deletion at N- |
| | | terminus |
| 116 | X₁₉VQLVESGGGLVQPGGSLRLSCAASGGTFSSYTMGWFRQAPGKE | Humanized 1C10 |
| | REYVGGISSSGYX₃X₄YX₅X₆SMKGRFTISRDNAKNTLYLQMNSL | co-binder with |
| | RAEDTAVYYCAAGTIGRPYDYWGQGTQVTVSSGGGSGGGGSGGGG | VHH1 N-terminal |
| | SGGGGSGGGGSGGGGSGGGGSGMTGVQLVESGGGLVQPGGSLRLS | consensus residue |
| | CAASGDISSIVAMGWYRQAPGKQRELVSAITSGGRTHYRDSVKGR | and consensus |
| | FTISRDNAKNTLYLQMNSLRAEDTAVYYCAADRGWTSVGEYDYWG | CDR2A |
| | QGTQVTVSS wherein X₃ is T or V, X₄ is L or M, | |
| | X₅ is K or N, X₆ is D or E, and X₁₉ is E or Q | |
| 117 | X₁₉VQLVESGGGLVQPGGSLRLSCAASGGTFSSYTMGWFRQAPGKE | Humanized 1C10 |
| | REYVGGISSSGYX₃X₄YX₅SX₆SMKGRFTISRDNAKNTLYLQMNSL | co-binder with |
| | RAEDTAVYYCAAGTIGRPYDYWGQGTQVTVSSGGGSGGGGSGGGG | VHH1 N-terminal |
| | SGGGGSGGGGSGGGGSGGGGSGMTGX₂₀VQLVESGGGLVQPGGSLR | consensus residue |
| | LSCAASGDISSIVAMGWYRQAPGKQRELVSAITSGGRTHYRDSVK | and consensus |
| | GRFTISRDNAKNTLYLQMNSLRAEDTAVYYCAADRGWTSVGEYDY | CDR2A, and |
| | WGQGTQVTVSS wherein X₃ is T or V, X₄ is L or | VHH2 with one |
| | M, X₅ is K or N, X₆ is D or E, X₁₉ is E or Q, | amino acid |
| | and X₂₀ is E or Q | consensus addition |
| | | at N-terminus |
| 118 | X₁₉VQLVESGGGLVQPGGSLRLSCAASGGTFSSYTMGWFRQAPGKE | Humanized 1C10 |
| | REYVGGISSSGYX₃X₄YX₅SX₆SMKGRFTISRDNAKNTLYLQMNSL | co-binder with |
| | RAEDTAVYYCAAGTIGRPYDYWGQGTQVTVSSGGGSGGGGSGGGG | VHH1 N-terminal |
| | SGGGGSGGGGSGGGGSGGGGSGMTGQLVESGGGLVQPGGSLRLSC | consensus residue |
| | AASGDISSIVAMGWYRQAPGKQRELVSAITSGGRTHYRDSVKGRF | and consensus |
| | TISRDNAKNTLYLQMNSLRAEDTAVYYCAADRGWTSVGEYDYWGQ | CDR2A, and |
| | GTQVTVSS wherein X₃ is T or V, X₄ is L or M, | VHH2 with one |
| | X₅ is K or N, X₆ is D or E, and X₁₉ is E or Q | amino acid |
| | | deletion at N- |
| | | terminus |
| 119 | X₁₉VQLVESGGGLVQPGGSLRLSCAASGGTFSSYTMGWFRQAPGKE | Humanized 1C10 |
| | REYVGGISSSGYX₃X₄YX₅X₆SMKGRFTISRDNAKNTLYLQMNSL | co-binder with |
| | RAEDTAVYYCAAGTIGRPYDYWGQGTQVTVSSGGGSGGGGSGGGG | VHH1 N-terminal |
| | SGGGGSGGGGSGGGGSGGGGSGMTGLVESGGGLVQPGGSLRLSCA | consensus residue |
| | ASGDISSIVAMGWYRQAPGKQRELVSAITSGGRTHYRDSVKGRFT | and consensus |
| | ISRDNAKNTLYLQMNSLRAEDTAVYYCAADRGWTSVGEYDYWGQG | CDR2A, and |
| | TQVTVSS wherein X₃ is T or V, X₄ is L or M, X₅ | VHH2 with two |
| | is K or N, X₆ is D or E, and X₁₉ is E or Q | amino acid |
| | | deletion at N- |
| | | terminus |
| 120 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGX₇X₈GRPYX₉YWGQGTQVTVSSGGGSGGGGSGGGGS | VHH1 N-terminal |
| | GGGGSGGGGSGGGGSGGGGSGMTGVQLVESGGGLVQPGGSLRLSC | consensus residue |
| | AASGDISSIVAMGWYRQAPGKQRELVSAITSGGRTHYRDSVKGRF | and consensus |
| | TISRDNAKNTLYLQMNSLRAEDTAVYYCAADRGWTSVGEYDYWGQ | CDR3A |
| | GTQVTVSS wherein X₇ is I or T, X₈ is F or I, | |
| | X₉ is E or D, and X₁₉ is E or Q | |
| 121 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGX₇X₈GRPYX₉YWGQGTQVTVSSGGGSGGGGSGGGGS | VHH1 N-terminal |
| | GGGGSGGGGSGGGGSGGGGSGMTGX₂₀VQLVESGGGLVQPGGSLRL | consensus residue |
| | SCAASGDISSIVAMGWYRQAPGKQRELVSAITSGGRTHYRDSVKG | and consensus |
| | RFTISRDNAKNTLYLQMNSLRAEDTAVYYCAADRGWTSVGEYDYW | CDR3A, and |
| | GQGTQVTVSS wherein X₇ is I or T, X₈ is F or I, | VHH2 with one |
| | X₉ is E or D, X₁₉ is E or Q, and X₂₀ is E or Q | amino acid |
| | | consensus addition |
| | | at N-terminus |
| 122 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGX₇X₈GRPYX₉YWGQGTQVTVSSGGGSGGGGSGGGGS | VHH1 N-terminal |
| | GGGGSGGGGSGGGGSGGGGSGMTGQLVESGGGLVQPGGSLRLSCA | consensus residue |
| | ASGDISSIVAMGWYRQAPGKQRELVSAITSGGRTHYRDSVKGRFT | and consensus |
| | ISRDNAKNTLYLQMNSLRAEDTAVYYCAADRGWTSVGEYDYWGQG | CDR3A, and |
| | TQVTVSS wherein X₇ is I or T, X₈ is F or I, X₉ | VHH2 with one |
| | is E or D, and X₁₉ is E or Q | amino acid |
| | | deletion at N- |
| | | terminus |
| 123 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGX₇X₈GRPYX₉YWGQGTQVTVSSGGGSGGGGSGGGGS | VHH1 N-terminal |
| | GGGGSGGGGSGGGGSGGGGSGMTGLVESGGGLVQPGGSLRLSCAA | consensus residue |
| | SGDISSIVAMGWYRQAPGKQRELVSAITSGGRTHYRDSVKGRFTI | and consensus |
| | SRDNAKNTLYLQMNSLRAEDTAVYYCAADRGWTSVGEYDYWGQGT | CDR3A, and |
| | QVTVSS wherein X₇ is I or T, X₈ is F or I, X₉ | VHH2 with two |
| | is E or D, and X₁₉ is E or Q | amino acid |
| | | deletion at N- |
| | | terminus |
| 124 | X₁₉VQLVESGGGLVQPGGSLRLSCAASGGTFSSYX₁MX₂WFRQAPG | Humanized 1C10 |
| | KEREYVGGISSSGYX₃X₄YX₅X₆SMKGRFTISRDNAKNTLYLQMN | co-binder with |
| | SLRAEDTAVYYCAAGTIGRPYDYWGQGTQVTVSSGGGSGGGGSGG | VHH1 N-terminal |
| | GGSGGGGSGGGGSGGGGSGGGGSGMTGVQLVESGGGLVQPGGSLR | consensus residue |
| | LSCAASGDISSIVAMGWYRQAPGKQRELVSAITSGGRTHYRDSVK | and consensus |
| | GRFTISRDNAKNTLYLQMNSLRAEDTAVYYCAADRGWTSVGEYDY | CDRs1 and 2A |
| | WGQGTQVTVSS wherein X₁ is A or T, X₂ is A or | |
| | G, X₃ is T or V, X₄ is L or M, X₅ is K or N, | |
| | X₆ is D or E, and X₁₉ is E or Q | |
| 125 | X₁₉VQLVESGGGLVQPGGSLRLSCAASGGTFSSYX₁MX₂WFRQAPG | Humanized 1C10 |
| | KEREYVGGISSSGYX₃X₄YX₅SX₆SMKGRFTISRDNAKNTLYLQMN | co-binder with |
| | SLRAEDTAVYYCAAGTIGRPYDYWGQGTQVTVSSGGGSGGGGSGG | VHH1 N-terminal |
| | GGSGGGGSGGGGSGGGGSGGGGSGMTGX₂₀VQLVESGGGLVQPGGS | consensus residue |
| | LRLSCAASGDISSIVAMGWYRQAPGKQRELVSAITSGGRTHYRDS | and consensus |
| | VKGRFTISRDNAKNTLYLQMNSLRAEDTAVYYCAADRGWTSVGEY | CDRs1 and 2A, |
| | DYWGQGTQVTVSS wherein X₁ is A or T, and X₂ is | and VHH2 with |
| | A or G, X₃ is T or V, X₄ is L or M, X₅ is K or | one amino acid |
| | N, X₆ is D or E, X₁₉ is E or Q, and X₂₀ is E or | consensus addition |
| | Q | at N-terminus |
| 126 | X₁₉VQLVESGGGLVQPGGSLRLSCAASGGTFSSYX₁MX₂WFRQAPG | Humanized 1C10 |
| | KEREYVGGISSSGYX₃X₄YX₅SX₆SMKGRFTISRDNAKNTLYLQMN | co-binder with |
| | SLRAEDTAVYYCAAGTIGRPYDYWGQGTQVTVSSGGGSGGGGSGG | VHH1 N-terminal |
| | GGSGGGGSGGGGSGGGGSGGGGSGMTGQLVESGGGLVQPGGSLRL | consensus residue |
| | SCAASGDISSIVAMGWYRQAPGKQRELVSAITSGGRTHYRDSVKG | and consensus |
| | RFTISRDNAKNTLYLQMNSLRAEDTAVYYCAADRGWTSVGEYDYW | CDRs1 and 2A, |
| | GQGTQVTVSS wherein X₁ is A or T, X₂ is A or G, | and VHH2 with |
| | X₃ is T or V, X₄ is L or M, X₅ is K or N, X₆ | one amino acid |
| | is D or E, and X₁₉ is E or Q | deletion at N- |
| | | terminus |
| 127 | X₁₉VQLVESGGGLVQPGGSLRLSCAASGGTFSSYX₁MX₂WFRQAPG | Humanized 1C10 |
| | KEREYVGGISSSGYX₃X₄YX₅SX₆SMKGRFTISRDNAKNTLYLQMN | co-binder with |
| | SLRAEDTAVYYCAAGTIGRPYDYWGQGTQVTVSSGGGSGGGGSGG | VHH1 N-terminal |
| | GGSGGGGSGGGGSGGGGSGGGGSGMTGLVESGGGLVQPGGSLRLS | consensus residue |
| | CAASGDISSIVAMGWYRQAPGKQRELVSAITSGGRTHYRDSVKGR | and consensus |
| | FTISRDNAKNTLYLQMNSLRAEDTAVYYCAADRGWTSVGEYDYWG | CDRs1 and 2A, |
| | QGTQVTVSS wherein X₁ is A or T, X₂ is A or G, | and VHH2 with |
| | X₃ is T or V, X₄ is L or M, X₅ is K or N, X₆ | two amino acid |
| | is D or E, and X₁₉ is E or Q | deletion at N- |
| | | terminus |
| 128 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGX₇X₈GRPYX₉YWGQGTQVTVSSGGGSGGGGSGGG | VHH1 N-terminal |
| | GSGGGGSGGGGSGGGGSGGGGSGMTGVQLVESGGGLVQPGGSLRL | consensus residue |
| | SCAASGDISSIVAMGWYRQAPGKQRELVSAITSGGRTHYRDSVKG | and consensus |
| | RFTISRDNAKNTLYLQMNSLRAEDTAVYYCAADRGWTSVGEYDYW | CDRs1 and 3A |
| | GQGTQVTVSS wherein X₁ is A or T, X₂ is A or G, | |
| | X₇ is I or T, X₈ is F or I, X, is E or D, and | |
| | X₁₉ is E or Q | |
| 129 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGX₇X₈GRPYX₉YWGQGTQVTVSSGGGSGGGGSGGG | VHH1 N-terminal |
| | GSGGGGSGGGGSGGGGSGGGGSGMTGX₂₀VQLVESGGGLVQPGGSL | consensus residue |
| | RLSCAASGDISSIVAMGWYRQAPGKQRELVSAITSGGRTHYRDSV | and consensus |
| | KGRFTISRDNAKNTLYLQMNSLRAEDTAVYYCAADRGWTSVGEYD | CDRs1 and 3A, |
| | YWGQGTQVTVSS wherein X₁ is A or T, X₂ is A or | and VHH2 with |
| | G, X₇ is I or T, X₈ is F or I, X₉ is E or D, | one amino acid |
| | X₁₉ is E or Q, and X₂₀ is E or Q | consensus addition |
| | | at N-terminus |
| 130 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGX₇X₈GRPYX₉YWGQGTQVTVSSGGGSGGGGSGGG | VHH1 N-terminal |
| | GSGGGGSGGGGSGGGGSGGGGSGMTGQLVESGGGLVQPGGSLRLS | consensus residue |
| | CAASGDISSIVAMGWYRQAPGKQRELVSAITSGGRTHYRDSVKGR | and consensus |
| | FTISRDNAKNTLYLQMNSLRAEDTAVYYCAADRGWTSVGEYDYWG | CDRs1 and 3A, |
| | QGTQVTVSS wherein X₁ is A or T, X₂ is A or G, | and VHH2 with |
| | X₇ is I or T, X₈ is F or I, X₉ is E or D, and | one amino acid |
| | X₁₉ is E or Q | deletion at N- |
| | | terminus |
| 131 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGX₇X₈GRPYX₉YWGQGTQVTVSSGGGSGGGGSGGG | VHH1 N-terminal |
| | GSGGGGSGGGGSGGGGSGGGGSGMTGLVESGGGLVQPGGSLRLSC | consensus residue |
| | AASGDISSIVAMGWYRQAPGKQRELVSAITSGGRTHYRDSVKGRF | and consensus |
| | TISRDNAKNTLYLQMNSLRAEDTAVYYCAADRGWTSVGEYDYWGQ | CDRs1 and 3A, |
| | GTQVTVSS wherein X₁ is A or T, X₂ is A or G, | and VHH2 with |
| | X₇ is I or T, X₈ is F or I, X₉ is E or D, and | two amino acid |
| | X₁₉ is E or Q | deletion at N- |
| | | terminus |
| 132 | X₁₉VQLVESGGGLVQPGGSLRLSCAASGGTFSSYTMGWFRQAPGKE | Humanized 1C10 |
| | REYVGGISSSGYX₃X₄YX₅SX₆SMKGRFTISRDNAKNTLYLQMNSL | co-binder with |
| | RAEDTAVYYCAAGX₇X₈GRPYX₉YWGQGTQVTVSSGGGSGGGGSGG | VHH1 N-terminal |
| | GGSGGGGSGGGGSGGGGSGGGGSGMTGVQLVESGGGLVQPGGSLR | consensus residue |
| | LSCAASGDISSIVAMGWYRQAPGKQRELVSAITSGGRTHYRDSVK | and consensus |
| | GRFTISRDNAKNTLYLQMNSLRAEDTAVYYCAADRGWTSVGEYDY | CDRs2A and 3A |
| | WGQGTQVTVSS wherein X₃ is T or V, X₄ is L or | |
| | M, X₅ is K or N, and X₆ is D or E, X₇ is I or | |
| | T, X₈ is F or I, X, is E or D, and X₁₉ is E or | |
| | Q | |
| 133 | X₁₉VQLVESGGGLVQPGGSLRLSCAASGGTFSSYTMGWFRQAPGKE | Humanized 1C10 |
| | REYVGGISSSGYX₃X₄YX₅SX₆SMKGRFTISRDNAKNTLYLQMNSL | co-binder with |
| | RAEDTAVYYCAAGX₇X₈GRPYX₉YWGQGTQVTVSSGGGSGGGGSGG | VHH1 N-terminal |
| | GGSGGGGSGGGGSGGGGSGGGGSGMTGX₂₀VQLVESGGGLVQPGGS | consensus residue |
| | LRLSCAASGDISSIVAMGWYRQAPGKQRELVSAITSGGRTHYRDS | and consensus |
| | VKGRFTISRDNAKNTLYLQMNSLRAEDTAVYYCAADRGWTSVGEY | CDRs2A and 3A, |
| | DYWGQGTQVTVSS wherein X₃ is T or V, X₄ is L or | and VHH2 with |
| | M, X₅ is K or N, X₆ is D or E, X₇ is I or T, | one amino acid |
| | X₈ is F or I, X₉ is E or D, X₁₉ is E or Q, and | consensus addition |
| | X₂₀ is E or Q | at N-terminus |
| 134 | X₁₉VQLVESGGGLVQPGGSLRLSCAASGGTFSSYTMGWFRQAPGKE | Humanized 1C10 |
| | REYVGGISSSGYX₃X₄YX₅SX₆SMKGRFTISRDNAKNTLYLQMNSL | co-binder with |
| | RAEDTAVYYCAAGX₇X₈GRPYX₉YWGQGTQVTVSSGGGSGGGGSGG | VHH1 N-terminal |
| | GGSGGGGSGGGGSGGGGSGGGGSGMTGQLVESGGGLVQPGGSLRL | consensus residue |
| | SCAASGDISSIVAMGWYRQAPGKQRELVSAITSGGRTHYRDSVKG | and consensus |
| | RFTISRDNAKNTLYLQMNSLRAEDTAVYYCAADRGWTSVGEYDYW | CDRs2A and 3A, |
| | GQGTQVTVSS wherein X₃ is T or V, X₄ is L or M, | and VHH2 with |
| | X₅ is K or N, X₆ is D or E, X₇ is I or T, X₈ | one amino acid |
| | is F or I, X₉ is E or D, and X₁₉ is E or Q | deletion at N- |
| | | terminus |
| 135 | X₁₉VQLVESGGGLVQPGGSLRLSCAASGGTFSSYTMGWFRQAPGKE | Humanized 1C10 |
| | REYVGGISSSGYX₃X₄YX₅SX₆SMKGRFTISRDNAKNTLYLQMNSL | co-binder with |
| | RAEDTAVYYCAAGX₇X₈GRPYX₉YWGQGTQVTVSSGGGSGGGGSGG | VHH1 N-terminal |
| | GGSGGGGSGGGGSGGGGSGGGGSGMTGLVESGGGLVQPGGSLRLS | consensus residue |
| | CAASGDISSIVAMGWYRQAPGKQRELVSAITSGGRTHYRDSVKGR | and consensus |
| | FTISRDNAKNTLYLQMNSLRAEDTAVYYCAADRGWTSVGEYDYWG | CDRs2A and 3A, |
| | QGTQVTVSS wherein X₃ is T or V, X₄ is L or M, | and VHH2 with |
| | X₅ is K or N, X₆ is D or E, X₇ is I or T, X₈ | two amino acid |
| | is F or I, X₉ is E or D, and X₁₉ is E or Q | deletion at N- |
| | | terminus |
| 136 | X₁₉VQLVESGGGLVQPGGSLRLSCAASGGTFSSYX₁MX₂WFRQAPG | Humanized 1C10 |
| | KEREYVGGISSSGYX₃X₄YX₅SX₆SMKGRFTISRDNAKNTLYLQMN | co-binder with |
| | SLRAEDTAVYYCAAGX₇X₈GRPYX₉YWGQGTQVTVSSGGGSGGGGS | VHH1 N-terminal |
| | GGGGSGGGGSGGGGSGGGGSGGGGSGMTGVQLVESGGGLVQPGGS | consensus residue |
| | LRLSCAASGDISSIVAMGWYRQAPGKQRELVSAITSGGRTHYRDS | and consensus |
| | VKGRFTISRDNAKNTLYLQMNSLRAEDTAVYYCAADRGWTSVGEY | CDRs1, 2A and |
| | DYWGQGTQVTVSS wherein X₁ is A or T, X₂ is A or | 3A |
| | G, X₃ is T or V, X₄ is L or M, X₅ is K or N, | |
| | X₆ is D or E, X₇ is I or T, X₈ is F or I, X₉ | |
| | is E or D, and X₁₉ is E or Q | |
| 137 | X₁₉VQLVESGGGLVQPGGSLRLSCAASGGTFSSYX₁MX₂WFRQAPG | Humanized 1C10 |
| | KEREYVGGISSSGYX₃X₄YX₅SX₆SMKGRFTISRDNAKNTLYLQMN | co-binder with |
| | SLRAEDTAVYYCAAGX₇X₈GRPYX₉YWGQGTQVTVSSGGGSGGGGS | VHH1 N-terminal |
| | GGGGSGGGGSGGGGSGGGGSGGGGSGMTGX₂₀VQLVESGGGLVQPG | consensus residue |
| | GSLRLSCAASGDISSIVAMGWYRQAPGKQRELVSAITSGGRTHYR | and consensus |
| | DSVKGRFTISRDNAKNTLYLQMNSLRAEDTAVYYCAADRGWTSVG | CDRs1, 2A and |
| | EYDYWGQGTQVTVSS wherein X₁ is A or T, X₂ is A | 3A, and VHH2 |
| | or G, X₃ is T or V, X₄ is L or M, X₅ is K or | with one amino |
| | N, X₆ is D or E, X₇ is I or T, X₈ is F or I, | acid consensus |
| | X₉ is E or D, X₁₉ is E or Q, and X₂₀ is E or Q | addition at N- |
| | | terminus |
| 138 | X₁₉VQLVESGGGLVQPGGSLRLSCAASGGTFSSYX₁MX₂WFRQAPG | Humanized 1C10 |
| | KEREYVGGISSSGYX₃X₄YX₅SX₆SMKGRFTISRDNAKNTLYLQMN | co-binder with |
| | SLRAEDTAVYYCAAGX₇X₈GRPYX₉YWGQGTQVTVSSGGGSGGGGS | VHH1 N-terminal |
| | GGGGSGGGGSGGGGSGGGGSGGGGSGMTGQLVESGGGLVQPGGSL | consensus residue |
| | RLSCAASGDISSIVAMGWYRQAPGKQRELVSAITSGGRTHYRDSV | and consensus |
| | KGRFTISRDNAKNTLYLQMNSLRAEDTAVYYCAADRGWTSVGEYD | CDRs1, 2A and |
| | YWGQGTQVTVSS wherein X₁ is A or T, X₂ is A or | 3A, and VHH2 |
| | G, X₃ is T or V, X₄ is L or M, X, is K or N, | with one amino |
| | X₆ is D or E, X₇ is I or T, X₈ is F or I, X₉ | acid deletion at N- |
| | is E or D, and X₁₉ is E or Q | terminus |
| 139 | X₁₉VQLVESGGGLVQPGGSLRLSCAASGGTFSSYX₁MX₂WFRQAPG | Humanized 1C10 |
| | KEREYVGGISSSGYX₃X₄YX₅SX₆SMKGRFTISRDNAKNTLYLQMN | co-binder with |
| | SLRAEDTAVYYCAAGX₇X₈GRPYX₉YWGQGTQVTVSSGGGSGGGGS | VHH1 N-terminal |
| | GGGGSGGGGSGGGGSGGGGSGGGGSGMTGLVESGGGLVQPGGSLR | consensus residue |
| | LSCAASGDISSIVAMGWYRQAPGKQRELVSAITSGGRTHYRDSVK | and consensus |
| | GRFTISRDNAKNTLYLQMNSLRAEDTAVYYCAADRGWTSVGEYDY | CDRs1, 2A and |
| | WGQGTQVTVSS wherein X₁ is A or T, X₂ is A or | 3A, and VHH2 |
| | G, X₃ is T or V, X₄ is L or M, X₅ is K or N, | with two amino |
| | X₆ is D or E, X₇ is I or T, X₈ is F or I, X₉ | acid deletion at N- |
| | is E or D, and X₁₉ is E or Q | terminus |
| 140 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | VHH1 with N- |
| | NSLRAEDTAVYYCAAGTIGRPYDYWGQGTQVTVSS wherein | terminal consensus |
| | X₁₀ is T or V, X₁₁ is L or M, X₁₂ is K or N, X₁₃ | residue, and |
| | is S or A, X₁₄ is D or E, and X₁₉ is E or Q | consensus CDR2B |
| 141 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | VHH1 with N- |
| | DTAVYYCAAGX₁₅X₁₆GRPYX₁₇X₁₈WGQGTQVTVSS wherein X₁₅ | terminal consensus |
| | is I, T, or V, X₁₆ is F or I, X₁₇ is E or D, | residue, and |
| | X₁₈ is Y or F, and X₁₉ is E or Q | consensus CDR3B |
| 142 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | VHH1 with N- |
| | QMNSLRAEDTAVYYCAAGTIGRPYDYWGQGTQVTVSS wherein | terminal consensus |
| | X₁ is A or T, X₂ is A or G, X₁₀ is T or V, X₁₁ | residue, and |
| | is L or M, X₁₂ is K or N, X₁₃ is S or A, X₁₄ is | consensus CDRs1 |
| | D or E, and X₁₉ is E or Q | and 2B |
| 143 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | VHH1 with N- |
| | AEDTAVYYCAAGX₁₅X₁₆GRPYX₁₇X₁₈WGQGTQVTVSS wherein | terminal consensus |
| | X₁ is A or T, X₂ is A or G, X₁₅ is I, T, or V, | residue, and |
| | X₁₆ is F or I, X₁₇ is E or D, X₁₈ is Y or F, | consensus CDRs1 |
| | and X₁₉ is E or Q | and 3B |
| 144 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | VHH1 with N- |
| | NSLRAEDTAVYYCAAGX₁₅X₁₆GRPYX₁₇X₁₈WGQGTQVTVSS | terminal consensus |
| | wherein X₁₀ is T or V, X₁₁ is L or M, X₁₂ is K | residue, and |
| | or N, X₁₃ is S or A, X₁₄ is D or E, X₁₅ is I, | consensus |
| | T, or V, X₁₆ is F or I, X₁₇ is E or D, X₁₈ is Y | CDRs2B and 3B |
| | or F, and X₁₉ is E or Q | |
| 145 | X₁₉VQLVESGGGLVQPGGSLRLSCAASGGTFSSYXMX₂WFRQAPG | Humanized 1C10 |
| | KEREYVGGISSSGYX₁₀X₁₁YX₁₂X₁₃X₁₄SMKGRFTISRDNAKNTLYL | VHH1 with N- |
| | QMNSLRAEDTAVYYCAAGX₁₅X₁₆GRPYX₁₇X₁₈WGQGTQVTVSS | terminal consensus |
| | wherein X₁ is A or T, X₂ is A or G, X₁₀ is T | residue, and |
| | or V, X₁₁ is L or M, X₁₂ is K or N, X₁₃ is S or | consensus CDRs1, |
| | A, X₁₄ is D or E, X₁₅ is I, T, or V, X₁₆ is F | 2B and 3B |
| | or I, X₁₇ is E or D, X₁₈ is Y or F, and X₁₉ is | |
| | E or Q | |
| 146 | X₂₀VX₂₁LVESGGGLVQPGGSLRLSCAASGDISSIVAMGWYRQAPG | Humanized 1C10 |
| | KQRELVSAITSGGRTHYRDSVKGRFTISRDNAKNTLYLQMNSLRA | VHH2 with N- |
| | EDTAVYYCAADRGWTSVGEYDYWGQGTQVTVSS wherein X₂₀ | terminal consensus |
| | is E or Q, and X₂₁ is Q or K | residues at position |
| | | 1 and 3 |
| 147 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | co-binder with |
| | NSLRAEDTAVYYCAAGTIGRPYDYWGQGTQVTVSSGGGSGGGGSG | VHH1 N-terminal |
| | GGGSGGGGSGGGGSGGGGSGGGGSGMTGVQLVESGGGLVQPGGSL | consensus residue, |
| | RLSCAASGDISSIVAMGWYRQAPGKQRELVSAITSGGRTHYRDSV | and consensus |
| | KGRFTISRDNAKNTLYLQMNSLRAEDTAVYYCAADRGWTSVGEYD | CDR2B |
| | YWGQGTQVTVSS wherein X₁₀ is T or V, X₁₁ is L | |
| | or M, X₁₂ is K or N, X₁₃ is S or A, X₁₄ is D or | |
| | E, and X₁₉ is E or Q | |
| 148 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | co-binder with |
| | NSLRAEDTAVYYCAAGTIGRPYDYWGQGTQVTVSSGGGSGGGGSG | VHH1 N-terminal |
| | GGGSGGGGSGGGGSGGGGSGGGGSGMTGX₂₀VQLVESGGGLVQPGG | consensus residue |
| | SLRLSCAASGDISSIVAMGWYRQAPGKQRELVSAITSGGRTHYRD | and consensus |
| | SVKGRFTISRDNAKNTLYLQMNSLRAEDTAVYYCAADRGWTSVGE | CDR2B, and |
| | YDYWGQGTQVTVSS wherein X₁₀ is T or V, X₁₁ is L | VHH2 with one |
| | or M, X₁₂ is K or N, X₁₃ is S or A, X₁₄ is D or | amino acid |
| | E, X₁₉ is E or Q, and X₂₀ is E or Q | consensus addition |
| | | at N-terminus |
| 149 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | co-binder with |
| | NSLRAEDTAVYYCAAGTIGRPYDYWGQGTQVTVSSGGGSGGGGSG | VHH1 N-terminal |
| | GGGSGGGGSGGGGSGGGGSGGGGSGMTGQLVESGGGLVQPGGSLR | consensus residue |
| | LSCAASGDISSIVAMGWYRQAPGKQRELVSAITSGGRTHYRDSVK | and consensus |
| | GRFTISRDNAKNTLYLQMNSLRAEDTAVYYCAADRGWTSVGEYDY | CDR2B, and |
| | WGQGTQVTVSS wherein X₁₀ is T or V, X₁₁ is L or | VHH2 with one |
| | M, X₁₂ is K or N, X₁₃ is S or A, X₁₄ is D or E, | amino acid |
| | and X₁₉ is E or Q | deletion at N- |
| | | terminus |
| 150 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | co-binder with |
| | NSLRAEDTAVYYCAAGTIGRPYDYWGQGTQVTVSSGGGSGGGGSG | VHH1 N-terminal |
| | GGGSGGGGSGGGGSGGGGSGGGGSGMTGLVESGGGLVQPGGSLRL | consensus residue |
| | SCAASGDISSIVAMGWYRQAPGKQRELVSAITSGGRTHYRDSVKG | and consensus |
| | RFTISRDNAKNTLYLQMNSLRAEDTAVYYCAADRGWTSVGEYDYW | CDR2B, and |
| | GQGTQVTVSS wherein X₁₀ is T or V, X₁₁ is L or | VHH2 with two |
| | M, X₁₂ is K or N, X₁₃ is S or A, X₁₄ is D or E, | amino acid |
| | and X₁₉ is E or Q | deletion at N- |
| | | terminus |
| 151 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | co-binder with |
| | NSLRAEDTAVYYCAAGTIGRPYDYWGQGTQVTVSSGGGSGGGGSG | VHH1 N-terminal |
| | GGGSGGGGSGGGGSGGGGSGGGGSGMTGX₂₀VX₂₁LVESGGGLVQP | consensus residue |
| | GGSLRLSCAASGDISSIVAMGWYRQAPGKQRELVSAITSGGRTHY | and consensus |
| | RDSVKGRFTISRDNAKNTLYLQMNSLRAEDTAVYYCAADRGWTSV | CDR2B, and |
| | GEYDYWGQGTQVTVSS wherein X₁₀ is T or V, X₁₁ is | VHH2 with N- |
| | L or M, X₁₂ is K or N, X₁₃ is S or A, X₁₄ is D | terminal consensus |
| | or E, X₁₉ is E or Q, X₂₀ is E or Q, and X₂₁ is | residues at position |
| | Q or K | 1 and 3 |
| 152 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGX₁₅X₁₆GRPYX₁₇X₁₈WGQGTQVTVSSGGGSGGGGSGG | VHH1 N-terminal |
| | GGSGGGGSGGGGSGGGGSGGGGSGMTGVQLVESGGGLVQPGGSLR | consensus residue, |
| | LSCAASGDISSIVAMGWYRQAPGKQRELVSAITSGGRTHYRDSVK | and consensus |
| | GRFTISRDNAKNTLYLQMNSLRAEDTAVYYCAADRGWTSVGEYDY | CDR3B |
| | WGQGTQVTVSS wherein X₁₅ is I, T, or V, X₁₆ is | |
| | F or I, X₁₇ is E or D, X₁₈ is Y or F, and X₁₉ | |
| | is E or Q | |
| 153 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGX₁₅X₁₆GRPYX₁₇X₁₈WGQGTQVTVSSGGGSGGGGSGG | VHH1 N-terminal |
| | GGSGGGGSGGGGSGGGGSGGGGSGMTGX₂₀VQLVESGGGLVQPGGS | consensus residue |
| | LRLSCAASGDISSIVAMGWYRQAPGKQRELVSAITSGGRTHYRDS | and consensus |
| | VKGRFTISRDNAKNTLYLQMNSLRAEDTAVYYCAADRGWTSVGEY | CDR3B, and |
| | DYWGQGTQVTVSS wherein X₁₅ is I, T, or V, X₁₆ | VHH2 with one |
| | is F or I, X₁₇ is E or D, X₁₈ is Y or F, X₁₉ is | amino acid |
| | E or Q, and X₂₀ is E or Q | consensus addition |
| | | at N-terminus |
| 154 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGX₁₅X₁₆GRPYX₁₇X₁₈WGQGTQVTVSSGGGSGGGGSGG | VHH1 N-terminal |
| | GGSGGGGSGGGGSGGGGSGGGGSGMTGQLVESGGGLVQPGGSLRL | consensus residue |
| | SCAASGDISSIVAMGWYRQAPGKQRELVSAITSGGRTHYRDSVKG | and consensus |
| | RFTISRDNAKNTLYLQMNSLRAEDTAVYYCAADRGWTSVGEYDYW | CDR3B, and |
| | GQGTQVTVSS wherein X₁₅ is I, T, or V, X₁₆ is F | VHH2 with one |
| | or I, X₁₇ is E or D, X₁₈ is Y or F, and X₁₉ is | amino acid |
| | E or Q | deletion at N- |
| | | terminus |
| 155 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGX₁₅X₁₆GRPYX₁₇X₁₈WGQGTQVTVSSGGGSGGGGSGG | VHH1 N-terminal |
| | GGSGGGGSGGGGSGGGGSGGGGSGMTGLVESGGGLVQPGGSLRLS | consensus residue |
| | CAASGDISSIVAMGWYRQAPGKQRELVSAITSGGRTHYRDSVKGR | and consensus |
| | FTISRDNAKNTLYLQMNSLRAEDTAVYYCAADRGWTSVGEYDYWG | CDR3B, and |
| | QGTQVTVSS wherein X₁₅ is I, T, or V, X₁₆ is F | VHH2 with two |
| | or I, X₁₇ is E or D, X₁₈ is Y or F, and X₁₉ is | amino acid |
| | E or Q | deletion at N- |
| | | terminus |
| 156 | X₁₉VQLVESGGGLVQPGGSLRLSCAASGGTFSSYTMGWFRQAPGKE | Humanized 1C10 |
| | REYVGGISSSGYVMYNSESMKGRFTISRDNAKNTLYLQMNSLRAE | co-binder with |
| | DTAVYYCAAGX₁₅X₁₆GRPYX₁₇X₁₈WGQGTQVTVSSGGGSGGGGSGG | VHH1 N-terminal |
| | GGSGGGGSGGGGSGGGGSGGGGSGMTGX₂₀VX₂₁LVESGGGLVQPG | consensus residue |
| | GSLRLSCAASGDISSIVAMGWYRQAPGKQRELVSAITSGGRTHYR | and consensus |
| | DSVKGRFTISRDNAKNTLYLQMNSLRAEDTAVYYCAADRGWTSVG | CDR3B, and |
| | EYDYWGQGTQVTVSS wherein X₁₅ is I, T, or V, X₁₆ | VHH2 with N- |
| | is F or I, X₁₇ is E or D, X₁₈ is Y or F, X₁₉ is | terminal consensus |
| | E or Q, X₂₀ is E or Q, and X₂₁ is Q or K | residues at position |
| | | 1 and 3 |
| 157 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | co-binder with |
| | QMNSLRAEDTAVYYCAAGTIGRPYDYWGQGTQVTVSSGGGSGGGG | VHH1 N-terminal |
| | SGGGGSGGGGSGGGGSGGGGSGGGGSGMTGVQLVESGGGLVQPGG | consensus residue, |
| | SLRLSCAASGDISSIVAMGWYRQAPGKQRELVSAITSGGRTHYRD | and consensus |
| | SVKGRFTISRDNAKNTLYLQMNSLRAEDTAVYYCAADRGWTSVGE | CDRs1 and 2B |
| | YDYWGQGTQVTVSS wherein X₁ is A or T, X₂ is A | |
| | or G, X₁₀ is T or V, X₁₁ is L or M, X₁₂ is K or | |
| | N, X₁₃ is S or A, X₁₄ is D or E, and X₁₉ is E | |
| | or Q | |
| 158 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | co-binder with |
| | QMNSLRAEDTAVYYCAAGTIGRPYDYWGQGTQVTVSSGGGSGGGG | VHH1 N-terminal |
| | SGGGGSGGGGSGGGGSGGGGSGGGGSGMTGX₂₀VQLVESGGGLVQP | consensus residue |
| | GGSLRLSCAASGDISSIVAMGWYRQAPGKQRELVSAITSGGRTHY | and consensus |
| | RDSVKGRFTISRDNAKNTLYLQMNSLRAEDTAVYYCAADRGWTSV | CDRs1 and 2B, |
| | GEYDYWGQGTQVTVSS wherein X₁ is A or T, X₂ is A | and VHH2 with |
| | or G, X₁₀ is T or V, X₁₁ is L or M, X₁₂ is K or | one amino acid |
| | N, X₁₃ is S or A, X₁₄ is D or E, X₁₉ is E or Q, | consensus addition |
| | and X₂₀ is E or Q | at N-terminus |
| 159 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | co-binder with |
| | QMNSLRAEDTAVYYCAAGTIGRPYDYWGQGTQVTVSSGGGSGGGG | VHH1 N-terminal |
| | SGGGGSGGGGSGGGGSGGGGSGGGGSGMTGQLVESGGGLVQPGGS | consensus residue |
| | LRLSCAASGDISSIVAMGWYRQAPGKQRELVSAITSGGRTHYRDS | and consensus |
| | VKGRFTISRDNAKNTLYLQMNSLRAEDTAVYYCAADRGWTSVGEY | CDRs1 and 2B, |
| | DYWGQGTQVTVSS wherein X₁ is A or T, X₂ is A or | and VHH2 with |
| | G, X₁₀ is T or V, X₁₁ is L or M, X₁₂ is K or N, | one amino acid |
| | X₁₃ is S or A, X₁₄ is D or E, and X₁₉ is E or Q | deletion at N- |
| | | terminus |
| 160 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | co-binder with |
| | QMNSLRAEDTAVYYCAAGTIGRPYDYWGQGTQVTVSSGGGSGGGG | VHH1 N-terminal |
| | SGGGGSGGGGSGGGGSGGGGSGGGGSGMTGLVESGGGLVQPGGSL | consensus residue |
| | RLSCAASGDISSIVAMGWYRQAPGKQRELVSAITSGGRTHYRDSV | and consensus |
| | KGRFTISRDNAKNTLYLQMNSLRAEDTAVYYCAADRGWTSVGEYD | CDRs1 and 2B, |
| | YWGQGTQVTVSS wherein X₁ is A or T, X₂ is A or | and VHH2 with |
| | G, X₁₀ is T or V, X₁₁ is L or M, X₁₂ is K or N, | two amino acid |
| | X₁₃ is S or A, X₁₄ is D or E, and X₁₉ is E or Q | deletion at N- |
| | | terminus |
| 161 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | co-binder with |
| | QMNSLRAEDTAVYYCAAGTIGRPYDYWGQGTQVTVSSGGGSGGGG | VHH1 N-terminal |
| | SGGGGSGGGGSGGGGSGGGGSGGGGSGMTGX₂₀VX₂₁LVESGGGLV | consensus residue |
| | QPGGSLRLSCAASGDISSIVAMGWYRQAPGKQRELVSAITSGGRT | and consensus |
| | HYRDSVKGRFTISRDNAKNTLYLQMNSLRAEDTAVYYCAADRGWT | CDRs1 and 2B, |
| | SVGEYDYWGQGTQVTVSS wherein X₁ is A or T, X₂ is | and VHH2 with N- |
| | A or G, X₁₀ is T or V, X₁₁ is L or M, X₁₂ is K | terminal consensus |
| | or N, X₁₃ is S or A, X₁₄ is D or E, X₁₉ is E or | residues at position |
| | Q, X₂₀ is E or Q, and X₂₁ is Q or K | 1 and 3 |
| 162 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGX₁₅X₁₆GRPYX₁₇X₁₈WGQGTQVTVSSGGGSGGGGS | VHH1 N-terminal |
| | GGGGSGGGGSGGGGSGGGGSGGGGSGMTGVQLVESGGGLVQPGGS | consensus residue, |
| | LRLSCAASGDISSIVAMGWYRQAPGKQRELVSAITSGGRTHYRDS | and consensus |
| | VKGRFTISRDNAKNTLYLQMNSLRAEDTAVYYCAADRGWTSVGEY | CDRs1 and 3B |
| | DYWGQGTQVTVSS wherein X₁ is A or T, X₂ is A or | |
| | G, X₁₅ is I, T, or V, X₁₆ is F or I, X₁₇ is E | |
| | or D, X₁₈ is Y or F, and X₁₉ is E or Q | |
| 163 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGX₁₅X₁₆GRPYX₁₇X₁₈WGQGTQVTVSSGGGSGGGGS | VHH1 N-terminal |
| | GGGGSGGGGSGGGGSGGGGSGGGGSGMTGX₂₀VQLVESGGGLVQPG | consensus residue, |
| | GSLRLSCAASGDISSIVAMGWYRQAPGKQRELVSAITSGGRTHYR | and consensus |
| | DSVKGRFTISRDNAKNTLYLQMNSLRAEDTAVYYCAADRGWTSVG | CDRs1 and 3B, |
| | EYDYWGQGTQVTVSS wherein X₁ is A or T, X₂ is A | and VHH2 with |
| | or G, X₁₅ is I, T, or V, X₁₆ is F or I, X₁₇ is | one amino acid |
| | E or D, X₁₈ is Y or F, X₁₉ is E or Q, and X₂₀ | consensus addition |
| | is E or Q | at N-terminus |
| 164 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGX₁₅X₁₆GRPYX₁₇X₁₈WGQGTQVTVSSGGGSGGGGS | VHH1 N-terminal |
| | GGGGSGGGGSGGGGSGGGGSGGGGSGMTGQLVESGGGLVQPGGSL | consensus residue, |
| | RLSCAASGDISSIVAMGWYRQAPGKQRELVSAITSGGRTHYRDSV | and consensus |
| | KGRFTISRDNAKNTLYLQMNSLRAEDTAVYYCAADRGWTSVGEYD | CDRs1 and 3B, |
| | YWGQGTQVTVSS wherein X₁ is A or T, X₂ is A or | and VHH2 with |
| | G, X₁₅ is I, T, or V, X₁₆ is F or I, X₁₇ is E | one amino acid |
| | or D, X₁₈ is Y or F, and X₁₉ is E or Q | deletion at N- |
| | | terminus |
| 165 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGX₁₅X₁₆GRPYX₁₇X₁₈WGQGTQVTVSSGGGSGGGGS | VHH1 N-terminal |
| | GGGGSGGGGSGGGGSGGGGSGGGGSGMTGLVESGGGLVQPGGSLR | consensus residue, |
| | LSCAASGDISSIVAMGWYRQAPGKQRELVSAITSGGRTHYRDSVK | and consensus |
| | GRFTISRDNAKNTLYLQMNSLRAEDTAVYYCAADRGWTSVGEYDY | CDRs1 and 3B, |
| | WGQGTQVTVSS wherein X₁ is A or T, X₂ is A or | and VHH2 with |
| | G, X₁₅ is I, T, or V, X₁₆ is F or I, X₁₇ is E | two amino acid |
| | or D, X₁₈ is Y or F, and X₁₉ is E or Q | deletion at N- |
| | | terminus |
| 166 | X₁₉VQLVESGGGLVQPGGSLRLSCAASGGTFSSYX₁MX₂WFRQAPG | Humanized 1C10 |
| | KEREYVGGISSSGYVMYNSESMKGRFTISRDNAKNTLYLQMNSLR | co-binder with |
| | AEDTAVYYCAAGX₁₅X₁₆GRPYX₁₇X₁₈WGQGTQVTVSSGGGSGGGGS | VHH1 N-terminal |
| | GGGGSGGGGSGGGGSGGGGSGGGGSGMTGX₂₀VX₂₁LVESGGGLVQ | consensus residue, |
| | PGGSLRLSCAASGDISSIVAMGWYRQAPGKQRELVSAITSGGRTH | and consensus |
| | YRDSVKGRFTISRDNAKNTLYLQMNSLRAEDTAVYYCAADRGWTS | CDRs1 and 3B, |
| | VGEYDYWGQGTQVTVSS wherein X₁ is A or T, X₂ is | and VHH2 with N- |
| | A or G, X₁₅ is I, T, or V, X₁₆ is F or I, X₁₇ | terminal consensus |
| | is E or D, X₁₈ is Y or F, X₁₉ is E or Q, X₂₀ is | residues at position |
| | E or Q, and X₂₁ is Q or K | 1 and 3 |
| 167 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | co-binder with |
| | NSLRAEDTAVYYCAAGX₁₅X₁₆GRPYX₁₇X₁₈WGQGTQVTVSSGGGSG | VHH1 N-terminal |
| | GGGSGGGGSGGGGSGGGGSGGGGSGGGGSGMTGVQLVESGGGLVQ | consensus residue, |
| | PGGSLRLSCAASGDISSIVAMGWYRQAPGKQRELVSAITSGGRTH | and consensus |
| | YRDSVKGRFTISRDNAKNTLYLQMNSLRAEDTAVYYCAADRGWTS | CDRs2B and 3B |
| | VGEYDYWGQGTQVTVSS wherein X₁₀ is T or V, X₁₁ | |
| | is L or M, X₁₂ is K or N, X₁₃ is S or A, X₁₄ is | |
| | D or E, X₁₅ is I, T, or V, X₁₆ is F or I, X₁₇ | |
| | is E or D, X₁₈ is Y or F, and X₁₉ is E or Q | |
| 168 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | co-binder with |
| | NSLRAEDTAVYYCAAGX₁₅X₁₆GRPYX₁₇X₁₈WGQGTQVTVSSGGGSG | VHH1 N-terminal |
| | GGGSGGGGSGGGGSGGGGSGGGGSGGGGSGMTGX₂₀VQLVESGGGL | consensus residue, |
| | VQPGGSLRLSCAASGDISSIVAMGWYRQAPGKQRELVSAITSGGR | and consensus |
| | THYRDSVKGRFTISRDNAKNTLYLQMNSLRAEDTAVYYCAADRGW | CDRs2B and 3B, |
| | TSVGEYDYWGQGTQVTVSS wherein X₁₀ is T or V, X₁₁ | and VHH2 with |
| | is L or M, X₁₂ is K or N, X₁₃ is S or A, X₁₄ is | one amino acid |
| | D or E, X₁₅ is I, T, or V, X₁₆ is F or I, X₁₇ | consensus addition |
| | is E or D, X₁₈ is Y or F, X₁₉ is E or Q, and | at N-terminus |
| | X₂₀ is E or Q | |
| 169 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | co-binder with |
| | NSLRAEDTAVYYCAAGX₁₅X₁₆GRPYX₁₇X₁₈WGQGTQVTVSSGGGSG | VHH1 N-terminal |
| | GGGSGGGGSGGGGSGGGGSGGGGSGGGGSGMTGQLVESGGGLVQP | consensus residue, |
| | GGSLRLSCAASGDISSIVAMGWYRQAPGKQRELVSAITSGGRTHY | and consensus |
| | RDSVKGRFTISRDNAKNTLYLQMNSLRAEDTAVYYCAADRGWTSV | CDRs2B and 3B, |
| | GEYDYWGQGTQVTVSS wherein X₁₀ is T or V, X₁₁ is | and VHH2 with |
| | L or M, X₁₂ is K or N, X₁₃ is S or A, X₁₄ is D | one amino acid |
| | or E, X₁₅ is I, T, or V, X₁₆ is F or I, X₁₇ is | deletion at N- |
| | E or D, X₁₈ is Y or F, and X₁₉ is E or Q | terminus |
| 170 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | co-binder with |
| | NSLRAEDTAVYYCAAGX₁₅X₁₆GRPYX₁₇X₁₈WGQGTQVTVSSGGGSG | VHH1 N-terminal |
| | GGGSGGGGSGGGGSGGGGSGGGGSGGGGSGMTGLVESGGGLVQPG | consensus residue, |
| | GSLRLSCAASGDISSIVAMGWYRQAPGKQRELVSAITSGGRTHYR | and consensus |
| | DSVKGRFTISRDNAKNTLYLQMNSLRAEDTAVYYCAADRGWTSVG | CDRs2B and 3B, |
| | EYDYWGQGTQVTVSS wherein X₁₀ is T or V, X₁₁ is | and VHH2 with |
| | L or M, X₁₂ is K or N, X₁₃ is S or A, X₁₄ is D | two amino acid |
| | or E, X₁₅ is I, T, or V, X₁₆ is F or I, X₁₇ is | deletion at N- |
| | E or D, X₁₈ is Y or F, and X₁₉ is E or Q | terminus |
| 171 | X₁₉VQLVESGGGLVQPGGSLRLSCAASGGTFSSYTMGWFRQAPGKE | Humanized 1C10 |
| | REYVGGISSSGYX₁₀X₁₁YX₁₂X₁₃X₁₄SMKGRFTISRDNAKNTLYLQM | co-binder with |
| | NSLRAEDTAVYYCAAGX₁₅X₁₆GRPYX₁₇X₁₈WGQGTQVTVSSGGGSG | VHH1 N-terminal |
| | GGGSGGGGSGGGGSGGGGSGGGGSGGGGSGMTGX₂₀VX₂₁LVESGG | consensus residue, |
| | GLVQPGGSLRLSCAASGDISSIVAMGWYRQAPGKQRELVSAITSG | and consensus |
| | GRTHYRDSVKGRFTISRDNAKNTLYLQMNSLRAEDTAVYYCAADR | CDRs2B and 3B, |
| | GWTSVGEYDYWGQGTQVTVSS wherein X₁₀ is T or V, | and VHH2 with N- |
| | X₁₁ is L or M, X₁₂ is K or N, X₁₃ is S or A, X₁₄ | terminal consensus |
| | is D or E, X₁₅ is I, T, or V, X₁₆ is F or I, | residues at position |
| | X₁₇ is E or D, X₁₈ is Y or F, X₁₉ is E or Q, X₂₀ | 1 and 3 |
| | is E or Q, and X₂₁ is Q or K | |
| 172 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | co-binder with |
| | QMNSLRAEDTAVYYCAAGX₁₅X₁₆GRPYX₁₇X₁₈WGQGTQVTVSSGGG | VHH1 N-terminal |
| | SGGGGSGGGGSGGGGSGGGGSGGGGSGGGGSGMTGVQLVESGGGL | consensus residue, |
| | VQPGGSLRLSCAASGDISSIVAMGWYRQAPGKQRELVSAITSGGR | and consensus |
| | THYRDSVKGRFTISRDNAKNTLYLQMNSLRAEDTAVYYCAADRGW | CDRs1, 2B and 3B |
| | TSVGEYDYWGQGTQVTVSS wherein X₁ is A or T, X₂ | |
| | is A or G, X₁₀ is T or V, X₁₁ is L or M, X₁₂ is | |
| | K or N, X₁₃ is S or A, X₁₄ is D or E, X₁₅ is I, | |
| | T, or V, X₁₆ is F or I, X₁₇ is E or D, X₁₈ is Y | |
| | or F, and X₁₉ is E or Q | |
| 173 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | co-binder with |
| | QMNSLRAEDTAVYYCAAGX₁₅X₁₆GRPYX₁₇X₁₈WGQGTQVTVSSGGG | VHH1 N-terminal |
| | SGGGGSGGGGSGGGGSGGGGSGGGGSGGGGSGMTGX₂₀VQLVESGG | consensus residue, |
| | GLVQPGGSLRLSCAASGDISSIVAMGWYRQAPGKQRELVSAITSG | and consensus |
| | GRTHYRDSVKGRFTISRDNAKNTLYLQMNSLRAEDTAVYYCAADR | CDRs1, 2B and |
| | GWTSVGEYDYWGQGTQVTVSS wherein X₁ is A or T, X₂ | 3B, and VHH2 |
| | is A or G, X₁₀ is T or V, X₁₁ is L or M, X₁₂ is | with one amino |
| | K or N, X₁₃ is S or A, X₁₄ is D or E, X₁₅ is I, | acid consensus |
| | T, or V, X₁₆ is F or I, X₁₇ is E or D, X₁₈ is Y | addition at N- |
| | or F, X₁₉ is E or Q, and X₂₀ is E or Q | terminus |
| 174 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | co-binder with |
| | QMNSLRAEDTAVYYCAAGX₁₅X₁₆GRPYX₁₇X₁₈WGQGTQVTVSSGGG | VHH1 N-terminal |
| | SGGGGSGGGGSGGGGSGGGGSGGGGSGGGGSGMTGQLVESGGGLV | consensus residue, |
| | QPGGSLRLSCAASGDISSIVAMGWYRQAPGKQRELVSAITSGGRT | and consensus |
| | HYRDSVKGRFTISRDNAKNTLYLQMNSLRAEDTAVYYCAADRGWT | CDRs1, 2B and |
| | SVGEYDYWGQGTQVTVSS wherein X₁ is A or T, X₂ is | 3B, and VHH2 |
| | A or G, X₁₀ is T or V, X₁₁ is L or M, X₁₂ is K | with one amino |
| | or N, X₁₃ is S or A, X₁₄ is D or E, X₁₅ is I, | acid deletion at N- |
| | T, or V, X₁₆ is F or I, X₁₇ is E or D, X₁₈ is Y | terminus |
| | or F, and X₁₉ is E or Q | |
| 175 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | co-binder with |
| | QMNSLRAEDTAVYYCAAGX₁₅X₁₆GRPYX₁₇X₁₈WGQGTQVTVSSGGG | VHH1 N-terminal |
| | SGGGGSGGGGSGGGGSGGGGSGGGGSGGGGSGMTGLVESGGGLVQ | consensus residue, |
| | PGGSLRLSCAASGDISSIVAMGWYRQAPGKQRELVSAITSGGRTH | and consensus |
| | YRDSVKGRFTISRDNAKNTLYLQMNSLRAEDTAVYYCAADRGWTS | CDRs1, 2B and |
| | VGEYDYWGQGTQVTVSS wherein X₁ is A or T, X₂ is | 3B, and VHH2 |
| | A or G, X₁₀ is T or V, X₁₁ is L or M, X₁₂ is K | with two amino |
| | or N, X₁₃ is S or A, X₁₄ is D or E, X₁₅ is I, | acid deletion at N- |
| | T, or V, X₁₆ is F or I, X₁₇ is E or D, X₁₈ is Y | terminus |
| | or F, and X₁₉ is E or Q | |
| 176 | X₁₉VQLVESGGGLVQPGGSLRLSCAASGGTFSSYX₁MX₂WFRQAPG | Humanized 1C10 |
| | KEREYVGGISSSGYX₁₀X₁₁YX₁₂X₁₃X₁₄SMKGRFTISRDNAKNTLYL | co-binder with |
| | QMNSLRAEDTAVYYCAAGX₁₅X₁₆GRPYX₁₇X₁₈WGQGTQVTVSSGGG | VHH1 N-terminal |
| | SGGGGSGGGGSGGGGSGGGGSGGGGSGGGGSGMTGX₂₀VX₂₁LVES | consensus residue, |
| | GGGLVQPGGSLRLSCAASGDISSIVAMGWYRQAPGKQRELVSAIT | and consensus |
| | SGGRTHYRDSVKGRFTISRDNAKNTLYLQMNSLRAEDTAVYYCAA | CDRs1, 2B and |
| | DRGWTSVGEYDYWGQGTQVTVSS wherein X₁ is A or T, | 3B, and VHH2 |
| | X₂ is A or G, X₁₀ is T or V, X₁₁ is L or M, X₁₂ | with N-terminal |
| | is K or N, X₁₃ is S or A, X₁₄ is D or E, X₁₅ is | consensus residues |
| | I, T, or V, X₁₆ is F or I, X₁₇ is E or D, X₁₈ | at position 1 and 3 |
| | is Y or F, X₁₉ is E or Q, X₂₀ is E or Q, and | |
| | X₂₁ is Q or K | |
| 177 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | VHH1 with N- |
| | DTAVYYCAAGTIGRPYDYWGQGTQVTVSS wherein X₁₉ is E | terminal consensus |
| | or Q | residue |
| 178 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | VHH1 with N- |
| | AEDTAVYYCAAGTIGRPYDYWGQGTQVTVSS wherein X₁ is | terminal consensus |
| | A or T, X₂ is A or G, and X₁₉ is E or Q | residue, and |
| | | consensus CDR1 |
| 179 | X₁₉VQLVESGGGLVQPGGSLRLSCAASGGTFSSYTMGWFRQAPGKE | Humanized 21H7 |
| | REYVGGISSSGYX₃X₄YX₅SX₆SMKGRFTISRDNAKNTLYLQMNSL | VHH1 with N- |
| | RAEDTAVYYCAAGTIGRPYDYWGQGTQVTVSS wherein X₃ | terminal consensus |
| | is T or V, X₄ is L or M, X₅ is K or N, X₆ is D | residue, and |
| | or E, and X₁₉ is E or Q | consensus CDR2A |
| 180 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | VHH1 with N- |
| | DTAVYYCAAGX₇X₈GRPYX₉YWGQGTQVTVSS wherein X₇ is | terminal consensus |
| | I or T, X₈ is F or I, X₉ is E or D, and X₁₉ is | residue, and |
| | E or Q | consensus CDR3A |
| 181 | X₁₉VQLVESGGGLVQPGGSLRLSCAASGGTFSSYX₁MX₂WFRQAPG | Humanized 21H7 |
| | KEREYVGGISSSGYX₃X₄YX₅SX₆SMKGRFTISRDNAKNTLYLQMN | VHH1 with N- |
| | SLRAEDTAVYYCAAGTIGRPYDYWGQGTQVTVSS wherein X₁ | terminal consensus |
| | is A or T, X₂ is A or G, X₃ is T or V, X₄ is L | residue, and |
| | or M, X₅ is K or N, X₆ is D or E, and X₁₉ is E | consensus CDRs1 |
| | or Q | and 2A |
| 182 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | VHH1 with N- |
| | AEDTAVYYCAAGX₇X₈GRPYX₉YWGQGTQVTVSS wherein X₁ | terminal consensus |
| | is A or T, X₂ is A or G, X₇ is I or T, X₈ is F | residue, and |
| | or I, X₉ is E or D, and X₁₉ is E or Q | consensus CDRs1 |
| | | and 3A |
| 183 | X₁₉VQLVESGGGLVQPGGSLRLSCAASGGTFSSYTMGWFRQAPGKE | Humanized 21H7 |
| | REYVGGISSSGYX₃X₄YX₅SX₆SMKGRFTISRDNAKNTLYLQMNSL | VHH1 with N- |
| | RAEDTAVYYCAAGX₇X₈GRPYX₉YWGQGTQVTVSS wherein X₃ | terminal consensus |
| | is T or V, X₄ is L or M, X₅ is K or N, X₆ is D | residue, and |
| | or E, X₇ is I or T, X₈ is F or I, X₉ is E or | consensus |
| | D, and X₁₉ is E or Q | CDRs2A and 3A |
| 184 | X₁₉VQLVESGGGLVQPGGSLRLSCAASGGTFSSYX₁MX₂WFRQAPG | Humanized 21H7 |
| | KEREYVGGISSSGYX₃X₄YX₅SX₆SMKGRFTISRDNAKNTLYLQMN | VHH1 with N- |
| | SLRAEDTAVYYCAAGX₇X₈GRPYX₉YWGQGTQVTVSS wherein | terminal consensus |
| | X₁ is A or T, X₂ is A or G, X₃ is T or V, X₄ | residue, and |
| | is L or M, X₅ is K or N, X₆ is D or E, X₇ is I | consensus CDRs1, |
| | or T, X₈ is F or I, X₉ is E or D, and X₁₉ is E | 2A and 3A |
| | or Q | |
| 185 | X₂₁LVESGGGLVQPGGSLRLSCAASGRTFSTYIIGWFRQAPGKERE | Humanized 21H7 |
| | FVASISWGGRGTYYADSVKDRFTISRDNPKNMVYLQMNSLRAEDT | VHH2 with one |
| | AVYYCAAGDLPGGRRALDYDYWGQGTLVTVSS wherein X₂₁ | amino acid |
| | is Q or K | consensus addition |
| | | at N-terminus |
| 186 | X₂₂X₂₁LVESGGGLVQPGGSLRLSCAASGRTFSTYIIGWFRQAPGK | Humanized 21H7 |
| | EREFVASISWGGRGTYYADSVKDRFTISRDNPKNMVYLQMNSLRA | VHH2 with two |
| | EDTAVYYCAAGDLPGGRRALDYDYWGQGTLVTVSS wherein | amino acid |
| | X₂₁ is Q or K, and X₂₂ is V | consensus addition |
| | | at N-terminus |
| 187 | X₂₀X₂₂X₂₁LVESGGGLVQPGGSLRLSCAASGRTFSTYIIGWFRQAP | Humanized 21H7 |
| | GKEREFVASISWGGRGTYYADSVKDRFTISRDNPKNMVYLQMNSL | VHH2 with three |
| | RAEDTAVYYCAAGDLPGGRRALDYDYWGQGTLVTVSS wherein | amino acid |
| | X₂₀ is E or Q, X₂₁ is Q or K, and X₂₂ is V | consensus addition |
| | | at N-terminus |
| 188 | GGGSGGGGSGGGGSGGGGSGGGGSGGGGSGGGGSEGVG | Humanized 21H7 |
| | | linker connecting |
| | | VHH1 and VHH2 |
| 189 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGTIGRPYDYWGQGTQVTVSSGGGSGGGGSGGGGSGG | VHH1 N-terminal |
| | GGSGGGGSGGGGSGGGGSEGVGLVESGGGLVQPGGSLRLSCAASG | consensus residue |
| | RTFSTYIIGWFRQAPGKEREFVASISWGGRGTYYADSVKDRFTIS | |
| | RDNPKNMVYLQMNSLRAEDTAVYYCAAGDLPGGRRALDYDYWGQG | |
| | TLVTVSS wherein X₁₉ is E or Q | |
| 190 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGTIGRPYDYWGQGTQVTVSSGGGSGGGGSGGGGSGG | VHH1 N-terminal |
| | GGSGGGGSGGGGSGGGGSEGVGX₂₁LVESGGGLVQPGGSLRLSCAA | consensus residue, |
| | SGRTFSTYIIGWFRQAPGKEREFVASISWGGRGTYYADSVKDRFT | and VHH2 with |
| | ISRDNPKNMVYLQMNSLRAEDTAVYYCAAGDLPGGRRALDYDYWG | one amino acid |
| | QGTLVTVSS wherein X₁₉ is E or Q, and X₂₁ is Q | consensus addition |
| | or K | at N-terminus |
| 191 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGTIGRPYDYWGQGTQVTVSSGGGSGGGGSGGGGSGG | VHH1 N-terminal |
| | GGSGGGGSGGGGSGGGGSEGVGX₂₂X₂₁LVESGGGLVQPGGSLRLS | consensus residue, |
| | CAASGRTFSTYIIGWFRQAPGKEREFVASISWGGRGTYYADSVKD | and VHH2 with |
| | RFTISRDNPKNMVYLQMNSLRAEDTAVYYCAAGDLPGGRRALDYD | two amino acid |
| | YWGQGTLVTVSS wherein X₁₉ is E or Q, X₂₁ is Q | consensus addition |
| | or K, and X₂₂ is V | at N-terminus |
| 192 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGTIGRPYDYWGQGTQVTVSSGGGSGGGGSGGGGSGG | VHH1 N-terminal |
| | GGSGGGGSGGGGSGGGGSEGVGX₂₀X₂₂X₂₁LVESGGGLVQPGGSLR | consensus residue, |
| | LSCAASGRTFSTYIIGWFRQAPGKEREFVASISWGGRGTYYADSV | and VHH2 with |
| | KDRFTISRDNPKNMVYLQMNSLRAEDTAVYYCAAGDLPGGRRALD | three amino acid |
| | YDYWGQGTLVTVSS wherein X₁₉ is E or Q, X₂₀ is E | consensus addition |
| | or Q, X₂₁ is Q or K, and X₂₂ is V | at N-terminus |
| 193 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGTIGRPYDYWGQGTQVTVSSGGGSGGGGSGGGGS | VHH1 N-terminal |
| | GGGGSGGGGSGGGGSGGGGSEGVGLVESGGGLVQPGGSLRLSCAA | consensus residue |
| | SGRTFSTYIIGWFRQAPGKEREFVASISWGGRGTYYADSVKDRFT | and consensus |
| | ISRDNPKNMVYLQMNSLRAEDTAVYYCAAGDLPGGRRALDYDYWG | CDR1 |
| | QGTLVTVSS wherein X₁ is A or T, X₂ is A or G, | |
| | and X₁₉ is E or Q | |
| 194 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGTIGRPYDYWGQGTQVTVSSGGGSGGGGSGGGGS | VHH1 N-terminal |
| | GGGGSGGGGSGGGGSGGGGSEGVGX₂₁LVESGGGLVQPGGSLRLSC | consensus residue |
| | AASGRTFSTYIIGWFRQAPGKEREFVASISWGGRGTYYADSVKDR | and consensus |
| | FTISRDNPKNMVYLQMNSLRAEDTAVYYCAAGDLPGGRRALDYDY | CDR1, and VHH2 |
| | WGQGTLVTVSS wherein X₁ is A or T, X₂ is A or | with one amino |
| | G, X₁₉ is E or Q, and X₂₁ is Q or K | acid consensus |
| | | addition at N- |
| | | terminus |
| 195 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGTIGRPYDYWGQGTQVTVSSGGGSGGGGSGGGGS | VHH1 N-terminal |
| | GGGGSGGGGSGGGGSGGGGSEGVGX₂₂X₂₁LVESGGGLVQPGGSLR | consensus residue |
| | LSCAASGRTFSTYIIGWFRQAPGKEREFVASISWGGRGTYYADSV | and consensus |
| | KDRFTISRDNPKNMVYLQMNSLRAEDTAVYYCAAGDLPGGRRALD | CDR1, and VHH2 |
| | YDYWGQGTLVTVSS wherein X₁ is A or T, X₂ is A | with two amino |
| | or G, X₁₉ is E or Q, X₂₁ is Q or K, and X₂₂ is | acid consensus |
| | V | addition at N- |
| | | terminus |
| 196 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGTIGRPYDYWGQGTQVTVSSGGGSGGGGSGGGGS | VHH1 N-terminal |
| | GGGGSGGGGSGGGGSGGGGSEGVGX₂₀X₂₂X₂₁LVESGGGLVQPGGS | consensus residue |
| | LRLSCAASGRTFSTYIIGWFRQAPGKEREFVASISWGGRGTYYAD | and consensus |
| | SVKDRFTISRDNPKNMVYLQMNSLRAEDTAVYYCAAGDLPGGRRA | CDR1, and VHH2 |
| | LDYDYWGQGTLVTVSS wherein X₁ is A or T, X₂ is A | with three amino |
| | or G, X₁₉ is E or Q, X₂₀ is E or Q, X₂₁ is Q or | acid consensus |
| | K, and X₂₂ is V | addition at N- |
| | | terminus |
| 197 | X₁₉VQLVESGGGLVQPGGSLRLSCAASGGTFSSYTMGWFRQAPGKE | Humanized 21H7 |
| | REYVGGISSSGYX₃X₄YX₅SX₆SMKGRFTISRDNAKNTLYLQMNSL | co-binder with |
| | RAEDTAVYYCAAGTIGRPYDYWGQGTQVTVSSGGGSGGGGSGGGG | VHH1 N-terminal |
| | SGGGGSGGGGSGGGGSGGGGSEGVGLVESGGGLVQPGGSLRLSCA | consensus residue |
| | ASGRTFSTYIIGWFRQAPGKEREFVASISWGGRGTYYADSVKDRF | and consensus |
| | TISRDNPKNMVYLQMNSLRAEDTAVYYCAAGDLPGGRRALDYDYW | CDR2A |
| | GQGTLVTVSS | |
| | wherein X₃ is T or V, X₄ is L or M, X₅ is K or | |
| | N, X₆ is D or E, and X₁₉ is E or Q | |
| 198 | X₁₉VQLVESGGGLVQPGGSLRLSCAASGGTFSSYTMGWFRQAPGKE | Humanized 21H7 |
| | REYVGGISSSGYX₃X₄YX₅X₆SMKGRFTISRDNAKNTLYLQMNSL | co-binder with |
| | RAEDTAVYYCAAGTIGRPYDYWGQGTQVTVSSGGGSGGGGSGGGG | VHH1 N-terminal |
| | SGGGGSGGGGSGGGGSGGGGSEGVGX₂₁LVESGGGLVQPGGSLRLS | consensus residue |
| | CAASGRTFSTYIIGWFRQAPGKEREFVASISWGGRGTYYADSVKD | and consensus |
| | RFTISRDNPKNMVYLQMNSLRAEDTAVYYCAAGDLPGGRRALDYD | CDR2A, and |
| | YWGQGTLVTVSS wherein X₃ is T or V, X₄ is L or | VHH2 with one |
| | M, X₅ is K or N, X₆ is D or E, X₁₉ is E or Q, | amino acid |
| | and X₂₁ is Q or K | consensus addition |
| | | at N-terminus |
| 199 | X₁₉VQLVESGGGLVQPGGSLRLSCAASGGTFSSYTMGWFRQAPGKE | Humanized 21H7 |
| | REYVGGISSSGYX₃X₄YX₅SX₆SMKGRFTISRDNAKNTLYLQMNSL | co-binder with |
| | RAEDTAVYYCAAGTIGRPYDYWGQGTQVTVSSGGGSGGGGSGGGG | VHH1 N-terminal |
| | SGGGGSGGGGSGGGGSGGGGSEGVGX₂₂X₂₁LVESGGGLVQPGGSL | consensus residue |
| | RLSCAASGRTFSTYIIGWFRQAPGKEREFVASISWGGRGTYYADS | and consensus |
| | VKDRFTISRDNPKNMVYLQMNSLRAEDTAVYYCAAGDLPGGRRAL | CDR2A, and |
| | DYDYWGQGTLVTVSS wherein X₃ is T or V, X₄ is L | VHH2 with two |
| | or M, X₅ is K or N, X₆ is D or E, X₁₉ is E or | amino acid |
| | Q, X₂₁ is Q or K , and X₂₂ is V | consensus addition |
| | | at N-terminus |
| 200 | X₁₉VQLVESGGGLVQPGGSLRLSCAASGGTFSSYTMGWFRQAPGKE | Humanized 21H7 |
| | REYVGGISSSGYX₃X₄YX₅SX₆SMKGRFTISRDNAKNTLYLQMNSL | co-binder with |
| | RAEDTAVYYCAAGTIGRPYDYWGQGTQVTVSSGGGSGGGGSGGGG | VHH1 N-terminal |
| | SGGGGSGGGGSGGGGSGGGGSEGVGX₂₀X₂₂X₂₁LVESGGGLVQPGG | consensus residue |
| | SLRLSCAASGRTFSTYIIGWFRQAPGKEREFVASISWGGRGTYYA | and consensus |
| | DSVKDRFTISRDNPKNMVYLQMNSLRAEDTAVYYCAAGDLPGGRR | CDR2A, and |
| | ALDYDYWGQGTLVTVSS wherein X₃ is T or V, X₄ is | VHH2 with three |
| | L or M, X₅ is K or N, X₆ is D or E, X₁₉ is E | amino acid |
| | or Q, X₂₀ is E or Q, X₂₁ is Q or K, and X₂₂ is | consensus addition |
| | V | at N-terminus |
| 201 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGX₇X₈GRPYX₉YWGQGTQVTVSSGGGSGGGGSGGGGS | VHH1 N-terminal |
| | GGGGSGGGGSGGGGSGGGGSEGVGLVESGGGLVQPGGSLRLSCAA | consensus residue |
| | SGRTFSTYIIGWFRQAPGKEREFVASISWGGRGTYYADSVKDRFT | and consensus |
| | ISRDNPKNMVYLQMNSLRAEDTAVYYCAAGDLPGGRRALDYDYWG | CDR3A |
| | QGTLVTVSS wherein X₇ is I or T, X₈ is F or I, | |
| | X₉ is E or D, and X₁₉ is E or Q | |
| 202 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGX₇X₈GRPYX₉YWGQGTQVTVSSGGGSGGGGSGGGGS | VHH1 N-terminal |
| | GGGGSGGGGGGGGSGGGGSEGVGX₂₁LVESGGGLVQPGGSLRLSC | consensus residue |
| | AASGRTFSTYIIGWFRQAPGKEREFVASISWGGRGTYYADSVKDR | and consensus |
| | FTISRDNPKNMVYLQMNSLRAEDTAVYYCAAGDLPGGRRALDYDY | CDR3A, and |
| | WGQGTLVTVSS wherein X₇ is I or T, X₈ is F or | VHH2 with one |
| | I, X₉ is E or D, X₁₉ is E or Q, and X₂₁ is Q or | amino acid |
| | K | consensus addition |
| | | at N-terminus |
| 203 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGX₇X₈GRPYX₉YWGQGTQVTVSSGGGSGGGGSGGGGS | VHH1 N-terminal |
| | GGGGSGGGGSGGGGSGGGGSEGVGX₂₂X₂₁LVESGGGLVQPGGSLR | consensus residue |
| | LSCAASGRTFSTYIIGWFRQAPGKEREFVASISWGGRGTYYADSV | and consensus |
| | KDRFTISRDNPKNMVYLQMNSLRAEDTAVYYCAAGDLPGGRRALD | CDR3A, and |
| | YDYWGQGTLVTVSS wherein X₇ is I or T, X₈ is F | VHH2 with two |
| | or I, X₉ is E or D, X₁₉ is E or Q, X₂₁ is Q or | amino acid |
| | K, and X₂₂ is V | consensus addition |
| | | at N-terminus |
| 204 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGX₇X₈GRPYX₉YWGQGTQVTVSSGGGSGGGGSGGGGS | VHH1 N-terminal |
| | GGGGSGGGGSGGGGSGGGGSEGVGX₂₀X₂₂X₂₁LVESGGGLVQPGGS | consensus residue |
| | LRLSCAASGRTFSTYIIGWFRQAPGKEREFVASISWGGRGTYYAD | and consensus |
| | SVKDRFTISRDNPKNMVYLQMNSLRAEDTAVYYCAAGDL PGGRRA | CDR3A, and |
| | LDYDYWGQGTLVTVSS wherein X₇ is I or T, X₈ is F | VHH2 with three |
| | or I, X₉ is E or D, X₁₉ is E or Q, X₂₀ is E or | amino acid |
| | Q, X₂₁ is Q or K, and X₂₂ is V | consensus addition |
| | | at N-terminus |
| 205 | X₁₉VQLVESGGGLVQPGGSLRLSCAASGGTFSSYX₁MX₂WFRQAPG | Humanized 21H7 |
| | KEREYVGGISSSGYX₃X₄YX₅X₆SMKGRFTISRDNAKNTLYLQMN | co-binder with |
| | SLRAEDTAVYYCAAGTIGRPYDYWGQGTQVTVSSGGGSGGGGSGG | VHH1 N-terminal |
| | GGSGGGGSGGGGSGGGGSGGGGSEGVGLVESGGGLVQPGGSLRLS | consensus residue |
| | CAASGRTFSTYIIGWFRQAPGKEREFVASISWGGRGTYYADSVKD | and consensus |
| | RFTISRDNPKNMVYLQMNSLRAEDTAVYYCAAGDLPGGRRALDYD | CDRs1 and 2A |
| | YWGQGTLVTVSS wherein X₁ is A or T, X₂ is A or | |
| | G, X₃ is T or V, X₄ is L or M, X₅ is K or N, | |
| | X₆ is D or E, and X₁₉ is E or Q | |
| 206 | X₁₉VQLVESGGGLVQPGGSLRLSCAASGGTFSSYX₁MX₂WFRQAPG | Humanized 21H7 |
| | KEREYVGGISSSGYX₃X₄YX₅SX₆SMKGRFTISRDNAKNTLYLQMN | co-binder with |
| | SLRAEDTAVYYCAAGTIGRPYDYWGQGTQVTVSSGGGSGGGGSGG | VHH1 N-terminal |
| | GGSGGGGSGGGGSGGGGSGGGGSEGVGX₂₁LVESGGGLVQPGGSLR | consensus residue |
| | LSCAASGRTFSTYIIGWFRQAPGKEREFVASISWGGRGTYYADSV | and consensus |
| | KDRFTISRDNPKNMVYLQMNSLRAEDTAVYYCAAGDLPGGRRALD | CDRs1 and 2A, |
| | YDYWGQGTLVTVSS wherein X₁ is A or T, X₂ is A | and VHH2 with |
| | or G, X₃ is T or V, X₄ is L or M, X₅ is K or | one amino acid |
| | N, X₆ is D or E, X₁₉ is E or Q, and X₂₁ is Q or | consensus addition |
| | K | at N-terminus |
| 207 | X₁₉VQLVESGGGLVQPGGSLRLSCAASGGTFSSYX₁MX₂WFRQAPG | Humanized 21H7 |
| | KEREYVGGISSSGYX₃X₄YX₅SX₆SMKGRFTISRDNAKNTLYLQMN | co-binder with |
| | SLRAEDTAVYYCAAGTIGRPYDYWGQGTQVTVSSGGGSGGGGSGG | VHH1 N-terminal |
| | GGSGGGGSGGGGSGGGGSGGGGSEGVGX₂₂X₂₁LVESGGGLVQPGG | consensus residue |
| | SLRLSCAASGRTFSTYIIGWFRQAPGKEREFVASISWGGRGTYYA | and consensus |
| | DSVKDRFTISRDNPKNMVYLQMNSLRAEDTAVYYCAAGDLPGGRR | CDRs1 and 2A, |
| | ALDYDYWGQGTLVTVSS wherein X₁ is A or T, X₂ is | and VHH2 with |
| | A or G, X₃ is T or V, X₄ is L or M, X₅ is K or | two amino acid |
| | N, X₆ is D or E, X₁₉ is E or Q, X₂₁ is Q or K, | consensus addition |
| | and X₂₂ is V | at N-terminus |
| 208 | X₁₉VQLVESGGGLVQPGGSLRLSCAASGGTFSSYX₁MX₂WFRQAPG | Humanized 21H7 |
| | KEREYVGGISSSGYX₃X₄YX₅SX₆SMKGRFTISRDNAKNTLYLQMN | co-binder with |
| | SLRAEDTAVYYCAAGTIGRPYDYWGQGTQVTVSSGGGSGGGGSGG | VHH1 N-terminal |
| | GGSGGGGSGGGGSGGGGSGGGGSEGVGX₂₀X₂₂X₂₁LVESGGGLVQP | consensus residue |
| | GGSLRLSCAASGRTFSTYIIGWFRQAPGKEREFVASISWGGRGTY | and consensus |
| | YADSVKDRFTISRDNPKNMVYLQMNSLRAEDTAVYYCAAGDLPGG | CDRs1 and 2A, |
| | RRALDYDYWGQGTLVTVSS wherein X₁ is A or T, X₂ | and VHH2 with |
| | is A or G, X₃ is T or V, X₄ is L or M, X₅ is K | three amino acid |
| | or N, X₆ is D or E, X₁₉ is E or Q, X₂₀ is E or | consensus addition |
| | Q, X₂₁ is Q or K, and X₂₂ is V | at N-terminus |
| 209 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGX₇X₈GRPYX₉YWGQGTQVTVSSGGGSGGGGSGGG | VHH1 N-terminal |
| | GSGGGGSGGGGSGGGGSGGGGSEGVGLVESGGGLVQPGGSLRLSC | consensus residue |
| | AASGRTFSTYIIGWFRQAPGKEREFVASISWGGRGTYYADSVKDR | and consensus |
| | FTISRDNPKNMVYLQMNSLRAEDTAVYYCAAGDLPGGRRALDYDY | CDRs1 and 3A |
| | WGQGTLVTVSS wherein X₁ is A or T, X₂ is A or | |
| | G, X₇ is I or T, X₈ is F or I, X₉ is E or D, | |
| | and X₁₉ is E or Q | |
| 210 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGX₇X₈GRPYX₉YWGQGTQVTVSSGGGSGGGGSGGG | VHH1 N-terminal |
| | GSGGGGSGGGGSGGGGSGGGGSEGVGX₂₁LVESGGGLVQPGGSLRL | consensus residue |
| | SCAASGRTFSTYIIGWFRQAPGKEREFVASISWGGRGTYYADSVK | and consensus |
| | DRFTISRDNPKNMVYLQMNSLRAEDTAVYYCAAGDLPGGRRALDY | CDRs1 and 3A, |
| | DYWGQGTLVTVSS wherein X₁ is A or T, X₂ is A or | and VHH2 with |
| | G, X₇ is I or T, X₈ is F or I, X₉ is E or D, | one amino acid |
| | X₁₉ is E or Q, and X₂₁ is Q or K | consensus addition |
| | | at N-terminus |
| 211 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGX₇X₈GRPYX₉YWGQGTQVTVSSGGGSGGGGSGGG | VHH1 N-terminal |
| | GSGGGGSGGGGSGGGGSGGGGSEGVGX₂₂X₂₁LVESGGGLVQPGGS | consensus residue |
| | LRLSCAASGRTFSTYIIGWFRQAPGKEREFVASISWGGRGTYYAD | and consensus |
| | SVKDRFTISRDNPKNMVYLQMNSLRAEDTAVYYCAAGDLPGGRRA | CDRs1 and 3A, |
| | LDYDYWGQGTLVTVSS wherein X₁ is A or T, X₂ is A | and VHH2 with |
| | or G, X₇ is I or T, X₈ is F or I, X, is E or | two amino acid |
| | D, X₁₉ is E or Q, X₂₁ is Q or K, and X₂₂ is V | consensus addition |
| | | at N-terminus |
| 212 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGX₇X₈GRPYX₉YWGQGTQVTVSSGGGSGGGGSGGG | VHH1 N-terminal |
| | GSGGGGSGGGGSGGGGSGGGGSEGVGX₂₀X₂₂X₂₁LVESGGGLVQPG | consensus residue |
| | GSLRLSCAASGRTFSTYIIGWFRQAPGKEREFVASISWGGRGTYY | and consensus |
| | ADSVKDRFTISRDNPKNMVYLQMNSLRAEDTAVYYCAAGDLPGGR | CDRs1 and 3A, |
| | RALDYDYWGQGTLVTVSS wherein X₁ is A or T, X₂ is | and VHH2 with |
| | A or G, X₇ is I or T, X₈ is F or I, X₉ is E or | three amino acid |
| | D, X₁₉ is E or Q, X₂₀ is E or Q, X₂₁ is Q or K, | consensus addition |
| | and X₂₂ is V | at N-terminus |
| 213 | X₁₉VQLVESGGGLVQPGGSLRLSCAASGGTFSSYTMGWFRQAPGKE | Humanized 21H7 |
| | REYVGGISSSGYX₃X₄YX₅SX₆SMKGRFTISRDNAKNTLYLQMNSL | co-binder with |
| | RAEDTAVYYCAAGX₇X₈GRPYX₉YWGQGTQVTVSSGGGSGGGGSGG | VHH1 N-terminal |
| | GGSGGGGSGGGGSGGGGSGGGGSEGVGLVESGGGLVQPGGSLRLS | consensus residue |
| | CAASGRTFSTYIIGWFRQAPGKEREFVASISWGGRGTYYADSVKD | and consensus |
| | RFTISRDNPKNMVYLQMNSLRAEDTAVYYCAAGDLPGGRRALDYD | CDRs2A and 3A |
| | YWGQGTLVTVSS wherein X₃ is T or V, X₄ is L or | |
| | M, X₅ is K or N, X₆ is D or E, X₇ is I or T, | |
| | X₈ is F or I, X₉ is E or D, and X₁₉ is E or Q | |
| 214 | X₁₉VQLVESGGGLVQPGGSLRLSCAASGGTFSSYTMGWFRQAPGKE | Humanized 21H7 |
| | REYVGGISSSGYX₃X₄YX₅SX&SMKGRFTISRDNAKNTLYLQMNSL | co-binder with |
| | RAEDTAVYYCAAGX₇X₈GRPYX₉YWGQGTQVTVSSGGGSGGGGSGG | VHH1 N-terminal |
| | GGSGGGGSGGGGSGGGGSGGGGSEGVGX₂₁LVESGGGLVQPGGSLR | consensus residue |
| | LSCAASGRTFSTYIIGWFRQAPGKEREFVASISWGGRGTYYADSV | and consensus |
| | KDRFTISRDNPKNMVYLQMNSLRAEDTAVYYCAAGDLPGGRRALD | CDRs2A and 3A, |
| | YDYWGQGTLVTVSS wherein X₃ is T or V, X₄ is L | and VHH2 with |
| | or M, X₅ is K or N, X₆ is D or E, X₇ is I or | one amino acid |
| | T, X₈ is F or I, X₉ is E or D, X₁₉ is E or Q, | consensus addition |
| | and X₂₁ is Q or K | at N-terminus |
| 215 | X₁₉VQLVESGGGLVQPGGSLRLSCAASGGTFSSYTMGWFRQAPGKE | Humanized 21H7 |
| | REYVGGISSSGYX₃X₄YX₅SX₆SMKGRFTISRDNAKNTLYLQMNSL | co-binder with |
| | RAEDTAVYYCAAGX₇X₈GRPYX₉YWGQGTQVTVSSGGGSGGGGSGG | VHH1 N-terminal |
| | GGSGGGGSGGGGSGGGGSGGGGSEGVGX₂₂X₂₁LVESGGGLVQPGG | consensus residue |
| | SLRLSCAASGRTFSTYIIGWFRQAPGKEREFVASISWGGRGTYYA | and consensus |
| | DSVKDRFTISRDNPKNMVYLQMNSLRAEDTAVYYCAAGDLPGGRR | CDRs2A and 3A, |
| | ALDYDYWGQGTLVTVSS wherein X₃ is T or V, X₄ is | and VHH2 with |
| | L or M, X₅ is K or N, X₆ is D or E, X₇ is I or | two amino acid |
| | T, X₈ is F or I, X₉ is E or D, X₁₉ is E or Q, | consensus addition |
| | X₂₁ is Q or K, and X₂₂ is V | at N-terminus |
| 216 | X₁₉VQLVESGGGLVQPGGSLRLSCAASGGTFSSYTMGWFRQAPGKE | Humanized 21H7 |
| | REYVGGISSSGYX₃X₄YX₅SX₆SMKGRFTISRDNAKNTLYLQMNSL | co-binder with |
| | RAEDTAVYYCAAGX₇X₈GRPYX₉YWGQGTQVTVSSGGGSGGGGSGG | VHH1 N-terminal |
| | GGSGGGGSGGGGSGGGGSGGGGSEGVGX₂₀X₂₂X₂₁LVESGGGLVQP | consensus residue |
| | GGSLRLSCAASGRTFSTYIIGWFRQAPGKEREFVASISWGGRGTY | and consensus |
| | YADSVKDRFTISRDNPKNMVYLQMNSLRAEDTAVYYCAAGDLPGG | CDRs2A and 3A, |
| | RRALDYDYWGQGTLVTVSS wherein X₃ is T or V, X₄ | and VHH2 with |
| | is L or M, X₅ is K or N, X₆ is D or E, X₇ is I | three amino acid |
| | or T, X₈ is F or I, X₉ is E or D, X₁₉ is E or | consensus addition |
| | Q, X₂₀ is E or Q, X₂₁ is Q or K, and X₂₂ is V | at N-terminus |
| 217 | X₁₉VQLVESGGGLVQPGGSLRLSCAASGGTFSSYX₁MX₂WFRQAPG | Humanized 21H7 |
| | KEREYVGGISSSGYX₃X₄YX₅X₆SMKGRFTISRDNAKNTLYLQMN | co-binder with |
| | SLRAEDTAVYYCAAGX₇X₈GRPYX₉YWGQGTQVTVSSGGGSGGGGS | VHH1 N-terminal |
| | GGGGSGGGGSGGGGSGGGGSGGGGSEGVGLVESGGGLVQPGGSLR | consensus residue |
| | LSCAASGRTFSTYIIGWFRQAPGKEREFVASISWGGRGTYYADSV | and consensus |
| | KDRFTISRDNPKNMVYLQMNSLRAEDTAVYYCAAGDLPGGRRALD | CDRs1, 2A and |
| | YDYWGQGTLVTVSS wherein X₁ is A or T, X₂ is A | 3A |
| | or G, X₃ is T or V, X₄ is L or M, X₅ is K or | |
| | N, X₆ is D or E, X₇ is I or T, X₈ is F or I, | |
| | X₉ is E or D, and X₁₉ is E or Q | |
| 218 | X₁₉VQLVESGGGLVQPGGSLRLSCAASGGTFSSYX₁MX₂WFRQAPG | Humanized 21H7 |
| | KEREYVGGISSSGYX₃X₄YX₅SX₆SMKGRFTISRDNAKNTLYLQMN | co-binder with |
| | SLRAEDTAVYYCAAGX₇X₈GRPYX₉YWGQGTQVTVSSGGGSGGGGS | VHH1 N-terminal |
| | GGGGSGGGGSGGGGSGGGGSGGGGSEGVGX₂₁LVESGGGLVQPGGS | consensus residue |
| | LRLSCAASGRTFSTYIIGWFRQAPGKEREFVASISWGGRGTYYAD | and consensus |
| | SVKDRFTISRDNPKNMVYLQMNSLRAEDTAVYYCAAGDLPGGRRA | CDRs1, 2A and |
| | LDYDYWGQGTLVTVSS wherein X₁ is A or T, X₂ is A | 3A, and VHH2 |
| | or G, X₃ is T or V, X₄ is L or M, X₅ is K or | with one amino |
| | N, X₆ is D or E, X₇ is I or T, X₈ is F or I, | acid consensus |
| | X₉ is E or D, X₁₉ is E or Q, and X₂₁ is Q or K | addition at N- |
| | | terminus |
| 219 | X₁₉VQLVESGGGLVQPGGSLRLSCAASGGTFSSYX₁MX₂WFRQAPG | Humanized 21H7 |
| | KEREYVGGISSSGYX₃X₄YX₅SX₆SMKGRFTISRDNAKNTLYLQMN | co-binder with |
| | SLRAEDTAVYYCAAGX₇X₈GRPYX₉YWGQGTQVTVSSGGGSGGGGS | VHH1 N-terminal |
| | GGGGSGGGGSGGGGSGGGGSGGGGSEGVGX₂₂X₂₁LVESGGGLVQP | consensus residue |
| | GGSLRLSCAASGRTFSTYIIGWFRQAPGKEREFVASISWGGRGTY | and consensus |
| | YADSVKDRFTISRDNPKNMVYLQMNSLRAEDTAVYYCAAGDLPGG | CDRs1, 2A and |
| | RRALDYDYWGQGTLVTVSS wherein X₁ is A or T, X₂ | 3A, and VHH2 |
| | is A or G, X₃ is T or V, X₄ is L or M, X₅ is K | with two amino |
| | or N, X₆ is D or E, X₇ is I or T, X₈ is F or | acid consensus |
| | I, X₉ is E or D, X₁₉ is E or Q, X₂₁ is Q or K, | addition at N- |
| | and X₂₂ is V | terminus |
| 220 | X₁₉VQLVESGGGLVQPGGSLRLSCAASGGTFSSYX₁MX₂WFRQAPG | Humanized 21H7 |
| | KEREYVGGISSSGYX₃X₄YX₅SX₆SMKGRFTISRDNAKNTLYLQMN | co-binder with |
| | SLRAEDTAVYYCAAGX₇X₈GRPYX₉YWGQGTQVTVSSGGGSGGGGS | VHH1 N-terminal |
| | GGGGGGGGSGGGGSGGGGSGGGGSEGVGX₂₀X₂₂X₂₁LVESGGGLV | consensus residue |
| | QPGGSLRLSCAASGRTFSTYIIGWFRQAPGKEREFVASISWGGRG | and consensus |
| | TYYADSVKDRFTISRDNPKNMVYLQMNSLRAEDTAVYYCAAGDLP | CDRs1, 2A and |
| | GGRRALDYDYWGQGTLVTVSS wherein X₁ is A or T, X₂ | 3A, and VHH2 |
| | is A or G, X₃ is T or V, X₄ is L or M, X₅ is K | with three amino |
| | or N, X₆ is D or E, X₇ is I or T, X₈ is F or | acid consensus |
| | I, X₉ is E or D, X₁₉ is E or Q, X₂₀ is E or Q, | addition at N- |
| | X₂₁ is Q or K, and X₂₂ is V | terminus |
| 221 | X₁₉VQLVESGGGLVQPGGSLRLSCAASGGTFSSYTMGWFRQAPGKE | Humanized 21H7 |
| | REYVGGISSSGYX₁₀X₁₁YX₁₂X₁₃X₁₄SMKGRFTISRDNAKNTLYLQM | VHH1 with N- |
| | NSLRAEDTAVYYCAAGTIGRPYDYWGQGTQVTVSS wherein | terminal consensus |
| | X₁₀ is T or V, X₁₁ is L or M, X₁₂ is K or N, X₁₃ | residue, and |
| | is S or A, X₁₄ is D or E, and X₁₉ is E or Q | consensus CDR2B |
| 222 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | VHH1 with N- |
| | DTAVYYCAAGX₁₅X₁₆GRPYX₁₇X₁₈WGQGTQVTVSS wherein X₁₅ | terminal consensus |
| | is I, T, or V, X₁₆ is F or I, X₁₇ is E or D, | residue, and |
| | X₁₈ is Y or F, and X₁₉ is E or Q | consensus CDR3B |
| 223 | X₁₉VQLVESGGGLVQPGGSLRLSCAASGGTFSSYX₁MX₂WFRQAPG | Humanized 21H7 |
| | KEREYVGGISSSGYX₁₀X₁₁YX₁₂X₁₃X₁₄SMKGRFTISRDNAKNTLYL | VHH1 with N- |
| | QMNSLRAEDTAVYYCAAGTIGRPYDYWGQGTQVTVSS wherein | terminal consensus |
| | X₁ is A or T, X₂ is A or G, X₁₀ is T or V, X₁₁ | residue, and |
| | is L or M, X₁₂ is K or N, X₁₃ is S or A, X₁₄ is | consensus CDRs1 |
| | D or E, and X₁₉ is E or Q | and 2B |
| 224 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | VHH1 with N- |
| | AEDTAVYYCAAGX₁₅X₁₆GRPYX₁₇X₁₈WGQGTQVTVSS wherein | terminal consensus |
| | X₁ is A or T, X₂ is A or G, X₁₅ is I, T, or V, | residue, and |
| | X₁₆ is F or I, X₁₇ is E or D, X₁₈ is Y or F, | consensus CDRs1 |
| | and X₁₉ is E or Q | and 3B |
| 225 | X₁₉VQLVESGGGLVQPGGSLRLSCAASGGTFSSYTMGWFRQAPGKE | Humanized 21H7 |
| | REYVGGISSSGYX₁₀X₁₁YX₁₂X₁₃X₁₄SMKGRFTISRDNAKNTLYLQM | VHH1 with N- |
| | NSLRAEDTAVYYCAAGX₁₅X₁₆GRPYX₁₇X₁₈WGQGTQVTVSS | terminal consensus |
| | wherein X₁₀ is T or V, X₁₁ is L or M, X₁₂ is K | residue, and |
| | or N, X₁₃ is S or A, X₁₄ is D or E, X₁₅ is I, | consensus |
| | T, or V, X₁₆ is F or I, X₁₇ is E or D, X₁₈ is Y | CDRs2B and 3B |
| | or F, and X₁₉ is E or Q | |
| 226 | X₁₉VQLVESGGGLVQPGGSLRLSCAASGGTFSSYX₁MX₂WFRQAPG | Humanized 21H7 |
| | KEREYVGGISSSGYX₁₀X₁₁YX₁₂X₁₃X₁₄SMKGRFTISRDNAKNTLYL | VHH1 with N- |
| | QMNSLRAEDTAVYYCAAGX₁₅X₁₆GRPYX₁₇X₁₈WGQGTQVTVSS | terminal consensus |
| | wherein X₁ is A or T, X₂ is A or G, X₁₀ is T | residue, and |
| | or V, X₁₁ is L or M, X₁₂ is K or N, X₁₃ is S or | consensus CDRs1, |
| | A, X₁₄ is D or E, X₁₅ is I, T, or V, X₁₆ is F | 2B and 3B |
| | or I, X₁₇ is E or D, X₁₈ is Y or F, and X₁₉ is | |
| | E or Q | |
| 227 | X₁₉VQLVESGGGLVQPGGSLRLSCAASGGTFSSYTMGWFRQAPGKE | Humanized 21H7 |
| | REYVGGISSSGYX₁₀X₁₁YX₁₂X₁₃X₁₄SMKGRFTISRDNAKNTLYLQM | co-binder with |
| | NSLRAEDTAVYYCAAGTIGRPYDYWGQGTQVTVSSGGGSGGGGSG | VHH1 N-terminal |
| | GGGSGGGGSGGGGSGGGGSGGGGSEGVGLVESGGGLVQPGGSLRL | consensus residue, |
| | SCAASGRTFSTYIIGWFRQAPGKEREFVASISWGGRGTYYADSVK | and consensus |
| | DRFTISRDNPKNMVYLQMNSLRAEDTAVYYCAAGDLPGGRRALDY | CDR2B |
| | DYWGQGTLVTVSS wherein X₁₀ is T or V, X₁₁ is L | |
| | or M, X₁₂ is K or N, X₁₃ is S or A, X₁₄ is D or | |
| | E, and X₁₉ is E or Q | |
| 228 | X₁₉VQLVESGGGLVQPGGSLRLSCAASGGTFSSYTMGWFRQAPGKE | Humanized 21H7 |
| | REYVGGISSSGYX₁₀X₁₁YX₁₂X₁₃X₁₄SMKGRFTISRDNAKNTLYLQM | co-binder with |
| | NSLRAEDTAVYYCAAGTIGRPYDYWGQGTQVTVSSGGGSGGGGSG | VHH1 N-terminal |
| | GGGSGGGGSGGGGSGGGGSGGGGSEGVGX₂₁LVESGGGLVQPGGSL | consensus residue, |
| | RLSCAASGRTFSTYIIGWFRQAPGKEREFVASISWGGRGTYYADS | and consensus |
| | VKDRFTISRDNPKNMVYLQMNSLRAEDTAVYYCAAGDLPGGRRAL | CDR2B, and |
| | DYDYWGQGTLVTVSS wherein X₁₀ is T or V, X₁₁ is | VHH2 with one |
| | L or M, X₁₂ is K or N, X₁₃ is S or A, X₁₄ is D | amino acid |
| | or E, X₁₉ is E or Q, and X₂₁ is Q or K | consensus addition |
| | | at N-terminus |
| 229 | X₁₉VQLVESGGGLVQPGGSLRLSCAASGGTFSSYTMGWFRQAPGKE | Humanized 21H7 |
| | REYVGGISSSGYX₁₀X₁₁YX₁₂X₁₃X₁₄SMKGRFTISRDNAKNTLYLQM | co-binder with |
| | NSLRAEDTAVYYCAAGTIGRPYDYWGQGTQVTVSSGGGSGGGGSG | VHH1 N-terminal |
| | GGGSGGGGSGGGGSGGGGSGGGGSEGVGX₂₂X₂₁LVESGGGLVQPG | consensus residue, |
| | GSLRLSCAASGRTFSTYIIGWFRQAPGKEREFVASISWGGRGTYY | and consensus |
| | ADSVKDRFTISRDNPKNMVYLQMNSLRAEDTAVYYCAAGDLPGGR | CDR2B, and |
| | RALDYDYWGQGTLVTVSS wherein X₁₀ is T or V, X₁₁ | VHH2 with two |
| | is L or M, X₁₂ is K or N, X₁₃ is S or A, X₁₄ is | amino acid |
| | D or E, X₁₉ is E or Q, X₂₁ is Q or K, and X₂₂ | consensus addition |
| | is V | at N-terminus |
| 230 | X₁₉VQLVESGGGLVQPGGSLRLSCAASGGTFSSYTMGWFRQAPGKE | Humanized 21H7 |
| | REYVGGISSSGYX₁₀X₁₁YX₁₂X₁₃X₁₄SMKGRFTISRDNAKNTLYLQM | co-binder with |
| | NSLRAEDTAVYYCAAGTIGRPYDYWGQGTQVTVSSGGGSGGGGSG | VHH1 N-terminal |
| | GGGSGGGGSGGGGSGGGGGGGGSEGVGX₂₀X₂₂X₂₁LVESGGGLVQ | consensus residue, |
| | PGGSLRLSCAASGRTFSTYIIGWFRQAPGKEREFVASISWGGRGT | and consensus |
| | YYADSVKDRFTISRDNPKNMVYLQMNSLRAEDTAVYYCAAGDLPG | CDR2B, and |
| | GRRALDYDYWGQGTLVTVSS wherein X₁₀ is T or V, | VHH2 with three |
| | X₁₁ is L or M, X₁₂ is K or N, X₁₃ is S or A, X₁₄ | amino acid |
| | is D or E, X₁₉ is E or Q, X₂₀ is E or Q, X₂₁ is | consensus addition |
| | Q or K, and X₂₂ is V | at N-terminus |
| 231 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGX₁₅X₁₆GRPYX₁₇X₁₈WGQGTQVTVSSGGGSGGGGSGG | VHH1 N-terminal |
| | GGSGGGGSGGGGSGGGGSGGGGSEGVGLVESGGGLVQPGGSLRLS | consensus residue, |
| | CAASGRTFSTYIIGWFRQAPGKEREFVASISWGGRGTYYADSVKD | and consensus |
| | RFTISRDNPKNMVYLQMNSLRAEDTAVYYCAAGDLPGGRRALDYD | CDR3B |
| | YWGQGTLVTVSS wherein X₁₅ is I, T, or V, X₁₆ is | |
| | F or I, X₁₇ is E or D, X₁₈ is Y or F, and X₁₉ | |
| | is E or Q | |
| 232 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGX₁₅X₁₆GRPYX₁₇X₁₈WGQGTQVTVSSGGGSGGGGSGG | |
| | GGSGGGGSGGGGSGGGGSGGGGSEGVGX₂₁LVESGGGLVQPGGSLR | VHH1 N-terminal |
| | LSCAASGRTFSTYIIGWFRQAPGKEREFVASISWGGRGTYYADSV | consensus residue, |
| | KDRFTISRDNPKNMVYLQMNSLRAEDTAVYYCAAGDLPGGRRALD | and consensus |
| | YDYWGQGTLVTVSS wherein X₁₅ is I, T, or V, X₁₆ | CDR3B, and |
| | is F or I, X₁₇ is E or D, X₁₈ is Y or F, X₁₉ is | VHH2 with one |
| | E or Q, and X₂₁ is Q or K | amino acid |
| | | consensus addition |
| | | at N-terminus |
| 233 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGX₁₅X₁₆GRPYX₁₇X₁₈WGQGTQVTVSSGGGSGGGGSGG | VHH1 N-terminal |
| | GGSGGGGSGGGGSGGGGSGGGGSEGVGX₂₂X₂₁LVESGGGLVQPGG | consensus residue, |
| | SLRLSCAASGRTFSTYIIGWFRQAPGKEREFVASISWGGRGTYYA | and consensus |
| | DSVKDRFTISRDNPKNMVYLQMNSLRAEDTAVYYCAAGDLPGGRR | CDR3B, and |
| | ALDYDYWGQGTLVTVSS wherein X₁₅ is I, T, or V, | VHH2 with two |
| | X₁₆ is F or I, X₁₇ is E or D, X₁₈ is Y or F, X₁₉ | amino acid |
| | is E or Q, X₂₁ is Q or K, and X₂₂ is V | consensus addition |
| | | at N-terminus |
| 234 | X₁₉VQLVESGGGLVQPGGSLRLSCAASGGTFSSYTMGWFRQAPGKE | Humanized 21H7 |
| | REYVGGISSSGYVMYNSESMKGRFTISRDNAKNTLYLQMNSLRAE | co-binder with |
| | DTAVYYCAAGX₁₅X₁₆GRPYX₁₇X₁₈WGQGTQVTVSSGGGSGGGGSGG | VHH1 N-terminal |
| | GGSGGGGSGGGGSGGGGSGGGGSEGVGX₂₀X₂₂X₂₁LVESGGGLVQP | consensus residue, |
| | GGSLRLSCAASGRTFSTYIIGWFRQAPGKEREFVASISWGGRGTY | and consensus |
| | YADSVKDRFTISRDNPKNMVYLQMNSLRAEDTAVYYCAAGDLPGG | CDR3B, and |
| | RRALDYDYWGQGTLVTVSS wherein X₁₅ is I, T, or V, | VHH2 with three |
| | X₁₆ is F or I, X₁₇ is E or D, X₁₈ is Y or F, X₁₉ | amino acid |
| | is E or Q, X₂₀ is E or Q, X₂₁ is Q or K, and | consensus addition |
| | X₂₂ is V | at N-terminus |
| 235 | X₁₉VQLVESGGGLVQPGGSLRLSCAASGGTFSSYX₁MX₂WFRQAPG | Humanized 21H7 |
| | KEREYVGGISSSGYX₁₀X₁₁YX₁₂X₁₃X₁₄SMKGRFTISRDNAKNTLYL | co-binder with |
| | QMNSLRAEDTAVYYCAAGTIGRPYDYWGQGTQVTVSSGGGSGGGG | VHH1 N-terminal |
| | SGGGGSGGGGSGGGGSGGGGSGGGGSEGVGLVESGGGLVQPGGSL | consensus residue, |
| | RLSCAASGRTFSTYIIGWFRQAPGKEREFVASISWGGRGTYYADS | and consensus |
| | VKDRFTISRDNPKNMVYLQMNSLRAEDTAVYYCAAGDLPGGRRAL | CDRs1 and 2B |
| | DYDYWGQGTLVTVSS wherein X₁ is A or T, X₂ is A | |
| | or G, X₁₀ is T or V, X₁₁ is L or M, X₁₂ is K or | |
| | N, X₁₃ is S or A, X₁₄ is D or E, and X₁₉ is E | |
| | or Q | |
| 236 | X₁₉VQLVESGGGLVQPGGSLRLSCAASGGTFSSYX₁MX₂WFRQAPG | Humanized 21H7 |
| | KEREYVGGISSSGYX₁₀X₁₁YX₁₂X₁₃X₁₄SMKGRFTISRDNAKNTLYL | co-binder with |
| | QMNSLRAEDTAVYYCAAGTIGRPYDYWGQGTQVTVSSGGGSGGGG | VHH1 N-terminal |
| | SGGGGSGGGGSGGGGSGGGGSGGGGSEGVGX₂₁LVESGGGLVQPGG | consensus residue, |
| | SLRLSCAASGRTFSTYIIGWFRQAPGKEREFVASISWGGRGTYYA | and consensus |
| | DSVKDRFTISRDNPKNMVYLQMNSLRAEDTAVYYCAAGDLPGGRR | CDRs1 and 2B, |
| | ALDYDYWGQGTLVTVSS wherein X₁ is A or T, X₂ is | and VHH2 with |
| | A or G, X₁₀ is T or V, X₁₁ is L or M, X₁₂ is K | one amino acid |
| | or N, X₁₃ is S or A, X₁₄ is D or E, X₁₉ is E or | consensus addition |
| | Q, and X₂₁ is Q or K | at N-terminus |
| 237 | X₁₉VQLVESGGGLVQPGGSLRLSCAASGGTFSSYX₁MX₂WFRQAPG | Humanized 21H7 |
| | KEREYVGGISSSGYX₁₀X₁₁YX₁₂X₁₃X₁₄SMKGRFTISRDNAKNTLYL | co-binder with |
| | QMNSLRAEDTAVYYCAAGTIGRPYDYWGQGTQVTVSSGGGSGGGG | VHH1 N-terminal |
| | SGGGGSGGGGSGGGGSGGGGSGGGGSEGVGX₂₂X₂₁LVESGGGLVQ | consensus residue, |
| | PGGSLRLSCAASGRTFSTYIIGWFRQAPGKEREFVASISWGGRGT | and consensus |
| | YYADSVKDRFTISRDNPKNMVYLQMNSLRAEDTAVYYCAAGDLPG | CDRs1 and 2B, |
| | GRRALDYDYWGQGTLVTVSS wherein X₁ is A or T, X₂ | and VHH2 with |
| | is A or G, X₁₀ is T or V, X₁₁ is L or M, X₁₂ is | two amino acid |
| | K or N, X₁₃ is S or A, X₁₄ is D or E, X₁₉ is E | consensus addition |
| | or Q, X₂₁ is Q or K, and X₂₂ is V | at N-terminus |
| 238 | X₁₉VQLVESGGGLVQPGGSLRLSCAASGGTFSSYX₁MX₂WFRQAPG | Humanized 21H7 |
| | KEREYVGGISSSGYX₁₀X₁₁YX₁₂X₁₃X₁₄SMKGRFTISRDNAKNTLYL | co-binder with |
| | QMNSLRAEDTAVYYCAAGTIGRPYDYWGQGTQVTVSSGGGSGGGG | VHH1 N-terminal |
| | SGGGGSGGGGSGGGGSGGGGSGGGGSEGVGX₂₀X₂₂X₂₁LVESGGGL | consensus residue, |
| | VQPGGSLRLSCAASGRTFSTYIIGWFRQAPGKEREFVASISWGGR | and consensus |
| | GTYYADSVKDRFTISRDNPKNMVYLQMNSLRAEDTAVYYCAAGDL | CDRs1 and 2B, |
| | PGGRRALDYDYWGQGTLVTVSS wherein X₁ is A or T, | and VHH2 with |
| | X₂ is A or G, X₁₀ is T or V, X₁₁ is L or M, X₁₂ | three amino acid |
| | is K or N, X₁₃ is S or A, X₁₄ is D or E, X₁₉ is | consensus addition |
| | E or Q, X₂₀ is E or Q, X₂₁ is Q or K, and X₂₂ | at N-terminus |
| | is V | |
| 239 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGX₁₅X₁₆GRPYX₁₇X₁₈WGQGTQVTVSSGGGSGGGGS | VHH1 N-terminal |
| | GGGGSGGGGSGGGGSGGGGSGGGGSEGVGLVESGGGLVQPGGSLR | consensus residue, |
| | LSCAASGRTFSTYIIGWFRQAPGKEREFVASISWGGRGTYYADSV | and consensus |
| | KDRFTISRDNPKNMVYLQMNSLRAEDTAVYYCAAGDLPGGRRALD | CDRs1 and 3B |
| | YDYWGQGTLVTVSS wherein X₁ is A or T, X₂ is A | |
| | or G, X₁₅ is I, T, or V, X₁₆ is F or I, X₁₇ is | |
| | E or D, X₁₈ is Y or F, and X₁₉ is E or Q | |
| 240 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGX₁₅X₁₆GRPYX₁₇X₁₈WGQGTQVTVSSGGGSGGGGS | VHH1 N-terminal |
| | GGGGSGGGGSGGGGSGGGGSGGGGSEGVGX₂₁LVESGGGLVQPGGS | consensus residue, |
| | LRLSCAASGRTFSTYIIGWFRQAPGKEREFVASISWGGRGTYYAD | and consensus |
| | SVKDRFTISRDNPKNMVYLQMNSLRAEDTAVYYCAAGDLPGGRRA | CDRs1 and 3B, |
| | LDYDYWGQGTLVTVSS wherein X₁ is A or T, X₂ is A | and VHH2 with |
| | or G, X₁₅ is I, T, or V, X₁₆ is F or I, X₁₇ is | one amino acid |
| | E or D, X₁₈ is Y or F, X₁₉ is E or Q, and X₂₁ | consensus addition |
| | is Q or K | at N-terminus |
| 241 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGX₁₅X₁₆GRPYX₁₇X₁₈WGQGTQVTVSSGGGSGGGGS | VHH1 N-terminal |
| | GGGGSGGGGSGGGGSGGGGSGGGGSEGVGX₂₂X₂₁LVESGGGLVQP | consensus residue, |
| | GGSLRLSCAASGRTFSTYIIGWFRQAPGKEREFVASISWGGRGTY | and consensus |
| | YADSVKDRFTISRDNPKNMVYLQMNSLRAEDTAVYYCAAGDLPGG | CDRs1 and 3B, |
| | RRALDYDYWGQGTLVTVSS wherein X₁ is A or T, X₂ | and VHH2 with |
| | is A or G, X₁₅ is I, T, or V, X₁₆ is F or I, | two amino acid |
| | X₁₇ is E or D, X₁₈ is Y or F, X₁₉ is E or Q, X₂₁ | consensus addition |
| | is Q or K, and X₂₂ is V | at N-terminus |
| 242 | X₁₉VQLVESGGGLVQPGGSLRLSCAASGGTFSSYX₁MX₂WFRQAPG | Humanized 21H7 |
| | KEREYVGGISSSGYVMYNSESMKGRFTISRDNAKNTLYLQMNSLR | co-binder with |
| | AEDTAVYYCAAGX₁₅X₁₆GRPYX₁₇X₁₈WGQGTQVTVSSGGGSGGGGS | |
| | GGGGSGGGGSGGGGSGGGGSGGGGSEGVGX₂₀X₂₂X₂₁LVESGGGLV | VHH1 N-terminal |
| | QPGGSLRLSCAASGRTFSTYIIGWFRQAPGKEREFVASISWGGRG | consensus residue, |
| | TYYADSVKDRFTISRDNPKNMVYLQMNSLRAEDTAVYYCAAGDLP | and consensus |
| | GGRRALDYDYWGQGTLVTVSS wherein X₁ is A or T, X₂ | CDRs1 and 3B, |
| | is A or G, X₁₅ is I, T, or V, X₁₆ is F or I, | and VHH2 with |
| | X₁₇ is E or D, X₁₈ is Y or F, X₁₉ is E or Q, X₂₀ | three amino acid |
| | is E or Q, X₂₁ is Q or K, and X₂₂ is V | consensus addition |
| | | at N-terminus |
| 243 | X₁₉VQLVESGGGLVQPGGSLRLSCAASGGTFSSYTMGWFRQAPGKE | Humanized 21H7 |
| | REYVGGISSSGYX₁₀X₁₁YX₁₂X₁₃X₁₄SMKGRFTISRDNAKNTLYLQM | co-binder with |
| | NSLRAEDTAVYYCAAGX₁₅X₁₆GRPYX₁₇X₁₈WGQGTQVTVSSGGGSG | VHH1 N-terminal |
| | GGGSGGGGSGGGGSGGGGSGGGGSGGGGSEGVGLVESGGGLVQPG | consensus residue, |
| | GSLRLSCAASGRTFSTYIIGWFRQAPGKEREFVASISWGGRGTYY | and consensus |
| | ADSVKDRFTISRDNPKNMVYLQMNSLRAEDTAVYYCAAGDLPGGR | CDRs2B and 3B |
| | RALDYDYWGQGTLVTVSS wherein X₁₀ is T or V, X₁₁ | |
| | is L or M, X₁₂ is K or N, X₁₃ is S or A, X₁₄ is | |
| | D or E, X₁₅ is I, T, or V, X₁₆ is F or I, X₁₇ | |
| | is E or D, X₁₈ is Y or F, and X₁₉ is E or Q | |
| 244 | X₁₉VQLVESGGGLVQPGGSLRLSCAASGGTFSSYTMGWFRQAPGKE | Humanized 21H7 |
| | REYVGGISSSGYX₁₀X₁₁YX₁₂X₁₃X₁₄SMKGRFTISRDNAKNTLYLQM | co-binder with |
| | NSLRAEDTAVYYCAAGX₁₅X₁₆GRPYX₁₇X₁₈WGQGTQVTVSSGGGSG | VHH1 N-terminal |
| | GGGSGGGGSGGGGSGGGGSGGGGSGGGGSEGVGX₂₁LVESGGGLVQ | consensus residue, |
| | PGGSLRLSCAASGRTFSTYIIGWFRQAPGKEREFVASISWGGRGT | and consensus |
| | YYADSVKDRFTISRDNPKNMVYLQMNSLRAEDTAVYYCAAGDLPG | CDRs2B and 3B, |
| | GRRALDYDYWGQGTLVTVSS wherein X₁₀ is T or V, | and VHH2 with |
| | X₁₁ is L or M, X₁₂ is K or N, X₁₃ is S or A, X₁₄ | one amino acid |
| | is D or E, X₁₅ is I, T, or V, X₁₆ is F or I, | consensus addition |
| | and X₁₇ is E or D, X₁₈ is Y or F, X₁₉ is E or | at N-terminus |
| | Q, and X₂₁ is Q or K | |
| 245 | X₁₉VQLVESGGGLVQPGGSLRLSCAASGGTFSSYTMGWFRQAPGKE | Humanized 21H7 |
| | REYVGGISSSGYX₁₀X₁₁YX₁₂X₁₃X₁₄SMKGRFTISRDNAKNTLYLQM | co-binder with |
| | NSLRAEDTAVYYCAAGX₁₅X₁₆GRPYX₁₇X₁₈WGQGTQVTVSSGGGSG | VHH1 N-terminal |
| | GGGSGGGGSGGGGSGGGGSGGGGSGGGGSEGVGX₂₂X₂₁LVESGGG | consensus residue, |
| | LVQPGGSLRLSCAASGRTFSTYIIGWFRQAPGKEREFVASISWGG | and consensus |
| | RGTYYADSVKDRFTISRDNPKNMVYLQMNSLRAEDTAVYYCAAGD | CDRs2B and 3B, |
| | LPGGRRALDYDYWGQGTLVTVSS wherein X₁₀ is T or V, | and VHH2 with |
| | X₁₁ is L or M, X₁₂ is K or N, X₁₃ is S or A, X₁₄ | two amino acid |
| | is D or E, X₁₅ is I, T, or V, X₁₆ is F or I, | consensus addition |
| | X₁₇ is E or D, X₁₈ is Y or F, X₁₉ is E or Q, X₂₁ | at N-terminus |
| | is Q or K, and X₂₂ is V | |
| 246 | X₁₉VQLVESGGGLVQPGGSLRLSCAASGGTFSSYTMGWFRQAPGKE | Humanized 21H7 |
| | REYVGGISSSGYX₁₀X₁₁YX₁₂X₁₃X₁₄SMKGRFTISRDNAKNTLYLQM | co-binder with |
| | NSLRAEDTAVYYCAAGX₁₅X₁₆GRPYX₁₇X₁₈WGQGTQVTVSSGGGSG | VHH1 N-terminal |
| | GGGSGGGGSGGGGSGGGGSGGGGSGGGGSEGVGX₂₀X₂₂X₂₁LVESG | consensus residue, |
| | GGLVQPGGSLRLSCAASGRTFSTYIIGWFRQAPGKEREFVASISW | and consensus |
| | GGRGTYYADSVKDRFTISRDNPKNMVYLQMNSLRAEDTAVYYCAA | CDRs2B and 3B, |
| | GDLPGGRRALDYDYWGQGTLVTVSS wherein X₁₀ is T or | and VHH2 with |
| | V, X₁₁ is L or M, X₁₂ is K or N, X₁₃ is S or A, | three amino acid |
| | X₁₄ is D or E, X₁₅ is I, T, or V, X₁₆ is F or | consensus addition |
| | I, X₁₇ is E or D, X₁₈ is Y or F, X₁₉ is E or Q, | at N-terminus |
| | X₂₀ is E or Q, X₂₁ is Q or K, and X₂₂ is V | |
| 247 | X₁₉VQLVESGGGLVQPGGSLRLSCAASGGTFSSYX₁MX₂WFRQAPG | Humanized 21H7 |
| | KEREYVGGISSSGYX₁₀X₁₁YX₁₂X₁₃X₁₄SMKGRFTISRDNAKNTLYL | co-binder with |
| | QMNSLRAEDTAVYYCAAGX₁₅X₁₆GRPYX₁₇X₁₈WGQGTQVTVSSGGG | VHH1 N-terminal |
| | SGGGGSGGGGSGGGGSGGGGSGGGGSGGGGSEGVGLVESGGGLVQ | consensus residue, |
| | PGGSLRLSCAASGRTFSTYIIGWFRQAPGKEREFVASISWGGRGT | and consensus |
| | YYADSVKDRFTISRDNPKNMVYLQMNSLRAEDTAVYYCAAGDLPG | CDRs1, 2B and 3B |
| | GRRALDYDYWGQGTLVTVSS wherein X₁ is A or T, X₂ | |
| | is A or G, X₁₀ is T or V, X₁₁ is L or M, X₁₂ is | |
| | K or N, X₁₃ is S or A, X₁₄ is D or E, X₁₅ is I, | |
| | T, or V, X₁₆ is F or I, X₁₇ is E or D, X₁₈ is Y | |
| | or F, and X₁₉ is E or Q | |
| 248 | X₁₉VQLVESGGGLVQPGGSLRLSCAASGGTFSSYX₁MX₂WFRQAPG | Humanized 21H7 |
| | KEREYVGGISSSGYX₁₀X₁₁YX₁₂X₁₃X₁₄SMKGRFTISRDNAKNTLYL | co-binder with |
| | QMNSLRAEDTAVYYCAAGX₁₅X₁₆GRPYX₁₇X₁₈WGQGTQVTVSSGGG | VHH1 N-terminal |
| | SGGGGSGGGGSGGGGSGGGGSGGGGSGGGGSEGVGX₂₁LVESGGGL | consensus residue, |
| | VQPGGSLRLSCAASGRTFSTYIIGWFRQAPGKEREFVASISWGGR | and consensus |
| | GTYYADSVKDRFTISRDNPKNMVYLQMNSLRAEDTAVYYCAAGDL | CDRs1, 2B and |
| | PGGRRALDYDYWGQGTLVTVSS wherein X₁ is A or T, | 3B, VHH2 with |
| | X₂ is A or G, X₁₀ is T or V, X₁₁ is L or M, X₁₂ | one amino acid |
| | is K or N, X₁₃ is S or A, X₁₄ is D or E, X₁₅ is | consensus addition |
| | I, T, or V, X₁₆ is F or I, X₁₇ is E or D, X₁₈ | at N-terminus |
| | is Y or F, X₁₉ is E or Q, and X₂₁ is Q or K | |
| 249 | X₁₉VQLVESGGGLVQPGGSLRLSCAASGGTFSSYX₁MX₂WFRQAPG | Humanized 21H7 |
| | KEREYVGGISSSGYX₁₀X₁₁YX₁₂X₁₃X₁₄SMKGRFTISRDNAKNTLYL | co-binder with |
| | QMNSLRAEDTAVYYCAAGX₁₅X₁₆GRPYX₁₇X₁₈WGQGTQVTVSSGGG | VHH1 N-terminal |
| | SGGGGSGGGGSGGGGSGGGGSGGGGSGGGGSEGVGX₂₂X₂₁LVESG | consensus residue, |
| | GGLVQPGGSLRLSCAASGRTFSTYIIGWFRQAPGKEREFVASISW | and consensus |
| | GGRGTYYADSVKDRFTISRDNPKNMVYLQMNSLRAEDTAVYYCAA | CDRs1, 2B and |
| | GDLPGGRRALDYDYWGQGTLVTVSS wherein X₁ is A or | 3B, and VHH2 |
| | T, X₂ is A or G, X₁₀ is T or V, X₁₁ is L or M, | with two amino |
| | X₁₂ is K or N, X₁₃ is S or A, X₁₄ is D or E, X₁₅ | acid consensus |
| | is I, T, or V, X₁₆ is F or I, X₁₇ is E or D, | addition at N- |
| | X₁₈ is Y or F, X₁₉ is E or Q, X₂₁ is Q or K, | terminus |
| | and X₂₂ is V | |
| 250 | X₁₉VQLVESGGGLVQPGGSLRLSCAASGGTFSSYX₁MX₂WFRQAPG | Humanized 21H7 |
| | KEREYVGGISSSGYX₁₀X₁₁YX₁₂X₁₃X₁₄SMKGRFTISRDNAKNTLYL | co-binder with |
| | QMNSLRAEDTAVYYCAAGX₁₅X₁₆GRPYX₁₇X₁₈WGQGTQVTVSSGGG | VHH1 N-terminal |
| | SGGGGSGGGGSGGGGSGGGGSGGGGSGGGGSEGVGX₂₀X₂₂X₂₁LVE | consensus residue, |
| | SGGGLVQPGGSLRLSCAASGRTFSTYIIGWFRQAPGKEREFVASI | and consensus |
| | SWGGRGTYYADSVKDRFTISRDNPKNMVYLQMNSLRAEDTAVYYC | CDRs1, 2B and |
| | AAGDLPGGRRALDYDYWGQGTLVTVSS wherein X₁ is A | 3B, and VHH2 |
| | or T, X₂ is A or G, X₁₀ is T or V, X₁₁ is L or | with three amino |
| | M, X₁₂ is K or N, X₁₃ is S or A, X₁₄ is D or E, | acid consensus |
| | X₁₅ is I, T, or V, X₁₆ is F or I, X₁₇ is E or | addition at N- |
| | D, X₁₈ is Y or F, X₁₉ is E or Q, X₂₀ is E or Q, | terminus |
| | X₂₁ is Q or K, and X₂₂ is V | |
| 251 | IX₂₃AMG, X₂₃ is V, H, or Q. | Exemplary VHH2 |
| | | consensus CDR1 |
| | | (Kabat numbering) |
| 252 | AITSGGRX₂₄HYRDSVKG, X₂₄ is T or R. | Exemplary VHH2 |
| | | consensus CDR2 |
| | | (Kabat numbering) |
| 253 | DX₂₅GX₂₆X₂₇X₂₈VGEYDY | Exemplary VHH2 |
| | X₂₅ is R, M, or T | consensus CDR3 |
| | X₂₆ is W, L, or R | (Kabat numbering) |
| | X₂₇ is T or V | |
| | X₂₈ is S or A | |
| 254 | VQLVESGGGLVQAGGSLRLSCAASGSISSIYAMGWYRQAPGKQRE | 20C7 VHH2 single |
| | LVAAITYGGRSHYRDSVKGRFTISGNSDNSALYLHMNSLKPEDTA | binder |
| | VYYCAADRGLTAVGEYDYWGQGTQVTVSS | |
| 255 | EVQLVESGGGSVQPGGSLRLSCAASGSIFILNVMGWYRQAPGNQR | 1H4 VHH1 single |
| | ELVALMSSGGSTKYADSVKGRFTISRDNVRDTVYLQMNSLKPEDT | binder |
| | AVYYCTLRTENDYWGQGTQVTVSS | |
| 256 | QVQLVESGGGLVQAGGSLRLSCAASGRTFSSYAMGWFRQGPGKER | 1C2 VHH1 single |
| | EFVAAISWSGGGTYYADSVKGRFTISRDNAKNTVYLQMNSLKPED | binder |
| | TAVYYCAAGDLPWSRSDLEYDYWGQGTLVTVSS | |
| 257 | LEESGGGSVQAGGSLRLSCAASGRTFSTYAMGWLRQAPGKEREFV | 1E3 VHH2 single |
| | AGISWGGGRTDYADSVKDRFTISRDNPKNTVYLQMNSLKPEDTAV | binder |
| | YYCAAKDLPSGWGRTSEYDYWGQGTQVTVSS | |
| 258 | EVQLVESGGGSVQPGGSLRLSCAGSGSIFPLNVMGWYRQAPGKER | 1D12 VHH2 single |
| | EFIALITSGGSTKHADSVKGRFTISRDNFKNTVYLQMNSLKPEDT | binder |
| | AVYYCYLDQSRQYWGQGTLVTVSS | |
| 259 | LVESGGGSVQPGGSLRLSCAASGSIFVLNSMGWYREAPGKERELV | 2H2 VHH2 single |
| | AVSTSGGSTGYADSVKGRFTISRENFKNTVYLQMNSLKPEDTAVY | binder |
| | YCYLETDRRFWGQGTLVTVSS | |
| 260 | LVESGGGSVQAGGSLRLSCAASGRTFSSYAMGWFRLAPGKEREFV | 1H2 VHH2 single |
| | AAISWGGRGTYYADSVKGRFTISRDNAKNTVYLQMNSLKPEDTAV | binder |
| | YYCAAGDLPVVAGTEYEYWGQGTQVTVSS | |
| 261 | VQLVESGGGLVQPGGSLRLSCAASGFTFSTYAMGWFRQAPGKERE | 1C7 VHH2 single |
| | FVAAISWGGRGTYYADFAKGRFTISRDNAKNTVYLQMNSLKPEDT | binder |
| | AAYYCGAGDLPGATVQEEYNYWGQGTQVTVSS | |
| 262 | EVQLVESGGGSVQPGGSLRLSCAGSGSIFPLNVMGWYRQAPGKER | 1B10 VHH2 single |
| | EFIALITSGGSTKHADSVKGRFTISRDNFKNTVYLQMNSLKPEDT | binder |
| | AVYYCYLDQSRQYWGQGTLVTVSS | |
| 263 | QLVESGGCSVQPGESLELSCAASGRIFSLNVMGWYRQVPGKQREL | 1G6 VHH2 single |
| | VARISSGGSTSYADSVKGRFTISRGNARDNALNTVFLQMNSVKPE | binder |
| | DTAVYYCKLEDFTGGYAMDYWGKGTLVTVSS | |
| 264 | EVQLVESVGGSVQAGGSLRLSCAASGRTFSTYAMGWFRQAPGKER | 1E2 VHH2 single |
| | EFVASISWGGRATYYADSVKGRFTISRDNSKNTAYLQMNSLKPED | binder |
| | TAVYYCAAADLPTRVSTAYTFWGQGTQVTVSS | |
| 265 | EVQLVESGGGSVQAGGSLRLSCAASGSIFILNVMGWYRQAPGNQR | 1B12 VHH1 single |
| | ELVALMSSGGSTKYADSVKGRFTISRDNVRNTVYLQMNSLKPEDT | binder |
| | AVYYCTLRTENDYWGQGTQVTVSS | |
| 266 | QLVESGGGSVQAGGSLILSCAASGDIPSIQAMGWYRQAPGKQREL | 1C3 VHH2 single |
| | VAAITSGGRTHYRDSVKGRFTISGSNDKSALYLQMNSLKPEDTAV | binder |
| | YYCAADRGLTAVGEYDYWGQGTQVTVSS | |
| 267 | EVQLVESGGG SVQAGGSLRL SCAASGGTFS SYTMGWFRQA | 21A4 VHH1 single |
| | PGKEREYVGG ISSSGYVMYN SESMKGRFTI SRENAKNMVY | binder |
| | LQMNSLKPED TAVYYCAAGT IGRPYDYWGQ GTQVTVSS | |
| 268 | SYTMG | 21A4 VHH1 |
| | | CDR1 (Kabat |
| | | numbering) |
| 269 | GISSSGYVMY NSESMKG | 21A4 VHH1 |
| | | CDR2 (Kabat |
| | | numbering) |
| 270 | GTIGRPYDY | 21A4 VHH1 |
| | | CDR3 (Kabat |
| | | numbering) |
| 271 | VQLVESGGGL VQPGGSLRLS CAASGSIFRL NVMGWYRQAP | 21A4 VHH2 single |
| | GEQRELVAAI STGGSTKYAD SVKGRFTISR DNVKNTVYLE | binder |
| | MNSLKPEDAA VYFCNVRLVG AYDYWGQGTL VTVSS | |
| 272 | LNVMG | 21A4 VHH2 |
| | | CDR1 (Kabat |
| | | numbering) |
| 273 | AISTGGSTKY ADSVKG | 21A4 VHH2 |
| | | CDR2 (Kabat |
| | | numbering) |
| 274 | RLVGAYDY | 21A4 VHH2 |
| | | CDR3 (Kabat |
| | | numbering) |
| 275 | EVQLVESGGG LVQAGGSLRL SCAASGGTFS SYTMGWFRQA | 21C11 VHH1 |
| | PGKEREYVGG ISSSGYVMYN SESMKGRFTI SRENAKNMVY | single binder |
| | LQMNSLKPED TAVYYCAAGT IGRPYDYWGQ GTQVTVSS | |
| 276 | SYTMG | 21C11 VHH1 |
| | | CDR1 (Kabat |
| | | numbering) |
| 277 | GISSSGYVMY NSESMKG | 21C11 VHH1 |
| | | CDR2 (Kabat |
| | | numbering) |
| 278 | GTIGRPYDY | 21C11 VHH1 |
| | | CDR3 (Kabat |
| | | numbering) |
| 279 | VQLVESGGDL VQAGGSLRLS CAASGRTFST YIIGWFRQAP | 21C11 VHH2 |
| | GKEREFVTAI SWGGRGTYYA DSVKGRFTIS RDNAKNTVYL | single binder |
| | QMNSLRPEDT AVYFCAAGDL MVSTRSQYDY WGQGTLVTVS | |
| | S | |
| 280 | TYIIG | 21C11 VHH2 |
| | | CDR1 (Kabat |
| | | numbering) |
| 281 | AISWGGRGTY YADSVKG | 21C11 VHH2 |
| | | CDR2 (Kabat |
| | | numbering) |
| 282 | GDLMVSTRSQ YDY | 21C11 VHH2 |
| | | CDR3 (Kabat |
| | | numbering) |
| 283 | EVQLVESGGG SVQAGGSLRL SCAASGGTFS SYTMGWFRQA | 21C8 VHH1 single |
| | PGKEREYVGG ISSSGYVMYN SESMKGRFTI SRENAKNMVY | binder |
| | LQMNSLKPED TAVYYCAAGT IGRPYDYWGQ GTLVTVSS | |
| 284 | SYTMG | 21C8 VHH1 |
| | | CDR1 (Kabat |
| | | numbering) |
| 285 | GISSSGYVMY NSESMKG | 21C8 VHH1 |
| | | CDR2 (Kabat |
| | | numbering) |
| 286 | GTIGRPYDY | 21C8 VHH1 |
| | | CDR3 (Kabat |
| | | numbering) |
| 287 | QLVESGGDLV QPGGSLRLSC AASGRTESTY AMGWFRQAPG | 21C8 VHH2 single |
| | KEREFVAAIS WGGRGTYYAD SVRGRFTISR DNPKNTVFLQ | binder |
| | MNSLKPDDTA VYYCAAGDLP GGRRALDYDY WGQGTQVTVS | |
| | S | |
| 288 | TYAMG | 21C8 VHH2 |
| | | CDR1 (Kabat |
| | | numbering) |
| 289 | AISWGGRGTY YADSVRG | 21C8 VHH2 |
| | | CDR2 (Kabat |
| | | numbering) |
| 290 | GDLPGGRRAL DYDY | 21C8 VHH2 |
| | | CDR3 (Kabat |
| | | numbering) |
| 291 | EVQLVESGGG LVQPGGSLSL SCTASGSITS IVAMGWYRQT | 21B11 VHH1 |
| | PGKQRELVAA ITSGGRTHYR DSVKGRFTIS GNNDNSALYL | single binder |
| | HMNSLKPEDT AVYYCAADRG WTSVGEYDYW GQGTLVTVSS | |
| 292 | IVAMG | 21B11 VHH1 |
| | | CDR1 (Kabat |
| | | numbering) |
| 293 | AITSGGRTHY RDSVKG | 21B11 VHH1 |
| | | CDR2 (Kabat |
| | | numbering) |
| 294 | DRGWTSVGEY DY | 21B11 VHH1 |
| | | CDR3 (Kabat |
| | | numbering) |
| 295 | QLVESGGGSV QAGASLRLSC VSSGPTYGFY VTAWFRQAPG | 21B11 VHH2 |
| | KEREFVAAVR GIASRVNYAD SVKGRFTISR DNAANTIFLQ | single binder |
| | MNSLKPEDTA VYYCALRRQY STNYDSSTAY DVWGQGTLVT | |
| | VSS | |
| 296 | FYVTA | 21B11 VHH2 |
| | | CDR1 (Kabat |
| | | numbering) |
| 297 | AVRGIASRVN YADSVKG | 21B11 VHH2 |
| | | CDR2 (Kabat |
| | | numbering) |
| 298 | RRQYSTNYDS STAYDV | 21B11 VHH2 |
| | | CDR3 (Kabat |
| | | numbering) |
| 299 | EVQLVESGGG SVQAGASLRL SCVSSGPTYG FYVTAWFRQA | 21G5 VHH1 single |
| | PGKEREFVAA VRGIASRVNY ADSVKGRFTI SRDNAANTIF | binder |
| | LQMNSLKPED TAVYYCALRR QYSTNYDSST AYDVWGQGTL | |
| | VTVSS | |
| 300 | FYVTA | 21G5 VHH1 |
| | | CDR1 (Kabat |
| | | numbering) |
| 301 | AVRGIASRVN YADSVKG | 21G5 VHH1 |
| | | CDR2 (Kabat |
| | | numbering) |
| 302 | RRQYSTNYDS STAYDV | 21G5 VHH1 |
| | | CDR3 (Kabat |
| | | numbering) |
| 303 | LEESGGGLVQ PGGSLILSCA ASGDIPSIVA MGWYRQAPGK | 21G5 VHH2 single |
| | QRELVAAITS GGRTHYRDSV KGRFTISGNN DNSALYLHMN | binder |
| | SLKPEDTAVY YCAADRGLTA VGEYDYWGQG TQVTVSS | |
| 304 | IVAMG | 21G5 VHH2 |
| | | CDR1 (Kabat |
| | | numbering) |
| 305 | AITSGGRTHY RDSVKG | 21G5 VHH2 |
| | | CDR2 (Kabat |
| | | numbering) |
| 306 | DRGLTAVGEY DY | 21G5 VHH2 |
| | | CDR3 (Kabat |
| | | numbering) |
| 307 | EVQLVESGGG LVQPGGSLRL SCAASGSGFG IGRMGWYRQA | 21G7 VHH1 single |
| | PGKQRELVAI ISSLTGTTYA DSVKGRFTGS RDSAKNMVYL | binder |
| | RMDSLKPEDT AVYYCYANRF TVDYWGQGTL VTVSS | |
| 308 | IGRMG | 21G7 VHH1 |
| | | CDR1 (Kabat |
| | | numbering) |
| 309 | IISSLTGTTY ADSVKG | 21G7 VHH1 |
| | | CDR2 (Kabat |
| | | numbering) |
| 310 | NRFTVDY | 21G7 VHH1 |
| | | CDR3 (Kabat |
| | | numbering) |
| 311 | EVQLVESGGG LVQAGGSLRL SCAASGDIPS IVAMGWYRQA | 21G7 VHH2 single |
| | PGKQRELVAA ITSGGRTHYR DSVKGRFTIS GNNDNSALYL | binder |
| | HMNSLKPEDT AVYYCAADRG LTAVGEYDYW GQGTLVTVSS | |
| 312 | IVAMG | 21G7 VHH2 |
| | | CDR1 (Kabat |
| | | numbering) |
| 313 | AITSGGRTHY RDSVKG | 21G7 VHH2 |
| | | CDR2 (Kabat |
| | | numbering) |
| 314 | DRGLTAVGEY DY | 21G7 VHH2 |
| | | CDR3 (Kabat |
| | | numbering) |
| 315 | EVQLVESGGG LVQAGGSLRL SCAASGRTSS VFSTAWFRRA | 21H2 VHH1 single |
| | PGKEREFVGN IRGIADRTDY ADSVKGRFTI SRDNAKNTVY | binder |
| | LQMNTLKPED TAVYYCAAKR SYTRDYVTDY NYDYWGQGTL | |
| | VTVSS | |
| 316 | VFSTA | 21H2 VHH1 |
| | | CDR1 (Kabat |
| | | numbering) |
| 317 | NIRGIADRTD YADSVKG | 21H2 VHH1 |
| | | CDR2 (Kabat |
| | | numbering) |
| 318 | KRSYTRDYVT DYNYDY | 21H2 VHH1 |
| | | CDR3 (Kabat |
| | | numbering) |
| 319 | LVESGGGLVQ AGGSLILSCA ASGDISSIVA MGWYRQAPGK | 21H2 VHH2 single |
| | QRELVAAITS GGRTHYRDSV KGRFTISGNN DNSALYLHMN | binder |
| | SLKPEDTAVY YCAADRGWTS VGEYDYWGQG TLVTVSS | |
| 320 | IVAMG | 21H2 VHH2 |
| | | CDR1 (Kabat |
| | | numbering) |
| 321 | AITSGGRTHY RDSVKG | 21H2 VHH2 |
| | | CDR2 (Kabat |
| | | numbering) |
| 322 | DRGWTSVGEY DY | 21H2 VHH2 |
| | | CDR3 (Kabat |
| | | numbering) |
| 323 | EVQLVESGGG LVQPGGSLRL SCAASGSGFG IGRMGWYRQA | 19B5 VHH1 single |
| | PGKQRELVAI ISSLSGTTYT DSVKGRFTIS RDIAKNMVYL | binder |
| | RMDSLKPEDT AVYYCYANRF TVDYWGQGTQ VTVSS | |
| 324 | IGRMG | 19B5 VHH1 |
| | | CDR1 (Kabat |
| | | numbering) |
| 325 | IISSLSGTTY TDSVKG | 19B5 VHH1 |
| | | CDR2 (Kabat |
| | | numbering) |
| 326 | NRFTVDY | 19B5 VHH1 |
| | | CDR3 (Kabat |
| | | numbering) |
| 327 | VQLVESVGGL VQAGGSLRLS CAASGDIPSI QAMGWYRQAP | 19B5 VHH2 single |
| | GKQRELVAAI TSGGRTHYRD SVKGRFTISG SNDKSALYLQ | binder |
| | MNSLKPEDTA VYYCAADRGL TAVGEYDYWG QGTLVTVSS | |
| 328 | IQAMG | 19B5 VHH2 |
| | | CDR1 (Kabat |
| | | numbering) |
| 329 | AITSGGRTHY RDSVKG | 19B5 VHH2 |
| | | CDR2 (Kabat |
| | | numbering) |
| 330 | DRGLTAVGEY DY | 19B5 VHH2 |
| | | CDR3 (Kabat |
| | | numbering) |
| 331 | EVQLVESGGG SVQPGGSLRL SCAASGSGFG VGRMGWYRQA | 22D1 VHH1 single |
| | PGNKRELVAI VSSLTGTTYA DSAKGRFTIS RDSAKNMVYL | binder |
| | RMDSLKPEDT AVYYCYANRF TVDYWGQGTL VTVSS | |
| 332 | VGRMG | 22D1 VHH1 |
| | | CDR1 (Kabat |
| | | numbering) |
| 333 | IVSSLTGTTY ADSAKG | 22D1 VHH1 |
| | | CDR2 (Kabat |
| | | numbering) |
| 334 | NRFTVDY | 22D1 VHH1 |
| | | CDR3 (Kabat |
| | | numbering) |
| 335 | EVQLVESGGG LVQPGGSLTL SCAASGDILS IVAMGWYRQA | 22D1 VHH2 single |
| | PGKQRELVAA ITSGGRTHYR DSVKGRFTIS GNNDNSALYL | binder |
| | HMNSLKPEDT AVYYCAADRG LTAVGEYDYW GQGTQVTVSS | |
| 336 | IVAMG | 22D1 VHH2 |
| | | CDR1 (Kabat |
| | | numbering) |
| 337 | AITSGGRTHY RDSVKG | 22D1 VHH2 |
| | | CDR2 (Kabat |
| | | numbering) |
| 338 | DRGLTAVGEY DY | 22D1 VHH2 |
| | | CDR3 (Kabat |
| | | numbering) |
| 339 | EVQLVESGGG SVQAGASLRL SCVSSGPTYG FYVTAWFRQA | 22D8 VHH1 single |
| | PGKEREFVAA VRGIASRVNY ADSVKGRFTI SRDNAANTIF | binder |
| | LQMNSLKPED TAVYYCALRR QYSTNYDSST AYDVWGQGTL | |
| | VTVSS | |
| 340 | FYVTA | 22D8 VHH1 |
| | | CDR1 (Kabat |
| | | numbering) |
| 341 | AVRGIASRVN YADSVKG | 22D8 VHH1 |
| | | CDR2 (Kabat |
| | | numbering) |
| 342 | RRQYSTNYDS STAYDV | 22D8 VHH1 |
| | | CDR3 (Kabat |
| | | numbering) |
| 343 | LVESGGGLVQ AGGSLILSCA ASGDIPSIVA MGWYRQAPGK | 22D8 VHH2 single |
| | QRELVAAITS GGRTHYRDSV KGRFTISGNN DNSALYLHMN | binder |
| | SLKPEDTAVY YCAADRGLTA VGEYDYWGQG TLVTVSS | |
| 344 | IVAMG | 22D8 VHH2 |
| | | CDR1 (Kabat |
| | | numbering) |
| 345 | AITSGGRTHY RDSVKG | 22D8 VHH2 |
| | | CDR2 (Kabat |
| | | numbering) |
| 346 | DRGLTAVGEY DY | 22D8 VHH2 |
| | | CDR3 (Kabat |
| | | numbering) |

### DESCRIPTION

### I. Definitions

The section headings used herein are for organizational purposes only and are not to be construed as limiting the subject matter described.

The complete disclosures of all publications cited herein are incorporated herein by reference in their entireties as if each were individually set forth in full herein and incorporated.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an antibody heavy chain is that of the EU index as in Kabat et al., Sequences of Proteins of Immunological Interest, 5th Ed. Public Health Service, National Institutes of Health, Bethesda, Md. (1991). Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The terms “multimer” and “multimeric” refer to a complex of two or more component molecules, such as two or more polypeptides. A multimer includes, but is not limited to, a dimer or a trimer. Within a multimer, the two or more component molecules may be the same (i.e., the multimer is a homomultimer), or one or more of the two or more component molecules may be different from the other component molecules (i.e., the multimer is a heteromultimer).

The terms “IL31,” and “interleukin-31” as used herein refer to any native, mature IL31 that results from processing of an IL31 precursor in a cell. The term includes IL31 from any vertebrate source, including mammals such as primates (e.g., humans and cynomolgus or rhesus monkeys) and rodents (e.g., mice and rats), unless otherwise indicated. The term also includes naturally occurring variants of IL31, such as splice variants or allelic variants. Nonlimiting exemplary human IL31 amino acid sequences are shown in Table 1 (see SEQ ID NOs: 1 and 2; signal sequence is underlined). See also UniProtKB/Swiss-Prot Accession: Q6EBC2.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to a IL31 epitope is a sdAb or VHH-containing polypeptide that binds this epitope with greater affinity, avidity, more readily, and/or with greater duration than it binds to other IL31 epitopes or non-IL31 epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some aspects, by “reduce” or “inhibit” is meant the ability to cause an overall decrease of 10% or greater. In some aspects, by “reduce” or “inhibit” is meant the ability to cause an overall decrease of 50% or greater. In some aspects, by “reduce” or “inhibit” is meant the ability to cause an overall decrease of 75%, 85%, 90%, 95%, or greater. In some aspects, the amount noted above is inhibited or decreased over a period of time, relative to a control over the same period of time.

The term “antibody” is used in the broadest sense and encompasses various polypeptides that comprise antibody-like antigen-binding domains, including but not limited to conventional antibodies (typically comprising at least one heavy chain and at least one light chain), single-domain antibodies (sdAbs, comprising at least one variable heavy domain of heavy chain or “VHH” domain and an Fc region), VHH-containing polypeptides (polypeptides comprising at least one VHH domain), and fragments or multimers of any of the foregoing so long as they exhibit the desired antigen-binding activity. In some aspects, an antibody comprises a dimerization domain. Such dimerization domains include, but are not limited to, heavy chain constant domains (comprising CH1, hinge, CH2, and CH3 domains, where CH1 typically pairs with a light chain constant domain, CL, and where the hinge mediates dimerization) and Fc regions (comprising hinge, CH2, and CH3, where the hinge mediates dimerization).

As used herein, the term “binder” refers to a molecule or a portion of a molecule which binds a specific target molecule. A binder can comprise a protein, peptide, nucleic acid, carbohydrate, lipid, or small molecular weight compound. In some aspects, a binder comprises an antibody. In some aspects, a binder comprises an antigen-binding domain of an antibody. In some aspects, a binder comprises an antibody or an antigen-binding domain. In some aspects, a binder comprises a heavy chain variable region of an antibody. In some aspects, a binder comprises a light chain variable region of an antibody. In some aspects, a binder comprises a variable region of an antibody. In some aspects, a binder comprises an antibody mimetic. In some aspects, a binder comprises a small molecular weight component. In some aspects, a binding molecule (e.g., a polypeptide) has only one binder. In some aspects, a binding molecule (e.g., a polypeptide) has two binding moieties. In some aspects, a binding molecule (e.g., a polypeptide) has three or more binding moieties. In some aspects, the two or more binding moieties on one binding molecule (e.g., a polypeptide) are the same. In some aspects, the two or more binding moieties on one binding molecule (e.g., a polypeptide) are different. For example, a binding molecule (e.g., a polypeptide) can have two binding moieties, both being antigen binding domains, such as VHHs. For another example, a binding molecule (e.g., a polypeptide) can also have two binding moieties, one being a variable heavy domain of heavy chain “VHH”, and the other being scFv.

The term “antigen-binding domain” as used herein refers to a portion of an antibody sufficient to bind an antigen. In some aspects, an antigen binding domain of a conventional antibody comprises three heavy chain CDRs and three light chain CDRs. Thus, in some aspects, an antigen binding domain comprises a heavy chain variable region comprising CDR1-FR2-CDR2-FR3-CDR3, and any portions of FR1 and/or FR4 required to maintain binding to antigen, and a light chain variable region comprising CDR1-FR2-CDR2-FR3-CDR3, and any portions of FR1 and/or FR4 required to maintain binding to antigen. In some aspects, an antigen-binding domain of an sdAb or VHH-containing polypeptide comprises three CDRs of a VHH domain (e.g., CDR1, CDR2, and CDR3). Thus, in some aspects, an antigen binding domain of an sdAb or VHH-containing polypeptide comprises a VHH domain comprising CDR1-FR2-CDR2-FR3-CDR3, and any portions of FR1 and/or FR4 required to maintain binding to antigen.

The term “VHH” or “VHH domain” or “VHH antigen-binding domain” as used herein refers to the Variable Heavy domain of a Heavy chain, or the antigen-binding portion of a single-domain antibody, such as a camelid antibody or shark antibody. In some aspects, a VHH comprises three CDRs and four framework regions, designated FR1, CDR1, FR2, CDR2, FR3, CDR3, and FR4. In some aspects, a VHH may be truncated at the N-terminus or C-terminus such that it comprises only a partial FR1 and/or FR4, or lacks one or both of those framework regions, so long as the VHH substantially maintains antigen binding and specificity.

The terms “single domain antibody” and “sdAb” are used interchangeably herein to refer to an antibody comprising at least one monomeric domain, such as a VHH domain, without a light chain, and an Fc region. Single domain antibodies may be derived, for example, from a Camelid species, such as llamas, alpacas and camels. In some aspects, an sdAb is a dimer of two polypeptides wherein each polypeptide comprises at least one VHH domain and an Fc region. As used herein, the terms “single domain antibody” and “sdAb” encompass polypeptides that comprise multiple VHH domains, such as a polypeptide having the structure VHH1-VHH2-Fc or VHH1-linker-VHH2-Fc, wherein VHH1 and VHH2 may be the same or different.

The term “VHH-containing polypeptide” refers to a polypeptide that comprises at least one VHH domain. In some aspects, a VHH polypeptide comprises two, three, or four or more VHH domains, wherein each VHH domain may be the same or different. In some aspects, a VHH-containing polypeptide comprises an Fc region. In some such aspects, the VHH-containing polypeptide may be referred to as an sdAb. Further, in some such aspects, the VHH polypeptide may form a dimer. Nonlimiting structures of VHH-containing polypeptides, which are also sdAbs, include VHH1-VHH2-Fc, wherein VHH1 and VHH2 may be the same or different. In some aspects of such structures, one VHH may be connected to another VHH by a linker, or one VHH may be connected to the Fc by a linker. In some such aspects, the linker comprises 5-50 amino acids. In some such aspects, the linker comprises 8 to 40 amino acids. In some aspects, the linker comprises 12 to 37 amino acids. In some aspects, the linker is composed of glycine and serine. In some aspects, the linker is composed of glycine, serine, and other amino acids. Some examples of polypeptide linkers are described in WO2022147463, which is incorporated herein by reference in its entirety. In some aspects, when a VHH-containing polypeptide comprises an Fc, it forms a dimer. Thus, the structure VHH1-VHH2-Fc, if it forms a dimer, is considered to be tetravalent (i.e., the dimer has four VHH domains).

The term “single binder” refers to a single domain binder, e.g., VHH, capable of specifically binding a target antigen. A general structure, for example, of a single binder is as follows: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively.

The term “co-binder” refers to two single domain binding moieties, e.g., VHHs, joined together by an optional linker peptide in which each single binder is capable of simultaneously binding the same target antigen, e.g., IL-31, as the other single binder. A co-binder may comprise two biparatopic single binders. For example, each single binder is capable of specifically binding to the same target antigen (e.g., bind at a non-overlapping epitope) allowing each single binder to simultaneously bind the target. A general structure, for example, of a co-binder is as follows: 1st Single Binder-Linker-2nd Single Binder, wherein the N-terminal region of the 2nd single binder may be truncated, see WO 2022/147463. In some aspects, a IL31 co-binder is capable of binding to IL31 at a binding affinity at least about 2 folds, 3 folds, 4 folds, 5 folds, 6 folds, 7 folds, 8 folds, 9 folds, 10 folds, 11 folds, 12 folds, 13 folds, 14 folds, 15 folds 16 folds, 17 folds, 18 folds, 19 folds, or 20 folds stronger than each of the first IL31 and the second IL31 binders.

The term “biparatopic” refers to two or more antibodies, two or more “single binders”, one or more “co-binder(s)” that recognize and bind two different epitopes of the same target antigen. For example, each single binder, such as a VHH, of a co-binder binds to the same target antigen at nonoverlapping epitopes, which allows both single binders to bind the same target antigen simultaneously.

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts.

The term “CDR” denotes a complementarity determining region as defined by at least one manner of identification to one of skill in the art. In some aspects, CDRs can be defined in accordance with any of the IMGT numbering scheme, the Chothia numbering scheme, the Kabat numbering scheme, a combination of Kabat and Chothia, the AbM definition, and/or the contact definition. A VHH comprises three CDRs, designated CDR1, CDR2, and CDR3.

The term “heavy chain constant region” as used herein refers to a region comprising at least three heavy chain constant domains, CH1, hinge, CH2, and CH3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an a constant region is an IgA antibody. Further, an antibody comprising a u constant region is an IgM antibody, and an antibody comprising an ε constant region is an IgE antibody. Certain isotypes can be further subdivided into subclasses. For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgAQ1 (comprising an al constant region) and IgA2 (comprising an a2 constant region) antibodies; and IgM antibodies include, but are not limited to, IgM1 and IgM2.

A “Fc region” as used herein refers to a portion of a heavy chain constant region comprising CH2 and CH3. In some aspects, an Fc region comprises a hinge, CH2, and CH3, i.e., no CH1. In various aspects, when an Fc region comprises a hinge, wherein the hinge mediates dimerization between two Fc-containing polypeptides. In some aspects, the hinge region comprises a cysteine (C) to serine(S) mutation at position 220 as determined by EU numbering. An Fc region may be of any antibody heavy chain constant region isotype discussed herein. In some aspects, an Fc region is an IgG1, IgG2, IgG3, or IgG4.

An “acceptor human framework” as used herein is a framework comprising the amino acid sequence of a heavy chain variable domain (VH) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some aspects, the number of amino acid changes are fewer than 10, or fewer than 9, or fewer than 8, or fewer than 7, or fewer than 6, or fewer than 5, or fewer than 4, or fewer than 3, across all the human frameworks in a single antigen binding domain, such as a VHH.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to remove any potential liabilities (e.g., immunogenicity or safety concerns in administering the polypeptide to a subject, or polypeptide stability), or further refine and optimize sdAb VHH-containing polypeptide performance. In some aspects, a humanized sdAb or VHH-containing polypeptide comprises a human Fc region. As will be appreciated, a humanized sequence can be identified by its primary sequence and does not necessarily denote the process by which the antibody was created.

The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some aspects, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

“Affinity” refers to the strength of the sum total of noncovalent interactions between a single binding site of a molecule (for example, an antibody, such as an sdAb, or VHH-containing polypeptide) and its binding partner (for example, an antigen). The affinity or the apparent affinity of a molecule X for its partner Y can generally be represented by the dissociation constant (kD) or the kD-apparent, respectively. Affinity can be measured by common methods known in the art (such as, for example, ELISA KD, KinExA, flow cytometry, and/or surface plasmon resonance (SPR) devices), including those described herein. Such methods include, but are not limited to, methods involving BIAcore®, Octet®, or flow cytometry.

The terms “kD,” “K_{D},” “K_{d},” “Kd” or “Kd value” as used interchangeably to refer to the equilibrium dissociation constant of an antigen-binding molecule (e.g., an antibody or VHH) and antigen interaction. When the term “kD” is used herein, it includes kD and kD-apparent. The equilibrium dissociation constant (K_{d}) is calculated as the ratio of k_{off}/kₒₙ. The term “kon” refers to the rate constant for association of an antibody to an antigen and the term “koff′ refers to the rate constant for dissociation of an antibody from the antibody/antigen complex.

The term “binds” to an antigen or epitope is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA KD, KinEx A, surface plasmon resonance devices, or biolayer interferometry (BLI).

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51:19-26.

An “effector-positive Fc region” possesses an effector function of a native sequence Fc region.

An “effector null Fc region” lacks one or more effector function(s) of a native sequence Fc region.

Exemplary “effector functions” include Fc receptor binding; C1q binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “variant Fc region” comprises an amino acid sequence which differs from that of a native sequence Fc region by virtue of at least one amino acid modification. In some aspects, a “variant Fc region” comprises an amino acid sequence which differs from that of a native sequence Fc region by virtue of at least one amino acid modification, yet retains at least one effector function of the native sequence Fc region. In some aspects, a “variant Fc region” comprises an amino acid sequence which differs from that of a native sequence Fc region by virtue of at least one amino acid modification, and the variant Fc region has a modified effector function. In some aspects, a “variant Fc region” comprises an amino acid sequence which differs from that of a native sequence Fc region by virtue of at least one amino acid modification, and the variant Fc region is an effector null Fc region. In some aspects, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some aspects, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 2. Amino acid substitutions may be introduced into a polypeptide of interest and the products screened for a desired activity, for example, retained/improved antigen binding, decreased immunogenicity, or improved ADCC or CDC.

TABLE 2
Amino acid residues

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, IIe;
(2)neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) as provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example, a mammal. In some aspects, methods of treating mammals, including, but not limited to, humans, rodents, and simians, are provided. In some examples, an “individual” or “subject” refers to an individual or subject in need of treatment for a disease or disorder. In some aspects, the subject to receive the treatment can be a patient, designating the fact that the subject has been identified as having a disease or disorder of relevance to the treatment, or being at adequate risk of contracting the disease or disorder.

A “disease” or “disorder” as used herein refers to a condition where treatment is needed and/or desired.

As used herein, “IL31-associated condition” refers to any condition, disorder, or disease that involves aberrant expression, activity, or function of the IL31 protein within a biological context, such as in vivo in a subject. For example, an IL31-associated condition may involve increased IL31 expression, excessive IL31 activity, and/or any IL31 dysfunction that results in a condition where treatment is needed and/or desired.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” are used interchangeably and refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

### II. Exemplary IL31 Binding Polypeptides

Provided herein are polypeptides that bind IL31. In some aspects, the polypeptide that binds IL31 comprises one or more IL31 binders. In some aspects, the one or more IL31 binder comprises a VHH1. In some aspects, the two or more IL31 binders comprise a VHH1 and a VHH2. In some aspects, the polypeptide comprising a first IL31 binder (e.g., VHH1) and a second IL31 binder (e.g., VHH2) are referred to as a IL31 co-binder. Therefore, provided herein are various single binders (e.g., VHH1 or VHH2 disclosed herein) or co-binders (VHH1 and VHH2 in combination, optionally linked by a linker). Also provided herein is a polypeptide comprising a single IL31 binder (e.g., any VHH1 or VHH2 disclosed herein) and another binder that binds to IL31, but is not disclosed herein. In some aspects, provided herein is a polypeptide comprising a single IL31 binder (e.g., any VHH1 or VHH2 disclosed herein) and another binder that binds to an antigen other than IL31.

In some aspects, the VHH1 comprises a CDR1 comprising the amino acid sequence of SEQ ID NO:59; a CDR2 comprising the amino acid sequence of SEQ ID NO:63; and a CDR3 comprising the amino acid sequence of SEQ ID NO: 64. In some aspects, the VHH1 comprises a CDR1 comprising the amino acid sequence of SEQ ID NO: 59; a CDR2 comprising the amino acid sequence of SEQ ID NO: 63; and a CDR3 comprising the amino acid sequence of SEQ ID NO:64. In some aspects, the IL31 binder comprises a VHH comprising a CDR1 comprising the amino acid sequence SYX₁MX₂, wherein X₁ is A or T, and X₂ is A or G (SEQ ID NO:59); a CDR2 comprising the amino acid sequence GISSSGYX₁₀X₁₁YX₁₂X₁₃X₁₄SMKG, wherein X₁₀ is T or V, X₁₁ is L or M, X₁₂ is K or N, X₁₃ is S or A, and X₁₄ is D or E (SEQ ID NO:63); and a CDR3 comprising the amino acid sequence GX₁₅X₁₆GRPYX₁₇X₁₈, wherein X₁₅ is I, T, or V, X₁₆ is F or I, and X₁₇ is E or D, and X₁₈ is Y or F (SEQ ID NO:64).

In some aspects, the VHH1 comprises a CDR1 comprising the amino acid sequence of SEQ ID NO:59; a CDR2 comprising the amino acid sequence of SEQ ID NO:63; and a CDR3 comprising the amino acid sequence of SEQ ID NO:61. In some aspects, the VHH1 comprises a CDR1 comprising the amino acid sequence of SEQ ID NO:59; a CDR2 comprising the amino acid sequence of SEQ ID NO:63; and a CDR3 comprising the amino acid sequence of SEQ ID NO:61. In some aspects, the IL31 binder comprises a VHH comprising a CDR1 comprising the amino acid sequence SYX₁MX₂, wherein X₁ is A or T, and X₂ is A or G (SEQ ID NO:59); a CDR2 comprising the amino acid sequence GISSSGYX₁₀X₁₁YX₁₂X₁₃X₁₄SMKG, wherein X₁₀ is T or V, X₁₁ is L or M, X₁₂ is K or N, X₁₃ is S or A, and X₁₄ is D or E (SEQ ID NO:63); and a CDR3 comprising the amino acid sequence GX₇X₈GRPYX,Y, wherein X₇ is I or T, X₈ is F or I, and X₉ is E or D (SEQ ID NO: 61).

In some aspects, the VHH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61. In some aspects, the VHH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:61. In some aspects, the IL31 binder comprises a VHH comprising a CDR1 comprising the amino acid sequence SYX₁MX₂, wherein X₁ is A or T, and X₂ is A or G (SEQ ID NO:59); a CDR2 comprising the amino acid sequence GISSSGYX₃X₄YX₅SX₆SMKG, wherein X₃ is T or V, X₄ is L or M, X₅ is K or N, and X₆ is D or E (SEQ ID NO:60); and a CDR3 comprising the amino acid sequence GX₇X₈GRPYX₉Y, wherein X₇ is I or T, X₈ is F or I, and X₉ is E or D (SEQ ID NO:61).

In some aspects, the VHH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:64. In some aspects, the VHH1 comprises a CDR1 comprising the amino acid sequence of SEQ ID NO:59; a CDR2 comprising the amino acid sequence of SEQ ID NO:60; and a CDR3 comprising the amino acid sequence of SEQ ID NO:64. In some aspects, the IL31 binder comprises a VHH comprising a CDR1 comprising the amino acid sequence SYX₁MX₂, wherein X₁ is A or T, and X₂ is A or G (SEQ ID NO: 59); a CDR2 comprising the amino acid sequence GISSSGYX₃X₄YX₅SX₆SMKG, wherein X₃ is T or V, X₄ is L or M, X₅ is K or N, and X₆ is D or E (SEQ ID NO:60); and a CDR3 comprising the amino acid sequence GX₁₅X₁₆GRPYX₁₇X₁₈ wherein X₁₅ is I, T, or V, X₁₆ is F or I, and X₁₇ is E or D, and X₁₈ is Y or F (SEQ ID NO:64).

In some aspects, the polypeptide that binds IL31 comprises a first IL31 binder and a second IL31 binder. In some aspects, the first IL31 binder comprises a VHH1 comprising a CDR1 comprising the amino acid sequence of SEQ ID NO: 5, SEQ ID NO: 19, SEQ ID NO: 33, SEQ ID NO: 47, SEQ ID NO: 78, SEQ ID NO: 81, or SEQ ID NO: 59; a CDR2 comprising the amino acid sequence of SEQ ID NO: 6, SEQ ID NO: 20, SEQ ID NO: 34, SEQ ID NO: 48, SEQ ID NO: 79, SEQ ID NO: 82, SEQ ID NO: 60, or SEQ ID NO:63; and a CDR3 comprising the amino acid sequence of SEQ ID NO: 7, SEQ ID NO: 21, SEQ ID NO: 35, SEQ ID NO: 49, SEQ ID NO: 80, SEQ ID NO: 83, SEQ ID NO: 61 or SEQ ID NO:64, wherein one or more of the CDR1, CDR2, and CDR3 comprises 1, 2, 3, 4, or 5 amino acid substitutions, insertions or deletions. In some aspects, the first IL31 binder comprises a VHH1 comprising a CDR1 comprising the amino acid sequence of SEQ ID NO: 5, SEQ ID NO: 19, SEQ ID NO: 33, SEQ ID NO: 47, SEQ ID NO: 78, SEQ ID NO: 81, or SEQ ID NO: 59; a CDR2 comprising the amino acid sequence of SEQ ID NO: 6, SEQ ID NO: 20, SEQ ID NO: 34, SEQ ID NO: 48, SEQ ID NO: 79, SEQ ID NO: 82, SEQ ID NO: 60, or SEQ ID NO:63; and a CDR3 comprising the amino acid sequence of SEQ ID NO: 7, SEQ ID NO: 21, SEQ ID NO: 35, SEQ ID NO: 49, SEQ ID NO: 80, SEQ ID NO: 83, SEQ ID NO: 61, or SEQ ID NO:64. In some aspects, the second IL31 binder comprises a VHH2 comprising a CDR1 comprising the amino acid sequence of SEQ ID NO: 10, SEQ ID NO: 24, SEQ ID NO: 38, SEQ ID NO: 52, SEQ ID NO: 84, SEQ ID NO: 87, or SEQ ID NO:59; a CDR2 comprising the amino acid sequence of SEQ ID NO: 11, SEQ ID NO: 25, SEQ ID NO: 39, SEQ ID NO: 53, SEQ ID NO: 85, SEQ ID NO: 88, SEQ ID NO:60, or SEQ ID NO:63; and a CDR3 comprising the amino acid sequence of SEQ ID NO: 12, SEQ ID NO: 26, SEQ ID NO: 40, SEQ ID NO: 54, SEQ ID NO: 86, SEQ ID NO: 89, SEQ ID NO: 61, or SEQ ID NO:64, wherein one or more of the CDR1, CDR2, and CDR3 comprises 1, 2, 3, 4, or 5 amino acid substitutions, insertions or deletions. In some aspects, the second IL31 binder comprises a VHH2 comprising a CDR1 comprising the amino acid sequence of SEQ ID NO: 10, SEQ ID NO: 24, SEQ ID NO: 38, SEQ ID NO: 52, SEQ ID NO: 84, SEQ ID NO: 87, or SEQ ID NO:59; a CDR2 comprising the amino acid sequence of SEQ ID NO: 11, SEQ ID NO: 25, SEQ ID NO: 39, SEQ ID NO: 53, SEQ ID NO: 85, SEQ ID NO: 88, SEQ ID NO: 60, or SEQ ID NO:63; and a CDR3 comprising the amino acid sequence of SEQ ID NO: 12, SEQ ID NO: 26, SEQ ID NO: 40, SEQ ID NO: 54, SEQ ID NO: 86, SEQ ID NO: 89, SEQ ID NO: 61, or SEQ ID NO:64.

In some aspects, the VHH1 comprises a CDR1 comprising the amino acid sequence of SEQ ID NO: 5, a CDR2 comprising the amino acid sequence of SEQ ID NO: 6, and a CDR3 comprising the amino acid sequence of SEQ ID NO: 7, wherein one or more of the CDR1, CDR2, and CDR3 comprises 1, 2, 3, 4, or 5 amino acid substitutions, insertions or deletions. In some aspects, the VHH1 comprises a CDR1 comprising the amino acid sequence of SEQ ID NO: 5, a CDR2 comprising the amino acid sequence of SEQ ID NO: 6, and a CDR3 comprising the amino acid sequence of SEQ ID NO: 7. In some aspects, the VHH1 comprises a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21, wherein one or more of the CDR1, CDR2, and CDR3 comprises 1, 2, 3, 4, or 5 amino acid substitutions, insertions or deletions. In some aspects, the VHH1 comprises a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21. In some aspects, the VHH1 comprises a CDR1 comprising the amino acid sequence of SEQ ID NO: 33, a CDR2 comprising the amino acid sequence of SEQ ID NO: 34, and a CDR3 comprising the amino acid sequence of SEQ ID NO: 35, wherein one or more of the CDR1, CDR2, and CDR3 comprises 1, 2, 3, 4, or 5 amino acid substitutions, insertions or deletions. In some aspects, the VHH1 comprises a CDR1 comprising the amino acid sequence of SEQ ID NO: 33, a CDR2 comprising the amino acid sequence of SEQ ID NO: 34, and a CDR3 comprising the amino acid sequence of SEQ ID NO: 35. In some aspects, the VHH1 comprises a CDR1 comprising the amino acid sequence of SEQ ID NO: 47, a CDR2 comprising the amino acid sequence of SEQ ID NO: 48, and a CDR3 comprising the amino acid sequence of SEQ ID NO: 49, wherein one or more of the CDR1, CDR2, and CDR3 comprises 1, 2, 3, 4, or 5 amino acid substitutions, insertions or deletions. In some aspects, the VHH1 comprises a CDR1 comprising the amino acid sequence of SEQ ID NO: 47, a CDR2 comprising the amino acid sequence of SEQ ID NO: 48, and a CDR3 comprising the amino acid sequence of SEQ ID NO: 49. In some aspects, the VHH1 comprises a CDR1 comprising the amino acid sequence of SEQ ID NO: 78, a CDR2 comprising the amino acid sequence of SEQ ID NO: 79, and a CDR3 comprising the amino acid sequence of SEQ ID NO: 80, wherein one or more of the CDR1, CDR2, and CDR3 comprises 1, 2, 3, 4, or 5 amino acid substitutions, insertions or deletions. In some aspects, the VHH1 comprises a CDR1 comprising the amino acid sequence of SEQ ID NO: 78, a CDR2 comprising the amino acid sequence of SEQ ID NO: 79, and a CDR3 comprising the amino acid sequence of SEQ ID NO: 80. In some aspects, the VHH1 comprises a CDR1 comprising the amino acid sequence of SEQ ID NO: 81, a CDR2 comprising the amino acid sequence of SEQ ID NO: 82, and a CDR3 comprising the amino acid sequence of SEQ ID NO: 83, wherein one or more of the CDR1, CDR2, and CDR3 comprises 1, 2, 3, 4, or 5 amino acid substitutions, insertions or deletions. In some aspects, the VHH1 comprises a CDR1 comprising the amino acid sequence of SEQ ID NO: 81, a CDR2 comprising the amino acid sequence of SEQ ID NO: 82, and a CDR3 comprising the amino acid sequence of SEQ ID NO: 83. In some aspects, the VHH1 comprises a CDR1 comprising the amino acid sequence of SEQ ID NO: 59, a CDR2 comprising the amino acid sequence of SEQ ID NO: 60 or SEQ ID NO:63, and a CDR3 comprising the amino acid sequence of SEQ ID NO: 61 or SEQ ID NO:64, wherein one or more of the CDR1, CDR2, and CDR3 comprises 1, 2, 3, 4, or 5 amino acid substitutions, insertions or deletions. In some aspects, the VHH1 comprises a CDR1 comprising the amino acid sequence of SEQ ID NO: 59, a CDR2 comprising the amino acid sequence of SEQ ID NO: 60 or SEQ ID NO: 63, and a CDR3 comprising the amino acid sequence of SEQ ID NO: 61 or SEQ ID NO:64. In some aspects, the first IL31 binder comprises a VHH1 comprising a CDR1 comprising the amino acid sequence SYX₁MX₂, wherein X₁ is A or T, and X₂ is A or G (SEQ ID NO: 59); a CDR2 comprising the amino acid sequence GISSSGYX₃X₄YX₅SX₆SMKG, wherein X₃ is T or V, X₄ is L or M, X₅ is K or N, and X₆ is D or E (SEQ ID NO: 60), or GISSSGYX₁₀X₁₁YX₁₂X₁₃X₁₄SMKG, wherein X₁₀ is T or V, X₁₁ is L or M, X₁₂ is K or N, X₁₃ is S or A, and X₁₄ is D or E (SEQ ID NO:63); and a CDR3 comprising the amino acid sequence GX₇X₈GRPYX,Y, wherein X₇ is I or T, and X₈ is F or I, and X₉ is E or D (SEQ ID NO: 61), or GX₁₅X₁₆GRPYX₁₇X₁₈, wherein X₁₅ is I, T, or V, X₁₆ is F or I, and X₁₇ is E or D, and X₁₈ is Y or F.

In some aspects, the VHH2 comprises a CDR1 comprising the amino acid sequence of IX₂₃AMG, X₂₃ is V or H (SEQ ID NO: 251), a CDR2 comprising the amino acid sequence of AITSGGRX₂₄HYRDSVKG, X₂₄ is T or R (SEQ ID NO: 252), and a CDR3 comprising the amino acid sequence of DX₂₅GX₂₆X₂₇X₂₈VGEYDY, X₂₅ is R, M, or T, X₂₆ is W, L, or R, X₂₇ is T or V, and X₂₈ is S or A (SEQ ID NO: 253).

In some aspects, the VHH2 comprises a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12, wherein one or more of the CDR1, CDR2, and CDR3 comprises 1, 2, 3, 4, or 5 amino acid substitutions, insertions or deletions. In some aspects, the VHH2 comprises a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12. In some aspects, the VHH2 comprises a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26, wherein one or more of the CDR1, CDR2, and CDR3 comprises 1, 2, 3, 4, or 5 amino acid substitutions, insertions or deletions. In some aspects, the VHH2 comprises a CDR1 comprising the amino acid sequence of SEQ ID NO: 24, a CDR2 comprising the amino acid sequence of SEQ ID NO:25, and a CDR3 comprising the amino acid sequence of SEQ ID NO: 26. In some aspects, the VHH2 comprises a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40, wherein one or more of the CDR1, CDR2, and CDR3 comprises 1, 2, 3, 4, or 5 amino acid substitutions, insertions or deletions. In some aspects, the VHH2 comprises a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40. In some aspects, the VHH2 comprises a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54, wherein one or more of the CDR1, CDR2, and CDR3 comprises 1, 2, 3, 4, or 5 amino acid substitutions, insertions or deletions. In some aspects, the VHH2 comprises a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54. In some aspects, the VHH2 comprises a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86, wherein one or more of the CDR1, CDR2, and CDR3 comprises 1, 2, 3, 4, or 5 amino acid substitutions, insertions or deletions. In some aspects, the VHH2 comprises a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86. In some aspects, the VHH2 comprises a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89, wherein one or more of the CDR1, CDR2, and CDR3 comprises 1, 2, 3, 4, or 5 amino acid substitutions, insertions or deletions. In some aspects, the VHH2 comprises a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89.

In some aspects, (i) the VHH1 comprises a CDR1 comprising the amino acid sequence of SEQ ID NO: 59, a CDR2 comprising the amino acid sequence of SEQ ID NO: 60 or SEQ ID NO: 63, and a CDR3 comprising the amino acid sequence of SEQ ID NO: 61 or SEQ ID NO:64; and (ii) the VHH2 comprises a CDR1 comprising the amino acid sequence of SEQ ID NO: 10, SEQ ID NO: 24, SEQ ID NO: 38, SEQ ID NO: 52, SEQ ID NO: 84, SEQ ID NO: 87 or SEQ ID NO: 251, a CDR2 comprising the amino acid sequence of SEQ ID NO: 11, SEQ ID NO: 25, SEQ ID NO: 39, SEQ ID NO: 53, SEQ ID NO: 85, SEQ ID NO: 88, or SEQ ID NO:252, and a CDR3 comprising the amino acid sequence of SEQ ID NO: 12, SEQ ID NO: 26, SEQ ID NO: 40, SEQ ID NO: 54, SEQ ID NO: 86, SEQ ID NO: 89, or SEQ ID NO:253.

In some aspects, (i) the VHH1 comprises a CDR1 comprising the amino acid sequence of SEQ ID NO: 5, a CDR2 comprising the amino acid sequence of SEQ ID NO: 6, and a CDR3 comprising the amino acid sequence of SEQ ID NO: 7; and (ii) the VHH2 comprises a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12. In some aspects, (i) the VHH1 comprises a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21; and (ii) the VHH2 comprises a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26. In some aspects, (i) the VHH1 comprises a CDR1 comprising the amino acid sequence of SEQ ID NO: 33, a CDR2 comprising the amino acid sequence of SEQ ID NO: 34, and a CDR3 comprising the amino acid sequence of SEQ ID NO: 35; and (ii) the VHH2 comprises a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40. In some aspects, (i) the VHH1 comprises a CDR1 comprising the amino acid sequence of SEQ ID NO: 47, a CDR2 comprising the amino acid sequence of SEQ ID NO: 48, and a CDR3 comprising the amino acid sequence of SEQ ID NO: 49; and (ii) the VHH2 comprises a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54. In some aspects, (i) the VHH1 comprises a CDR1 comprising the amino acid sequence of SEQ ID NO: 78, a CDR2 comprising the amino acid sequence of SEQ ID NO: 79, and a CDR3 comprising the amino acid sequence of SEQ ID NO: 80; and (ii) the VHH2 comprises a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86. In some aspects, (i) the VHH1 comprises a CDR1 comprising the amino acid sequence of SEQ ID NO: 81, a CDR2 comprising the amino acid sequence of SEQ ID NO: 82, and a CDR3 comprising the amino acid sequence of SEQ ID NO: 83; and (ii) the VHH2 comprises a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89. In some aspects, one or more of the VHH1 and the VHH2 are VHH domains. In some aspects, the VHH1 and the VHH2 are VHH domains.

In some aspects, (i) the VHH1 comprises a CDR1 comprising the amino acid sequence of SEQ ID NO:268, a CDR2 comprising the amino acid sequence of SEQ ID NO:269, and a CDR3 comprising the amino acid sequence of SEQ ID NO:270; and (ii) the VHH2 comprises a CDR1 comprising the amino acid sequence of SEQ ID NO:272, a CDR2 comprising the amino acid sequence of SEQ ID NO:273, and a CDR3 comprising the amino acid sequence of SEQ ID NO: 275. In some aspects, (i) the VHH1 comprises a CDR1 comprising the amino acid sequence of SEQ ID NO:276, a CDR2 comprising the amino acid sequence of SEQ ID NO:277, and a CDR3 comprising the amino acid sequence of SEQ ID NO:278; and (ii) the VHH2 comprises a CDR1 comprising the amino acid sequence of SEQ ID NO:280, a CDR2 comprising the amino acid sequence of SEQ ID NO:281, and a CDR3 comprising the amino acid sequence of SEQ ID NO: 282. In some aspects, (i) the VHH1 comprises a CDR1 comprising the amino acid sequence of SEQ ID NO:284, a CDR2 comprising the amino acid sequence of SEQ ID NO:285, and a CDR3 comprising the amino acid sequence of SEQ ID NO:286; and (ii) the VHH2 comprises a CDR1 comprising the amino acid sequence of SEQ ID NO:288, a CDR2 comprising the amino acid sequence of SEQ ID NO:289, and a CDR3 comprising the amino acid sequence of SEQ ID NO: 290. In some aspects, (i) the VHH1 comprises a CDR1 comprising the amino acid sequence of SEQ ID NO:292, a CDR2 comprising the amino acid sequence of SEQ ID NO:293, and a CDR3 comprising the amino acid sequence of SEQ ID NO:294; and (ii) the VHH2 comprises a CDR1 comprising the amino acid sequence of SEQ ID NO:296, a CDR2 comprising the amino acid sequence of SEQ ID NO:297, and a CDR3 comprising the amino acid sequence of SEQ ID NO: 298. In some aspects, (i) the VHH1 comprises a CDR1 comprising the amino acid sequence of SEQ ID NO:300, a CDR2 comprising the amino acid sequence of SEQ ID NO:301, and a CDR3 comprising the amino acid sequence of SEQ ID NO:302; and (ii) the VHH2 comprises a CDR1 comprising the amino acid sequence of SEQ ID NO:304, a CDR2 comprising the amino acid sequence of SEQ ID NO:305, and a CDR3 comprising the amino acid sequence of SEQ ID NO: 306. In some aspects, (i) the VHH1 comprises a CDR1 comprising the amino acid sequence of SEQ ID NO:308, a CDR2 comprising the amino acid sequence of SEQ ID NO:309, and a CDR3 comprising the amino acid sequence of SEQ ID NO:310; and (ii) the VHH2 comprises a CDR1 comprising the amino acid sequence of SEQ ID NO:312, a CDR2 comprising the amino acid sequence of SEQ ID NO:313, and a CDR3 comprising the amino acid sequence of SEQ ID NO: 314. In some aspects, (i) the VHH1 comprises a CDR1 comprising the amino acid sequence of SEQ ID NO:316, a CDR2 comprising the amino acid sequence of SEQ ID NO:317, and a CDR3 comprising the amino acid sequence of SEQ ID NO:318; and (ii) the VHH2 comprises a CDR1 comprising the amino acid sequence of SEQ ID NO:320, a CDR2 comprising the amino acid sequence of SEQ ID NO:321, and a CDR3 comprising the amino acid sequence of SEQ ID NO: 322. In some aspects, (i) the VHH1 comprises a CDR1 comprising the amino acid sequence of SEQ ID NO:324, a CDR2 comprising the amino acid sequence of SEQ ID NO:325, and a CDR3 comprising the amino acid sequence of SEQ ID NO:326; and (ii) the VHH2 comprises a CDR1 comprising the amino acid sequence of SEQ ID NO:328, a CDR2 comprising the amino acid sequence of SEQ ID NO:329, and a CDR3 comprising the amino acid sequence of SEQ ID NO: 330. In some aspects, (i) the VHH1 comprises a CDR1 comprising the amino acid sequence of SEQ ID NO:332, a CDR2 comprising the amino acid sequence of SEQ ID NO:333, and a CDR3 comprising the amino acid sequence of SEQ ID NO:334; and (ii) the VHH2 comprises a CDR1 comprising the amino acid sequence of SEQ ID NO:336, a CDR2 comprising the amino acid sequence of SEQ ID NO:337, and a CDR3 comprising the amino acid sequence of SEQ ID NO: 338. In some aspects, (i) the VHH1 comprises a CDR1 comprising the amino acid sequence of SEQ ID NO:340, a CDR2 comprising the amino acid sequence of SEQ ID NO:341, and a CDR3 comprising the amino acid sequence of SEQ ID NO:342; and (ii) the VHH2 comprises a CDR1 comprising the amino acid sequence of SEQ ID NO:344, a CDR2 comprising the amino acid sequence of SEQ ID NO:345, and a CDR3 comprising the amino acid sequence of SEQ ID NO: 346.

In some aspects, the polypeptide that binds IL31 (e.g., VHH domain) may be humanized. Humanized binding molecules (such as sdAbs or VHH-containing polypeptides) are useful as therapeutic molecules because they reduce or eliminate the human immune response to non-human binding molecules. For example, a humanized antibody can comprise one or more variable domains in which CDRs, (or portions thereof) are derived from a non-human antibody, and FRs (or portions thereof) are derived from human antibody sequences. A humanized antibody optionally may also comprise at least a portion of a human constant region. In some aspects, some FR residues in a humanized binding molecule are substituted with corresponding residues from a non-human binding molecule (for example, the antibody from which the CDR residues are derived), for example, to restore or improve antibody specificity or affinity.

Humanized binding molecules and methods of making them are reviewed, for example, in Almagro and Fransson, (2008) Front. Biosci. 13:1619-1633, and are further described, for example, in Riechmann et al., (1988) Nature 332:323-329; Queen et al., (1989) Proc. Natl Acad. Sci. USA 86:10029-10033; US Patent Nos. 5, 821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272:10678-10684 and Rosok et al., (1996) J. Biol. Chem. 271:22611-22618). Typically, the FR regions of a VHH are replaced with human FR regions to make a humanized VHH. In some aspects, certain FR residues of the human FR are replaced in order to improve one or more properties of the humanized VHH. VHH domains with such replaced residues are still referred to herein as “humanized.”

In some aspects, the VHH1 comprises an amino acid sequence at least 85%, at least 90%, at least 95%, at least 96%, at least 97%, at least 98%, or at least 99% identical to the amino acid sequence of SEQ ID NO: 3, SEQ ID NO: 17, SEQ ID NO: 31, or SEQ ID NO: 45. In some aspects, the VHH1 comprises an amino acid sequence at least 85%, at least 90%, at least 95%, at least 96%, at least 97%, at least 98%, or at least 99% identical to the amino acid sequence of SEQ ID NO: 4, SEQ ID NO: 18, SEQ ID NO: 32, or SEQ ID NO: 46.

In some aspects, the VHH2 comprises an amino acid sequence at least 85%, at least 90%, at least 95%, at least 96%, at least 97%, at least 98%, or at least 99% identical to the amino acid sequence of SEQ ID NO: 8, SEQ ID NO: 22, SEQ ID NO: 36, or SEQ ID NO: 50. In some aspects, the VHH2 comprises an amino acid sequence at least 85%, at least 90%, at least 95%, at least 96%, at least 97%, at least 98%, or at least 99% identical to the amino acid sequence of SEQ ID NO: 9, SEQ ID NO: 23, SEQ ID NO: 37, or SEQ ID NO: 51.

In some aspects, the VHH1 comprises a CDR1 comprising the amino acid sequence of SEQ ID NO: 5, a CDR2 comprising the amino acid sequence of SEQ ID NO: 6, and a CDR3 comprising the amino acid sequence of SEQ ID NO: 7, wherein the VHH1 comprises an amino acid sequence at least 85%, at least 90%, at least 95%, at least 96%, at least 97%, at least 98%, or at least 99% identical to the amino acid sequence of SEQ ID NO: 3. In some aspects, the VHH1 comprises a CDR1 comprising the amino acid sequence of SEQ ID NO: 5, a CDR2 comprising the amino acid sequence of SEQ ID NO: 6, and a CDR3 comprising the amino acid sequence of SEQ ID NO: 7, wherein the VHH1 comprises an amino acid sequence at least 85%, at least 90%, at least 95%, at least 96%, at least 97%, at least 98%, or at least 99% identical to the amino acid sequence of SEQ ID NO: 4.

In some aspects, the VHH1 comprises a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21, wherein the VHH1 comprises an amino acid sequence at least 85%, at least 90%, at least 95%, at least 96%, at least 97%, at least 98%, or at least 99% identical to the amino acid sequence of SEQ ID NO: 17. In some aspects, the VHH1 comprises a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21, wherein the VHH1 comprises an amino acid sequence at least 85%, at least 90%, at least 95%, at least 96%, at least 97%, at least 98%, or at least 99% identical to the amino acid sequence of SEQ ID NO: 18.

In some aspects, the VHH1 comprises a CDR1 comprising the amino acid sequence of SEQ ID NO: 33, a CDR2 comprising the amino acid sequence of SEQ ID NO: 34, and a CDR3 comprising the amino acid sequence of SEQ ID NO: 35, wherein the VHH1 comprises an amino acid sequence at least 85%, at least 90%, at least 95%, at least 96%, at least 97%, at least 98%, or at least 99% identical to the amino acid sequence of SEQ ID NO: 31. In some aspects, the VHH1 comprises a CDR1 comprising the amino acid sequence of SEQ ID NO: 33, a CDR2 comprising the amino acid sequence of SEQ ID NO: 34, and a CDR3 comprising the amino acid sequence of SEQ ID NO: 35, wherein the VHH1 comprises an amino acid sequence at least 85%, at least 90%, at least 95%, at least 96%, at least 97%, at least 98%, or at least 99% identical to the amino acid sequence of SEQ ID NO: 32.

In some aspects, the VHH1 comprises a CDR1 comprising the amino acid sequence of SEQ ID NO: 47, a CDR2 comprising the amino acid sequence of SEQ ID NO: 48, and a CDR3 comprising the amino acid sequence of SEQ ID NO: 49, wherein the VHH1 comprises an amino acid sequence at least 85%, at least 90%, at least 95%, at least 96%, at least 97%, at least 98%, or at least 99% identical to the amino acid sequence of SEQ ID NO: 45. In some aspects, the VHH1 comprises a CDR1 comprising the amino acid sequence of SEQ ID NO: 47, a CDR2 comprising the amino acid sequence of SEQ ID NO: 48, and a CDR3 comprising the amino acid sequence of SEQ ID NO: 49, wherein the VHH1 comprises an amino acid sequence at least 85%, at least 90%, at least 95%, at least 96%, at least 97%, at least 98%, or at least 99% identical to the amino acid sequence of SEQ ID NO: 46.

In some aspects, the VHH1 comprises a CDR1 comprising the amino acid sequence of SEQ ID NO: 78, a CDR2 comprising the amino acid sequence of SEQ ID NO: 79, and a CDR3 comprising the amino acid sequence of SEQ ID NO: 80, wherein the VHH1 comprises an amino acid sequence at least 85%, at least 90%, at least 95%, at least 96%, at least 97%, at least 98%, or at least 99% identical to the amino acid sequence of SEQ ID NO: 3. In some aspects, the VHH1 comprises a CDR1 comprising the amino acid sequence of SEQ ID NO: 78, a CDR2 comprising the amino acid sequence of SEQ ID NO: 79, and a CDR3 comprising the amino acid sequence of SEQ ID NO: 80, wherein the VHH1 comprises an amino acid sequence at least 85%, at least 90%, at least 95%, at least 96%, at least 97%, at least 98%, or at least 99% identical to the amino acid sequence of SEQ ID NO: 4.

In some aspects, the VHH1 comprises a CDR1 comprising the amino acid sequence of SEQ ID NO: 81, a CDR2 comprising the amino acid sequence of SEQ ID NO: 82, and a CDR3 comprising the amino acid sequence of SEQ ID NO: 83, wherein the VHH1 comprises an amino acid sequence at least 85%, at least 90%, at least 95%, at least 96%, at least 97%, at least 98%, or at least 99% identical to the amino acid sequence of SEQ ID NO: 3. In some aspects, the VHH1 comprises a CDR1 comprising the amino acid sequence of SEQ ID NO: 81, a CDR2 comprising the amino acid sequence of SEQ ID NO: 82, and a CDR3 comprising the amino acid sequence of SEQ ID NO: 83, wherein the VHH1 comprises an amino acid sequence at least 85%, at least 90%, at least 95%, at least 96%, at least 97%, at least 98%, or at least 99% identical to the amino acid sequence of SEQ ID NO: 4.

In some aspects, the VHH2 comprises a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12, wherein the VHH2 comprises an amino acid sequence at least 85%, at least 90%, at least 95%, at least 96%, at least 97%, at least 98%, or at least 99% identical to the amino acid sequence of SEQ ID NO: 8. In some aspects, the VHH2 comprises a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12, wherein the VHH2 comprises an amino acid sequence at least 85%, at least 90%, at least 95%, at least 96%, at least 97%, at least 98%, or at least 99% identical to the amino acid sequence of SEQ ID NO: 9.

In some aspects, the VHH2 comprises a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26, wherein the VHH2 comprises an amino acid sequence at least 85%, at least 90%, at least 95%, at least 96%, at least 97%, at least 98%, or at least 99% identical to the amino acid sequence of SEQ ID NO: 22. In some aspects, the VHH2 comprises a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26, wherein the VHH2 comprises an amino acid sequence at least 85%, at least 90%, at least 95%, at least 96%, at least 97%, at least 98%, or at least 99% identical to the amino acid sequence of SEQ ID NO: 23.

In some aspects, the VHH2 comprises a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40, wherein the VHH2 comprises an amino acid sequence at least 85%, at least 90%, at least 95%, at least 96%, at least 97%, at least 98%, or at least 99% identical to the amino acid sequence of SEQ ID NO: 36. In some aspects, the VHH2 comprises a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40, wherein the VHH2 comprises an amino acid sequence at least 85%, at least 90%, at least 95%, at least 96%, at least 97%, at least 98%, or at least 99% identical to the amino acid sequence of SEQ ID NO: 37.

In some aspects, the VHH2 comprises a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54, wherein the VHH2 comprises an amino acid sequence at least 85%, at least 90%, at least 95%, at least 96%, at least 97%, at least 98%, or at least 99% identical to the amino acid sequence of SEQ ID NO: 50. In some aspects, the VHH2 comprises a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54, wherein the VHH2 comprises an amino acid sequence at least 85%, at least 90%, at least 95%, at least 96%, at least 97%, at least 98%, or at least 99% identical to the amino acid sequence of SEQ ID NO: 51.

In some aspects, the VHH2 comprises a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86, wherein the VHH2 comprises an amino acid sequence at least 85%, at least 90%, at least 95%, at least 96%, at least 97%, at least 98%, or at least 99% identical to the amino acid sequence of SEQ ID NO: 8. In some aspects, the VHH2 comprises a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86, wherein the VHH2 comprises an amino acid sequence at least 85%, at least 90%, at least 95%, at least 96%, at least 97%, at least 98%, or at least 99% identical to the amino acid sequence of SEQ ID NO: 9.

In some aspects, the VHH2 comprises a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89, wherein the VHH2 comprises an amino acid sequence at least 85%, at least 90%, at least 95%, at least 96%, at least 97%, at least 98%, or at least 99% identical to the amino acid sequence of SEQ ID NO: 8. In some aspects, the VHH2 comprises a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89, wherein the VHH2 comprises an amino acid sequence at least 85%, at least 90%, at least 95%, at least 96%, at least 97%, at least 98%, or at least 99% identical to the amino acid sequence of SEQ ID NO: 9.

In some aspects, the VHH1 comprises the amino acid sequence of SEQ ID NO: 3, SEQ ID NO: 17, SEQ ID NO: 31, or SEQ ID NO: 45. In some aspects, the VHH1 comprises the amino acid sequence of SEQ ID NO: 4, SEQ ID NO: 18, SEQ ID NO: 32, or SEQ ID NO: 46.

In some aspects, the VHH2 comprises the amino acid sequence of SEQ ID NO: 8, SEQ ID NO: 22, SEQ ID NO: 36, or SEQ ID NO: 50. In some aspects, the VHH2 comprises the amino acid sequence of SEQ ID NO: 9, SEQ ID NO: 23, or SEQ ID NO: 37, or SEQ ID NO: 51.

In some aspects, the VHH1 comprises a CDR1 comprising the amino acid sequence of SEQ ID NO: 5, a CDR2 comprising the amino acid sequence of SEQ ID NO: 6, and a CDR3 comprising the amino acid sequence of SEQ ID NO: 7, wherein the VHH1 comprises an amino acid sequence at least 85%, at least 90%, at least 95%, at least 96%, at least 97%, at least 98%, or at least 99% identical to the amino acid sequence of SEQ ID NO: 3; and the VHH2 comprises a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12, wherein the VHH2 comprises an amino acid sequence at least 85%, at least 90%, at least 95%, at least 96%, at least 97%, at least 98%, or at least 99% identical to the amino acid sequence of SEQ ID NO: 8.

In some aspects, the VHH1 comprises a CDR1 comprising the amino acid sequence of SEQ ID NO: 5, a CDR2 comprising the amino acid sequence of SEQ ID NO: 6, and a CDR3 comprising the amino acid sequence of SEQ ID NO: 7, wherein the VHH1 comprises an amino acid sequence at least 85%, at least 90%, at least 95%, at least 96%, at least 97%, at least 98%, or at least 99% identical to the amino acid sequence of SEQ ID NO: 4; and the VHH2 comprises a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12, wherein the VHH2 comprises an amino acid sequence at least 85%, at least 90%, at least 95%, at least 96%, at least 97%, at least 98%, or at least 99% identical to the amino acid sequence of SEQ ID NO: 9. In some aspects, provided herein is a polypeptide, wherein the VHH1 comprises the amino acid sequence of SEQ ID NO: 4 and the VHH2 comprises the amino acid sequence of SEQ ID NO: 9.

In some aspects, the VHH1 comprises a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21, wherein the VHH1 comprises an amino acid sequence at least 85%, at least 90%, at least 95%, at least 96%, at least 97%, at least 98%, or at least 99% identical to the amino acid sequence of SEQ ID NO: 17; and the VHH2 comprises a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26, wherein the VHH2 comprises an amino acid sequence at least 85%, at least 90%, at least 95%, at least 96%, at least 97%, at least 98%, or at least 99% identical to the amino acid sequence of SEQ ID NO: 22.

In some aspects, the VHH1 comprises a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21, wherein the VHH1 comprises an amino acid sequence at least 85%, at least 90%, at least 95%, at least 96%, at least 97%, at least 98%, or at least 99% identical to the amino acid sequence of SEQ ID NO: 18; and the VHH2 comprises a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26, wherein the VHH2 comprises an amino acid sequence at least 85%, at least 90%, at least 95%, at least 96%, at least 97%, at least 98%, or at least 99% identical to the amino acid sequence of SEQ ID NO: 23. In some aspects, the VHH1 comprises the amino acid sequence of SEQ ID NO: 18 and the VHH2 comprises the amino acid sequence of SEQ ID NO: 23.

In some aspects, the VHH1 comprises a CDR1 comprising the amino acid sequence of SEQ ID NO: 33, a CDR2 comprising the amino acid sequence of SEQ ID NO: 34, and a CDR3 comprising the amino acid sequence of SEQ ID NO: 35, wherein the VHH1 comprises an amino acid sequence at least 85%, at least 90%, at least 95%, at least 96%, at least 97%, at least 98%, or at least 99% identical to the amino acid sequence of SEQ ID NO: 31; and the VHH2 comprises a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40, wherein the VHH2 comprises an amino acid sequence at least 85%, at least 90%, at least 95%, at least 96%, at least 97%, at least 98%, or at least 99% identical to the amino acid sequence of SEQ ID NO: 36.

In some aspects, the VHH1 comprises a CDR1 comprising the amino acid sequence of SEQ ID NO: 33, a CDR2 comprising the amino acid sequence of SEQ ID NO: 34, and a CDR3 comprising the amino acid sequence of SEQ ID NO: 35, wherein the VHH1 comprises an amino acid sequence at least 85%, at least 90%, at least 95%, at least 96%, at least 97%, at least 98%, or at least 99% identical to the amino acid sequence of SEQ ID NO: 32; and the VHH2 comprises a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40, wherein the VHH2 comprises an amino acid sequence at least 85%, at least 90%, at least 95%, at least 96%, at least 97%, at least 98%, or at least 99% identical to the amino acid sequence of SEQ ID NO: 37. In some aspects, the VHH1 comprises the amino acid sequence of SEQ ID NO: 32 and the VHH2 comprises the amino acid sequence of SEQ ID NO: 37.

In some aspects, the VHH1 comprises a CDR1 comprising the amino acid sequence of SEQ ID NO: 47, a CDR2 comprising the amino acid sequence of SEQ ID NO: 48, and a CDR3 comprising the amino acid sequence of SEQ ID NO: 49, wherein the VHH1 comprises an amino acid sequence at least 85%, at least 90%, at least 95%, at least 96%, at least 97%, at least 98%, or at least 99% identical to the amino acid sequence of SEQ ID NO: 45; and the VHH2 comprises a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54, wherein the VHH2 comprises an amino acid sequence at least 85%, at least 90%, at least 95%, at least 96%, at least 97%, at least 98%, or at least 99% identical to the amino acid sequence of SEQ ID NO: 50.

In some aspects, the VHH1 comprises a CDR1 comprising the amino acid sequence of SEQ ID NO: 47, a CDR2 comprising the amino acid sequence of SEQ ID NO: 48, and a CDR3 comprising the amino acid sequence of SEQ ID NO: 49, wherein the VHH1 comprises an amino acid sequence at least 85%, at least 90%, at least 95%, at least 96%, at least 97%, at least 98%, or at least 99% identical to the amino acid sequence of SEQ ID NO: 46; and the VHH2 comprises a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54, wherein the VHH2 comprises an amino acid sequence at least 85%, at least 90%, at least 95%, at least 96%, at least 97%, at least 98%, or at least 99% identical to the amino acid sequence of SEQ ID NO: 51. In some aspects, the VHH1 comprises the amino acid sequence of SEQ ID NO: 46 and the VHH2 comprises the amino acid sequence of SEQ ID NO: 51.

In some aspects, disclosed herein is a polypeptide comprising a VHH1 which comprises: an amino acid sequence at least 85%, at least 90%, at least 95%, at least 96%, at least 97%, at least 98%, or at least 99% identical to the amino acid sequence of SEQ ID NO:3, SEQ ID NO:4, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 140, SEQ ID NO: 141, SEQ ID NO: 142, SEQ ID NO: 143, SEQ ID NO: 144, or SEQ ID NO: 145. In some aspects, disclosed herein is a polypeptide comprising a VHH2 which comprises an amino acid sequence at least 85%, at least 90%, at least 95%, at least 96%, at least 97%, at least 98%, or at least 99% identical to the amino acid sequence of SEQ ID NO:8, SEQ ID NO:9, SEQ ID NO: 101, SEQ ID NO: 102, SEQ ID NO: 103, or SEQ ID NO: 146. In some aspects, disclosed herein is a polypeptide comprising both the VHH1 and the VHH2.

In some aspects, disclosed herein is a polypeptide comprising a VHH1 which comprises the amino acid sequence of SEQ ID NO:3, SEQ ID NO:4, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 140, SEQ ID NO: 141, SEQ ID NO: 142, SEQ ID NO: 143, SEQ ID NO: 144, or SEQ ID NO: 145. In some aspects, disclosed herein is a polypeptide comprising a VHH2 which comprises the amino acid sequence of SEQ ID NO:8, SEQ ID NO:9, SEQ ID NO: 101, SEQ ID NO: 102, SEQ ID NO: 103, or SEQ ID NO: 146. In some aspects, disclosed herein is a polypeptide comprising (i) a VHH1 which comprises the amino acid sequence of SEQ ID NO:3, SEQ ID NO:4, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 140, SEQ ID NO: 141, SEQ ID NO: 142, SEQ ID NO: 143, SEQ ID NO: 144, or SEQ ID NO: 145 and a VHH2 which comprises the amino acid sequence of SEQ ID NO:8, SEQ ID NO:9, SEQ ID NO: 101, SEQ ID NO: 102, SEQ ID NO: 103, or SEQ ID NO: 146.

In some aspects, disclosed herein is a polypeptide comprising a VHH1 which comprises an amino acid sequence at least 85%, at least 90%, at least 95%, at least 96%, at least 97%, at least 98%, or at least 99% identical to the amino acid sequence of SEQ ID NO:45, SEQ ID NO:46, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 221, SEQ ID NO: 222, SEQ ID NO: 223, SEQ ID NO: 224, SEQ ID NO: 225, or SEQ ID NO: 226. In some aspects, disclosed herein is a polypeptide comprising a VHH2 which comprises an amino acid sequence at least 85%, at least 90%, at least 95%, at least 96%, at least 97%, at least 98%, or at least 99% identical to the amino acid sequence of SEQ ID NO:50, SEQ ID NO:51, SEQ ID NO: 185, SEQ ID NO: 186, or SEQ ID NO: 187.

In some aspects, disclosed herein is a polypeptide comprising a VHH1 which comprises the amino acid sequence of SEQ ID NO:45, SEQ ID NO:46, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 221, SEQ ID NO: 222, SEQ ID NO: 223, SEQ ID NO: 224, SEQ ID NO: 225, or SEQ ID NO: 226 and a VHH2 which comprises the amino acid sequence of SEQ ID NO: 50, SEQ ID NO:51, SEQ ID NO: 185, SEQ ID NO: 186, or SEQ ID NO: 187.

In some aspects, disclosed herein is a polypeptide comprising a VHH1 which comprises a CDR1 comprising the amino acid sequence of SEQ ID NO: 78, a CDR2 comprising the amino acid sequence of SEQ ID NO: 79, and a CDR3 comprising the amino acid sequence of SEQ ID NO: 80, wherein the VHH1 comprises an amino acid sequence at least 85%, at least 90%, at least 95%, at least 96%, at least 97%, at least 98%, or at least 99% identical to the amino acid sequence of SEQ ID NO: 3; and a VHH2 which comprises a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86, wherein the VHH2 comprises an amino acid sequence at least 85%, at least 90%, at least 95%, at least 96%, at least 97%, at least 98%, or at least 99% identical to the amino acid sequence of SEQ ID NO: 8.

In some aspects, the VHH1 comprises a CDR1 comprising the amino acid sequence of SEQ ID NO: 78, a CDR2 comprising the amino acid sequence of SEQ ID NO: 79, and a CDR3 comprising the amino acid sequence of SEQ ID NO: 80, wherein the VHH1 comprises an amino acid sequence at least 85%, at least 90%, at least 95%, at least 96%, at least 97%, at least 98%, or at least 99% identical to the amino acid sequence of SEQ ID NO: 4; and the VHH2 comprises a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86, wherein the VHH2 comprises an amino acid sequence at least 85%, at least 90%, at least 95%, at least 96%, at least 97%, at least 98%, or at least 99% identical to the amino acid sequence of SEQ ID NO: 9. In some aspects, provided herein is a polypeptide, wherein the VHH1 comprises the amino acid sequence of SEQ ID NO: 4 and the VHH2 comprises the amino acid sequence of SEQ ID NO: 9.

In some aspects, the VHH1 comprises a CDR1 comprising the amino acid sequence of SEQ ID NO: 81, a CDR2 comprising the amino acid sequence of SEQ ID NO: 82, and a CDR3 comprising the amino acid sequence of SEQ ID NO: 83, wherein the VHH1 comprises an amino acid sequence at least 85%, at least 90%, at least 95%, at least 96%, at least 97%, at least 98%, or at least 99% identical to the amino acid sequence of SEQ ID NO: 3; and the VHH2 comprises a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89, wherein the VHH2 comprises an amino acid sequence at least 85%, at least 90%, at least 95%, at least 96%, at least 97%, at least 98%, or at least 99% identical to the amino acid sequence of SEQ ID NO: 8.

In some aspects, the VHH1 comprises a CDR1 comprising the amino acid sequence of SEQ ID NO: 81, a CDR2 comprising the amino acid sequence of SEQ ID NO: 82, and a CDR3 comprising the amino acid sequence of SEQ ID NO: 83, wherein the VHH1 comprises an amino acid sequence at least 85%, at least 90%, at least 95%, at least 96%, at least 97%, at least 98%, or at least 99% identical to the amino acid sequence of SEQ ID NO:4; and the VHH2 comprises a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89, wherein the VHH2 comprises an amino acid sequence at least 85%, at least 90%, at least 95%, at least 96%, at least 97%, at least 98%, or at least 99% identical to the amino acid sequence of SEQ ID NO: 9. In some aspects, provided herein is a polypeptide, wherein the VHH1 comprises the amino acid sequence of SEQ ID NO: 4 and the VHH2 comprises the amino acid sequence of SEQ ID NO: 9.

In some aspects, the polypeptide provided herein comprises a linker. In some aspects, the linker is an amino acid linker. In some aspects, the amino acid linker connects two or more antibody variable domains together. In some aspects, the VHH1 is connected to the VHH2 by a linker. In some aspects, the polypeptide has the structure: [first IL31 binder]-linker-[second IL31 binder]. In some aspects, the first and second IL31 binding moieties are the same. In some aspects, the first and second IL31 binding moieties are different. In some aspects, the polypeptide has the structure: [VHH1]-linker-[VHH2]. In some aspects, VHH1 and VHH2 are the same. In some aspects, VHH1 and VHH2 are different.

In some aspects, the linker is from 5 to 50 amino acids long. In some aspects, the linker is between about 1-50, 8-40, 12-37, or 16-30 amino acids in length. In some aspects, the linker is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some aspects, the linker is between about 8 to 40 amino acids in length. In some aspects, the linker is between about 12 to 37 amino acids in length. In some aspect, the linker is a flexible linker. In some aspects, the linker is a rigid linker. In some aspects, the linker is a charged linker. In some aspects, the linker comprises glycine and/or serine residues. In some aspects, the linker comprises an amino acid sequence having at least 85%, at least 90%, at least 95%, at least 96%, at least 97%, at least 98%, at least 99%, or about 100% sequence identity to SEQ ID NO: 104 or SEQ ID NO: 188.

In some aspects, the polypeptide comprises an amino acid sequence at least 85%, at least 90%, at least 95%, at least 96%, at least 97%, at least 98%, or at least 99% identical to the amino acid sequence of SEQ ID NO: 13, SEQ ID NO: 27, SEQ ID NO: 41, SEQ ID NO: 55, SEQ ID NO: 14, SEQ ID NO: 28, SEQ ID NO: 42, or SEQ ID NO: 56. In some aspects, the polypeptide comprises an amino acid sequence at least 85%, at least 90%, at least 95%, at least 96%, at least 97%, at least 98%, or at least 99% identical to the amino acid sequence of SEQ ID NO: 13. In some aspects, the polypeptide comprises an amino acid sequence at least 85%, at least 90%, at least 95%, at least 96%, at least 97%, at least 98%, or at least 99% identical to the amino acid sequence of SEQ ID NO: 27. In some aspects, the polypeptide comprises an amino acid sequence at least 85%, at least 90%, at least 95%, at least 96%, at least 97%, at least 98%, or at least 99% identical to the amino acid sequence of SEQ ID NO: 41. In some aspects, the polypeptide comprises an amino acid sequence at least 85%, at least 90%, at least 95%, at least 96%, at least 97%, at least 98%, or at least 99% identical to the amino acid sequence of SEQ ID NO: 55. In some aspects, the polypeptide comprises an amino acid sequence at least 85%, at least 90%, at least 95%, at least 96%, at least 97%, at least 98%, or at least 99% identical to the amino acid sequence of SEQ ID NO: 14. In some aspects, the polypeptide comprises an amino acid sequence at least 85%, at least 90%, at least 95%, at least 96%, at least 97%, at least 98%, or at least 99% identical to the amino acid sequence of SEQ ID NO: 28. In some aspects, the polypeptide comprises an amino acid sequence at least 85%, at least 90%, at least 95%, at least 96%, at least 97%, at least 98%, or at least 99% identical to the amino acid sequence of SEQ ID NO: 42. In some aspects, the polypeptide comprises an amino acid sequence at least 85%, at least 90%, at least 95%, at least 96%, at least 97%, at least 98%, or at least 99% identical to the amino acid sequence of SEQ ID NO: 56.

In some aspects, the polypeptide comprises an amino acid sequence having at least 85%, at least 90%, at least 95%, at least 96%, at least 97%, at least 98%, at least 99%, or about 100% sequence identity to the amino acid sequence of SEQ ID NO:13, SEQ ID NO:14, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, or SEQ ID NO: 176.

In some aspects, the polypeptide comprises the amino acid sequence of SEQ ID NO:13, SEQ ID NO:14, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, or SEQ ID NO: 176.

In some aspects, the polypeptide comprises an amino acid sequence having at least 85%, at least 90%, at least 95%, at least 96%, at least 97%, at least 98%, at least 99%, or about 100% sequence identity to the amino acid sequence of SEQ ID NO:55, SEQ ID NO:56, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO: 211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ IDNO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249 or SEQ ID NO: 250.

In some aspects, the polypeptide comprises the amino acid sequence of SEQ ID NO:55, SEQ ID NO:56, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO:200, SEQ ID NO:201, SEQ ID NO:202, SEQ ID NO:203, SEQ ID NO: 204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO: 215, SEQ ID NO:216, SEQ ID NO:217, SEQ ID NO:218, SEQ ID NO:219, SEQ ID NO:220, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ IDNO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249 or SEQ ID NO: 250.

In some aspects, the polypeptide comprises an amino acid sequence at least 85% identical to the amino acid sequence of SEQ ID NO: 13. In some aspects, the polypeptide comprises an amino acid sequence at least 90% identical to the amino acid sequence of SEQ ID NO: 13. In some sequence of SEQ ID NO: 13. In some aspects, the polypeptide comprises an amino acid sequence at least 96% identical to the amino acid sequence of SEQ ID NO: 13. In some aspects, the polypeptide comprises an amino acid sequence at least 97% identical to the amino acid sequence of SEQ ID NO: 13. In some aspects, the polypeptide comprises an amino acid sequence at least 98% identical to the amino acid sequence of SEQ ID NO: 13. In some aspects, the polypeptide comprises an amino acid sequence at least 99% identical to the amino acid sequence of SEQ ID NO: 13.

In some aspects, the polypeptide comprises an amino acid sequence at least 85% identical to the amino acid sequence of SEQ ID NO: 27. In some aspects, the polypeptide comprises an amino acid sequence at least 90% identical to the amino acid sequence of SEQ ID NO: 27. In some aspects, the polypeptide comprises an amino acid sequence at least 95% identical to the amino acid sequence of SEQ ID NO: 27. In some aspects, the polypeptide comprises an amino acid sequence at least 96% identical to the amino acid sequence of SEQ ID NO: 27. In some aspects, the polypeptide comprises an amino acid sequence at least 97% identical to the amino acid sequence of SEQ ID NO: 27. In some aspects, the polypeptide comprises an amino acid sequence at least 98% identical to the amino acid sequence of SEQ ID NO: 27. In some aspects, the polypeptide comprises an amino acid sequence at least 99% identical to the amino acid sequence of SEQ ID NO: 27.

In some aspects, the polypeptide comprises an amino acid sequence at least 85% identical to the amino acid sequence of SEQ ID NO: 41. In some aspects, the polypeptide comprises an amino acid sequence at least 90% identical to the amino acid sequence of SEQ ID NO: 41. In some aspects, the polypeptide comprises an amino acid sequence at least 95% identical to the amino acid sequence of SEQ ID NO: 41. In some aspects, the polypeptide comprises an amino acid sequence at least 96% identical to the amino acid sequence of SEQ ID NO: 41. In some aspects, the polypeptide comprises an amino acid sequence at least 97% identical to the amino acid sequence of SEQ ID NO: 41. In some aspects, the polypeptide comprises an amino acid sequence at least 98% identical to the amino acid sequence of SEQ ID NO: 41. In some aspects, the polypeptide comprises an amino acid sequence at least 99% identical to the amino acid sequence of SEQ ID NO: 41.

In some aspects, the polypeptide comprises an amino acid sequence at least 85% identical to the amino acid sequence of SEQ ID NO: 55. In some aspects, the polypeptide comprises an amino acid sequence at least 90% identical to the amino acid sequence of SEQ ID NO: 55. In some sequence of SEQ ID NO: 55. In some aspects, the polypeptide comprises an amino acid sequence at least 96% identical to the amino acid sequence of SEQ ID NO: 55. In some aspects, the polypeptide comprises an amino acid sequence at least 97% identical to the amino acid sequence of SEQ ID NO: 55. In some aspects, the polypeptide comprises an amino acid sequence at least 98% identical to the amino acid sequence of SEQ ID NO: 55. In some aspects, the polypeptide comprises an amino acid sequence at least 99% identical to the amino acid sequence of SEQ ID NO: 55.

In some aspects, the polypeptide comprises an amino acid sequence at least 85% identical to the amino acid sequence of SEQ ID NO: 14. In some aspects, the polypeptide comprises an amino acid sequence at least 90% identical to the amino acid sequence of SEQ ID NO: 14. In some aspects, the polypeptide comprises an amino acid sequence at least 95% identical to the amino acid sequence of SEQ ID NO: 14. In some aspects, the polypeptide comprises an amino acid sequence at least 96% identical to the amino acid sequence of SEQ ID NO: 14. In some aspects, the polypeptide comprises an amino acid sequence at least 97% identical to the amino acid sequence of SEQ ID NO: 14. In some aspects, the polypeptide comprises an amino acid sequence at least 98% identical to the amino acid sequence of SEQ ID NO: 14. In some aspects, the polypeptide comprises an amino acid sequence at least 99% identical to the amino acid sequence of SEQ ID NO: 14.

In some aspects, the polypeptide comprises an amino acid sequence at least 85% identical to the amino acid sequence of SEQ ID NO: 28. In some aspects, the polypeptide comprises an amino acid sequence at least 90% identical to the amino acid sequence of SEQ ID NO: 28. In some aspects, the polypeptide comprises an amino acid sequence at least 95% identical to the amino acid sequence of SEQ ID NO: 28. In some aspects, the polypeptide comprises an amino acid sequence at least 96% identical to the amino acid sequence of SEQ ID NO: 28. In some aspects, the polypeptide comprises an amino acid sequence at least 97% identical to the amino acid sequence of SEQ ID NO: 28. In some aspects, the polypeptide comprises an amino acid sequence at least 98% identical to the amino acid sequence of SEQ ID NO: 28. In some aspects, the polypeptide comprises an amino acid sequence at least 99% identical to the amino acid sequence of SEQ ID NO: 28.

In some aspects, the polypeptide comprises an amino acid sequence at least 85% identical to the amino acid sequence of SEQ ID NO: 42. In some aspects, the polypeptide comprises an amino acid sequence at least 90% identical to the amino acid sequence of SEQ ID NO: 42. In some sequence of SEQ ID NO: 42. In some aspects, the polypeptide comprises an amino acid sequence at least 96% identical to the amino acid sequence of SEQ ID NO: 42. In some aspects, the polypeptide comprises an amino acid sequence at least 97% identical to the amino acid sequence of SEQ ID NO: 42. In some aspects, the polypeptide comprises an amino acid sequence at least 98% identical to the amino acid sequence of SEQ ID NO: 42. In some aspects, the polypeptide comprises an amino acid sequence at least 99% identical to the amino acid sequence of SEQ ID NO: 42.

In some aspects, the polypeptide comprises an amino acid sequence at least 85% identical to the amino acid sequence of SEQ ID NO: 56. In some aspects, the polypeptide comprises an amino acid sequence at least 90% identical to the amino acid sequence of SEQ ID NO: 56. In some aspects, the polypeptide comprises an amino acid sequence at least 95% identical to the amino acid sequence of SEQ ID NO: 56. In some aspects, the polypeptide comprises an amino acid sequence at least 96% identical to the amino acid sequence of SEQ ID NO: 56. In some aspects, the polypeptide comprises an amino acid sequence at least 97% identical to the amino acid sequence of SEQ ID NO: 56. In some aspects, the polypeptide comprises an amino acid sequence at least 98% identical to the amino acid sequence of SEQ ID NO: 56. In some aspects, the polypeptide comprises an amino acid sequence at least 99% identical to the amino acid sequence of SEQ ID NO: 56.

In some aspects, the polypeptide comprises the amino acid sequence of SEQ ID NO: 13, SEQ ID NO: 27, SEQ ID NO: 41, SEQ ID NO: 55, SEQ ID NO: 14, SEQ ID NO: 28, SEQ ID NO: 42, or SEQ ID NO: 56. In some aspects, the polypeptide comprises the amino acid sequence of SEQ ID NO: 14, SEQ ID NO: 28, SEQ ID NO: 42, or SEQ ID NO: 56. In some aspects, the polypeptide comprises the amino acid sequence of SEQ ID NO: 13. In some aspects, the polypeptide comprises the amino acid sequence of SEQ ID NO: 27. In some aspects, the polypeptide comprises the amino acid sequence of SEQ ID NO: 41. In some aspects, the polypeptide comprises the amino acid sequence of SEQ ID NO: 55. In some aspects, the polypeptide comprises the amino acid sequence of SEQ ID NO: 14. In some aspects, the polypeptide comprises the amino acid sequence of SEQ ID NO: 28. In some aspects, the polypeptide comprises the amino acid sequence of SEQ ID NO: 42. In some aspects, the polypeptide comprises the amino acid sequence of SEQ ID NO: 56.

In some aspects, provided herein is a polypeptide that binds IL31 comprising a Fc region. In some aspects, the polypeptide comprises an IgG1, IgG2, IgG3, or IgG4 Fc region. In some aspects, the Fc region is a human Fc region. In some aspects, the polypeptide comprises a human IgG1, IgG2, IgG3, or IgG4 Fc region. In some aspects, the polypeptide comprises an Fc region with a modified effector function. In some aspects, the polypeptide comprises an effector null Fc region. In some aspects, the Fc region comprises a full or partial hinge region. In some aspects, the Fc region comprises a full or partial hinge, no CH1 region, a full or partial CH2 region, and a full or partial CH3 region. In some aspects, the Fc region comprises a full hinge region, no CH1 region, full CH2 region and full CH3 region. In some aspects, the full or partial hinge region comprises a cysteine (C) to serine(S) mutation at position 220 according to EU numbering (C220S). In some aspects, the Fc region includes one or more of the following CH2 region mutations: L234A, L235A, M252Y, S254T, and T256E as determined by EU numbering. In some aspects, the Fc region includes one or more of the following CH3 mutations: M428L and N434S as determined by EU numbering. In some aspects, the Fc region comprises one or more of the following mutations or substitutions (EU numbering): C220S, L234A, L235A, M252Y, S254T, T256E, M428L, N434S or any combination thereof. In some aspects, the Fc region comprises the amino acid sequence of SEQ ID NO:62 or the amino acid residues 1-231 of SEQ ID NO:62. In some aspects, the Fc region comprises the amino acid sequence of SEQ ID NO:90 or the amino acid residues 1-231 of SEQ ID NO:90.

In some aspects, the polypeptide provided herein comprises a linker as described herein and an Fc region. In some aspects, the linker is an amino acid linker. In some aspects, the amino acid linker connects two or more antibody variable domains together. In some aspects, the VHH1 is connected to the VHH2 by a linker. In some aspects, the Fc region is connected to the C-terminus of the VHH2. In some aspects, the polypeptide has the structure: [first IL31 binder]-linker-[second IL31 binder]-Fc region. In some aspects, the first and second IL31 binding moieties are the same. In some aspects, the first and second IL3 1 binding moieties are different. In some aspects, the polypeptide has the structure: [VHH1]-linker-[VHH2]-Fc region. In some aspects, VHH1 and VHH2 are the same. In some aspects, VHH1 and VHH2 are different.

In some aspects, a polypeptide of the present disclosure binds to an antigen comprising at least two binding moieties that comprise VHH1, VHH2, and a linker, wherein the polypeptide has the structure: [VHH1]-linker-[VHH2], wherein the linker comprises an amino acid sequence comprising (X)n-G, and the VHH2 comprises at the N-terminus Xa, Xb, and Xc, wherein X is an amino acid, G is glycine, n is any integer between 0 and 100, Xa is E (Glutamic acid), Q (Glutamine), or missing, Xb is V (Valine) or missing, and Xc is Q (Glutamine), K (Lysine), or missing is provided herein. In some aspects, VHH1 is any VHH disclosed herein, and VHH2 is any VHH disclosed herein.

In some aspects, a polypeptide of the present disclosure comprises at least two binding moieties that comprise VHH1, VHH2, and a linker, wherein the polypeptide has the structure: [VHH1]-linker-[VHH2], wherein VHH1 comprises (a) a CDR1 comprising the amino acid sequence of SEQ ID NO: 5, a CDR2 comprising the amino acid sequence of SEQ ID NO: 6, and a CDR3 comprising the amino acid sequence of SEQ ID NO: 7; (b) a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21; (c) a CDR1 comprising the amino acid sequence of SEQ ID NO: 33, a CDR2 comprising the amino acid sequence of SEQ ID NO: 34, and a CDR3 comprising the amino acid sequence of SEQ ID NO: 35; (d) a CDR1 comprising the amino acid sequence of SEQ ID NO: 47, a CDR2 comprising the amino acid sequence of SEQ ID NO: 48, and a CDR3 comprising the amino acid sequence of SEQ ID NO: 49; (e) a CDR1 comprising the amino acid sequence of SEQ ID NO: 78, a CDR2 comprising the amino acid sequence of SEQ ID NO: 79, and a CDR3 comprising the amino acid sequence of SEQ ID NO: 80; or (f) a CDR1 comprising the amino acid sequence of SEQ ID NO: 81, a CDR2 comprising the amino acid sequence of SEQ ID NO: 82, and a CDR3 comprising the amino acid sequence of SEQ ID NO: 83.

In some aspects, a polypeptide of the present disclosure comprises at least two binding moieties that comprise VHH1, VHH2, and a linker, wherein the polypeptide has the structure: [VHH1]-linker-[VHH2], wherein VHH2 comprises (a) a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12; (b) a CDR1 comprising the amino acid sequence of SEQ ID NO: 24, a CDR2 comprising the amino acid sequence of SEQ ID NO:25, and a CDR3 comprising the amino acid sequence of SEQ ID NO: 26; (c) a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40; (d) a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54; (e) a CDR1 comprising the amino acid sequence of SEQ ID NO: 84, a CDR2 comprising the amino acid sequence of SEQ ID NO: 85, and a CDR3 comprising the amino acid sequence of SEQ ID NO: 86; or (f) a CDR1 comprising the amino acid sequence of SEQ ID NO: 87, a CDR2 comprising the amino acid sequence of SEQ ID NO: 88, and a CDR3 comprising the amino acid sequence of SEQ ID NO: 89.

In some aspects, the polypeptide of the present disclosure further comprises an Fc region. In some aspects, the linker (X) useful for the present disclosure comprises (GS)n, (G2S)n, (G3S)n, (G4S)n, (G5S)n, or any combination thereof, and n is an integer between 1 and 10. In some aspects, the linker (X) comprises (G4S)n, and n is 4, 5, or 6. In some aspects, the linker comprises the amino acid sequence of SEQ ID NO: 104 or SEQ ID NO: 188.

In some aspects, the polypeptide of the present disclosure comprises an IgG1, IgG2, IgG3, or IgG4 Fc region. In some aspects, the polypeptide of the present disclosure comprises a human Fc region. In some aspects, the Fc region comprises one or more substitutions, deletions, insertions, or any combination thereof. In some aspects, the one or more substitutions comprise C220S, M252Y, S254T, T256E, L234A, L235A, M428L, N434S, or any combination thereof, according to the EU numbering system. In some aspects, the C terminus of the Fc region is Lysine. In some aspects, the C terminus of the Fc region is not Lysine, e.g., the C-terminal lysine has been enzymatically removed.

In some aspects, the polypeptide that specifically binds to IL31 by a first binder and a second binder further comprises a binder that binds to a protein, but does not bind to IL31 (“third binder”). In some aspects, the third binder comprises a VHH (VHH3). In some aspects, the polypeptide that specifically binds to IL31 by a first binder and a second binder and that binds to a protein that is not IL31 by a third binder further comprises a binder that binds to the same protein as the third binder or a different protein from the third binder (“fourth binder”). In some aspects, the fourth binder comprises a VHH (VHH4). In some aspects, the VHH3 and the VHH4 are different. In some aspects, the VHH3 and the VHH4 are the same. In some aspects, VHH3 and VHH4 binders bind to the same target antigen. In some aspects, VHH3 and VHH4 binders bind to the same target antigen at the same epitope. In some aspects, VHH3 and VHH4 binders bind to the same target antigen at nonoverlapping epitopes allowing each of VHH3 and VHH4 to simultaneously bind the target antigen. In some aspects, VHH3 and VHH4 binders have the following structure: [VHH3]-linker-[VHH4]. In some aspects, the polypeptide has the following structure: [VHH1]-linker-[VHH2]-linker-[VHH3]-linker-[VHH4]. In some aspects, the polypeptide has the following structure: [VHH1]-linker-[VHH2]-linker-[VHH3]-linker-[VHH4]-Fc region or [VHH1]-linker-[VHH2]-linker-[VHH3]-linker-[VHH4]-linker-Fc region (“Tandem Format”). In other aspects, the polypeptide has the following structure: [VHH1]-linker-[VHH2]-Fc region-[VHH3]-linker-[VHH4] or [VHH1]-linker [VHH2]-Fc region-linker-[VHH3]-linker-[VHH4](“Morrison Format”).

In some aspects, the polypeptide is a multimeric polypeptide, such as a bispecific, trispecific or tetraspecific polypeptide. The multimeric polypeptide may further include an Fc region or portion thereof. A bispecific polypeptide, for example, can comprise two co-binders, wherein each co-binder comprises two biparatopic single binders, joined together by peptide linker, such as Tandem Format or Morrison Format. One co-binder (VHH1-linker-VHH2) is capable of specifically and simultaneously binding to IL-31, e.g., VHH1 and VHH2 specifically bind IL-31 at nonoverlapping epitopes, whereas the other co-binder (VHH3-linker-VHH4) is capable of specifically and simultaneously binding to a target antigen that is not IL-31. The non-IL31 antigen specifically targeted by the second or other co-binder may include, for example, IL31RA, IL-13, IL13Rα1, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2Ry, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12Rβ2, IL14, IL15, IL15Rα, IL16, IL18, IL18Rα, IL18RAcP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCE, PKC8, STAT3, Paxillin, IL33, IL34, CSF-1R, IL35, IL36A, IL36B, IL36G, IL36RA, IL37, IL 18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcaRI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, and CXCR2.

In one aspect, the bispecific polypeptide that binds IL31 and a target antigen comprises a first polypeptide that specifically binds IL31 comprising a first binder and a second binder, and a second polypeptide that specifically binds target antigen comprising a third binder and a fourth binder, wherein the first binder comprises a VHH1 comprising a CDR1 comprising an amino acid sequence of SEQ ID NO:5, a CDR2 comprising an amino acid sequence of SEQ ID NO:6, and a CDR3 comprising an amino acid sequence of SEQ ID NO:7; and the second binder comprises a VHH2 comprising a CDR1 comprising an amino acid sequence of SEQ ID NO:10, a CDR2 comprising an amino acid sequence of SEQ ID NO:11, and a CDR3 comprising an amino acid sequence of SEQ ID NO:12; wherein the first binder and second binder are joined by a peptide linker; wherein the third binder comprises a VHH3 and the fourth binder comprises a VHH4, wherein the first polypeptide and second polypeptide are joined by a peptide linker and/or Fc region, and wherein the target antigen is IL31RA, IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL 17Rβ, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2Ry, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12Rβ2, IL14, IL15, IL15Rα, IL16, IL18, IL18Rα, IL18RAcP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCs, PKCS, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcERI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the bispecific polypeptide that binds IL31 and a target antigen comprises a first polypeptide that specifically binds IL31 comprising a first binder and a second binder, and a second polypeptide that specifically binds target antigen comprising a third binder and a fourth binder, wherein the first binder comprises a VHH1 comprising a CDR1 comprising an amino acid sequence of SEQ ID NO:5 or SEQ ID NO:59, a CDR2 comprising an amino acid sequence of SEQ ID NO: 6, SEQ ID NO:60 or SEQ ID NO:63, and a CDR3 comprising an amino acid sequence of SEQ ID NO:7, SEQ ID NO:61 or SEQ ID NO:64; and the second binder comprises a VHH2 comprising a CDR1 comprising an amino acid sequence of SEQ ID NO:10 or SEQ ID NO:251, a CDR2 comprising an amino acid sequence of SEQ ID NO:11 or SEQ ID NO:252, and a CDR3 comprising an amino acid sequence of SEQ ID NO:12 or SEQ ID NO:253; wherein the first binder and second binder are joined by a peptide linker; wherein the third binder comprises a VHH3, and the fourth binder comprises a VHH4, wherein the first polypeptide and second polypeptide are joined by a peptide linker and/or Fc region, and wherein the target antigen is IL31RA, IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2Ry, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12Rβ2, IL14, IL15, IL15Rα, IL16, IL18, IL 18Rα, IL18RAcP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCE, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcaRI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the bispecific polypeptide that binds IL31 and a target antigen comprises a first polypeptide that specifically binds IL31 comprising a first binder and a second binder, and a second polypeptide that specifically binds target antigen comprising a third binder and a fourth binder, wherein the first binder comprises a VHH1 comprising a CDR1 comprising an amino acid sequence of SEQ ID NO:5, a CDR2 comprising an amino acid sequence of SEQ ID NO:6, and a CDR3 comprising an amino acid sequence of SEQ ID NO:7; and the second binder comprises a VHH2 comprising a CDR1 comprising an amino acid sequence of SEQ ID NO:10, a CDR2 comprising an amino acid sequence of SEQ ID NO:11, and a CDR3 comprising an amino acid sequence of SEQ ID NO:12; wherein the first binder and second binder are joined by a peptide linker; wherein the third binder comprises a VHH3 and the fourth binder comprises a VHH4, wherein the first polypeptide and second polypeptide are joined by a peptide linker and/or Fc region, and wherein the target antigen is IL-13.

In one aspect, the bispecific polypeptide that binds IL31 and a target antigen comprises a first polypeptide that specifically binds IL31 comprising a first binder and a second binder, and a second polypeptide that specifically binds target antigen comprising a third binder and a fourth binder, wherein the first binder comprises a VHH1 comprising a CDR1 comprising an amino acid sequence of SEQ ID NO:5 or SEQ ID NO:59, a CDR2 comprising an amino acid sequence of SEQ ID NO: 6, SEQ ID NO:60 or SEQ ID NO:63, and a CDR3 comprising an amino acid sequence of SEQ ID NO:7, SEQ ID NO:61 or SEQ ID NO:64; and the second binder comprises a VHH2 comprising a CDR1 comprising an amino acid sequence of SEQ ID NO:10 or SEQ ID NO:251, a CDR2 comprising an amino acid sequence of SEQ ID NO:11 or SEQ ID NO:252, and a CDR3 comprising an amino acid sequence of SEQ ID NO: 12 or SEQ ID NO:253; wherein the first binder and second binder are joined by a peptide linker; wherein the third binder comprises a VHH3 and the fourth binder comprises a VHH4, wherein the first polypeptide and second polypeptide are joined by a peptide linker and/or Fc region, and wherein the target antigen is IL-13.

In one aspect, the bispecific polypeptide that binds IL31 and a target antigen comprises a first polypeptide that specifically binds IL31 comprising a first binder and a second binder, and a second polypeptide that specifically binds target antigen comprising a third binder and a fourth binder, wherein the first binder comprises a VHH1 comprising a CDR1 comprising an amino acid sequence of SEQ ID NO:47, a CDR2 comprising an amino acid sequence of SEQ ID NO:48, and a CDR3 comprising an amino acid sequence of SEQ ID NO:49; and the second binder comprises a VHH2 comprising a CDR1 comprising an amino acid sequence of SEQ ID NO:52, a CDR2 comprising an amino acid sequence of SEQ ID NO:53, and a CDR3 comprising an amino acid sequence of SEQ ID NO:54; wherein the first binder and second binder are joined by a peptide linker; wherein the third binder comprises a VHH3 and the fourth binder comprises a VHH4, wherein the first polypeptide and second polypeptide are joined by a peptide linker and/or Fc region, and wherein the target antigen is IL31RA, IL-13, IL13Rα1, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, a4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2Ry, IL3, IL3Rα, CSF2RB, IL5, IL5Rα, IL7, IL7R, IL7Rα, IL7Δ, Y-chain, IL8, IL9, IL-9R, IL10, IL10Rα, IL10Rβ, IL11, IL11Rα, IL12, IL12A, IL12B, IL12Rβ1, IL12Rβ2, IL14, IL15, IL15Rα, IL16, IL18, IL18Rα, IL18RAcP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCs, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcaRI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the bispecific polypeptide that binds IL31 and a target antigen comprises a first polypeptide that specifically binds IL31 comprising a first binder and a second binder, and a second polypeptide that specifically binds target antigen comprising a third binder and a fourth binder, wherein the first binder comprises a VHH1 comprising a CDR1 comprising an amino acid sequence of SEQ ID NO:47 or SEQ ID NO:59, a CDR2 comprising an amino acid sequence of SEQ ID NO:48, SEQ ID NO:60 or SEQ ID NO:63, and a CDR3 comprising an amino acid sequence of SEQ ID NO:49, SEQ ID NO:61 or SEQ ID NO:64; and the second binder comprises a VHH2 comprising a CDR1 comprising an amino acid sequence of SEQ ID NO:52, a CDR2 comprising an amino acid sequence of SEQ ID NO:53, and a CDR3 comprising an amino acid sequence of SEQ ID NO:54; wherein the first binder and second binder are joined by a peptide linker; wherein the third binder comprises a VHH3 and the fourth binder comprises a VHH4, wherein the first polypeptide and second polypeptide are joined by a peptide linker and/or Fc region, and wherein the target antigen is IL31RA, IL-13, IL13Rα1, IL13Rα2, TL1A, IL4, IL4RA, TXLNA, IL13RA, OSMR-beta, Ox40, Ox40L, PD1, PDL1, TSLP, IL-17, IL17A, IL17B, IL 17Rβ, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2Ry, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12Rβ2, IL14, IL15, IL15Rα, IL16, IL18, IL18Rα, IL18RACP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCs, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcaRI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the bispecific polypeptide that binds IL31 and a target antigen comprises a first polypeptide that specifically binds IL31 comprising a first binder and a second binder, and a second polypeptide that specifically binds target antigen comprising a third binder and a fourth binder, wherein the first binder comprises a VHH1 comprising a CDR1 comprising an amino acid sequence of SEQ ID NO:47, a CDR2 comprising an amino acid sequence of SEQ ID NO:48, and a CDR3 comprising an amino acid sequence of SEQ ID NO:49; and the second binder comprises a VHH2 comprising a CDR1 comprising an amino acid sequence of SEQ ID NO:52, a CDR2 comprising an amino acid sequence of SEQ ID NO:53, and a CDR3 comprising an amino acid sequence of SEQ ID NO:54; wherein the first binder and second binder are joined by a peptide linker; wherein the third binder comprises a VHH3 and the fourth binder comprises a VHH4, wherein the first polypeptide and second polypeptide are joined by a peptide linker and/or Fc region, and wherein the target antigen is IL-13.

In one aspect, the bispecific polypeptide that binds IL31 and a target antigen comprises a first polypeptide that specifically binds IL31 comprising a first binder and a second binder, and a second polypeptide that specifically binds target antigen comprising a third binder and a fourth binder, wherein the first binder comprises a VHH1 comprising a CDR1 comprising an amino acid sequence of SEQ ID NO:47 or SEQ ID NO:59, a CDR2 comprising an amino acid sequence of SEQ ID NO:48, SEQ ID NO:60 or SEQ ID NO:63, and a CDR3 comprising an amino acid sequence of SEQ ID NO:49, SEQ ID NO:61 or SEQ ID NO:64; and the second binder comprises a VHH2 comprising a CDR1 comprising an amino acid sequence of SEQ ID NO:52, a CDR2 comprising an amino acid sequence of SEQ ID NO:53, and a CDR3 comprising an amino acid sequence of SEQ ID NO:54; wherein the first binder and second binder are joined by a peptide linker; wherein the third binder comprises a VHH3 and the fourth binder comprises a VHH4, wherein the first polypeptide and second polypeptide are joined by a peptide linker and/or Fc region, and wherein the target antigen is IL-13.

In one aspect, the multimeric polypeptide is a trispecific polypeptide. The trispecific polypeptide may further include an Fc region or portion thereof. A trispecific polypeptide, for example, can comprise three co-binders, wherein each co-binder comprises two biparatopic single binders joined together by peptide linker, such as Tandem Format, e.g., three co-binders joined by peptide linkers, which may include an Fc region N-or C-terminal to the three co-binders, or Morrison Format, e.g., 1^{st}CoBinder-Linker-2^{nd}CoBinder-Fc-3^{rd}Cobinder; 1^{st}CoBinder-Linker-2^{nd}CoBinder-Fc-Linker-3^{rd}Cobinder; 1^{st}CoBinder-Fc-Linker-2^{nd}Cobinder-Linker-3^{rd}Cobinder, 1^{st}CoBinder-Fc-2^{nd}Cobinder-Linker-3^{rd}Cobinder. Both binding moieties, e.g., VHHs, of one of the three co-binders will specifically target IL31, while the other two co-binders will target an antigen that is not IL31, and is selected from the group of IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2Ry, IL3, IL3Rα, CSF2RB, IL5, IL5Rα, IL7, IL7R, IL7Rα, IL7Δ, γ-chain, IL8, IL9, IL-9R, IL10, IL10Rα, IL10R, IL11, IL11Rα, IL12, IL12A, IL12B, IL12Rβ1, IL12Rβ2, IL14, IL15, IL15Rα, IL16, IL18, IL18Rα, IL18RAcP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCs, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcERI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, and CXCR2.

In one aspect, the trispecific polypeptide that binds IL31, a first target antigen, and a second target antigen comprises a first polypeptide that specifically binds IL31 comprising a first binder and a second binder, a second polypeptide that specifically binds the first target antigen comprising a third binder and a fourth binder, and a third polypeptide that specifically binds the second target antigen comprising a fifth binder and a sixth binder, wherein the first binder comprises a VHH1 comprising a CDR1 comprising an amino acid sequence of SEQ ID NO:5 or SEQ ID NO:59, a CDR2 comprising an amino acid sequence of SEQ ID NO:6, SEQ ID NO:60 or SEQ ID NO:63, and a CDR3 comprising an amino acid sequence of SEQ ID NO:7, SEQ ID NO:61 or SEQ ID NO:64, and the second binder comprises a VHH2 comprising a CDR1 comprising an amino acid sequence of SEQ ID NO:10 or SEQ ID NO:251, a CDR2 comprising an amino acid sequence of SEQ ID NO:11 or SEQ ID NO:252, and a CDR3 comprising an amino acid sequence of SEQ ID NO:12 or SEQ ID NO:253; wherein the first binder and second binder are joined by a peptide linker; wherein the third binder comprises a VHH3, and the fourth binder comprises a VHH4, wherein the third binder and fourth binder are joined by a peptide linker; wherein the fifth binder comprises a VHH5, and the sixth binder comprises a VHH6, wherein the fifth binder and sixth binder are joined by a peptide linker; wherein the first polypeptide, second polypeptide and third polypeptide are joined by a peptide linker and/or Fc region, and wherein the first target antigen and/or second target antigen is IL31RA, IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL 17Rβ, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2Ry, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12Rβ2, IL14, IL15, IL15Rα, IL16, IL18, IL18Rα, IL18RAcP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCE, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcERI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the trispecific polypeptide that binds IL31, a first target antigen, and a second target antigen comprises a first polypeptide that specifically binds IL31 comprising a first binder and a second binder, a second polypeptide that specifically binds the first target antigen comprising a third binder and a fourth binder, and a third polypeptide that specifically binds the second target antigen comprising a fifth binder and a sixth binder, wherein the first binder comprises a VHH1 comprising a CDR1 comprising an amino acid sequence of SEQ ID NO:5, a CDR2 comprising an amino acid sequence of SEQ ID NO:6, and a CDR3 comprising an amino acid sequence of SEQ ID NO:7, and the second binder comprises a VHH2 comprising a CDR1 comprising an amino acid sequence of SEQ ID NO: 10, a CDR2 comprising an amino acid sequence of SEQ ID NO:11, and a CDR3 comprising an amino acid sequence of SEQ ID NO:12; wherein the first binder and second binder are joined by a peptide linker; wherein the third binder comprises a VHH3, and the fourth binder comprises a VHH4, wherein the third binder and fourth binder are joined by a peptide linker; wherein the fifth binder comprises a VHH5, and the sixth binder comprises a VHH6, wherein the fifth binder and sixth binder are joined by a peptide linker; wherein the first polypeptide, second polypeptide and third polypeptide are joined by a peptide linker and/or Fc region; wherein the first target antigen and/or second target antigen is IL31RA, IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2Ry, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12R$2, IL14, IL15, IL15Rα, IL16, IL18, IL18Rα, IL18RAcP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCE, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcERI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the trispecific polypeptide that binds IL31, a first target antigen, and a second target antigen comprises a first polypeptide that specifically binds IL31 comprising a first binder and a second binder, a second polypeptide that specifically binds the first target antigen comprising a third binder and a fourth binder, and a third polypeptide that specifically binds the second target antigen comprising a fifth binder and a sixth binder, wherein the first binder comprises a VHH1 comprising a CDR1 comprising an amino acid sequence of SEQ ID NO:5 or SEQ ID NO:59, a CDR2 comprising an amino acid sequence of SEQ ID NO:6, SEQ ID NO:60 or SEQ ID NO:63, and a CDR3 comprising an amino acid sequence of SEQ ID NO:7, SEQ ID NO:61 or SEQ ID NO:64, and the second binder comprises a VHH2 comprising a CDR1 comprising an amino acid sequence of SEQ ID NO:10 or SEQ ID NO:251, a CDR2 comprising an amino acid sequence of SEQ ID NO:11 or SEQ ID NO:252, and a CDR3 comprising an amino acid sequence of SEQ ID NO:12 or SEQ ID NO:253; wherein the first binder and second binder are joined by a peptide linker; wherein the third binder comprises a VHH3, and the fourth binder comprises a VHH4, wherein the third binder and fourth binder are joined by a peptide linker; wherein the fifth binder comprises a VHH5, and the sixth binder comprises a VHH6, wherein the fifth binder and sixth binder are joined by a peptide linker; wherein the first polypeptide, second polypeptide and third polypeptide are joined by a peptide linker and/or Fc region; wherein the first target antigen is IL-13; and wherein second target antigen is IL31RA, IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2Ry, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12Rβ2, IL14, IL15, IL15Rα, IL16, IL18, IL18Rα, IL18RAcP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCs, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcεR1), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the trispecific polypeptide that binds IL31, a first target antigen, and a second target antigen comprises a first polypeptide that specifically binds IL31 comprising a first binder and a second binder, a second polypeptide that specifically binds the first target antigen comprising a third binder and a fourth binder, and a third polypeptide that specifically binds the second target antigen comprising a fifth binder and a sixth binder, wherein the first binder comprises a VHH1 comprising a CDR1 comprising an amino acid sequence of SEQ ID NO:5, a CDR2 comprising an amino acid sequence of SEQ ID NO:6, and a CDR3 comprising an amino acid sequence of SEQ ID NO:7, and the second binder comprises a VHH2 comprising a CDR1 comprising an amino acid sequence of SEQ ID NO: 10, a CDR2 comprising an amino acid sequence of SEQ ID NO:11, and a CDR3 comprising an amino acid sequence of SEQ ID NO:12; wherein the first binder and second binder are joined by a peptide linker; wherein the third binder comprises a VHH3, and the fourth binder comprises a VHH4, wherein the third binder and fourth binder are joined by a peptide linker; wherein the fifth binder comprises a VHH5, and the sixth binder comprises a VHH6, wherein the fifth binder and sixth binder are joined by a peptide linker; wherein the first polypeptide, second polypeptide and third polypeptide are joined by a peptide linker and/or Fc region; wherein the first target antigen is IL13; and wherein the second target antigen is IL31RA, IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAP, IL1R2, IL2, IL2R, IL2Rβ, IL2Ry, IL3, IL3Rα, CSF2RB, IL5, IL5Rα, IL7, IL7R, IL7Rα, IL7Δ, γ-chain, IL8, IL9, IL-9R, IL 10, IL10Rα, IL10Rβ, IL11, IL11Rα, IL12, IL12A, IL12B, IL12Rβ1, IL12R$2, IL14, IL15, IL15Rα, IL16, IL18, IL18Rα, IL18RAcP, IL18BP, IL19, IL20, IL20Rα, IL20Rβ, IL21, IL21R, IL22, IL22Rα1, IL22Rα2, IL24, IL25, IL25/IL17E, IL26, IL27, IL27Rα, IL28A, IL28B, IL28RA, IL29, IL32, PR3, PKC&, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcERI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the trispecific polypeptide that binds IL31, a first target antigen, and a second target antigen comprises a first polypeptide that specifically binds IL31 comprising a first binder and a second binder, a second polypeptide that specifically binds the first target antigen comprising a third binder and a fourth binder, and a third polypeptide that specifically binds the second target antigen comprising a fifth binder and a sixth binder, wherein the first binder comprises a VHH1 comprising a CDR1 comprising an amino acid sequence of SEQ ID NO:47 or SEQ ID NO:59, a CDR2 comprising an amino acid sequence of SEQ ID NO:48, SEQ ID NO:60 or SEQ ID NO:63, and a CDR3 comprising an amino acid sequence of SEQ ID NO:49, SEQ ID NO: 61 or SEQ ID NO:64, and the second binder comprises a VHH2 comprising a CDR1 comprising an amino acid sequence of SEQ ID NO:52, a CDR2 comprising an amino acid sequence of SEQ ID NO:53, and a CDR3 comprising an amino acid sequence of SEQ ID NO:54; wherein the first binder and second binder are joined by a peptide linker; wherein the third binder comprises a VHH3, and the fourth binder comprises a VHH4, wherein the third binder and fourth binder are joined by a peptide linker; wherein the fifth binder comprises a VHH5, and the sixth binder comprises a VHH6, wherein the fifth binder and sixth binder are joined by a peptide linker; wherein the first polypeptide, second polypeptide and third polypeptide are joined by a peptide linker and/or Fc region, and wherein the first target antigen and/or second target antigen is IL31RA, IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2RY, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12Rβ2, IL14, IL15, IL15Rα, IL16, IL18, IL 18Rα, IL18RAcP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCE, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcERI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the trispecific polypeptide that binds IL31, a first target antigen, and a second target antigen comprises a first polypeptide that specifically binds IL31 comprising a first binder and a second binder, a second polypeptide that specifically binds the first target antigen comprising a third binder and a fourth binder, and a third polypeptide that specifically binds the second target antigen comprising a fifth binder and a sixth binder, wherein the first binder comprises a VHH1 comprising a CDR1 comprising an amino acid sequence of SEQ ID NO:47, a CDR2 comprising an amino acid sequence of SEQ ID NO:48, and a CDR3 comprising an amino acid sequence of SEQ ID NO:49, and the second binder comprises a VHH2 comprising a CDR1 comprising an amino acid sequence of SEQ ID NO:52, a CDR2 comprising an amino acid sequence of SEQ ID NO:53, and a CDR3 comprising an amino acid sequence of SEQ ID NO:54; wherein the first binder and second binder are joined by a peptide linker; wherein the third binder comprises a VHH3, and the fourth binder comprises a VHH4, wherein the third binder and fourth binder are joined by a peptide linker; wherein the fifth binder comprises a VHH5, and the sixth binder comprises a VHH6, wherein the fifth binder and sixth binder are joined by a peptide linker; wherein the first polypeptide, second polypeptide and third polypeptide are joined by a peptide linker and/or Fc region, and wherein the first target antigen and/or second target antigen is IL31RA, IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL 17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2Ry, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12Rβ2, IL14, IL15, IL15Rα, IL16, IL18, IL 18Rα, IL18RAcP, IL18BP, IL19, IL20, IL20Rα, IL20Rβ, IL21, IL21R, IL22, IL22Rαl, IL22Rα2, IL24, IL25, IL25/IL17E, IL26, IL27, IL27Rα, IL28A, IL28B, IL28RA, IL29, IL32, PR3, PKCE, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcERI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the trispecific polypeptide that binds IL31, a first target antigen, and a second target antigen comprises a first polypeptide that specifically binds IL31 comprising a first binder and a second binder, a second polypeptide that specifically binds the first target antigen comprising a third binder and a fourth binder, and a third polypeptide that specifically binds the second target antigen comprising a fifth binder and a sixth binder, wherein the first binder comprises a VHH1 comprising a CDR1 comprising an amino acid sequence of SEQ ID NO:47 or SEQ ID NO:59, a CDR2 comprising an amino acid sequence of SEQ ID NO:48, SEQ ID NO:60 or SEQ ID NO:63, and a CDR3 comprising an amino acid sequence of SEQ ID NO:49, SEQ ID NO: 61 or SEQ ID NO:64, and the second binder comprises a VHH2 comprising a CDR1 comprising an amino acid sequence of SEQ ID NO:52, a CDR2 comprising an amino acid sequence of SEQ ID NO:53, and a CDR3 comprising an amino acid sequence of SEQ ID NO:54; wherein the first binder and second binder are joined by a peptide linker; wherein the third binder comprises a VHH3, and the fourth binder comprises a VHH4, wherein the third binder and fourth binder are joined by a peptide linker; wherein the fifth binder comprises a VHH5, and the sixth binder comprises a VHH6, wherein the fifth binder and sixth binder are joined by a peptide linker; wherein the first polypeptide, second polypeptide and third polypeptide are joined by a peptide linker and/or Fc region, wherein the first target antigen is IL 13, and wherein the second target antigen is IL31RA, IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2RY, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12R$2, IL14, IL15, IL15Rα, IL16, IL18, IL18Rα, IL18RAP, IL18BP, IL19, IL20, IL20Rα, IL20Rβ, IL21, IL21R, IL22, IL22Rα1, IL22Rα2, IL24, IL25, IL25/IL17E, IL26, IL27, IL27Rα, IL28A, IL28B, IL28RA, IL29, IL32, PR3, PKC&, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcERI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In one aspect, the trispecific polypeptide that binds IL31, a first target antigen, and a second target antigen comprises a first polypeptide that specifically binds IL31 comprising a first binder and a second binder, a second polypeptide that specifically binds the first target antigen comprising a third binder and a fourth binder, and a third polypeptide that specifically binds the second target antigen comprising a fifth binder and a sixth binder, wherein the first binder comprises a VHH1 comprising a CDR1 comprising an amino acid sequence of SEQ ID NO:47, a CDR2 comprising an amino acid sequence of SEQ ID NO:48, and a CDR3 comprising an amino acid sequence of SEQ ID NO:49, and the second binder comprises a VHH2 comprising a CDR1 comprising an amino acid sequence of SEQ ID NO:52, a CDR2 comprising an amino acid sequence of SEQ ID NO:53, and a CDR3 comprising an amino acid sequence of SEQ ID NO:54; wherein the first binder and second binder are joined by a peptide linker; wherein the third binder comprises a VHH3, and the fourth binder comprises a VHH4, wherein the third binder and fourth binder are joined by a peptide linker; wherein the fifth binder comprises a VHH5, and the sixth binder comprises a VHH6, wherein the fifth binder and sixth binder are joined by a peptide linker; wherein the first polypeptide, second polypeptide and third polypeptide are joined by a peptide linker and/or Fc region, wherein the first target antigen is IL13, and wherein the second target antigen is IL31RA, IL-13, IL13Rαl, IL13Rα2, TL1A, IL4, IL4RA, TXLNA, IL13RA, OSMR-beta, Ox40, Ox40L, PD1, PDL1, TSLP, IL-17, IL17A, IL17B, IL17RB, IL17C, IL17RA, IL17RB, IL17RE, IL17D, IL17E, IL17F, IL17RC, IL17A/A, IL-17A/F, IL-17F/F, TNFα, TNF-β, CD20, CD19, CD25, CD103, CD132, IL23, IL23R, IL-23p19, IgE, CD11Δ, IL6, IL6Rα, gp130, α4-β7, a4-Intergrin, IL1, IL1Δ, IL1β, IL1RA, IL1R1, IL1RL1, IL1RL2, IL1RAcP, IL1R2, IL2, IL2R, IL2Rβ, IL2RY, IL3, IL3Rα, CSF2RB, IL5, IL5Rα, IL7, IL7R, IL7Rα, IL7Δ, γ-chain, IL8, IL9, IL-9R, IL10, IL10Rα, IL10Rβ, IL11, IL11Rα, IL12, IL12A, IL12B, IL12Rβ1, IL12Rβ2, IL14, IL15, IL15Rα, IL16, IL18, IL18Rα, IL18RAcP, IL18BP, IL19, IL20, IL20Rα, IL20Rβ, IL21, IL21R, IL22, IL22Rα1, IL22Rα2, IL24, IL25, IL25/IL17E, IL26, IL27, IL27Rα, IL28A, IL28B, IL28RA, IL29, IL32, PR3, PKCE, PKC8, STAT3, Paxillin, IL33, IL34, CSF-1R, IL35, IL36A, IL36B, IL36G, IL36RA, IL37, IL18R1, IL38, EPO, GM-CSF, G-CSF, M-CSF, Eotaxin, IL1β, BlyS, APRIL, BAFF-R, TACI, CKIT, MRGPRX2, Fc epsilon receptor (FcERI), SIGLEC-6, CD28, CTLA-4, CD80, CD86, SIGLEC-8, TGF-β, CD40L, MCP-1, BASFF, MMPs, Elastase, MIF, Cathepsin G, Cathepsin K, IFN-γ, ROS, NO, RANTES, RANKL, PGE2, ADAMTSs, NGF, BDNF, NPY, MIG, BLC, IP-10, PDGF, CXCR1, or CXCR2.

In some aspects, the polypeptide comprises a Fc region, wherein the Fc region comprises an amino acid sequence at least 85%, at least 90%, at least 95%, at least 96%, at least 97%, at least 98%, or at least 99% identical to the amino acid sequence of SEQ ID NO: 62 or the amino acid residues 1-231 of SEQ ID NO: 62. In some aspects, the polypeptide comprises a Fc region, wherein the Fc region comprises an amino acid sequence at least 85% identical to the amino acid sequence of SEQ ID NO: 62 or the amino acid residues 1-231 of SEQ ID NO: 62. In some aspects, the polypeptide comprises a Fc region, wherein the Fc region comprises an amino acid sequence at least 90% identical to the amino acid sequence of SEQ ID NO: 62 or the amino acid residues 1-231 of SEQ ID NO: 62. In some aspects, the polypeptide comprises a Fc region, wherein the Fc region comprises an amino acid sequence at least 95% identical to the amino acid sequence of SEQ ID NO: 62 or the amino acid residues 1-231 of SEQ ID NO: 62. In some aspects, the polypeptide comprises a Fc region, wherein the Fc region comprises an amino acid sequence at least 96% identical to the amino acid sequence of SEQ ID NO: 62 or the amino acid residues 1-231 of SEQ ID NO: 62. In some aspects, the polypeptide comprises a Fc region, wherein the Fc region comprises an amino acid sequence at least 97% identical to the amino acid sequence of SEQ ID NO: 62 or the amino acid residues 1-231 of SEQ ID NO: 62. In some aspects, the polypeptide comprises a Fc region, wherein the Fc region comprises an amino acid sequence at least 98% identical to the amino acid sequence of SEQ ID NO: 62 or the amino acid residues 1-231 of SEQ ID NO: 62. In some aspects, the polypeptide comprises a Fc region, wherein the Fc region comprises an amino acid sequence at least 99% identical to the amino acid sequence of SEQ ID NO: 62 or the amino acid residues 1-231 of SEQ ID NO: 62.

In some aspects, the Fc region comprises an amino acid sequence at least 85%, at least 90%, at least 95%, at least 96%, at least 97%, at least 98%, or at least 99% identical to the amino acid sequence of SEQ ID NO: 62 or the amino acid residues 1-231 of SEQ ID NO: 62, and wherein residue 5 is serine, residue 37 is tyrosine, residue 39 is threonine, and/or residue 41 is glutamate according to SEQ ID NO: 62 or the amino acid residues 1-231 of SEQ ID NO: 62, or wherein residue 220 is serine, residue 252 is tyrosine, residue 254 is threonine, and/or residue 256 is glutamate according to EU numbering. In some aspects, the Fc region comprises an amino acid sequence at least 85% identical to the amino acid sequence of SEQ ID NO: 62 or the amino acid residues 1-231 of SEQ ID NO: 62, and wherein residue 5 is serine, residue 37 is tyrosine, residue 39 is threonine, and/or residue 41 is glutamate according to SEQ ID NO: 62 or the amino acid residues 1-231 of SEQ ID NO: 62, or wherein residue 220 is serine, residue 252 is tyrosine, residue 254 is threonine, and/or residue 256 is glutamate according to EU numbering. In some aspects, the Fc region comprises an amino acid sequence at least 90% identical to the amino acid sequence of SEQ ID NO: 62 or the amino acid residues 1-231 of SEQ ID NO: 62, and wherein residue 5 is serine, residue 37 is tyrosine, residue 39 is threonine, and/or residue 41 is glutamate according to SEQ ID NO: 62 or the amino acid residues 1-231 of SEQ ID NO: 62, or wherein residue 220 is serine, residue 252 is tyrosine, residue 254 is threonine, and/or residue 256 is glutamate according to EU numbering. In some aspects, the Fc region comprises an amino acid sequence at least 95% identical to the amino acid sequence of SEQ ID NO: 62 or the amino acid residues 1-231 of SEQ ID NO: 62, and wherein residue 5 is serine, residue 37 is tyrosine, residue 39 is threonine, and/or residue 41 is glutamate according to SEQ ID NO: 62 or the amino acid residues 1-231 of SEQ ID NO: 62, or wherein residue 220 is serine, residue 252 is tyrosine, residue 254 is threonine, and/or residue 256 is glutamate according to EU numbering. In some aspects, the Fc region comprises an amino acid sequence at least 96% identical to the amino acid sequence of SEQ ID NO: 62 or the amino acid residues 1-231 of SEQ ID NO: 62, and wherein residue 5 is serine, residue 37 is tyrosine, residue 39 is threonine, and/or residue 41 is glutamate according to SEQ ID NO: 62 or the amino acid residues 1-231 of SEQ ID NO: 62, or wherein residue 220 is serine, residue 252 is tyrosine, residue 254 is threonine, and/or residue 256 is glutamate according to EU numbering. In some aspects, the Fc region comprises an amino acid sequence at least 97% identical to the amino acid sequence of SEQ ID NO: 62 or the amino acid residues 1-231 of SEQ ID NO: 62, and wherein residue 5 is serine, residue 37 is tyrosine, residue 39 is threonine, and/or residue 41 is glutamate according to SEQ ID NO: 62 or the amino acid residues 1-231 of SEQ ID NO: 62, or wherein residue 220 is serine, residue 252 is tyrosine, residue 254 is threonine, and/or residue 256 is glutamate according to EU numbering. In some aspects, the Fc region comprises an amino acid sequence at least 98% identical to the amino acid sequence of SEQ ID NO: 62 or the amino acid residues 1-231 of SEQ ID NO: 62, and wherein residue 5 is serine, residue 37 is tyrosine, residue 39 is threonine, and/or residue 41 is glutamate according to SEQ ID NO: 62 or the amino acid residues 1-231 of SEQ ID NO: 62, or wherein residue 220 is serine, residue 252 is tyrosine, residue 254 is threonine, and/or residue 256 is glutamate according to EU numbering. In some aspects, the Fc region comprises an amino acid sequence at least 99% identical to the amino acid sequence of SEQ ID NO: 62 or the amino acid residues 1-231 of SEQ ID NO: 62, and wherein residue 5 is serine, residue 37 is tyrosine, residue 39 is threonine, and/or residue 41 is glutamate according to SEQ ID NO: 62 or the amino acid residues 1-231 of SEQ ID NO: 62, or wherein residue 220 is serine, residue 252 is tyrosine, residue 254 is threonine, and/or residue 256 is glutamate according to EU numbering. In some aspects, the Fc region comprises the amino acid sequence of SEQ ID NO: 62 or the amino acid residues 1-231 of SEQ ID NO: 62.

In some aspects, the polypeptide that binds IL31 comprises an Fc region comprising the amino acid sequence of SEQ ID NO: 62 or the amino acid residues 1-231 of SEQ ID NO: 62.

In some aspects, the polypeptide comprises a Fc region, wherein the Fc region comprises an amino acid sequence at least 85%, at least 90%, at least 95%, at least 96%, at least 97%, at least 98%, or at least 99% identical to the amino acid sequence of SEQ ID NO: 90 or the amino acid residues 1-231 of SEQ ID NO: 90. In some aspects, the polypeptide comprises a Fc region, wherein the Fc region comprises an amino acid sequence at least 85% identical to the amino acid sequence of SEQ ID NO: 90 or the amino acid residues 1-231 of SEQ ID NO: 90. In some aspects, the polypeptide comprises a Fc region, wherein the Fc region comprises an amino acid sequence at least 90% identical to the amino acid sequence of SEQ ID NO: 90 or the amino acid residues 1-231 of SEQ ID NO: 90. In some aspects, the polypeptide comprises a Fc region, wherein the Fc region comprises an amino acid sequence at least 95% identical to the amino acid sequence of SEQ ID NO: 90 or the amino acid residues 1-231 of SEQ ID NO: 90. In some aspects, the polypeptide comprises a Fc region, wherein the Fc region comprises an amino acid sequence at least 96% identical to the amino acid sequence of SEQ ID NO: 90 or the amino acid residues 1-231 of SEQ ID NO: 90. In some aspects, the polypeptide comprises a Fc region, wherein the Fc region comprises an amino acid sequence at least 97% identical to the amino acid sequence of SEQ ID NO: 90 or the amino acid residues 1-231 of SEQ ID NO: 90. In some aspects, the polypeptide comprises a Fc region, wherein the Fc region comprises an amino acid sequence at least 98% identical to the amino acid sequence of SEQ ID NO: 90 or the amino acid residues 1-231 of SEQ ID NO: 90. In some aspects, the polypeptide comprises a Fc region, wherein the Fc region comprises an amino acid sequence at least 99% identical to the amino acid sequence of SEQ ID NO: 90 or the amino acid residues 1-231 of SEQ ID NO: 90.

In some aspects, the Fc region comprises an amino acid sequence at least 85%, at least 90%, at least 95%, at least 96%, at least 97%, at least 98%, or at least 99% identical to the amino acid sequence of SEQ ID NO: 90 or the amino acid residues 1-231 of SEQ ID NO: 90, and wherein residue 5 is serine, residue 19 is alanine, residue 20 is alanine, residue 37 is tyrosine, residue 39 is threonine, and/or residue 41 is glutamate according to SEQ ID NO: 90 or the amino acid residues 1-231 of SEQ ID NO: 90, or wherein residue 220 is serine, residue 234 is alanine, residue 235 is alanine, residue 252 is tyrosine, residue 254 is threonine, and/or residue 256 is glutamate according to EU numbering. In some aspects, the Fc region comprises an amino acid sequence at least 85% identical to the amino acid sequence of SEQ ID NO: 90 or the amino acid residues 1-231 of SEQ ID NO: 90, and wherein residue 5 is serine, residue 19 is alanine, residue 20 is alanine, residue 37 is tyrosine, residue 39 is threonine, and/or residue 41 is glutamate according to SEQ ID NO: 90 or the amino acid residues 1-231 of SEQ ID NO: 90, or wherein residue 220 is serine, residue 234 is alanine, residue 235 is alanine, residue 252 is tyrosine, residue 254 is threonine, and/or residue 256 is glutamate according to EU numbering. In some aspects, the Fc region comprises an amino acid sequence at least 90% identical to the amino acid sequence of SEQ ID NO: 90 or the amino acid residues 1-231 of SEQ ID NO: 90, and wherein residue 5 is serine, residue 19 is alanine, residue 20 is alanine, residue 37 is tyrosine, residue 39 is threonine, and/or residue 41 is glutamate according to SEQ ID NO: 90 or the amino acid residues 1-231 of SEQ ID NO: 90, or wherein residue 220 is serine, residue 234 is alanine, residue 235 is alanine, residue 252 is tyrosine, residue 254 is threonine, and/or residue 256 is glutamate according to EU numbering. In some aspects, the Fc region comprises an amino acid sequence at least 95% identical to the amino acid sequence of SEQ ID NO: 90 or the amino acid residues 1-231 of SEQ ID NO: 90, and wherein residue 5 is serine, residue 19 is alanine, residue 20 is alanine, residue 37 is tyrosine, residue 39 is threonine, and/or residue 41 is glutamate according to SEQ ID NO: 90 or the amino acid residues 1-231 of SEQ ID NO: 90, or wherein residue 220 is serine, residue 234 is alanine, residue 235 is alanine, residue 252 is tyrosine, residue 254 is threonine, and/or residue 256 is glutamate according to EU numbering. In some aspects, the Fc region comprises an amino acid sequence at least 96% identical to the amino acid sequence of SEQ ID NO: 90 or the amino acid residues 1-231 of SEQ ID NO: 90, and wherein residue 5 is serine, residue 19 is alanine, residue 20 is alanine, residue 37 is tyrosine, residue 39 is threonine, and/or residue 41 is glutamate according to SEQ ID NO: 90 or the amino acid residues 1-231 of SEQ ID NO: 90, or wherein residue 220 is serine, residue 234 is alanine, residue 235 is alanine, residue 252 is tyrosine, residue 254 is threonine, and/or residue 256 is glutamate according to EU numbering. In some aspects, the Fc region comprises an amino acid sequence at least 97% identical to the amino acid sequence of SEQ ID NO: 90 or the amino acid residues 1-231 of SEQ ID NO: 90, and wherein residue 5 is serine, residue 19 is alanine, residue 20 is alanine, residue 37 is tyrosine, residue 39 is threonine, and/or residue 41 is glutamate according to SEQ ID NO: 90 or the amino acid residues 1-231 of SEQ ID NO: 90, or wherein residue 220 is serine, residue 234 is alanine, residue 235 is alanine, residue 252 is tyrosine, residue 254 is threonine, and/or residue 256 is glutamate according to EU numbering. In some aspects, the Fc region comprises an amino acid sequence at least 98% identical to the amino acid sequence of SEQ ID NO: 90 or the amino acid residues 1-231 of SEQ ID NO: 90, and wherein residue 5 is serine, residue 19 is alanine, residue 20 is alanine, residue 37 is tyrosine, residue 39 is threonine, and/or residue 41 is glutamate according to SEQ ID NO: 90 or the amino acid residues 1-231 of SEQ ID NO: 90, or wherein residue 220 is serine, residue 234 is alanine, residue 235 is alanine, residue 252 is tyrosine, residue 254 is threonine, and/or residue 256 is glutamate according to EU numbering. In some aspects, the Fc region comprises an amino acid sequence at least 99% identical to the amino acid sequence of SEQ ID NO: 90 or the amino acid residues 1-231 of SEQ ID NO: 90, and wherein residue 5 is serine, residue 19 is alanine, residue 20 is alanine, residue 37 is tyrosine, residue 39 is threonine, and/or residue 41 is glutamate according to SEQ ID NO: 90 or the amino acid residues 1-231 of SEQ ID NO: 90, or wherein residue 220 is serine, residue 234 is alanine, residue 235 is alanine, residue 252 is tyrosine, residue 254 is threonine, and/or residue 256 is glutamate according to EU numbering. In some aspects, the Fc region comprises the amino acid sequence of SEQ ID NO: 90 or the amino acid residues 1-231 of SEQ ID NO: 90.

In some aspects, the polypeptide that binds IL31 comprises an Fc region comprising the amino acid sequence of SEQ ID NO: 90 or the amino acid residues 1-231 of SEQ ID NO: 90.

In some aspects, the polypeptide that binds IL31 comprises an amino acid sequence at least 85%, at least 90%, at least 95%, at least 96%, at least 97%, at least 98%, or at least 99% identical to the amino acid sequence of SEQ ID NO: 15 or the amino acid residues 1-506 of SEQ ID NO: 15, SEQ ID NO: 29 or the amino acid residues 1-496 of SEQ ID NO: 29, SEQ ID NO: 43 or the amino acid residues 1-487 of SEQ ID NO: 43, SEQ ID NO: 57 or the amino acid residues 1-507 of SEQ ID NO: 57, SEQ ID NO: 91 or the amino acid residues 1-506 of SEQ ID NO: 91, SEQ ID NO: 16 or the amino acid residues 1-506 of SEQ ID NO: 16, SEQ ID NO: 30 or the amino acid residues 1-496 of SEQ ID NO: 30, SEQ ID NO: 44 or the amino acid residues 1-487 of SEQ ID NO: 44, SEQ ID NO: 58 or the amino acid residues 1-507 of SEQ ID NO: 58, or SEQ ID NO: 92 or the amino acid residues 1-506 of SEQ ID NO: 92.

In some aspects, the polypeptide comprises an amino acid sequence at least 85% identical to the amino acid sequence of SEQ ID NO: 15 or the amino acid residues 1-506 of SEQ ID NO: 15. In some aspects, the polypeptide comprises an amino acid sequence at least 90% identical to the amino acid sequence of SEQ ID NO: 15 or the amino acid residues 1-506 of SEQ ID NO: 15. In some aspects, the polypeptide comprises an amino acid sequence at least 95% identical to the amino acid sequence of SEQ ID NO: 15 or the amino acid residues 1-506 of SEQ ID NO: 15. In some aspects, the polypeptide comprises an amino acid sequence at least 96% identical to the amino acid sequence of SEQ ID NO: 15 or the amino acid residues 1-506 of SEQ ID NO: 15. In some aspects, the polypeptide comprises an amino acid sequence at least 97% identical to the amino acid sequence of SEQ ID NO: 15 or the amino acid residues 1-506 of SEQ ID NO: 15. In some aspects, the polypeptide comprises an amino acid sequence at least 98% identical to the amino acid sequence of SEQ ID NO: 15 or the amino acid residues 1-506 of SEQ ID NO: 15. In some aspects, the polypeptide comprises an amino acid sequence at least 99% identical to the amino acid sequence of SEQ ID NO: 15 or the amino acid residues 1-506 of SEQ ID NO: 15.

In some aspects, the polypeptide comprises an amino acid sequence at least 85% identical to the amino acid sequence of SEQ ID NO: 29 or the amino acid residues 1-496 of SEQ ID NO: 29. In some aspects, the polypeptide comprises an amino acid sequence at least 90% identical to the amino acid sequence of SEQ ID NO: 29 or the amino acid residues 1-496 of SEQ ID NO: 29. In some aspects, the polypeptide comprises an amino acid sequence at least 95% identical to the amino acid sequence of SEQ ID NO: 29 or the amino acid residues 1-496 of SEQ ID NO: 29. In some aspects, the polypeptide comprises an amino acid sequence at least 96% identical to the amino acid sequence of SEQ ID NO: 29 or the amino acid residues 1-496 of SEQ ID NO: 29. In some aspects, the polypeptide comprises an amino acid sequence at least 97% identical to the amino acid sequence of SEQ ID NO: 29 or the amino acid residues 1-496 of SEQ ID NO: 29. In some aspects, the polypeptide comprises an amino acid sequence at least 98% identical to the amino acid sequence of SEQ ID NO: 29 or the amino acid residues 1-496 of SEQ ID NO: 29. In some aspects, the polypeptide comprises an amino acid sequence at least 99% identical to the amino acid sequence of SEQ ID NO: 29 or the amino acid residues 1-496 of SEQ ID NO: 29.

In some aspects, the polypeptide comprises an amino acid sequence at least 85% identical to the amino acid sequence of SEQ ID NO: 43 or the amino acid residues 1-487 of SEQ ID NO: 43. In some aspects, the polypeptide comprises an amino acid sequence at least 90% identical to the amino acid sequence of SEQ ID NO: 43 or the amino acid residues 1-487 of SEQ ID NO: 43. In some aspects, the polypeptide comprises an amino acid sequence at least 95% identical to the amino acid sequence of SEQ ID NO: 43 or the amino acid residues 1-487 of SEQ ID NO: 43. In some aspects, the polypeptide comprises an amino acid sequence at least 96% identical to the amino acid sequence of SEQ ID NO: 43 or the amino acid residues 1-487 of SEQ ID NO: 43. In some aspects, the polypeptide comprises an amino acid sequence at least 97% identical to the amino acid sequence of SEQ ID NO: 43 or the amino acid residues 1-487 of SEQ ID NO: 43. In some aspects, the polypeptide comprises an amino acid sequence at least 98% identical to the amino acid sequence of SEQ ID NO: 43 or the amino acid residues 1-487 of SEQ ID NO: 43. In some aspects, the polypeptide comprises an amino acid sequence at least 99% identical to the amino acid sequence of SEQ ID NO: 43 or the amino acid residues 1-487 of SEQ ID NO: 43.

In some aspects, the polypeptide comprises an amino acid sequence at least 85% identical to the amino acid sequence of SEQ ID NO: 57 or the amino acid residues 1-507 of SEQ ID NO: 57. In some aspects, the polypeptide comprises an amino acid sequence at least 90% identical to the amino acid sequence of SEQ ID NO: 57 or the amino acid residues 1-507 of SEQ ID NO: 57. In some aspects, the polypeptide comprises an amino acid sequence at least 95% identical to the amino acid sequence of SEQ ID NO: 57 or the amino acid residues 1-507 of SEQ ID NO: 57. In some aspects, the polypeptide comprises an amino acid sequence at least 96% identical to the amino acid sequence of SEQ ID NO: 57 or the amino acid residues 1-507 of SEQ ID NO: 57. In some aspects, the polypeptide comprises an amino acid sequence at least 97% identical to the amino acid sequence of SEQ ID NO: 57 or the amino acid residues 1-507 of SEQ ID NO: 57. In some aspects, the polypeptide comprises an amino acid sequence at least 98% identical to the amino acid sequence of SEQ ID NO: 57 or the amino acid residues 1-507 of SEQ ID NO: 57. In some aspects, the polypeptide comprises an amino acid sequence at least 99% identical to the amino acid sequence of SEQ ID NO: 57 or the amino acid residues 1-507 of SEQ ID NO: 57.

In some aspects, the polypeptide comprises an amino acid sequence at least 85% identical to the amino acid sequence of SEQ ID NO: 91 or the amino acid residues 1-506 of SEQ ID NO: 91. In some aspects, the polypeptide comprises an amino acid sequence at least 90% identical to the amino acid sequence of SEQ ID NO: 91 or the amino acid residues 1-506 of SEQ ID NO: 91. In some aspects, the polypeptide comprises an amino acid sequence at least 95% identical to the amino acid sequence of SEQ ID NO: 91 or the amino acid residues 1-506 of SEQ ID NO: 91. In some aspects, the polypeptide comprises an amino acid sequence at least 96% identical to the amino acid sequence of SEQ ID NO: 91 or the amino acid residues 1-506 of SEQ ID NO: 91. In some aspects, the polypeptide comprises an amino acid sequence at least 97% identical to the amino acid sequence of SEQ ID NO: 91 or the amino acid residues 1-506 of SEQ ID NO: 91. In some aspects, the polypeptide comprises an amino acid sequence at least 98% identical to the amino acid sequence of SEQ ID NO: 91 or the amino acid residues 1-506 of SEQ ID NO: 91. In some aspects, the polypeptide comprises an amino acid sequence at least 99% identical to the amino acid sequence of SEQ ID NO: 91 or the amino acid residues 1-506 of SEQ ID NO: 91.

In some aspects, the polypeptide comprises an amino acid sequence at least 85% identical to the amino acid sequence of SEQ ID NO: 16 or the amino acid residues 1-506 of SEQ ID NO: 16. In some aspects, the polypeptide comprises an amino acid sequence at least 90% identical to the amino acid sequence of SEQ ID NO: 16 or the amino acid residues 1-506 of SEQ ID NO: 16. In some aspects, the polypeptide comprises an amino acid sequence at least 95% identical to the amino acid sequence of SEQ ID NO: 16 or the amino acid residues 1-506 of SEQ ID NO: 16. In some aspects, the polypeptide comprises an amino acid sequence at least 96% identical to the amino acid sequence of SEQ ID NO: 16 or the amino acid residues 1-506 of SEQ ID NO: 16. In some aspects, the polypeptide comprises an amino acid sequence at least 97% identical to the amino acid sequence of SEQ ID NO: 16 or the amino acid residues 1-506 of SEQ ID NO: 16. In some aspects, the polypeptide comprises an amino acid sequence at least 98% identical to the amino acid sequence of SEQ ID NO: 16 or the amino acid residues 1-506 of SEQ ID NO: 16. In some aspects, the polypeptide comprises an amino acid sequence at least 99% identical to the amino acid sequence of SEQ ID NO: 16 or the amino acid residues 1-506 of SEQ ID NO: 16.

In some aspects, the polypeptide comprises an amino acid sequence at least 85% identical to the amino acid sequence of SEQ ID NO: 30 or the amino acid residues 1-496 of SEQ ID NO: 30. In some aspects, the polypeptide comprises an amino acid sequence at least 90% identical to the amino acid sequence of SEQ ID NO: 30 or the amino acid residues 1-496 of SEQ ID NO: 30. In some aspects, the polypeptide comprises an amino acid sequence at least 95% identical to the amino acid sequence of SEQ ID NO: 30 or the amino acid residues 1-496 of SEQ ID NO: 30. In some aspects, the polypeptide comprises an amino acid sequence at least 96% identical to the amino acid sequence of SEQ ID NO: 30 or the amino acid residues 1-496 of SEQ ID NO: 30. In some aspects, the polypeptide comprises an amino acid sequence at least 97% identical to the amino acid sequence of SEQ ID NO: 30 or the amino acid residues 1-496 of SEQ ID NO: 30. In some aspects, the polypeptide comprises an amino acid sequence at least 98% identical to the amino acid sequence of SEQ ID NO: 30 or the amino acid residues 1-496 of SEQ ID NO: 30. In some aspects, the polypeptide comprises an amino acid sequence at least 99% identical to the amino acid sequence of SEQ ID NO: 30 or the amino acid residues 1-496 of SEQ ID NO: 30.

In some aspects, the polypeptide comprises an amino acid sequence at least 85% identical to the amino acid sequence of SEQ ID NO: 44 or the amino acid residues 1-487 of SEQ ID NO: 44. In some aspects, the polypeptide comprises an amino acid sequence at least 90% identical to the amino acid sequence of SEQ ID NO: 44 or the amino acid residues 1-487 of SEQ ID NO: 44. In some aspects, the polypeptide comprises an amino acid sequence at least 95% identical to the amino acid sequence of SEQ ID NO: 44 or the amino acid residues 1-487 of SEQ ID NO: 44. In some aspects, the polypeptide comprises an amino acid sequence at least 96% identical to the amino acid sequence of SEQ ID NO: 44 or the amino acid residues 1-487 of SEQ ID NO: 44. In some aspects, the polypeptide comprises an amino acid sequence at least 97% identical to the amino acid sequence of SEQ ID NO: 44 or the amino acid residues 1-487 of SEQ ID NO: 44. In some aspects, the polypeptide comprises an amino acid sequence at least 98% identical to the amino acid sequence of SEQ ID NO: 44 or the amino acid residues 1-487 of SEQ ID NO: 44. In some aspects, the polypeptide comprises an amino acid sequence at least 99% identical to the amino acid sequence of SEQ ID NO: 44 or the amino acid residues 1-487 of SEQ ID NO: 44.

In some aspects, the polypeptide comprises an amino acid sequence at least 85% identical to the amino acid sequence of SEQ ID NO: 58 or the amino acid residues 1-507 of SEQ ID NO: 58. In some aspects, the polypeptide comprises an amino acid sequence at least 90% identical to the amino acid sequence of SEQ ID NO: 58 or the amino acid residues 1-507 of SEQ ID NO: 58. In some aspects, the polypeptide comprises an amino acid sequence at least 95% identical to the amino acid sequence of SEQ ID NO: 58 or the amino acid residues 1-507 of SEQ ID NO: 58. In some aspects, the polypeptide comprises an amino acid sequence at least 96% identical to the amino acid sequence of SEQ ID NO: 58 or the amino acid residues 1-507 of SEQ ID NO: 58. In some aspects, the polypeptide comprises an amino acid sequence at least 97% identical to the amino acid sequence of SEQ ID NO: 58 or the amino acid residues 1-507 of SEQ ID NO: 58. In some aspects, the polypeptide comprises an amino acid sequence at least 98% identical to the amino acid sequence of SEQ ID NO: 58 or the amino acid residues 1-507 of SEQ ID NO: 58. In some aspects, the polypeptide comprises an amino acid sequence at least 99% identical to the amino acid sequence of SEQ ID NO: 58 or the amino acid residues 1-507 of SEQ ID NO: 58.

In some aspects, the polypeptide comprises an amino acid sequence at least 85% identical to the amino acid sequence of SEQ ID NO: 92 or the amino acid residues 1-506 of SEQ ID NO: 92. In some aspects, the polypeptide comprises an amino acid sequence at least 90% identical to the amino acid sequence of SEQ ID NO: 92 or the amino acid residues 1-506 of SEQ ID NO: 92. In some aspects, the polypeptide comprises an amino acid sequence at least 95% identical to the amino acid sequence of SEQ ID NO: 92 or the amino acid residues 1-506 of SEQ ID NO: 92. In some aspects, the polypeptide comprises an amino acid sequence at least 96% identical to the amino acid sequence of SEQ ID NO: 92 or the amino acid residues 1-506 of SEQ ID NO: 92. In some aspects, the polypeptide comprises an amino acid sequence at least 97% identical to the amino acid sequence of SEQ ID NO: 92 or the amino acid residues 1-506 of SEQ ID NO: 92. In some aspects, the polypeptide comprises an amino acid sequence at least 98% identical to the amino acid sequence of SEQ ID NO: 92 or the amino acid residues 1-506 of SEQ ID NO: 92. In some aspects, the polypeptide comprises an amino acid sequence at least 99% identical to the amino acid sequence of SEQ ID NO: 92 or the amino acid residues 1-506 of SEQ ID NO: 92.

In some aspects, the polypeptide comprises the amino acid sequence of SEQ ID NO: 15 or the amino acid residues 1-506 of SEQ ID NO: 15, SEQ ID NO: 29 or the amino acid residues 1-496 of SEQ ID NO: 29, SEQ ID NO: 43 or the amino acid residues 1-487 of SEQ ID NO: 43, SEQ ID NO: 57 or the amino acid residues 1-507 of SEQ ID NO: 57, SEQ ID NO: 91 or the amino acid residues 1-506 of SEQ ID NO: 91, SEQ ID NO: 16 or the amino acid residues 1-506 of SEQ ID NO: 16, SEQ ID NO: 30 or the amino acid residues 1-496 of SEQ ID NO: 30, SEQ ID NO: 44 or the amino acid residues 1-487 of SEQ ID NO: 44, SEQ ID NO: 58 or the amino acid residues 1-507 of SEQ ID NO: 58, or SEQ ID NO: 92 or the amino acid residues 1-506 of SEQ ID NO: 92. In some aspects, the polypeptide comprises the amino acid sequence of SEQ ID NO: 16 or the amino acid residues 1-506 of SEQ ID NO: 16, SEQ ID NO: 30 or the amino acid residues 1-496 of SEQ ID NO: 30, SEQ ID NO: 44 or the amino acid residues 1-487 of SEQ ID NO: 44, SEQ ID NO: 58 or the amino acid residues 1-507 of SEQ ID NO: 58, or SEQ ID NO: 92 or the amino acid residues 1-506 of SEQ ID NO: 92. In some aspects, the polypeptide comprises the amino acid sequence of SEQ ID NO: 15 or the amino acid residues 1-506 of SEQ ID NO: 15. In some aspects, the polypeptide comprises the amino acid sequence of SEQ ID NO: 29 or the amino acid residues 1-496 of SEQ ID NO: 29. In some aspects, the polypeptide comprises the amino acid sequence of SEQ ID NO: 43 or the amino acid residues 1-487 of SEQ ID NO: 43. In some aspects, the polypeptide comprises the amino acid sequence of SEQ ID NO: 57 or the amino acid residues 1-507 of SEQ ID NO: 57. In some aspects, the polypeptide comprises the amino acid sequence of SEQ ID NO: 91 or the amino acid residues 1-506 of SEQ ID NO: 91. In some aspects, the polypeptide comprises the amino acid sequence of SEQ ID NO: 16 or the amino acid residues 1-506 of SEQ ID NO: 16. In some aspects, the polypeptide comprises the amino acid sequence of SEQ ID NO: 30 or the amino acid residues 1-496 of SEQ ID NO: 30. In some aspects, the polypeptide comprises the amino acid sequence of SEQ ID NO: 44 or the amino acid residues 1-487 of SEQ ID NO: 44. In some aspects, the polypeptide comprises the amino acid sequence of SEQ ID NO: 58 or the amino acid residues 1-507 of SEQ ID NO: 58. In some aspects, the polypeptide comprises the amino acid sequence of SEQ ID NO: 92 or the amino acid residues 1-506 of SEQ ID NO: 92.

In some aspects, the polypeptide that binds IL31 binds to human IL31. In some aspects, the human IL31 comprises a signal sequence. In some aspects, the human IL31 does not comprise a signal sequence. In some aspects, the human IL31 comprises an amino acid sequence at least 95%, at least 96%, at least 97%, at least 98%, or at least 99% identical to the amino acid sequence of the amino acid sequence of SEQ ID NO: 1 or SEQ ID NO: 2. In some aspects, the human IL31 comprises the amino acid sequence of SEQ ID NO: 1 or SEQ ID NO: 2.

The binding affinity of the polypeptide that binds IL31 can be determined by means of surface plasmon resonance. In some aspects, an equilibrium dissociation constant of an antigen-binding molecule and antigen interaction is determined, wherein the antigen is human IL31. In some aspects, the polypeptide binds to human IL31 at a kD of less than or equal to 1×10⁻⁹ M, less than or equal to 5×10⁻¹⁰ M, less than or equal to 1×10⁻¹⁰ M, less than or equal to 5×10⁻¹¹ M, less than or equal to 1×10⁻¹¹ M, less than or equal to 5×10⁻¹² M, or less than or equal to 1×10⁻¹² M, as measured by surface plasmon resonance. In some aspects, the polypeptide binds to human IL31 at a kD of no more than or equal to 1×10⁻⁹ M, no more than or equal to 5×10⁻¹⁰ M, no more than or equal to 1×10⁻¹⁰ M, no more than or equal to 5×10⁻¹¹ M, no more than or equal to 1×10⁻¹¹ M, no more than or equal to 5×10⁻¹² M, or no more than or equal to 1×10⁻¹² M, as measured by surface plasmon resonance. In some aspects, the polypeptide binds to human IL31 with a kD between about 1×10⁻⁹ M and about 1×10⁻¹² M, about 5×10⁻¹⁰ M and about 5×10⁻¹¹ M, or about 1×10⁻¹⁰ M and about 1×10⁻¹¹ M, or about 5×10⁻¹¹ M, as measured by surface plasmon resonance. In some aspects, the polypeptide binds to human IL31 at a kD of about 1×10⁻⁹ M, about 9×10⁻¹⁰ M, about 8×10⁻¹⁰ M, about 7×10⁻¹⁰ M, about 6×10⁻¹⁰ M, about 5×10⁻¹⁰ M, about 4×10⁻¹⁰ M, about 3×10⁻¹⁰ M, about 2×10⁻¹⁰ M, about 1×10⁻¹⁰ M, about 9×10⁻¹¹ M, about 8×10⁻¹¹ M, about 7×10⁻¹¹ M, about 6×10⁻¹¹ M, about 5×10⁻¹¹ M, about 4×10⁻¹¹ M, about 3×10⁻¹¹ M, about 2×10⁻¹¹ M, about 1×10⁻¹¹ M, about 9×10⁻¹² M, about 8×12⁻¹⁰ M, about 7×10⁻¹² M, about 6×10⁻¹² M, about 5×10⁻¹² M, about 4×10⁻¹² M, about 3×10⁻¹² M, about 2×10⁻¹² M, or about 1×10⁻¹² M, as measured by surface plasmon resonance.

In some aspects, the polypeptide binds to human IL31 at a kD of about 1×10⁻⁹ M, about 5×10⁻¹⁰ M, about 1×10⁻¹⁰ M, about 5×10⁻¹¹ M, about 1×10⁻¹¹ M, about 5×10⁻¹² M, or about 1×10⁻¹² M, as measured by surface plasmon resonance. In some aspects, the polypeptide binds to human IL31 at a kD of about 1×10⁻⁹ M. In some aspects, the polypeptide binds to human IL31 at a kD of about 5×10⁻¹⁰ M. In some aspects, the polypeptide binds to human IL31 at a kD of about 1×10⁻¹⁰ M. In some aspects, the polypeptide binds to human IL31 at a kD of about 5×10⁻¹¹ M. In some aspects, the polypeptide binds to human IL31 at a kD of about 1×10⁻¹¹ M. In some aspects, the polypeptide binds to human IL31 at a kD of about 5×10⁻¹² M. In some aspects, the polypeptide binds to human IL31 at a kD of about 1×10⁻¹² M.

In some aspects, the polypeptide binds to human IL31 at a kD of 1×10⁻⁹ M, 5×10⁻¹⁰ M, 1×10⁻¹⁰ M, 5×10⁻¹¹ M, 1×10⁻¹¹ M, 5×10⁻¹² M, or 1×10⁻¹² M, as measured by surface plasmon resonance. In some aspects, the polypeptide binds to human IL31 at a kD of 1×10⁻⁹ M. In some aspects, the polypeptide binds to human IL31 at a kD of 5×10⁻¹⁰ M. In some aspects, the polypeptide binds to human IL31 at a kD of 1×10⁻¹⁰ M. In some aspects, the polypeptide binds to human IL31 at a kD of 5×10⁻¹¹ M. In some aspects, the polypeptide binds to human IL31 at a kD of 1×10⁻¹¹ M. In some aspects, the polypeptide binds to human IL31 at a kD of 5×10⁻¹² M. In some aspects, the polypeptide binds to human IL31 at a kD of 1×10⁻¹² M.

In some aspects, the polypeptide that binds IL31 has a slow off-rate, wherein the off-rate (koff) is the rate of dissociation between polypeptide and human IL31.

In some aspects, the polypeptide binds to human IL31 with a k_{off} rate of less than or equal to 5×10⁻⁴ M per second, less than or equal to 1×1⁻⁴ M per second, less than or equal to 5×10⁻⁵ M per second, less than or equal to 1×10⁻⁵ M per second, less than or equal to 5×10⁻⁶ M per second, or less than or equal to 1×10⁻⁶ M per second, as measured by surface plasmon resonance. In some aspects, the polypeptide binds to human IL31 with a k_{off} rate of no more than or equal to 5×10⁻⁴ M per second, no more than or equal to 1×1⁻⁴ M per second, no more than or equal to 5×10⁻⁵ M per second, no more than or equal to 1×10⁻⁵ M per second, no more than or equal to 5×106 M per second, or no more than or equal to 1×10⁶ M per second, as measured by surface plasmon resonance. In some aspects, the polypeptide binds to human IL31 with a k_{off} rate of between about 5×10⁻⁴ M per second and 1×10⁻⁶ M per second, between about 1×1⁻⁴ M per second and 5×10⁻⁶ M per second, or between about 5×10⁻⁵ M per second and 1×10⁻⁵ M per second, as measured by surface plasmon resonance. In some aspects, the polypeptide binds to human IL31 with a k_{off} rate of about 9×10⁻⁴ M per second, about 8×10⁻⁴ M per second, about 7×10⁻⁴ M per second, about 6×10⁻⁴ M per second, about 5×10⁻⁴ M per second, about 4×10⁻⁴ M per second, about 3×10⁻⁴ M per second, about 2×10⁻⁴ M per second, about 1×1⁻⁴ M per second, about 9×10⁻⁵ M per second, about 8×10⁻⁵ M per second, about 7×10⁵ M per second, about 6×10⁻⁵ M per second, about 5×10⁻⁵ M per second, about 4×10⁻⁵ M per second, about 3×10⁻⁵ M per second, about 2×10⁻⁵ M per second, about 1×10⁻⁵ M per second, about 9×10⁻⁶ M per second, about 8×10⁻⁶ M per second, about 7×10⁻⁶ M per second, about 6×10⁻⁶ M per second, about 5×10⁻⁶ M per second, about 4×10⁻⁶ M per second, about 3×10⁻⁶ M per second, about 2×10⁻⁶ M per second, or about 1×10⁻⁶ M per second, as measured by surface plasmon resonance.

In some aspects, the polypeptide binds to human IL31 with a k_{off} rate of about 5×10⁻⁴ M per second, about 1×1⁻⁴ M per second, about 5×10⁻⁵ M per second, about 1×10⁻⁵ M per second, about 5×10⁻⁶ M per second, or about 1×10⁻⁶ M per second, as measured by surface plasmon resonance. In some aspects, the polypeptide binds to human IL31 with a k_{off} rate of about 5×10⁻⁴ M per second, as measured by surface plasmon resonance. In some aspects, the polypeptide binds to human IL31 with a k_{off} rate of about 1×10⁻⁴ M per second, as measured by surface plasmon resonance. In some aspects, the polypeptide binds to human IL31 with a k_{off} rate of about 5×10⁻⁵ M per second, as measured by surface plasmon resonance. In some aspects, the polypeptide binds to human IL31 with a k_{off} rate of about 1×10⁻⁵ M per second, as measured by surface plasmon resonance. In some aspects, the polypeptide binds to human IL31 with a k_{off} rate of about 5×10⁻⁶ M per second, as measured by surface plasmon resonance. In some aspects, the polypeptide binds to human IL31 with a k_{off} rate of about 1×10⁻⁶ M per second, as measured by surface plasmon resonance.

In some aspects, the polypeptide binds to human IL31 with a k_{off} rate of 5×10⁻⁴ M per second, 1×1⁻⁴ M per second, 5×10⁻⁵ M per second, 1×10⁻⁵ M per second, 5×10⁻⁶ M per second, or 1×10⁻⁶ M per second, as measured by surface plasmon resonance. In some aspects, the polypeptide binds to human IL31 with a k_{off} rate of 5×10⁴ M per second, as measured by surface plasmon resonance. In some aspects, the polypeptide binds to human IL31 with a k_{off} rate of 1×10⁻⁴ M per second, as measured by surface plasmon resonance. In some aspects, the polypeptide binds to human IL31 with a k_{off} rate of 5×10⁵ M per second, as measured by surface plasmon resonance. In some aspects, the polypeptide binds to human IL31 with a k_{off} rate of 1×10⁻⁵ M per second, as measured by surface plasmon resonance. In some aspects, the polypeptide binds to human IL31 with a k_{off} rate of 5×10⁻⁶ M per second, as measured by surface plasmon resonance. In some aspects, the polypeptide binds to human IL31 with a k_{off} rate of 1×10⁻⁶ M per second, as measured by surface plasmon resonance.

Also provided herein are multimeric polypeptides, each comprising two or more of the any one of the polypeptides provided herein. In some aspects, the present disclosure comprises a dimeric polypeptide comprising at least one polypeptide disclosed herein. In some aspects, the dimer polypeptide of the present disclosure comprises two polypeptides disclosed herein of SEQ ID NO: 90.

In some aspects, the polypeptide of the present disclosure is divalent, trivalent, tetravalent, pentavalent, hexavalent, heptavalent, octavalent, nonavalent, or decavalent. In some aspects, the polypeptide of the present disclosure is octavalent. In some aspects, the polypeptide of the present disclosure is tetravalent.

### III. Exemplary Polypeptide Expression and Production

Nucleic acid molecules comprising polynucleotides that encode a polypeptide that binds IL31 described herein (e.g., Section II and the Examples) are provided. In some aspects, an isolated nucleic acid that encodes the polypeptide is provided. In some aspects, the nucleic acid molecule may also encode a leader sequence that directs secretion of the polypeptide that binds IL31, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some aspects, a nucleic acid molecule is an expression vector that is suitable for expression in a selected host cell. In some aspects, a vector comprising the nucleic acid is provided.

Vectors comprising nucleic acids that encode the polypeptide that binds IL31 described herein are provided. Such vectors include, but are not limited to, DNA vectors, phage vectors, viral vectors, retroviral vectors, etc. In some aspects, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In some aspects, an in vitro or ex vivo cell comprising the described nucleic acid is provided herein. In some aspects, an in vitro or ex vivo cell that expresses the polypeptide is provided herein. In some aspects, the in vitro or ex vivo cells express chimeric antigen receptor, T cell receptor, or any combination thereof.

In some aspects, a polypeptide that binds IL31 may be expressed in host cells, e.g., prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44, Lec13 CHO cells, and FUT8 CHO cells; PER.C6® cells (Crucell); and NSO cells. In some aspects, the polypeptide that binds IL31 may be expressed in yeast. See, e.g., U.S. Publication No. US 2006/0270045 A1. In some aspects, a particular eukaryotic host cell is selected based on its ability to make desired post-translational modifications to the polypeptide. For example, in some aspects, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

Host cells comprising any of the nucleic acids or vectors described herein are also provided. In some aspects, a host cell that expresses a polypeptide that binds IL31 described herein (e.g., Section II and the Examples) is provided. The IL31-binding polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify the polypeptide that binds IL31 that comprises an Fc region. Hydrophobic interactive chromatography, for example, a butyl or phenyl column, may also be suitable for purifying some polypeptides such as antibodies. Ion exchange chromatography (for example anion exchange chromatography and/or cation exchange chromatography) may also be suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also be suitable for purifying some polypeptides such as antibodies. Many methods of purifying polypeptides are known in the art.

Also provided is a host cell comprising a nucleic acid encoding any of the polypeptides described herein. Suitable host cells for cloning or expression of polypeptide-encoding vectors include prokaryotic or eukaryotic cells described herein. For example, the polypeptide may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254, describing expression of antibody fragments in E. coli.) After expression, the polypeptide may be isolated from the bacterial cell paste in a soluble fraction and can be further purified.

In some aspects, provided herein is a host cell comprising a vector comprising a nucleic acid(s) encoding a polypeptide of the present disclosure.

Suitable host cells for the expression of glycosylated polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al, J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3 A); human lung cells (W138); human liver cells (Hep G2); mouse mammary tumor (MMT 060562); TRI cells, as described, e.g., in Mather et al, Annals N Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al, Proc. Natl. Acad. Sci. USA 77:4216 (1980)); and myeloma cell lines such as Y0, NSO and Sp2/0. For a review of certain mammalian host cell lines suitable for polypeptide production, see, e.g., Yazaki and Wu, Methods in Molecular Biology, Vol. 248 (B.K.C. Lo, ed., Humana Press, Totowa, NJ), pp. 255-268 (2003).

In some aspects, the polypeptide that binds IL31 is produced in a cell-free system. Nonlimiting exemplary cell-free systems are described, for example, in Sitaraman et al., Methods Mol. Biol. 498:229-44 (2009); Spirin, Trends Biotechnol. 22:538-45 (2004); Endo et al., Biotechnol. Adv. 21:695-713 (2003).

In some aspects, a method of producing the polypeptide described herein (e.g., Section II and the Examples) is provided. In some aspects, the method comprises culturing a host cell under conditions suitable for expression of the polypeptide. In some aspects, the method further comprises isolating the polypeptide.

In some aspects, a polypeptide that binds IL31 is prepared by the methods described above. In some aspects, the polypeptide that binds IL31 is prepared in a host cell. In some aspects, the polypeptide that binds IL31 is prepared in a cell-free system. In some aspects, the polypeptide that binds IL31 is purified.

### IV. Exemplary Pharmaceutical Compositions

Pharmaceutical compositions comprising a polypeptide that binds IL31 are provided. In some aspects, the polypeptide that binds IL31 is any one of the polypeptides described herein (e.g., Section II and the Examples). The pharmaceutical compositions can additionally contain other therapeutic agents that are suitable for treating or preventing a given skin disease or disorder (e.g., see Section V). In some aspects, the pharmaceutical composition further comprises a pharmaceutically acceptable carrier.

Accordingly, described herein are pharmaceutical compositions comprising a polypeptide that binds IL31. In some aspects, the polypeptide that binds IL31 comprises one or more IL31 binder. In some aspects, the one or more IL31 binder comprises a VHH1. In some aspects, the one or more IL31 binder comprises a VHH1 and a VHH2. In some aspects, the VHH1 or the VHH2 comprises a CDR1 comprising the amino acid sequence of SEQ ID NO: 59; a CDR2 comprising the amino acid sequence of SEQ ID NO:60 or SEQ ID NO: 63; and a CDR3 comprising the amino acid sequence of SEQ ID NO:61 or SEQ ID NO: 64. In some aspects, the antibody variable domain is a VHH domain. In some aspects, the two or more antibody variable domains are VHH domains. In some aspects, the polypeptide that binds IL31 comprises a first IL31 binder and a second IL31 binder. In some aspects, the first IL31 binder comprises a VHH1 comprising a CDR1 comprising the amino acid sequence of SEQ ID NO: 5, SEQ ID NO: 19, SEQ ID NO: 33, SEQ ID NO: 47, SEQ ID NO: 78, SEQ ID NO: 81, or SEQ ID NO: 59; a CDR2 comprising the amino acid sequence of SEQ ID NO: 6, SEQ ID NO: 20, SEQ ID NO: 34, SEQ ID NO: 48, SEQ ID NO: 79, SEQ ID NO: 82, SEQ ID NO: 60, or SEQ ID NO:63; and a CDR3 comprising the amino acid sequence of SEQ ID NO: 7, SEQ ID NO: 21, SEQ ID NO: 35, SEQ ID NO: 49, SEQ ID NO: 80, SEQ ID NO: 83, SEQ ID NO: 61, or SEQ ID NO:64. In some aspects, the second IL31 binder comprises a VHH2 comprising a CDR1 comprising the amino acid sequence of SEQ ID NO: 10, SEQ ID NO: 24, SEQ ID NO: 38, SEQ ID NO: 52, SEQ ID NO: 84, SEQ ID NO: 87, SEQ ID NO:59, or SEQ ID NO:251; a CDR2 comprising the amino acid sequence of SEQ ID NO: 11, SEQ ID NO: 25, SEQ ID NO: 39, SEQ ID NO: 53, SEQ ID NO: 85, SEQ ID NO: 88, SEQ ID NO: 60, SEQ ID NO:63, or SEQ ID NO:252; and a CDR3 comprising the amino acid sequence of SEQ ID NO: 12, SEQ ID NO: 26, SEQ ID NO: 40, SEQ ID NO: 54, SEQ ID NO: 86, SEQ ID NO: 89, SEQ ID NO:61, SEQ ID NO:64, or SEQ ID NO:253.

In some aspects, the pharmaceutical composition comprises a polypeptide that binds IL31, wherein (i) the VHH1 comprises a CDR1 comprising the amino acid sequence of SEQ ID NO: 59, a CDR2 comprising the amino acid sequence of SEQ ID NO: 60, or SEQ ID NO:63, and a CDR3 comprising the amino acid sequence of SEQ ID NO: 61, or SEQ ID NO:64; and (ii) the VHH2 comprises a CDR1 comprising the amino acid sequence of SEQ ID NO: 10, SEQ ID NO: 24, SEQ ID NO: 38, SEQ ID NO: 52, SEQ ID NO: 84, SEQ ID NO: 87, SEQ ID NO:59, or SEQ ID NO: 251, a CDR2 comprising the amino acid sequence of SEQ ID NO: 11, SEQ ID NO: 25, SEQ ID NO: 39, SEQ ID NO: 53, SEQ ID NO: 85, SEQ ID NO: 88, SEQ ID NO:60, SEQ ID NO: 63, or SEQ ID NO:252, and a CDR3 comprising the amino acid sequence of SEQ ID NO: 12, SEQ ID NO: 26, SEQ ID NO: 40, SEQ ID NO: 54, SEQ ID NO: 86, SEQ ID NO: 89, SEQ ID NO: 61, SEQ ID NO:64, or SEQ ID NO:253.

In some aspects, the pharmaceutical composition comprises a polypeptide that binds IL31, wherein (i) the VHH1 comprises an amino acid sequence at least 85%, at least 90%, at least 95%, at least 96%, at least 97%, at least 98%, at least 99%, or 100% identical to the amino acid sequence of SEQ ID NO: 3, SEQ ID NO: 17, SEQ ID NO: 31, or SEQ ID NO: 45; and/or (ii) the VHH2 comprises an amino acid sequence at least 85%, at least 90%, at least 95%, at least 96%, at least 97%, at least 98%, at least 99%, or 100% identical to the amino acid sequence of SEQ ID NO: 8, SEQ ID NO: 22, SEQ ID NO: 36, or SEQ ID NO: 50.

In some aspects, the pharmaceutical composition comprises a polypeptide that binds IL31, wherein (i) the VHH1 comprises an amino acid sequence at least 85%, at least 90%, at least 95%, at least 96%, at least 97%, at least 98%, at least 99%, or 100% identical to the amino acid sequence of SEQ ID NO: 4, SEQ ID NO: 18, SEQ ID NO: 32, or SEQ ID NO: 46; and/or (ii) the VHH2 comprises an amino acid sequence at least 85%, at least 90%, at least 95%, at least 96%, at least 97%, at least 98%, at least 99%, or 100% identical to the amino acid sequence of SEQ ID NO: 9, SEQ ID NO: 23, SEQ ID NO: 37, or SEQ ID NO: 51.

In some aspects, the pharmaceutical composition comprises a polypeptide that binds IL31, wherein the polypeptide comprises an amino acid sequence at least 85%, at least 90%, at least 95%, at least 96%, at least 97%, at least 98%, at least 99%, or 100% identical to the amino acid sequence of SEQ ID NO: 13, SEQ ID NO: 27, SEQ ID NO: 41, SEQ ID NO: 55, SEQ ID NO: 14, SEQ ID NO: 28, SEQ ID NO: 42, or SEQ ID NO: 56.

In some aspects, the pharmaceutical composition comprises a polypeptide that binds IL31, wherein the polypeptide comprises a Fc region, wherein the Fc region comprises an amino acid sequence at least 85%, at least 90%, at least 95%, at least 96%, at least 97%, at least 98%, at least 99%, or 100% identical to the amino acid sequence of SEQ ID NO: 62 or the amino acid residues 1-231 of SEQ ID NO: 62.

In some aspects, the pharmaceutical composition comprises a polypeptide that binds IL31, wherein the polypeptide comprises a Fc region, wherein the Fc region comprises an amino acid sequence at least 85%, at least 90%, at least 95%, at least 96%, at least 97%, at least 98%, at least 99%, or 100% identical to the amino acid sequence of SEQ ID NO: 90 or the amino acid residues 1-231 of SEQ ID NO: 90.

In some aspects, the pharmaceutical composition comprises a polypeptide that binds IL31, wherein the polypeptide comprises an amino acid sequence at least 85%, at least 90%, at least 95%, at least 96%, at least 97%, at least 98%, at least 99%, or 100% identical to the amino acid sequence of SEQ ID NO: 15 or the amino acid residues 1-506 of SEQ ID NO: 15, SEQ ID NO: 29 or the amino acid residues 1-496 of SEQ ID NO: 29, SEQ ID NO: 43 or the amino acid residues 1-487 of SEQ ID NO: 43, SEQ ID NO: 57 or the amino acid residues 1-507 of SEQ ID NO: 57, SEQ ID NO: 91 or the amino acid residues 1-506 of SEQ ID NO: 91, SEQ ID NO: 16 or the amino acid residues 1-506 of SEQ ID NO: 16, SEQ ID NO: 30 or the amino acid residues 1-496 of SEQ ID NO: 30, SEQ ID NO: 44 or the amino acid residues 1-487 of SEQ ID NO: 44, SEQ ID NO: 58 or the amino acid residues 1-507 of SEQ ID NO: 58, SEQ ID NO: 92 or the amino acid residues 1-506 of SEQ ID NO: 92.

A pharmaceutical composition of the present disclosure can be administered by a variety of methods known in the art. The route and/or mode of administration vary depending upon the desired results. In some aspects, the pharmaceutical composition is administered parenterally, intramuscularly, intraperitoneally, intravenously, or subcutaneously. In some aspects, the pharmaceutical composition is administered proximal to the site of the target. The pharmaceutically acceptable carrier should be suitable for parenterally, intramuscular, intraperitoneal, intravenous, subcutaneous, parenteral, spinal, or epidermal administration (e.g., by injection or infusion).

Pharmaceutical compositions can be manufactured under GMP conditions. Typically, a therapeutically effective dose or efficacious dose of the polypeptide that binds IL31 is employed in the pharmaceutical compositions of the disclosure. The polypeptide that bind to IL31 can be formulated into pharmaceutically acceptable dosage forms. Dosage regimens are adjusted to provide a desired response (e.g., a therapeutic response).

### V. Exemplary Methods of Using IL31 Polypeptides

### A. Exemplary Uses and Methods of Treatment

Provided herein are polypeptides that bind IL31 (e.g., VHH) that may have therapeutic effects against chronic inflammatory skin diseases such as atopic dermatitis (AD) and prurigo nodularis (PN), which lead to intensely pruritic skin lesions resulting in severe scratching and can be extremely disabling (e.g., major psychological problems, significant sleep loss, and impaired quality of life (QOL) that lead to a high socioeconomic cost). In some instances, the polypeptides that bind IL31 may be used to treat a subject having an IL31-associated condition, such as a disease or disorder (e.g., inflammatory skin disease). In some instances, the methods or uses provided herein are for treating a subject having an IL31-associated condition comprising administering to the subject a therapeutically effective amount of any one the polypeptides or multimeric polypeptides described herein (e.g., Section II and the Examples) or the pharmaceutical composition thereof (e.g., Section IV). Uses of the polypeptide that binds IL31, multimeric polypeptides, or pharmaceutical compositions thereof for the manufacture of a medicament, wherein the medicament is for treating a subject having an IL31-associated condition, such as an inflammatory skin disease (e.g., a pruritic condition) are provided herein. Also provided herein are polypeptides that bind IL31, multimeric polypeptides, or pharmaceutical compositions thereof for use in treating a subject having an IL31-associated condition, such as an inflammatory skin disease (e.g., a pruritic condition).

In some aspects, provided herein is a method for treating a subject having an IL31-associated condition, comprising administering to the subject a therapeutically effective amount of any of the polypeptides, multimeric polypeptides, or pharmaceutical compositions thereof disclosed herein.

In some aspects, provided herein is a use of any of the polypeptides, multimeric polypeptides, or pharmaceutical compositions thereof disclosed herein for treating a subject having an IL31-associated condition.

In some aspects, provided herein is a use of any of the polypeptides, multimeric polypeptides, or pharmaceutical compositions thereof disclosed herein for the manufacture of a medicament, wherein the medicament is for treating a subject having an IL31-associated condition.

In some aspects, the method comprises uses of a polypeptide that binds IL31. In some aspects, the polypeptide that binds IL31 comprises one or more IL31 binder. In some aspects, the one or more IL31 binder comprises a VHH1. In some aspects, the one or more IL31 binder comprises a VHH1 and a VHH2. In some aspects, the VHH1 or the VHH2 comprises a CDR1 comprising the amino acid sequence of SEQ ID NO: 59 or SEQ ID NO:251; a CDR2 comprising the amino acid sequence of SEQ ID NO:60, SEQ ID NO: 63, or SEQ ID NO:252; and a CDR3 comprising the amino acid sequence of SEQ ID NO:61, SEQ ID NO: 64, or SEQ ID NO:253.

In some aspects, the one or more IL31 binder comprises a VHH1 and optionally a VHH2, wherein the VHH1 comprises a CDR1 comprising the amino acid sequence of SEQ ID NO: 59; a CDR2 comprising the amino acid sequence of SEQ ID NO: 60 or SEQ ID NO: 63; and a CDR3 comprising the amino acid sequence of SEQ ID NO: 61 or SEQ ID NO: 64. In some aspects, the VHH1 comprises a CDR1 comprising the amino acid sequence of SEQ ID NO: 59; a CDR2 comprising the amino acid sequence of SEQ ID NO: 63; and a CDR3 comprising the amino acid sequence of SEQ ID NO: 61. In some aspects, the VHH1 comprises a CDR1 comprising the amino acid sequence of SEQ ID NO: 59; a CDR2 comprising the amino acid sequence of SEQ ID NO: 60; and a CDR3 comprising the amino acid sequence of SEQ ID NO: 61. In some aspects, the VHH1 comprises a CDR1 comprising the amino acid sequence of SEQ ID NO: 59; a CDR2 comprising the amino acid sequence of SEQ ID NO: 60; and a CDR3 comprising the amino acid sequence of SEQ ID NO: 64. In some aspects, the VHH1 comprises a CDR1 comprising the amino acid sequence of SEQ ID NO:59; a CDR2 comprising the amino acid sequence of SEQ ID NO:63; and a CDR3 comprising the amino acid sequence of SEQ ID NO:64.

In some aspects, the polypeptide that binds IL31 comprises a first IL31 binder and a second IL31 binder. In some aspects, the first IL31 binder comprises a VHH1 comprising a CDR1 comprising the amino acid sequence of SEQ ID NO: 5, SEQ ID NO: 19, SEQ ID NO: 33, SEQ ID NO: 47, SEQ ID NO: 78, SEQ ID NO: 81, SEQ ID NO: 59, SEQ ID NO:268, SEQ ID NO:276, SEQ ID NO:284, SEQ ID NO:292, SEQ ID NO:300, SEQ ID NO:308, SEQ ID NO:316, SEQ ID NO: 324, SEQ ID NO:332, or SEQ ID NO:340; a CDR2 comprising the amino acid sequence of SEQ ID NO: 6, SEQ ID NO: 20, SEQ ID NO: 34, SEQ ID NO: 48, SEQ ID NO: 79, SEQ ID NO: 82, SEQ ID NO: 60, SEQ ID NO:63, SEQ ID NO:269, SEQ ID NO:277, SEQ ID NO:285, SEQ ID NO: 293, SEQ ID NO:301, SEQ ID NO:309, SEQ ID NO:317, SEQ ID NO:325, SEQ ID NO: 333, or SEQ ID NO:341; and a CDR3 comprising the amino acid sequence of SEQ ID NO: 7, SEQ ID NO: 21, SEQ ID NO: 35, SEQ ID NO: 49, SEQ ID NO: 80, SEQ ID NO: 83, SEQ ID NO: 61, SEQ ID NO:64, SEQ ID NO:270, SEQ ID NO:278, SEQ ID NO:286, SEQ ID NO:294, SEQ ID NO: 302, SEQ ID NO:310, SEQ ID NO:318, SEQ ID NO:326, SEQ ID NO:334, or SEQ ID NO: 342. In some aspects, the second IL31 binder comprises a VHH2 comprising a CDR1 comprising the amino acid sequence of SEQ ID NO: 10, SEQ ID NO: 24, SEQ ID NO: 38, SEQ ID NO: 52, SEQ ID NO: 84, SEQ ID NO: 87, SEQ ID NO:59, SEQ ID NO:251, SEQ ID NO:272, SEQ ID NO:280, SEQ ID NO:288, SEQ ID NO:296, SEQ ID NO:304, SEQ ID NO:312, SEQ ID NO: 320, SEQ ID NO:328, SEQ ID NO:336, or SEQ ID NO:344; a CDR2 comprising the amino acid sequence of SEQ ID NO: 11, SEQ ID NO: 25, SEQ ID NO: 39, SEQ ID NO: 53, SEQ ID NO: 85, SEQ ID NO: 88, SEQ ID NO:60, SEQ ID NO:63, SEQ ID NO:252, SEQ ID NO: SEQ ID NO: 273, SEQ ID NO:281, SEQ ID NO:289, SEQ ID NO:297, SEQ ID NO:305, SEQ ID NO:313, SEQ ID NO:321, SEQ ID NO:329, SEQ ID NO:337, or SEQ ID NO:345; and a CDR3 comprising the amino acid sequence of SEQ ID NO: 12, SEQ ID NO: 26, SEQ ID NO: 40, SEQ ID NO: 54, SEQ ID NO: 86, SEQ ID NO: 89, SEQ ID NO:61, SEQ ID NO:64, SEQ ID NO:253, or SEQ ID NO: 274, SEQ ID NO:282, SEQ ID NO:290, SEQ ID NO:298, SEQ ID NO:306, SEQ ID NO:314, SEQ ID NO:322, SEQ ID NO:330, SEQ ID NO:338, or SEQ ID NO:346.

In some aspects, the method comprises use of a polypeptide that binds IL31, wherein (i) the VHH1 comprises a CDR1 comprising the amino acid sequence of SEQ ID NO: 59, a CDR2 comprising the amino acid sequence of SEQ ID NO: 60 or SEQ ID NO:63, and a CDR3 comprising the amino acid sequence of SEQ ID NO: 61 or SEQ ID NO:64; and (ii) the VHH2 comprises a CDR1 comprising the amino acid sequence of SEQ ID NO: 10, SEQ ID NO: 24, SEQ ID NO: 38, SEQ ID NO: 52, SEQ ID NO: 84, SEQ ID NO: 87, SEQ ID NO:59, or SEQ ID NO:251, a CDR2 comprising the amino acid sequence of SEQ ID NO: 11, SEQ ID NO: 25, SEQ ID NO: 39, SEQ ID NO: 53, SEQ ID NO: 85, SEQ ID NO: 88, SEQ ID NO: 60, SEQ ID NO:63, or SEQ ID NO:252, and a CDR3 comprising the amino acid sequence of SEQ ID NO: 12, SEQ ID NO: 26, SEQ ID NO: 40, SEQ ID NO: 54, SEQ ID NO: 86, SEQ ID NO: 89, SEQ ID NO:61, SEQ ID NO:64, or SEQ ID NO:253.

In some aspects, the method comprises use of a polypeptide that binds IL31, wherein (i) the VHH1 comprises an amino acid sequence at least 85%, at least 90%, at least 95%, at least 96%, at least 97%, at least 98%, at least 99%, or 100% identical to the amino acid sequence of SEQ ID NO: 3, SEQ ID NO: 17, SEQ ID NO: 31, or SEQ ID NO: 45; and/or (ii) the VHH2 comprises an amino acid sequence at least 85%, at least 90%, at least 95%, at least 96%, at least 97%, at least 98%, at least 99%, or 100% identical to the amino acid sequence of SEQ ID NO: 8, SEQ ID NO: 22, SEQ ID NO: 36, or SEQ ID NO: 50.

In some aspects, the method comprises use of a polypeptide that binds IL31, wherein (i) the VHH1 comprises an amino acid sequence at least 85%, at least 90%, at least 95%, at least 96%, at least 97%, at least 98%, at least 99%, or 100% identical to the amino acid sequence of SEQ ID NO: 4, SEQ ID NO: 18, SEQ ID NO: 32, or SEQ ID NO: 46; and/or (ii) the VHH2 comprises an amino acid sequence at least 85%, at least 90%, at least 95%, at least 96%, at least 97%, at least 98%, at least 99%, or 100% identical to the amino acid sequence of SEQ ID NO: 9, SEQ ID NO: 23, SEQ ID NO: 37, or SEQ ID NO: 51.

In some aspects, the method comprises use of a polypeptide that binds IL31, wherein the polypeptide comprises an amino acid sequence at least 85%, at least 90%, at least 95%, at least 96%, at least 97%, at least 98%, at least 99%, or 100% identical to the amino acid sequence of SEQ ID NO: 13, SEQ ID NO: 27, SEQ ID NO: 41, SEQ ID NO: 55, SEQ ID NO: 14, SEQ ID NO: 28, SEQ ID NO: 42, or SEQ ID NO: 56.

In some aspects, the method comprises use of a polypeptide that binds IL31, wherein the polypeptide comprises a Fc region, wherein the Fc region comprises an amino acid sequence at least 85%, at least 90%, at least 95%, at least 96%, at least 97%, at least 98%, at least 99%, or 100% identical to the amino acid sequence of SEQ ID NO: 62 or the amino acid residues 1-231 of SEQ ID NO: 62.

In some aspects, the method comprises use of a polypeptide that binds IL31, wherein the polypeptide comprises a Fc region, wherein the Fc region comprises an amino acid sequence at least 85%, at least 90%, at least 95%, at least 96%, at least 97%, at least 98%, at least 99%, or 100% identical to the amino acid sequence of SEQ ID NO: 90 or the amino acid residues 1-231 of SEQ ID NO: 90.

In some aspects, the method comprises use of a polypeptide that binds IL31, wherein the polypeptide comprises an amino acid sequence at least 85%, at least 90%, at least 95%, at least 96%, at least 97%, at least 98%, at least 99%, or 100% identical to the amino acid sequence of SEQ ID NO: 15 or the amino acid residues 1-506 of SEQ ID NO: 15, SEQ ID NO: 29 or the amino acid residues 1-496 of SEQ ID NO: 29, SEQ ID NO: 43 or the amino acid residues 1-487 of SEQ ID NO: 43, SEQ ID NO: 57 or the amino acid residues 1-507 of SEQ ID NO: 57, SEQ ID NO: 91 or the amino acid residues 1-506 of SEQ ID NO: 91, SEQ ID NO: 16 or the amino acid residues 1-506 of SEQ ID NO: 16, SEQ ID NO: 30 or the amino acid residues 1-496 of SEQ ID NO: 30, SEQ ID NO: 44 or the amino acid residues 1-487 of SEQ ID NO: 44, SEQ ID NO: 58 or the amino acid residues 1-507 of SEQ ID NO: 58, SEQ ID NO: 92 or the amino acid residues 1-506 of SEQ ID NO: 92.

In some aspects, the method comprises using a multimeric polypeptide, which comprises two or more of any one of the polypeptides or co-binders provided herein.

Exemplary inflammatory skin diseases include, but are not limited to, IL31-associated skin diseases, such as atopic dermatitis, prurigo nodularis, chronic spontaneous urticaria, chronic pruritus of unknown origin, psoriasis, and eczema. Rare skin disorders having similar features include, for example, epidermolysis bullosa, toxic epidermal necrolysis, and folliculitis. In other instances, skin diseases involving inflammation and/or immune system dysregulation can result in mobility issues, partial disability, and even death if left untreated. For example, psoriatic arthritis, diabetic skin ulcers, and melanoma are skin diseases requiring medical attention. In some instances, the polypeptide that binds IL31, multimeric polypeptide, or pharmaceutical composition thereof as described herein is used for treatment of atopic dermatitis, prurigo nodularis, chronic spontaneous urticaria, chronic pruritus of unknown origin, psoriasis, and eczema. In some instances, the polypeptide that binds IL31, multimeric polypeptide, or pharmaceutical composition thereof is used for treating any of the inflammatory skin diseases described herein.

In some aspects, the IL31-associated condition is a pruritic condition. Uses of a polypeptide that binds IL31, multimeric polypeptide, or pharmaceutical composition thereof to inhibit, reduce, prevent, or minimize the effects of a pruritic condition are described herein. In some aspects, the IL31-associated condition is atopic dermatitis (AD). In some aspects, the IL31-associated condition is prurigo nodularis (PN). In some aspects, the IL31-associated condition is chronic spontaneous urticaria. In some aspects, the IL31-associated condition is chronic pruritus of unknown origin. In some aspects, the IL31-associated condition is eczema. In some aspects, the IL31-associated condition is psoriasis. In some aspects, the IL31-associated condition is uremic pruritus (chronic kidney disease-associated pruritus). In some aspects, the IL31-associated condition is bile acid induced urticaria.

The polypeptide that binds IL31, multimeric polypeptide, or pharmaceutical composition thereof can be administered as needed to subjects in need thereof. Determination of the frequency of administration can be made by persons skilled in the art, such as an attending physician based on considerations of the condition being treated, age of the subject being treated, severity of the condition being treated, general state of health of the subject being treated and the like. In some aspects, the method comprises administering to a subject having an IL31-associated condition a single dose of the polypeptide that binds IL31, multimeric polypeptide, or pharmaceutical composition thereof. In some aspects, the method comprises administering to a subject having an IL31-associated condition multiple doses of the polypeptide that binds IL31, multimeric polypeptide, or pharmaceutical composition thereof.

In some aspects, the polypeptide that binds IL31, multimeric polypeptide, or pharmaceutical composition thereof is administered in a therapeutically effective amount for treating an inflammatory skin disease (e.g., a pruritic condition). In some aspects, the polypeptide that binds IL31, multimeric polypeptide, or pharmaceutical composition thereof is administered in a therapeutically effective amount for prophylactic treatment of an inflammatory skin disease (e.g., a pruritic condition). In some aspects, use of the polypeptide that binds IL31, multimeric polypeptide, or pharmaceutical compositions thereof for treating an inflammatory skin disease (e.g., a pruritic condition), including prophylactic treatment is described, wherein the polypeptide that binds IL31, multimeric polypeptide, or pharmaceutical compositions thereof is administered in a therapeutically effective amount.

In some aspects, the method and uses provided herein comprises administering the polypeptide, multimeric polypeptide, or the pharmaceutical composition in combination with one or more therapeutic agent. In some aspects, the one or more therapeutic agent is a drug substance useful in treating an inflammatory skin disease (e.g., a pruritic condition).

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

In some aspects, the one or more therapeutic agent comprises, for example, an antibody, a small molecule inhibitor, and/or a systemic immunosuppressant agent.

In some aspects, the methods and uses comprise administering a polypeptide that binds IL31, multimeric polypeptide, or a pharmaceutical composition thereof in combination with, for example, a topical corticosteroid, a calcineurin inhibitor, an anti-IL4RA antibody, an anti-IL13RA antibody, an anti-OSMR antibody, an anti-Ox40 antibody, an anti-Ox40L antibody, an anti-TSLP antibody an anti-IL17 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IL31 antibody, an anti-IgE antibody, an anti-CD11a antibody, anti-IL6R antibody, anti-α4-Intergrin antibody, an anti-IL12 antibody, an anti-IL1β antibody, an anti-BlyS antibody, an anti-CKIT antibody, an anti-MRGPRX2 antibody, an anti-Fc epsilon receptor (FcERI) antibody, an anti-SIGLEC-6 antibody, or an anti-SIGLEC-8 antibody. In some aspects, the methods and uses comprise administering a polypeptide that binds IL31 or a pharmaceutical composition thereof in combination with, for example, a small molecule inhibitor of IL4RA, IL13RA, IL13, IL31, OSMR, Ox40, Ox40L, TSLP, IL17, TNFα, CD20, CD19, CD25, IL4, IL23, IgE, CD11Δ, IL6R, a4-Intergrin, IL12, IL1β, BlyS, CKIT, MRGPRX2, Fc epsilon receptor (FcERI), SIGLEC-6, and/or SIGLEC-8. In some aspects, the methods and uses provided herein comprise administering a polypeptide that binds IL31 or a pharmaceutical composition thereof in combination with, for example, a STAT inhibitor, a JAK inhibitor, a PI3K inhibitor, an AKT inhibitor, a MAPK inhibitor, a MRGPRX2 inhibitor, a topical corticosteroid, a calcineurin inhibitor, and/or a fusion protein comprising a portion of CTLA-4 and an Fc region of an immunoglobulin, optionally wherein the fusion protein is abatacept or belatacept. In some aspects, the methods and uses comprise administering a polypeptide that binds IL31, multimeric polypeptide, or a pharmaceutical composition thereof in combination with, for example, a topical steroid or a calcineurin inhibitor. In some aspects, the methods and uses comprise administering a polypeptide that binds IL31, multimeric polypeptide, or a pharmaceutical composition thereof in combination with systemic immunosuppressant, for example, cyclosporine A, methotrexate, mycophenolate mofetil, azathioprine, an oral corticosteroid, and interferon-gamma.

A therapeutic agent of the disclosure may be mixed with the other drug substance in a fixed pharmaceutical composition, or it may be administered separately, before, simultaneously with or after the other drug substance. Accordingly, the disclosure includes a combination of an agent described herein with one or more therapeutic agent in the same or different pharmaceutical composition.

In some aspects, the methods, uses, or pharmaceutical compositions for use described herein are for treating an IL31-associated skin disease such as atopic dermatitis, prurigo nodularis, chronic spontaneous urticaria, chronic pruritus of unknown origin, psoriasis, eczema, epidermolysis bullosa, epidermolysis bullosa simplex, seborrheic dermatitis, rosacea, toxic epidermal necrolysis, Stevens Johnson syndrome, Lyell syndrome, erythema multiforme, necrobiosis lipoidica, peeling skin syndrome, ichthyosis, neurodermatitis, pemphigus, folliculitis, uremic pruritus (chronic kidney disease-associated pruritus), bile acid induced urticaria, psoriatic arthritis, diabetic skin ulcers, and/or melanoma.

In some aspects, the polypeptide that binds IL31, multimeric polypeptide, or pharmaceutical composition thereof can be administered in vivo by various routes, including, but not limited to, parenteral, intramuscular, intraperitoneal, intravenous, intra-arterial, or subcutaneous. The appropriate route of administration may be selected according to the intended application. In some aspects, the polypeptide, multimeric polypeptide, or the pharmaceutical composition is administered parenterally. In some aspects, the polypeptide, multimeric polypeptide, or the pharmaceutical composition is administered by a subcutaneous route. In some aspects, the polypeptide, multimeric polypeptide, or the pharmaceutical composition is administered by an intravenous route. In some aspects, the polypeptide, multimeric polypeptide, or the pharmaceutical composition is administered by an intramuscular route. In some aspects, the polypeptide, multimeric polypeptide, or the pharmaceutical composition is administered by an intraperitoneal route.

In some aspects, treatment according to the methods, uses, and pharmaceutical compositions for use provided herein results in improved skin health comprising reduced skin redness, smaller skin lesions, and reduced skin itching

In some instances, the subject is a mammal. In some instances, the subject is a human. Subjects treated by methods described herein may be infants, adults, or children.

### B. Exemplary Indications and Patient Populations

Disclosed methods, uses, or pharmaceutical compositions for use herein comprise treating an IL31-associated condition in a subject in need thereof. The subject may have one or more indication that may benefit from the application of any one of the polypeptides described herein or pharmaceutical compositions thereof.

In some aspects, the subject receiving treatment has one or more of an inflammatory skin disease, a pruritic condition, atopic dermatitis, prurigo nodularis, chronic spontaneous urticaria, chronic pruritus of unknown origin, psoriasis, eczema, epidermolysis bullosa, epidermolysis bullosa simplex, seborrheic dermatitis, rosacea, toxic epidermal necrolysis, Stevens Johnson syndrome, Lyell syndrome, erythema multiforme, necrobiosis lipoidica, peeling skin syndrome, ichthyosis, neurodermatitis, pemphigus, folliculitis, uremic pruritus (chronic kidney disease-associated pruritus), bile acid induced urticaria, psoriatic arthritis, diabetic skin ulcers, and/or melanoma.

In some aspects, the subject receiving treatment has an inflammatory skin disease. In some aspects, the subject receiving treatment has a pruritic condition. In some aspects, the subject receiving treatment has atopic dermatitis. In some aspects, the subject receiving treatment has prurigo nodularis. In some aspects, the subject receiving treatment has chronic spontaneous urticaria. In some aspects, the subject receiving treatment has chronic pruritus of unknown origin. In some aspects, the subject receiving treatment has psoriasis. In some aspects, the subject receiving treatment has eczema. In some aspects, the subject receiving treatment has epidermolysis bullosa. In some aspects, the subject receiving treatment has epidermolysis bullosa simplex. In some aspects, the subject receiving treatment has seborrheic dermatitis. In some aspects, the subject receiving treatment has rosacea. In some aspects, the subject receiving treatment has toxic epidermal necrolysis. In some aspects, the subject receiving treatment has Stevens Johnson syndrome. In some aspects, the subject receiving treatment has Lyell syndrome. In some aspects, the subject receiving treatment has erythema multiforme. In some aspects, the subject receiving treatment has Necrobiosis lipoidica. In some aspects, the subject receiving treatment has peeling skin syndrome. In some aspects, the subject receiving treatment has ichthyosis. In some aspects, the subject receiving treatment has neurodermatitis. In some aspects, the subject receiving treatment has pemphigus. In some aspects, the subject receiving treatment has folliculitis. In some aspects, the subject receiving treatment has uremic pruritus (chronic kidney disease-associated pruritus). In some aspects, the subject receiving treatment has bile acid induced urticaria. In some aspects, the subject receiving treatment has psoriatic arthritis. In some aspects, the subject receiving treatment has diabetic skin ulcers. In some aspects, the subject receiving treatment has melanoma. In some aspects, the subject receiving treatment has airway hyperresponsiveness. In some aspects, the subject receiving treatment has allergic asthma. In some aspects, the subject receiving treatment has allergic conjunctivitis. In some aspects, the subject receiving treatment has allergic contact dermatitis. In some aspects, the subject receiving treatment has allergic lung disease. In some aspects, the subject receiving treatment has allergic rhinitis. In some aspects, the subject receiving treatment has alopecia areata. In some aspects, the subject receiving treatment has Alzheimer's disease. In some aspects, the subject receiving treatment has aspirin-exacerbated respiratory disease. In some aspects, the subject receiving treatment has asthma. In some aspects, the subject receiving treatment has atopic keratoconjunctivitis. In some aspects, the subject receiving treatment has bronchial asthma. In some aspects, the subject receiving treatment has bullous pemphigoid. In some aspects, the subject receiving treatment has chronic hand eczema. In some aspects, the subject receiving treatment has chronic inducible urticaria. In some aspects, the subject receiving treatment has chronic obstructive pulmonary disease. In some aspects, the subject receiving treatment has chronic Rhinosinusitis with nasal polyposis. In some aspects, the subject receiving treatment has colitis. In some aspects, the subject receiving treatment has dermatitis. In some aspects, the subject receiving treatment has eosinophilic COPD. In some aspects, the subject receiving treatment has eosinophilic esophagitis. In some aspects, the subject receiving treatment has eosinophilic gastritis. In some aspects, the subject receiving treatment has eosinophilic duodenitis. In some aspects, the subject receiving treatment has a food allergy. In some aspects, the subject receiving treatment has goblet cell metaplasia. In some aspects, the subject receiving treatment has hepatic fibrosis. In some aspects, the subject receiving treatment has Hodgkin's disease. In some aspects, the subject receiving treatment has idiopathic pulmonary fibrosis. In some aspects, the subject receiving treatment has Netherton syndrome. In some aspects, the subject receiving treatment has progressive systemic sclerosis. In some aspects, the subject receiving treatment has rheumatoid arthritis. In some aspects, the subject receiving treatment has sinusitis. In some aspects, the subject receiving treatment has Sjogren's syndrome. In some aspects, the subject receiving treatment has systemic lupus erythematosus. In some aspects, the subject receiving treatment has systemic sclerosis. In some aspects, the subject receiving treatment has type 1 diabetes. In some aspects, the subject receiving treatment has ulcerative colitis. In some aspects, the subject receiving treatment has chronic cholestatic pruritus. In some aspects, the subject receiving treatment has chronic kidney disease-associated pruritus. In some aspects, the subject receiving treatment has psoriasis-related itch. In some aspects, the subject receiving treatment has cutaneous T-cell lymphoma. In some aspects, the subject receiving treatment has acne rosacea. In some aspects, the subject receiving treatment has acne vulgaris. In some aspects, the subject receiving treatment has pruritus. In some aspects, the subject receiving treatment has arthritis. In some aspects, the subject receiving treatment has check-point inhibitor induced pruritus. In some aspects, the subject receiving treatment has chronic urticaria. In some aspects, the subject receiving treatment has contact dermatitis. In some aspects, the subject receiving treatment has contact hypersensitivity. In some aspects, the subject receiving treatment has Crohn's disease. In some aspects, the subject receiving treatment has cutaneous (lichen) amyloidosis. In some aspects, the subject receiving treatment has dermatomyositis. In some aspects, the subject receiving treatment has a drug-induced allergic reactions. In some aspects, the subject receiving treatment has inflammatory bowel disease. In some aspects, the subject receiving treatment has intrahepatic cholestasis of pregnancy. In some aspects, the subject receiving treatment has itch associated with wound healing. In some aspects, the subject receiving treatment has an itch associated with a burn. In some aspects, the subject receiving treatment has an itch associated with a sunburn. In some aspects, the subject receiving treatment has lichen planus. In some aspects, the subject receiving treatment has metabolic dysfunction-associated (non-alcoholic) steatohepatitis. In some aspects, the subject receiving treatment has osteoarthritis. In some aspects, the subject receiving treatment has osteoporosis. In some aspects, the subject receiving treatment has pemphigus herpetiformis. In some aspects, the subject receiving treatment has porokeratosis. In some aspects, the subject receiving treatment has primary biliary cholangitis. In some aspects, the subject receiving treatment has primary sclerosing cholangitis. In some aspects, the subject receiving treatment has pruritus. In some aspects, the subject receiving treatment has pruritus associated with cutaneous T-cell lymphoma. In some aspects, the subject receiving treatment has scleroderma. In some aspects, the subject receiving treatment has skin-tropic viruses and viral associated pruritus. In some aspects, the subject receiving treatment has spondyloarthritis. In some aspects, the subject receiving treatment has stasis dermatitis. In some aspects, the subject receiving treatment has wound healing pruritus.

In some aspects, the subject receiving treatment is an adult. In some aspects, the subject receiving treatment is a child. In some aspects, the subject has an inflammatory skin condition but is otherwise healthy. In some aspects, the subject has one or more comorbidities.

### C. Exemplary Methods of Detecting IL31

Also provided herein is a method of detecting human IL31 in a biological sample. Methods provided herein are useful for the detection, diagnosis, and monitoring of an IL31-associated condition. The methods may be useful in determining whether the polypeptides or multimeric polypeptides described herein (e.g., Section II and the Examples) are an appropriate treatment for the subject.

In some aspects, the method comprises contacting the biological sample with any one of the polypeptides or multimeric polypeptides described herein (e.g., Section II and the Examples). In some aspects, the biological sample is a serum sample. In some aspects, the biological ample is contacted with the polypeptide or multimeric polypeptide described herein under conditions permissive for binding of the polypeptide or multimeric polypeptide to a human IL31. In some aspects, the method comprises detecting whether a complex is formed between the polypeptide or multimeric polypeptide and the human IL31 in the biological sample. In some aspects, the polypeptide that binds to IL31 or multimeric polypeptide is labelled with a detectable label. In some aspects, the detectable label comprises a radioisotope or radionuclide. In some aspects, the detectable label comprises ³H, ¹⁴C, ³²P, ³⁵S, ⁹⁰Y, ⁹⁹Tc, ¹¹¹ In, ¹²⁵I, ¹³¹I, ¹⁷⁷Lu, ¹⁶⁶Ho, or ¹⁵³Sm. In some aspects, the detectable label comprises chromogen. In some aspects, the detectable label comprises a fluorescent label. In some aspects, the detectable label comprises FITC, rhodamine, lanthanide phosphors, phycoerythrin, GFP, or BFP. In some aspects, the detectable label comprises an enzymatic label. In some aspects, the detectable label comprises horseradish peroxidase, luciferase, alkaline phosphatase or p-galactosidase. In some aspects, the detectable label comprises a chemiluminescent marker. In some aspects, the detectable label comprises a biotinyl group. In some aspects, the detectable label comprises a predetermined polypeptide epitope recognized by a secondary reporter. In some aspects, the detectable label comprises a leucine zipper pair sequence, a binding site for a secondary antibody, a metal binding domain, or an epitope tag. In some aspects, the detectable label comprises a magnetic agent. In some aspects, the detectable label comprises a gadolinium chelate. Methods of conjugating labels to an antibody are known in the art. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety. In some aspects, the polypeptide that binds IL31 or multimeric polypeptide need not be labeled, and the presence thereof can be detected using a labeled antibody which binds to the polypeptide or multimeric polypeptide. In some aspects, the method of detecting human IL31 human comprises direct and/or indirect detection via a detectable label and one or more detectable moieties. In some aspects, the detectable label is detected with a method known in the art for detecting specific antigen-binding interactions.

Various methods known in the art for detecting specific antigen-binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the polypeptide or multimeric polypeptide and is selected so as to meet the needs of various uses of the method which are often dictated by the availability of assay equipment and compatible immunoassay procedures. General techniques to be used in performing the various immunoassays noted above are known to those of ordinary skill in the art.

### VI. Exemplary Kits

Also provided are articles of manufacture and kits that include any of the polypeptides that bind to IL31 provided herein (e.g., Section II and the Examples) and suitable packaging. In some aspects, the disclosure includes a kit with (i) a polypeptide or multimeric polypeptide that binds IL31, and (ii) instructions for using the kit to administer the polypeptide or multimeric polypeptide to an individual. In some aspects, the disclosure includes a kit with (i) a pharmaceutical composition comprising a polypeptide or multimer polypeptide that binds IL31 (e.g., Section IV and the Examples), and (ii) instructions for using the kit to administer the pharmaceutical composition to an individual.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Instructions supplied in the kits of the disclosure are typically written instructions on a label or package insert (e.g., a paper sheet included in the kit), but machine-readable instructions (e.g., instructions carried on a magnetic or optical storage disk) are also acceptable. The kit may further comprise a description of selecting an individual suitable or treatment.

The following examples illustrate particular aspects of the disclosure and are not intended in any way to limit the disclosure.

### EXAMPLES

### Example 1. Anti-IL31 Single Binder Library Construction

To identify IL31-specific single binders (VHHs), two immunization campaigns were carried out. In campaign 1, 4 llamas were immunized with His-tagged human IL31. In campaign 2, His-tagged human IL31 was crosslinked to itself or to keyhole limpet hemocyanin (KLH) or bovine serum albumin (BSA). Self-crosslinked IL31, non-crosslinked IL31, and KLH/BSA crosslinked IL31 were immunized to two animals each. Following repeated injections, plasma samples were collected from the llamas and analyzed for the presence of anti-IL31 single binders via ELISA. Specifically, MAXISORP™ 96-well plates were coated with human IL31 protein followed by incubation with plasma, anti-camelid VHH cocktail, and HRP-conjugated secondary antibody.

From the whole blood of animals demonstrating the presence of anti-IL31 single binders, red blood cells were lysed, whole RNA was extracted from the white blood cell using QIAGEN® RNeasy, and cDNA was synthesized by RevertAid H Minus First Strand cDNA Synthesis Kit (Thermo Fisher Scientific). The anti-IL31 single binder library was generated from cDNA by PCR using Q5® Hot Start DNA polymerase (New England Biolabs). Additionally, 5′-HR arm and 3′-HR arm, which are from upstream and downstream sequences of gene insertion site of yeast surface display vector respectively, were constructed by PCR using Q5® Hot Start DNA polymerase. The anti-IL31 single binder library was extended at 5′ and 3′ ends with 5′-HR arm and 3′-HR arm by overlap extension PCR using Q5® Hot Start DNA polymerase, and its product was further amplified in a PCR using ONETAQ® DNA polymerase (New England Biolabs) (WO2022147463A2, WO2022147463A3). The final PCR product was electroporated into EBY100 strain Saccharomyces cerevisiae together with the linearized yeast surface display plasmid for homologous recombination (Benatuil et al., Protein Eng Des Sel. 2010 April;23 (4): 155-9; Wang, Protein Eng Des Sel. 2005 July;18 (7): 337-43). The anti-IL31 single binder transformed cells were cultured in SDCAA (Synthetic Dextrose medium supplemented with Casamino Acids) medium.

### Example 2. Campaign 1 Single Binder Library Selection

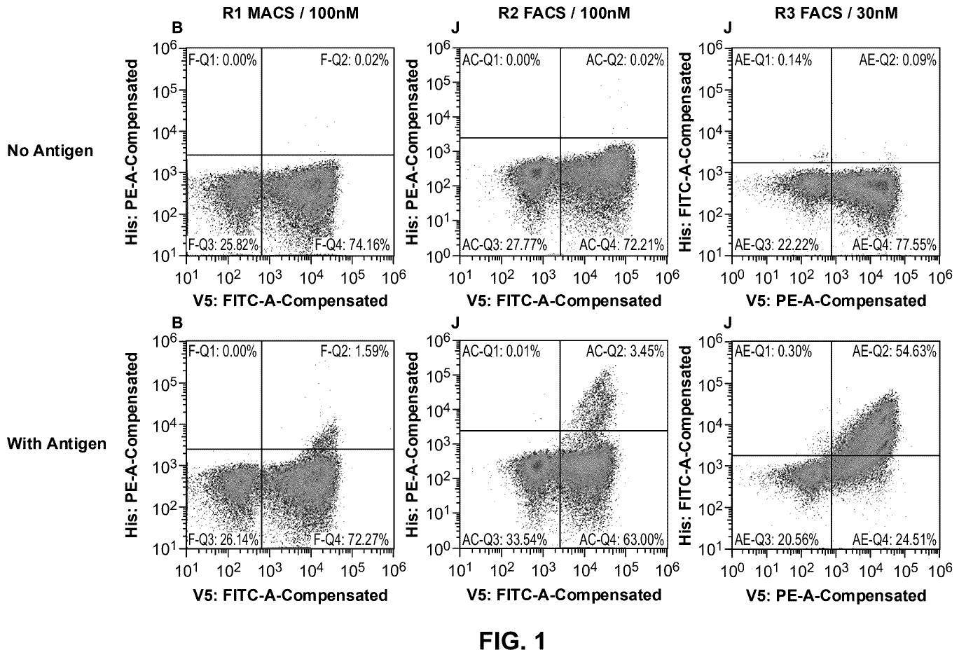

The anti-IL31 single binder yeast library was induced with 2% galactose (SGCAA, Synthetic Galactose medium supplemented with Casamino Acids) overnight. Library transformed cells were blocked in a blocking buffer (PBS containing 0.5% BSA). 100 nM His-tagged recombinant human IL31 was mixed with 2X mols (relative to His-tagged human IL31) of TrisNTA-Biotin and was used for binding the blocked cells. Cells were subsequently incubated with a mix of anti-biotin microbeads and streptavidin microbeads, and anti-IL31 displaying cells were enriched using magnetic activated cell sorting (MACS). Enrichment was confirmed via staining with phycoerythrin (PE) conjugated His antibody (Miltenyi Biotec, Inc. Auburn, CA) and fluorescein isothiocyanate (FITC) conjugated V5 antibody, followed by flow cytometric analysis (FIG. 1). Following MACS, anti-IL31 single binder displaying cells (second quadrant) were sorted via two consecutive rounds of FACS using 100 nM and 30 nM biotinylated human IL31, respectively. V5 tag is encoded downstream of single binder gene in the yeast surface display vector, and it is used to assess single binder expression. PE and FITC fluorophores were alternated between streptavidin and V5 antibody. Sorted cells were cultured in SDCAA medium for plasmid isolation.

For plasmid isolation, cultured cells were treated with Zymolyase, and plasmids were isolated using QIAGEN® Spin Miniprep Kits. Approximately 750 bases segment on the plasmid library covering anti-IL31 VHH and about 400 bases upstream segment of the anti-IL31 single binder was PCR amplified using Q5® Hot Start DNA polymerase with the first half of linker library sequences within reverse primer. Another 750 bases segment on the plasmid library covering anti-IL31 single binder and about 400 bases segment downstream of anti-IL31 single binder was PCR amplified using Q5® Hot Start DNA polymerase with the second half of linker library sequences within forward primer to generate VHH2. A linear co-binder library was constructed by GIBSON ASSEMBLY® between VHH1 and VHH2 (e.g., see WO2022147463 A2, WO2022147463A3). The assembled co-binder library was purified on a 1% agarose E-gel. The purified library was further amplified via PCR using ONETAQ® DNA polymerase. The amplified library was electroporated into EBY100 S. cerevisiae cells together with linearized yeast surface display plasmid, which has about 400 bases overlap at 5′ and 3′ ends with co-binder library DNA, for homologous recombination. Anti-IL31 co-binder transformed cells were cultured in SDCAA medium.

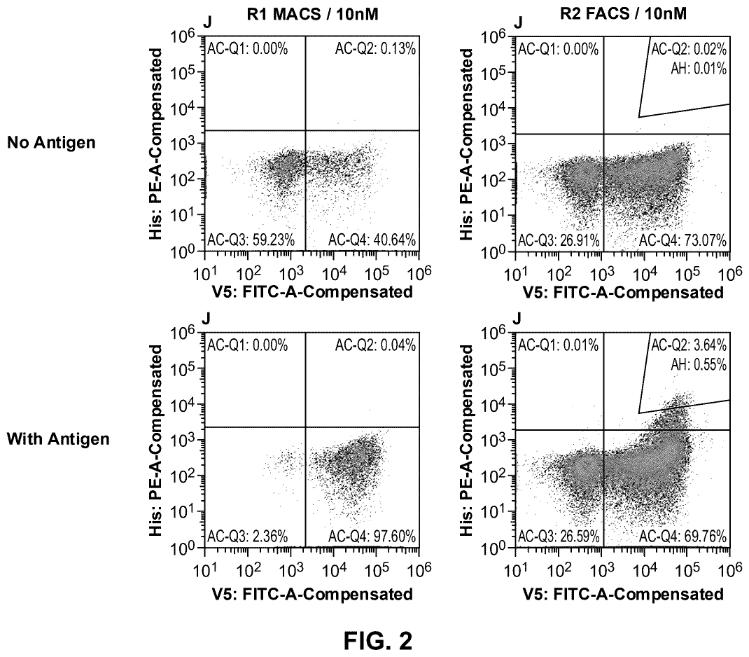

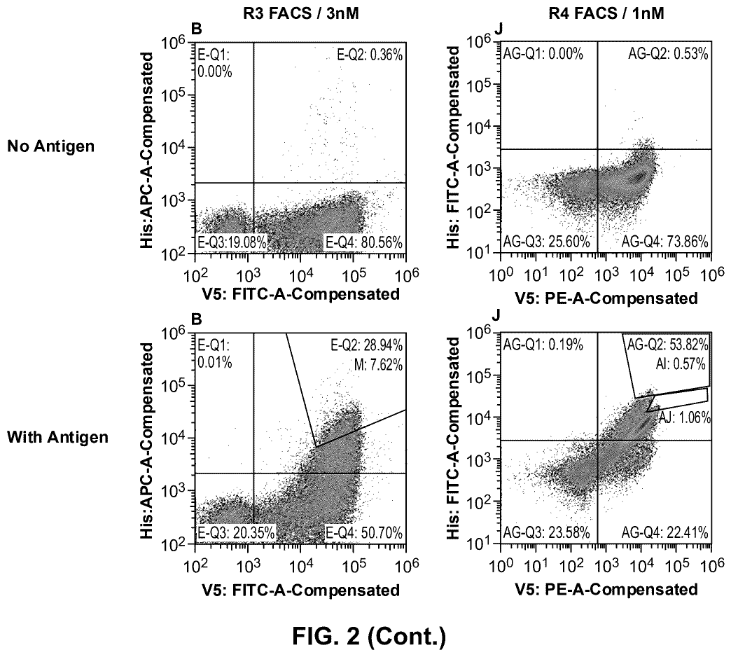

Anti-IL31 co-binder library-transformed yeast cells were induced with 2% galactose overnight. Induced yeast cells were blocked with blocking buffer as described above. His-tagged human IL31 was mixed with 2X concentration (relative to His-tagged human IL31) of Tris-NTA Biotin trifluoroacetate salt (Sigma-Aldrich, St. Louis, MO) and was used for incubating the blocked cells at final His-tagged human IL31 concentration of 10 nM in blocking buffer. After further incubation with mix of anti-biotin microbeads and streptavidin microbeads, anti-IL31 co-binder-displaying cells were enriched using MACS in round 1. Enrichment was confirmed via staining with PE conjugated His antibody (Miltenyi Biotec, Inc. Auburn, CA) and FITC conjugated V5 antibody, followed by flow cytometric analysis (FIG. 2). MACS-enriched cells were further sorted (AH gate in round 2 and M gate in round 3) via three consecutive rounds of FACS using 10 nM, 3 nM, and 1 nM his-tagged human IL31. In the fourth round of FACS, AI, a higher affinity gate, and AJ, a lower affinity gate, were separately sorted into 96-well plates with single cell each well. PE, FITC, and allophycocyanin (APC) were rotated as fluorophore for His antibody and V5 antibody. Colony PCR was performed with sorted cells and PCR products were used for sequencing.

### Example 3. Campaign 2 Single Binder Library Selection

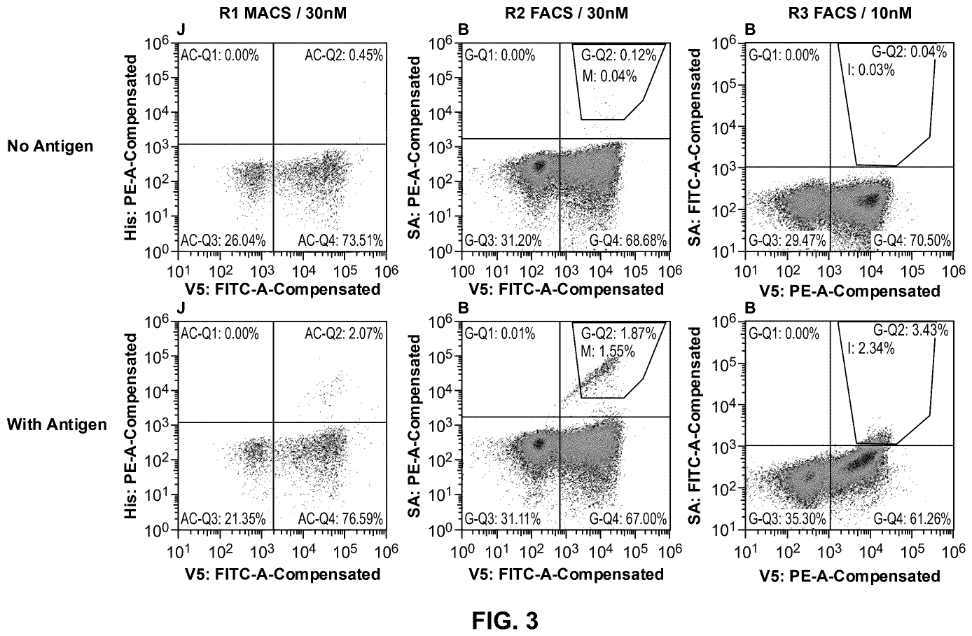

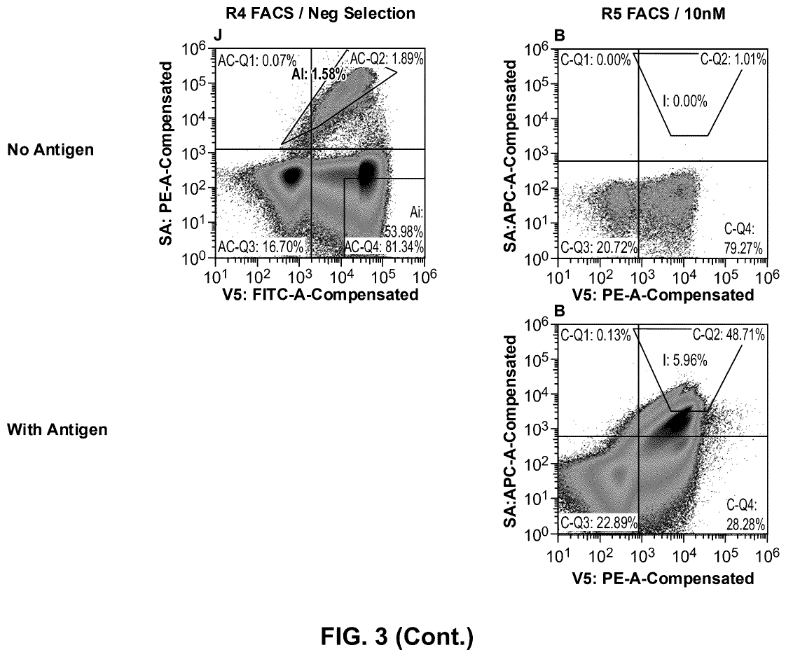

Campaign 2 derived yeast surface display IL31 single binder library was enriched through a single round (R1) of MACS using 30 nM His-tagged human IL31 following the method described above (FIG. 3). MACS-enriched cells were subsequently sorted through two rounds of positive selection (M and I gates for round 2 (R2) and round 3 (R3), respectively) via FACS with human IL31 at 30 nM and 10 nM. Round 3 sorted cells were negatively selected (AL gate) in round 4 (R4) after staining with PE conjugated streptavidin and FITC conjugated V5 antibody without human IL31 via FACS to reduce potential off-target binders. Finally, cells were sorted (I gate) in round 5 (R5) via FACS for binding to 10 nM human IL31, and sorted cells were cultured in SDCAA medium for plasmid isolation.

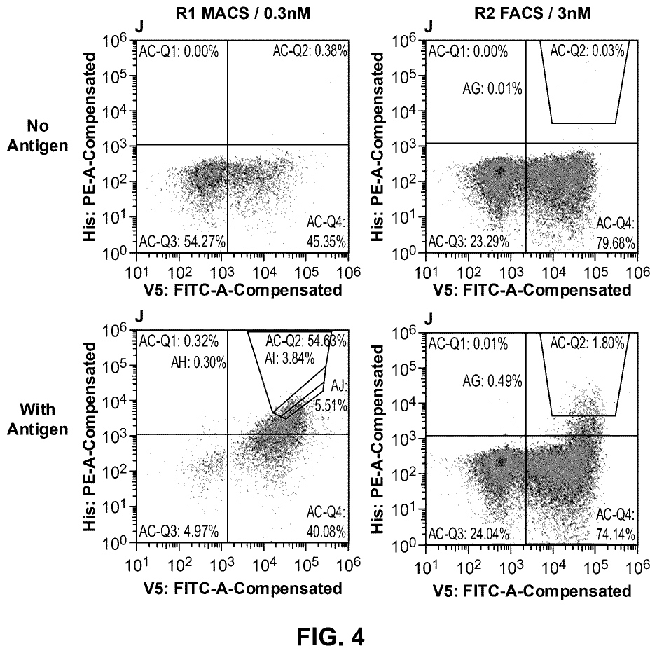

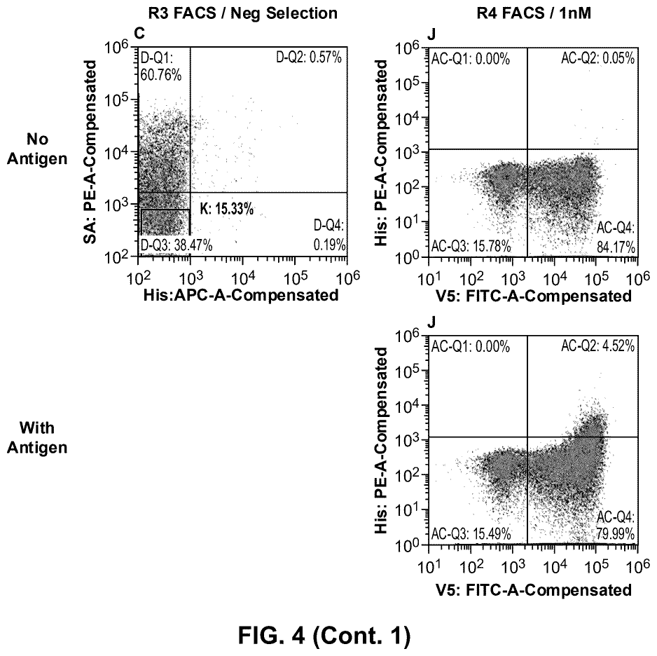

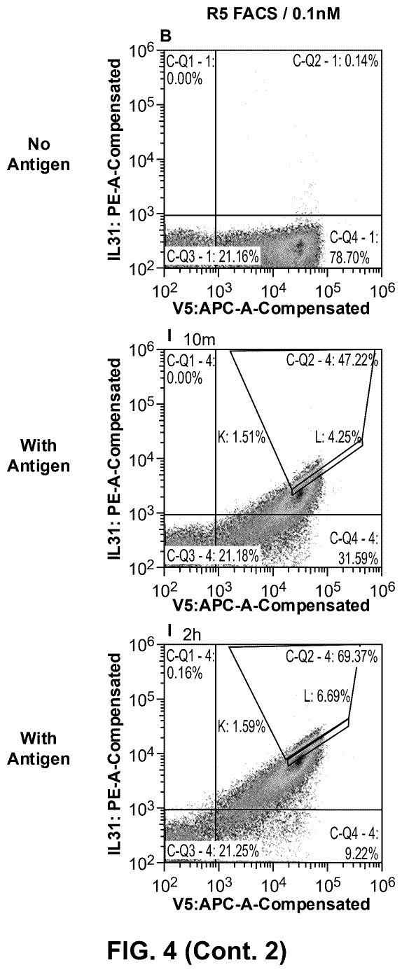

Plasmids from campaign 1 and campaign 2 were mixed and the co-binder library was constructed following the method described above. Anti-IL31 co-binder-displaying yeast cells were MACS enriched with human IL31 at 0.3 nM in blocking buffer in round 1 (R1) (FIG. 4). Cells were subsequently enriched further using FACS with 3 nM human IL31 in round 2 (R2). To reduce potential off-target binders, cells were then incubated with biotinylated polyspecificity reagent prepared from CHO-K1 cell lysate (Xu et al., Protein Eng Des Sel. 2013 October;26 (10): 663-70), PE conjugated streptavidin, APC conjugated His antibody, and FITC conjugated V5 antibody in blocking buffer. PE negative, APC negative, and FITC positive populations (K gate) were sorted in round 3 (R3), and anti-IL31 VHH-containing yeast cells were further enriched through two more rounds of FACS using 1 nM (round 4 (R4)) and 0.1 nM (round 5 (R5)) human IL31. Single cells with the higher affinity (K) and lower affinity (L) for IL31 were sorted into 96-well plates and colony PCR was performed with sorted cells and PCR products were used for sequencing.

### Example 4. Preliminary Assessments for Selection of Co-Binder Candidates

A total of 2006 co-binders from the two co-binder campaign libraries were sequenced and subjected to initial binding affinity testing. Fc fusions were prepared and subjected to one or more further assessments to determine affinity, potency, and developability, including polyspecificity, melting temperature, hydrophobicity, and thermal stability. As shown in Table 3, four co-binder candidates, 1C10, 1D1, 1H5, and 21H7 showed strong binding affinity and potency and desirable developability characteristics.

TABLE 3
Co-Binder Selection Characteristics

| | | | | | HIC | Thermal |
|---|---|---|---|---|---|---|
| | | | | | retention | Stability |
| | Activity | Affinity | Tm | BVP | time | (% Aggregation, |
| Clone | (IC50, nM) | (KD, pM) | (° C.) | binding | (min) | 2 d) |
| 1C10 | 8.5 ± 1.3 | 58.7 ± 50.5 | 66.5 | No | 16.36 | −0.3 |
| 1D1 | 6.7 ± 0.9 | 24 ± 29 | 60.7 | No | 15.93 | 0.2 |
| 1H5 | 6.1 ± 0.7 | 3.2 ± 1.3 | 58.7 | No | 15.24 | 0.9 |
| 21H7 | 8.4 | 14.8 | 61 | No | 15.76 | 0 |

Tm: melting temperature;
BVP: Baculovirus particle;
HIC: hydrophobic interaction chromatography

Binding affinity was measured using Biotin CAPture Kit (Cytiva Life Sciences) and the BIACORE™ T200 SPR system (Cytiva Life Sciences). Initially, Biotin CAPture Reagent (Cytiva Life Sciences) was flowed to immobilize 3 nM biotinylated IL31 (ligand). 3 nM, 9 nM, and 27 nM anti-IL31 co-binders were flowed as analyte with 180 seconds association and running buffer was flowed with 3000 seconds dissociation. The sensor chip was regenerated by guanidine hydrochloride and sodium hydroxide.

IC50 Assays (pSTAT assay and IL31 Dimerization Assay)

(1) pSTAT3 Assay. Candidate co-binders were screened by a cell-based potency assay that measured direct response of IL31 co-binders in A549 cells expressing IL31Rα. After binding to IL31Rα, IL31 activates JAK½ signaling molecules, which, in turn, activate phosphorylation of STAT3. A decrease in IL31 signaling is evidenced by a reduction in STAT3 phosphorylation as the concentration of IL31 co-binders exposed to the cells is increased. A549 cells were cultured in Ham's F12K+2 mM Glutamine+10% FBS at 37° C. with 5% CO₂. 24 hours before the experiment, A549 cells were detached and plated into 96-well plates at 2x10e4 cells/well in Ham's F12K+2 mM Glutamine+10% FBS and incubated overnight. The next day, the cell culture supernatant was removed from the plate and the cells were washed with 100 μl serum free medium followed by 1 hour serum starvation at 37° C. with 50 μl of serum free medium. Co-binders were diluted from 80 μg/ml with 2X dilution for 12 points and incubated with equal volume of 1 μg/ml IL-31 in serum free medium for 1 hour at room temperature. 50 μl of IL31 and co-binder complex were added to each well of the cell culture plate and incubated for 10 minutes at 37° C. The supernatant was then removed and the cells were lysed in 110 μl of lysis buffer. A pSTAT3 ELISA was performed using PATHSCAN® Phospho-Stat3 (Tyr705) Sandwich ELISA Kit #7300 (Cell signaling technologies, Danvers, MA).

(2) IL31 Dimerization Assay. IL-31 dimerization kits were purchased from Eurofins Scientific (Fremont, CA). The assay was performed according to the instructions. U2OS OSMRb/IL31R dimerization cells were seeded at 0.1 million/well in 80 μl assay complete cell plating 0 reagent medium into a 96-well Clear Flat Bottom White Plate and incubated at 37° C., 5% CO2 for overnight. Co-binders were diluted from 30 μg/ml with 3× dilution for 12 points and incubated with equal volume of 10 ng/ml IL-31 in assay complete cell plating 0 reagent for one hour at room temperature. 20 μl of IL31 and IL31 co-binders complex were added to each well of the cell culture plate and incubated for 16 hours. Plates and detection reagent were equilibrated to room temperature. 100 μl of detection reagent mix was added to each well of the plates and incubated at room temperature for 1 hour in the dark. RLU was read using VICTOR® Nivo™ Multimode Plate Reader.

Thermostability was also analyzed by differential scanning fluorimetry (DSF) according to the manufacturer's instructions using a CFX₉₀ RT-PCR instrument (Bio-Rad). In triplicate, co-binder samples (2.5 μg to 10 μg) were added to 5X final dye (INVITROGEN™ SYPRO™ Orange; Thermo Fisher Scientific) solution in 30 μL final volume. The hydrophobic dye binds to proteins as they unfold due to an increase in temperature. Temperature was increased from 10° C. to 95° C. in increments of 0.5° C. per second to develop a melting curve. Data was analyzed using CFX

Maestro Software (Bio-Rad) to find the first derivative, or Tm, of the melting curve. At Tm, 50% of co-binder is in native state and 50% is in denatured state. Tm≥55° C. was used as a minimal cutoff point to screen drug candidates.

To assess polyspecificity of co-binder candidates, baculovirus particle (BVP) binding was assessed by ELISA according to the manufacturer's protocol (MEDNA Scientific, Irving, TX). MAXISORP™ 96-well plates were coated with baculovirus particle in sodium carbonate buffer (pH9.6). Coated plates were blocked in blocking buffer (PBS with 0.5% BSA) and incubated with 1 μM samples. Horseradish peroxidase conjugated anti-human Fc was used for detection. Hits within ≤5x of the negative control (blank) were given a pass in the assay which is within the range of commercially viable mAb for polyspecificity.

Hydrophobicity was measured by hydrophobic interaction chromatography (HIC) using a published protocol (Jain et al., 2017 Proc Natl Acad Sci, 114 (5): 944-949.). This assay measures relative protein surface hydrophobicity which in turn can indicate protein tendency to self-associate under salt stress conditions. Co-binder samples (5 μg to 10 μg) were re-buffered to Buffer B (0.1 M sodium phosphate pH 6.5)+1M Ammonium Sulfate (AS) and then loaded on a 1.67 mL HPLC Proteomix HIC Butyl-NP5 column (4.6×10⁰ mm) at 0.75 mL/min. The column was equilibrated with Buffer A (0.1 M Sodium Phosphate pH 6.5+2 M AS). A linear gradient to 100% Buffer B was developed over 20 minutes. Buffer B concentration was held for 5 minutes at 100% before the column was re-equilibrated with buffer A. Detection was performed with a UV detector set at 280 nm. HIC retention time was measured for one minute.

HPLC Size Exclusion Chromatography (SEC) was used to assess thermal stability of co-binders as measured by percent aggregation after storage for two days at 40° C. Co-binder samples (10 μg) were loaded onto a ZENIX®-C SEC-300 gel filtration column (7.8×150 mm) (SEPAX Technologies). An isocratic gradient was performed with 150 mM Sodium Phosphate pH 7.0 according to the manufacturer's instructions (SEPAX Technologies) at 1 mL/min. Additionally, percent aggregates and percent monomer were measured (data not shown).

Heparin Chromatography was performed as an in vitro predictor for antibody and co-binder clearance rate through pinocytosis and was based on previous methods (Kraft et al., 2020, MAbs; 12 (1): 1683432-949). Co-binder protein samples (5 to 10 μg) were diluted at least 10-fold in 15 mM MES pH 5.5 and then loaded on a 1.7 mL HPLC POROS™ Heparin column (4.6×100 mm) (Thermo Fisher Scientific) at 1 mL/min. Buffer A is 50 mM Tris buffer pH 7.4 and Buffer B is Buffer A+1 M NaCl. After 4-minute post injection, a 55% B linear gradient was developed over 23 min. Buffer B concentration was increased to 100% and held for 2 minutes before the column was re-equilibrated with buffer A. Detection of co-binder protein level was performed with a UV detector set at 280 nm.

### Example 5. Sequencing and Alignment of Co-Binder Candidates

High-binder candidate co-binder clones were sequenced and decomposed into VHH1 and VHH2. VHH1 and VHH2 single binders exhibited low affinity in IL31 binding with Kds in the double digits to single digit nM range. In contrast, co-binder candidates were 100-1000-fold more potent. The bi-paratropic format of the co-binders led to significant synergy in binding.

Sequence alignments of candidate co-binder clones 1D1, 1H5, and 1C10 from the first campaign and 21H7 from the second campaign revealed similarities among the CDR sequences. In particular, the VHH1 CDRs of these candidates are very similar. In fact, the VHH1 CDRs of clone 1C10 from the first campaign and 21H7 from the second campaign are identical and the VHH1 CDRs of clone 1D1 and 1H5 from the first campaign are identical.

Table 1 provides amino acid sequences for clone 1C10 VHH1 single binder (SEQ ID NO: 3), 1C10 VHH2 single binder (SEQ ID NO: 8), and 1C10 co-binder (SEQ ID NO: 13); 1D1 VHH1 single binder (SEQ ID NO: 17), 1D1 VHH2 single binder (SEQ ID NO: 22), and 1D1 co-binder (SEQ ID NO: 27); clone 1H5 VHH1 single binder (SEQ ID NO: 31), 1H5 VHH2 single binder (SEQ ID NO: 36), and 1H5 co-binder (SEQ ID NO: 41); and clone 21H7 VHH1 single binder (SEQ ID NO: 45), 21H7 VHH2 single binder (SEQ ID NO: 50), and 21H7 co-binder (SEQ ID NO: 55). Also provided in Table 1 are clone 1C10 VHH1 CDR1, CDR2, and CDR3 (SEQ ID NOs: 5, 6, and 7, respectively), clone 1C10 VHH2 CDR1, CDR2, and CDR3 (SEQ ID NOs: 10, 11, and 12, respectively), clone 1D1 VHH1 CDR1, CDR2, and CDR3 (SEQ ID NOs: 19, 20, and 21, respectively), clone 1D1 VHH2 CDR1, CDR2, and CDR3 (SEQ ID NOs: 24, 25, and 26, respectively), clone 1H5 VHH1 CDR1, CDR2, and CDR3 (SEQ ID NOs: 33, 34, and 35, respectively), clone 1H5 VHH2 CDR1, CDR2, and CDR3 (SEQ ID NOs: 38, 39, and 40, respectively), clone 21H7 VHH1 CDR1, CDR2, and CDR3 (SEQ ID NOs: 47, 48, and 49, respectively), and clone 21H7 VHH2 CDR1, CDR2, and CDR3 (SEQ ID NOs: 52, 53, and 54).

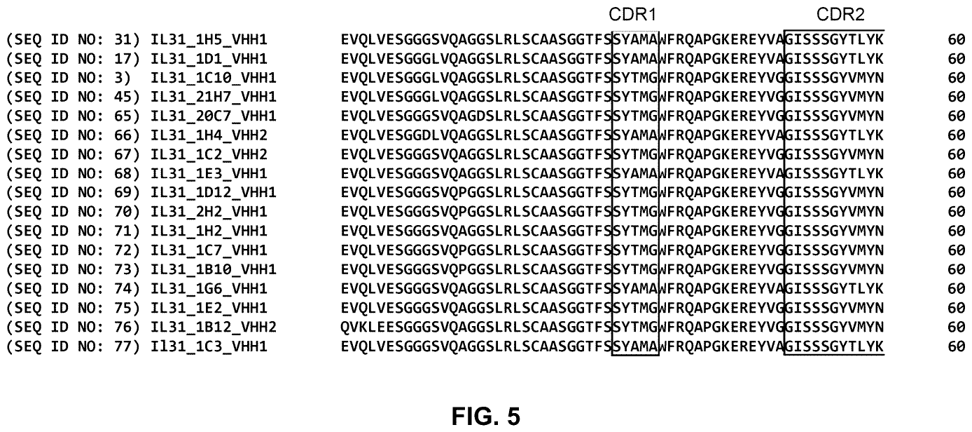

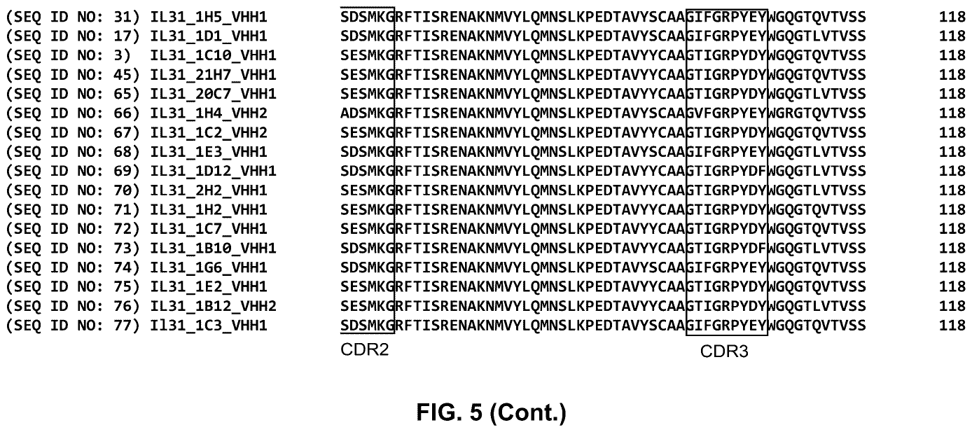

Interestingly, several other candidate clones had VHH sequences (either VHH1 or VHH2) similar or identical to the VHH1 sequences of the candidates 1C10, 1D1, 1H5, and 21H7. See FIG. 5. However, as shown in Table 4, below, while many of the candidate clones having the same or similar VHH sequences to the candidates 1C10, 1D1, 1H5, and 21H7 exhibited good binding affinity and/or potency, they did not exhibit favorable developability properties like 1C10, 1D1, 1H5, and 21H7. Only the unique co-binders 1C10, 1D1, 1H5, and 21H7 displayed desirable binding affinity, potency, and developability characteristics and were carried forward for further characterization and development.

TABLE 4
Co-Binder Selection Characteristics

| | Activity | Affinity | Tm | BVP | HIC retention | Thermal Stability |
|---|---|---|---|---|---|---|
| Clone | (IC50, nM) | (KD, pM) | (° C.) | binding | time (min) | (% Aggregation, 2 d) |
| 1H5 | 6.1 ± 0.7 | 3.2 ± 1.3 | 58.7 | No | 15.24 | 0.9 |
| | (P³) | | | | | |
| 1D1 | 6.7 ± 0.9 | 24 ± 29 | 60.7 | No | 15.93 | 0.2 |
| | (P³) | | | | | |
| 1C10 | 8.5 ± 1.3 | 58.7 ± 50.5 | 66.5 | No | 16.36 | −0.3 |
| | (P³) | | | | | |
| 21H7 | 8.4 (P³) | 14.8 | 61 | No | 15.76 | 0 |
| 20C7 | 9.8 (P³) | 27.1 | 58.5 | No | 18 | 0 |
| 1H4 | 9.1 ± 1.6 | 10.8 ± 9 | 53.5 | No | 16.15 | 49 |
| | (P³) | | | | | |
| 1C2 | 10.5 ± 0.8 | 109.9 ± 45.2^{##} | 66.5 | No | 17.9\hat{ } | 0 |
| | (P³) | | | | | |
| 1E3 | 12.1 ± 0.9 | 8.6 ± 4 | 58 | No | 15.32 | 0 |
| | (P³) | | | | | |
| 1D12 | 14.6 ± 0.2 | 152.3 ± 42.6^{##} | 60.5 | No | 18.09\hat{ } | 0 |
| | (P³) | | | | | |
| 2H2 | 24.3 ± 0.2^{#} | 97.7 ± 51.5 | 57.8 | No | 17.49 | 0 |
| | (P³) | | | | | |
| 1H2 | 34.9 ± 1.7^{#} | 3145 ± 3772.9^{##} | 60.5 | No | No peak | 5.6 |
| | (P³) | | | | | |
| 1C7 | 7.9 ± 1.0 | 202.3 ± 24.2^{##} | 62 | Yes* | 15.91 | 0 |
| | (P³) | | | | | |
| 1B10 | 12.6 ± 0.9 | 140.3 ± 12.9^{##} | 63.5 | Yes* | 18.06\hat{ } | 1.8 |
| | (P³) | | | | | |
| 1G6 | 13.4 ± 0.8 | 419.3 ± 9.3^{##} | 46.8** | Yes* | 17\hat{ } | 41.7 |
| | (P³) | | | | | |
| 1E2 | 18.1 ± 0.8 | 94.3 ± 12.4 | 64.3 | Yes* | 15.86 | 0 |
| | (P³) | | | | | |
| 1B12 | 19.8 ± 2.6 | 158.7 ± 8.3^{##} | 59 | Yes* | 16.18 | 5 |
| | (P³) | | | | | |
| 1C3 | 55.9 ± 1.6^{#} | 466.5 ± 285^{##} | 57.5 | Yes* | 18.42\hat{ } | 6.7 |
| | (P³) | | | | | |
| 21A4 | 0.121 (D) | 300 ± 140 | 57 | | 15.3 | 6 |
| 21C11 | 0.007 (D) | 1600 ± 100 | 55.5 | | 15.3 | 45 |
| 21C8 | 0.013 (D) | 10000 ± 210 | 59 | | 14.9 | 1.4 |
| 21B11 | 0.002 (D) | 42 ± 14 | 57.5 | | 14.7 + 15.7 | 36 |
| 21G5 | 0.011 (D) | 890 ± 110 | 58.5 | | 14.1 + 15.7 | 5.5 |
| 21G7 | 0.082 (D) | 2100 ± 120 | 55.5 | | 14.3 + 15.5 | 60 |
| 21H2 | 0.032 (D) | 71 ± 19 | 56.5 | | 15 + 16 + 18.6 | 18.6 |
| 19B5 | 0.039 (D) | 260 ± 3 | 58.5 | | 15.7 | 27.5 |
| 22D1 | 0.062 (D) | 280 ± 2.5 | 57.5 | | 13.8 + 16 | 10.2 |
| 22D8 | 0.006 (D) | 560 ± 35 | 59.5 | | 14.6 | 1.8 |

Tm: melting temperature;
BVP: baculovirus protein;
HIC: hydrophobic interaction chromatography
^{#}In vitro potency below the desired threshold;
^{##}Binding affinity below the desired threshold;
*Polyspecificity score above the desired threshold;
**Melting temperature below the desired threshold;
\hat{ }HIC retention time above the desired threshold
(P³) denotes pSTAT3 assay utilized for determining Activity (IC50)
(D) denotes IL31 Dimerization assay utilized for determining Activity (IC50)

### Example 6. Further Characterization of Co-Binders

Large-scale expression of 1H5, 1D1, 1C10, and 21H7 showed co-binders exhibited strong affinity and cell potency (data not shown), similar to data described in Example 4. These four candidates were also assessed for several manufacturability criteria. 1H5, 1D1, 1C10, and 21H7 demonstrated good stability in a freeze-thaw assay in optimized buffer formulation (data not shown).

As shown in Table 5, below, 1H5, 1D1, 1C10, and 21H7 retained strong affinity after repeated freeze-thaw.

TABLE 5
Co-binder Freeze-thaw Analysis

| Co-binder clone | Sample | ka (1/s*M) | kd (1/s) | KD (M) |
|---|---|---|---|---|
| 1H5 | Baseline | 1.01E+06 | 1.38E−04 | 1.36E−10 |
| | 3x F/T | 8.90E+05 | 5.51E−04 | 6.19E−10 |
| 1D1 | Baseline | 2.09E+05 | 4.05E−04 | 1.94E−09 |
| | 3x F/T | 1.95E+05 | 6.28E−04 | 3.22E−09 |
| 1C10 | Baseline | 2.20E+05 | 3.30E−04 | 1.50E−09 |
| | 3x F/T | 1.96E+05 | 6.75E−04 | 3.45E−09 |
| 21H7 | Baseline | 2.28E+05 | 4.23E−03 | 1.86E−08 |
| | 3x F/T | 2.24E+05 | 4.35E−03 | 1.94E−08 |

When expressed at large-scale, 1H5, 1D1, 1C10, and 21H7 retained good thermostability (Tm≥55° C.) as measured by DSF assay and showed good hydrophobicity properties (data not shown). Among the four co-binders, 1H5 showed stronger non-specific binding to BVP, insulin, and DNA versus the other three co-binders (data not shown) and was deprioritized. Overall, however, 1H5, 1D1, 1C10, and 21H7 demonstrated good overall developability.

1D1, 1C10, and 21H7 were concentrated to concentrations of approximately 200 mg/ml and higher in Buffer 1 (20 mM Histidine, 5% sucrose, 50 mM NaCl, pH 6.0) without evidence of aggregation or degradation (see Table 6).

TABLE 6
Co-binder Concentration and Recovery Percentage

| | 1D1Fc | 1C10Fc | 21H7Fc |
|---|---|---|---|
| mg/mL | 225 ± 21 | 185 ± 19 | 267 ± 9.5 |
| % Recovery | 105 | 92 | 92 |

### Example 7. Designs of Fc Format and Humanization of Co-Binders

Fc formats having a general structure of Hinge-CH2-CH3 for 1C10, 1D1, 1H5, 21H7 were designed using huIgG1Fc_YTE_C→S (SEQ ID NO: 62), which includes (1) the CH2 YTE mutations (M252Y, S254T, T256E as determined by EU Numbering) to reduce binding to IgG Fc receptors and diminished capacity for ADCC, and (2) a hinge cysteine (C) to serine(S) mutation (C220S as determined by EU Numbering).

The amino acid sequences for 1C10, 1D1, 1H5, and 21H7 co-binders with huIgG1Fc_YTE_C→S are provided in Table 1 as SEQ ID NOs: 15, 29, 43, and 57, respectively.

VHH sequences of 1H5, 1D1, 1C10, and 21H7 were subjected to humanization. Framework regions 1, 2, 3, and 4 were converted to human VH framework regions by searching human VH germline sequences. The selected human frames were grafted into the VHH sequences. In certain cases, a part of VHH frame 2 from Llama were solvent exposed as opposed to being in the interface between VH/VL in IgG format. Thus, back mutations were introduced. The three CDRs were left intact from the original sequences.

For example, during the 1C10 humanization process, frames 1, 2 and 3 were grafted with human germline IGHV3-74*02 frames, except that frame 2 was maintained from the original sequence (with 3 back mutations). VHH2 frames 1, 2 and 3 were grafted from IGHV3-74*01 where frame 2 was maintained from the original 3 amino acids. Both VHH1 and VHH2 FR4 were grafted with human JH1. As shown in Table 7, below, a humanized version was derived of 1D1, 1C10, and 21H7 that retained the properties of the wildtype co-binder.

TABLE 7
Properties of humanized co-binder candidates versus wild-type.

| | | | | BVP | BVP | % Loss | EC50 | | | Human- |
|---|---|---|---|---|---|---|---|---|---|---|
| Co- | | SEC_HPLC | Tm | Relative | Relative | at 40° C., | (pSTAT3) | | HIC | ization |
| binder | Mutation | Purity (%) | AVG | to blank | to WT | 2 d | nM | KD (M) | (min) | score |
| 1D1 | WT | 97.05 | 59.8 | 7.53 | 1.00 | Stable | 8.42 | 1.53E−11 | 16.4 | NA |
| 1D1 | M1M1M1M1 | 99.45 | 58.8 | 5.86 | 0.78 | Stable | 7.4 | 4.41E−11 | 16.7 | 0.965 |
| 1D1 | M1M2M1M1 | 99.26 | 59.0 | 7.23 | 0.96 | Stable | 9.57 | 5.97E−11 | 16.6 | 0.9675 |
| 1C10 | WT | 99.11 | 58.3 | 3.21 | 1.00 | Stable | 6.57 | 1.26E−11 | 16.9 | NA |
| 1C10 | M1M1M1M1 | 99.35 | 59.0 | 4.59 | 1.43 | Stable | 6.88 | 1.61E−11 | 17.3 | 0.9425 |
| 1C10 | M1MIM1M2 | 99.12 | 56.5 | 4.19 | 1.31 | Stable | 6.51 | 1.45E−11 | 17.1 | 0.9225 |
| 1C10 | M1M2M1M1 | 99.37 | 58.5 | 4.61 | 1.44 | Stable | 5.92 | 2.14E−11 | 17.1 | 0.9475 |
| 1C10 | M1M2M1M2 | 99.21 | 56.0 | 3.95 | 1.23 | Stable | 5.11 | 1.31E−11 | 17 | 0.9275 |
| 21H7 | WT | 93.22 | 59.8 | 4.41 | 1.00 | Stable | 6.65 | 1.04E−11 | 15.3 | NA |
| 21H7 | M1M1MIM1 | 98 | 57.8 | 3.13 | 0.71 | Stable | 13.7 | 7.95E−11 | 14.8 | 0.9375 |
| 21H7 | M1M1M1M2 | 96.52 | 60.0 | 3.17 | 0.72 | Stable | 6.39 | 1.28E−11 | 15 | 0.9525 |
| 21H7 | M1M2MIM1 | 95.43 | 59.3 | 3.53 | 0.80 | Stable | 7.79 | 1.13E−11 | 15 | 0.9425 |
| 21H7 | M1M2M1M2 | 95.01 | 59.5 | 4.06 | 0.92 | Stable | 6.7 | 1.14E−11 | 15 | 0.9575 |

Table 1 provides amino acid sequences for humanized 1C10 VHH1 single binder (SEQ ID NO: 4), humanized 1C10 VHH2 single binder (SEQ ID NO: 9), and humanized 1C10 co-binder (SEQ ID NO: 14); humanized 1D1 VHH1 single binder (SEQ ID NO: 18), humanized 1D1 VHH2 single binder (SEQ ID NO: 23), and humanized 1D1 co-binder (SEQ ID NO: 28); humanized 1H5 VHH1 single binder (SEQ ID NO: 32), humanized 1H5 VHH2 single binder (SEQ ID NO: 37), and humanized 1H5 co-binder (SEQ ID NO: 42); and humanized 21H7 VHH1 single binder (SEQ ID NO: 46), humanized 21H7 VHH2 single binder (SEQ ID NO: 51), and humanized 21H7 co-binder (SEQ ID NO: 56).

Fc formats of humanized 1C10, 1D1, 1H5, 21H7 were also designed using huIgG1Fc_YTE_C→S (SEQ ID NO: 62). The amino acid sequences for humanized 1C10, 1D1, 1H5, and 21H7 co-binders with huIgG1Fc_YTE_C→S are provided in Table 1 as SEQ ID NOs: 16, 30, 44, and 58, respectively.

### Example 8. In Vivo Efficacy in Pruritic Mouse Model

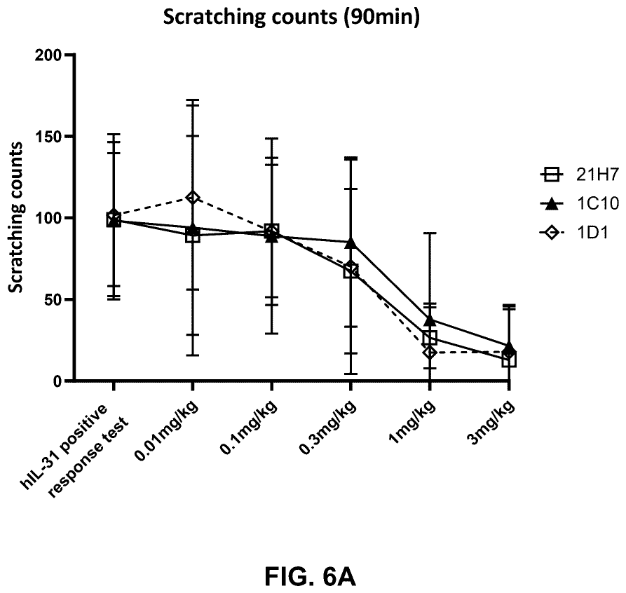

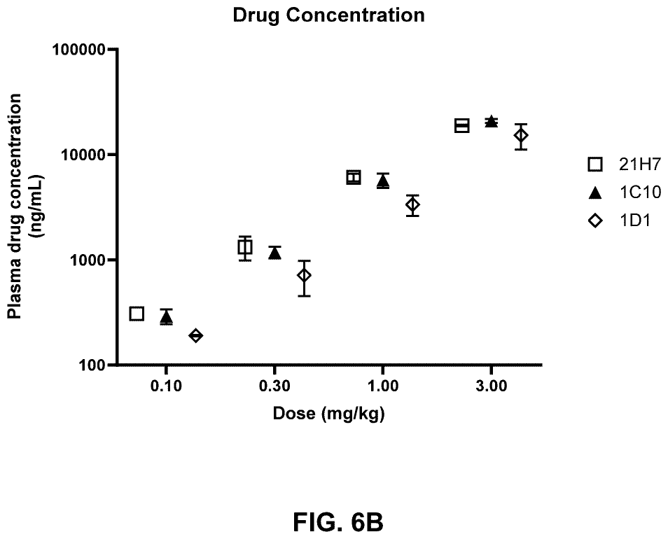

Female transgenic mice (6 to 8 weeks old) that have been engineered to express the human IL31, IL31RA, and oncostatin M receptor (OSMR) genes in place of the murine IL31, IL31RA, and OSMR genes were purchased from Biocytogen (Wakefield, MA) and were acclimated for one week before the start of the study. Prior to study initiation, all mice were administered recombinant hIL-31 (5 μg/mouse, Sino Biological, Beijing, China) via intravenous injection and observed for pruritic response for 1.5 hours. Mice that demonstrated a pruritic response to hIL31 injection were allocated randomly into 3 treatment groups. 21H7, 1C10, and 1D1 (SEQ ID NO: 58, 16, and 30, respectively) were administered on Study Day-1, Day 2, Day 6, Day 9, and Day 30 at 0.01, 0.1, 0.3, 1, and 3 mg/kg. Approximately 24 hours after test article administration, plasma was collected from all co-binder treated mice. Recombinant hIL31 was injected intravenously (5 μg/mouse) on Day 0, Day 3, Day 7, Day 10, and Day 31, and mouse scratching activity was recorded for 1.5 hours (FIG. 6A).

Scratching activity for all animals was normalized to scratching activity assessed prior to study initiation. Clear incidence of pruritic activity was observed in all treatment groups administered at 0.01 and 0.1 mg/kg. When test article was administered at 0.3 mg/kg a modest reduction in relative pruritic activity was observed for 2 of 3 test articles. When administered at 1 and 3 mg/kg, all test articles resulted in a marked reduction in IL31-induced pruritic activity. Importantly, as assessed by plasma drug concentration analysis, the amount of co-binder did increase systemically with increased dose level. When administered at the efficacious doses of 1 and 3 mg/kg, plasma co-binder concentrations were approximately 3-6 μg/mL and 15-19 μg/mL, respectively at the time of IL31 administration (FIG. 6B).

### Example 9. Anti-Pruritic Activity in Non-Human Primates

Male cynomolgus monkeys (3 to 5 years old) were selected from available stock animals at SNBL (Kagoshima, Japan). After two weeks of acclimation, all animals were administered recombinant cynomolgus IL-31 (1 μg/kg, Sino Biological, Beijing, China) via intravenous injection and observed for pruritic response for 3 hours. Animals that demonstrated a pruritic response to cyIL31 injection were allocated into 3 treatment groups of 3 monkeys each. 1C10 (SEQ ID NO: 16) was administered on Study Day 0 at 1 or 5 mg/kg. Approximately 24 hours after co-binder administration, serum was collected from all co-binder treated animals. Serum was also collected on Study Days 8, 15, 22, and 29. Recombinant cyIL31 was injected intravenously (1 μg/kg) on Study Days 1, 8, 15, and 29, and scratching activity was recorded to assess frequency and total duration over 3 hours.

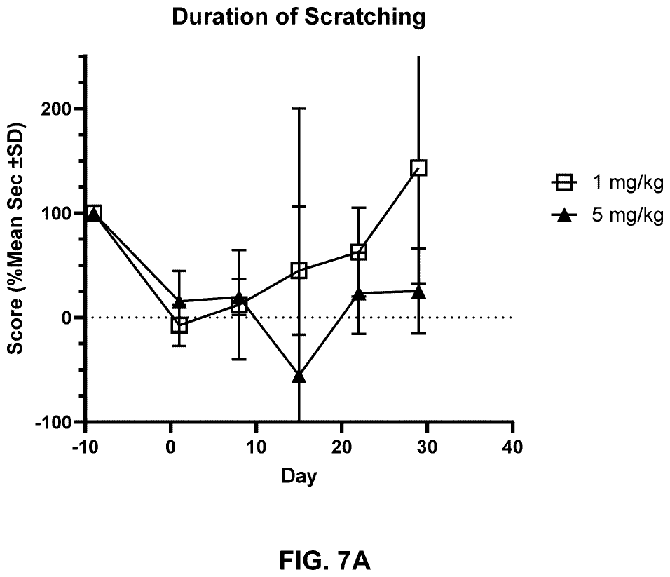

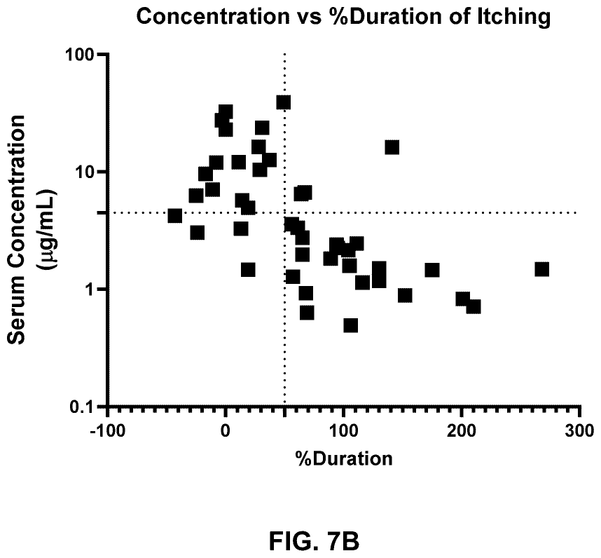

Pruritic activity for all animals was normalized to activity assessed upon cyIL31 treatment prior to study initiation. Clear incidence of pruritic activity was observed in all treatment groups prior to study initiation. As shown in FIG. 7A, when 1C10 co-binder was administered at 1 or 5 mg/kg a clear reduction in relative duration of pruritic activity was observed. The reduction in relative duration of pruritic activity persisted to Day 8. After Day 8, animals in the 1 mg/kg treatment group began demonstrating increased pruritic activity over time, increasing to pre-study levels by Day 29. In contrast, animals treated with 5 mg/kg of test article failed to show a clear resumption of cyIL31-induced pruritic activity throughout the course of the study.

Serum collected from all animals was assessed for 1C10 co-binder concentration by ELISA. All animals possessed quantifiable levels of 1C10 co-binder at all time points tested with a clear reduction in serum drug concentration over time. These data suggest time and drug concentration effects of 1C10 co-binder on suppressing IL31-induced pruritus. This is further supported when comparing the relative duration of pruritic activity to calculated serum drug concentration (FIG. 7B). Specifically, animals with the highest serum drug concentration had the lowest relative pruritic activity upon cyIL31 treatment, in contrast with the notably higher pruritic activity in animals demonstrating the lowest serum drug concentrations.

### EQUIVALENTS

The foregoing written specification is considered to be sufficient to enable one skilled in the art to practice the aspects. The foregoing description and Examples detail certain aspects and describe the best mode contemplated by the inventors. It will be appreciated, however, that no matter how detailed the foregoing may appear in text, the aspect may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all the values or ranges provided in the list. In some instances, the term about may include numerical values that are rounded to the nearest significant figure.

1-127. (canceled)

128. A co-binder polypeptide that binds human IL31 comprising a first IL31 binder and a second IL31 binder, wherein the first IL31 binder comprises a first variable heavy domain of heavy chain (VHH1) and the second IL31 binder comprises a second VHH (VHH2), wherein:
a) the VHH1 comprises a complimentary determining region (CDR) 1 comprising the amino acid sequence of SEQ ID NO:47, a CDR2 comprising the amino acid sequence of SEQ ID NO: 48, and a CDR3 comprising the amino acid sequence of SEQ ID NO:49; and wherein the VHH2 comprises a CDR1 comprising the amino acid sequence of SEQ ID NO:52, a CDR2 comprising the amino acid sequence of SEQ ID NO:53, and a CDR3 comprising the amino acid sequence of SEQ ID NO:54;
b) the VHH1 comprises a CDR1 comprising the amino acid sequence of SEQ ID NO: 59, a CDR2 comprising the amino acid sequence of SEQ ID NO:48, and a CDR3 comprising the amino acid sequence of SEQ ID NO:49; and (ii) the VHH2 comprises a CDR1 comprising the amino acid sequence of SEQ ID NO:52, a CDR2 comprising the amino acid sequence of SEQ ID NO: 53, and a CDR3 comprising the amino acid sequence of SEQ ID NO:54;
c) the VHH1 comprises a CDR1 comprising the amino acid sequence of SEQ ID NO: 47, a CDR2 comprising the amino acid sequence of SEQ ID NO:60, and a CDR3 comprising the amino acid sequence of SEQ ID NO:49; and (ii) the VHH2 comprises a CDR1 comprising the amino acid sequence of SEQ ID NO:52, a CDR2 comprising the amino acid sequence of SEQ ID NO: 53, and a CDR3 comprising the amino acid sequence of SEQ ID NO:54;
d) the VHH1 comprises a CDR1 comprising the amino acid sequence of SEQ ID NO: 47, a CDR2 comprising the amino acid sequence of SEQ ID NO:63, and a CDR3 comprising the amino acid sequence of SEQ ID NO:49; and (ii) the VHH2 comprises a CDR1 comprising the amino acid sequence of SEQ ID NO:52, a CDR2 comprising the amino acid sequence of SEQ ID NO: 53, and a CDR3 comprising the amino acid sequence of SEQ ID NO:54;
e) the VHH1 comprises a CDR1 comprising the amino acid sequence of SEQ ID NO: 47, a CDR2 comprising the amino acid sequence of SEQ ID NO:48, and a CDR3 comprising the amino acid sequence of SEQ ID NO:61; and (ii) the VHH2 comprises a CDR1 comprising the amino acid sequence of SEQ ID NO:52, a CDR2 comprising the amino acid sequence of SEQ ID NO: 53, and a CDR3 comprising the amino acid sequence of SEQ ID NO:54; or
f) the VHH1 comprises a CDR1 comprising the amino acid sequence of SEQ ID NO: 47, a CDR2 comprising the amino acid sequence of SEQ ID NO:48, and a CDR3 comprising the amino acid sequence of SEQ ID NO:64; and (ii) the VHH2 comprises a CDR1 comprising the amino acid sequence of SEQ ID NO:52, a CDR2 comprising the amino acid sequence of SEQ ID NO: 53, and a CDR3 comprising the amino acid sequence of SEQ ID NO:54.

129. The co-binder polypeptide of claim 128, wherein the VHH1 comprises the amino acid sequence of SEQ ID NO:45 or SEQ ID NO:46.

130. The co-binder polypeptide of claim 128, wherein the VHH2 comprises the amino acid sequence of SEQ ID NO:50 or SEQ ID NO:51.

131. The co-binder polypeptide of claim 128, wherein the VHH1 comprises the amino acid sequence of SEQ ID NO:46, and the VHH2 comprises the amino acid sequence of SEQ ID NO: 51.

132. The co-binder polypeptide of claim 128, wherein the polypeptide comprises the amino acid sequence of SEQ ID NO:55 or SEQ ID NO:56.

133. The co-binder polypeptide of claim 128, wherein the VHH1 is connected to the VHH2 by a linker, and wherein the co-binder polypeptide has the structure: [VHH1]-linker-[VHH2].

134. The co-binder polypeptide of claim 133, wherein the linker comprises an amino acid linker.

135. The co-binder polypeptide of claim 134, which further comprises a human IgG1, IgG2, IgG3, or IgG4 Fc region.

136. The co-binder polypeptide of claim 135, wherein the Fc region comprises one or more substitutions, deletions, insertions, or any combination thereof.

137. The co-binder polypeptide of claim 136, wherein the one or more substitutions comprise C220S, M252Y, S254T, T256E, L234A, L235A, M428L, N434S or any combination thereof, according to the EU numbering system.

138. The co-binder polypeptide of claim 135, wherein the Fc region comprises the amino acid sequence of SEQ ID NO:62, amino acid residues 1-231 of SEQ ID NO:62, the amino acid sequence of SEQ ID NO:90, or amino acid residues 1-231 of SEQ ID NO:90.

139. The co-binder polypeptide of claim 135, which comprises the amino acid sequence of SEQ ID NO:58 or amino acid residues 1-507 of SEQ ID NO:58.

140. The co-binder polypeptide of claim 128, which binds to human IL31 at a kD of less than or equal to 1×10⁻⁹ M, as measured by surface plasmon resonance, or which binds to human IL31 with a k_{off} rate of less than or equal to 5×10⁻⁴ M per second, as measured by surface plasmon resonance.

141. The co-binder polypeptide of claim 128, which comprises a bispecific, trispecific, tetraspecific, or multispecific polypeptide.

142. A pharmaceutical composition comprising the co-binder polypeptide of claim 128, and a pharmaceutically acceptable carrier.

143. An isolated nucleic acid encoding the co-binder polypeptide of claim 128.

144. A method of producing an IL31 co-binder polypeptide, comprising transfecting the isolated nucleic acid of claim 143 into a host cell under conditions suitable for expression of the polypeptide.

145. A method for treating a subject having an IL31-associated condition comprising administering to the subject a therapeutically effective amount of the co-binder polypeptide of claim 128.

146. The method of claim 145, wherein the IL31-associated condition is a pruritic skin condition.

147. The method of claim 146, wherein the pruritic skin condition is atopic dermatitis.

148. The method of claim 145, wherein the IL31-associated condition is acne rosacea, acne vulgaris, allergic asthma, allergic contact dermatitis, allergic rhinitis, alopecia areata, arthritis, atopic dermatitis, bile acid induced urticaria, bullous pemphigoid, check-point inhibitor induced pruritus, cholestatic pruritus, chronic hand eczema, chronic inducible urticaria, chronic kidney disease-associated pruritus, chronic pruritus of unknown origin, chronic spontaneous urticaria, chronic urticaria, contact dermatitis, contact hypersensitivity, Crohn's disease, cutaneous (lichen) amyloidosis, cutaneous T-cell lymphoma, dermatomyositis, drug-induced allergic reactions, eczema, epidermolysis bullosa, folliculitis, inflammatory bowel disease, intrahepatic cholestasis of pregnancy, itch associated with wound healing, lichen planus, metabolic dysfunction-associated (non-alcoholic) steatohepatitis (MASH), neurodermatitis, osteoarthritis, osteoporosis, pemphigus, pemphigus herpetiformis, porokeratosis, primary biliary cholangitis, primary sclerosing cholangitis, prurigo nodularis, pruritus, pruritus associated with cutaneous T-cell lymphoma, psoriasis, psoriatic arthritis, rheumatoid arthritis, scleroderma, skin-tropic viruses and viral associated pruritus, spondyloarthritis, stasis dermatitis, systemic lupus erythematosus, systemic sclerosis, toxic epidermal necrolysis, ulcerative colitis, uremic pruritus, or wound healing pruritus.

149. The co-binder of claim 131, which further comprises a human Fc region comprising the amino acid sequence of SEQ ID NO:62.
